(19) United States
(12) Patent Application Publication
(10) Pub. No.: US 2005/0123910 A1
(43) Pub. Date: Jun. 9, 2005

(54) Enzyme and snp marker for disease

(76) Inventors: William Osmond Charles Michael Cookson, Oxford (GB); Miriam Fleur Moffat, Bicester (GB); Maxine Allen, Oxfordshire (GB); Nick Lench, Cardiff (GB)

Correspondence Address: FISH & RICHARDSON P.C., 3300 DAIN RAUSCHER PLAZA, 60 SOUTH SIXTH STREET, MINNEAPOLIS, MN, 55402, US

(21) Appl. No.: 10/476,264

(22) PCT Filed: Apr. 24, 2002
(86) PCT No.: PCT/GB02/01887

(30) Foreign Application Priority Data
Apr. 24, 2001 (GB) 0110044.5
Apr. 24, 2001 (GB) 0110046.0
Oct. 12, 2001 (GB) 0124594.3
Oct. 12, 2001 (GB) 0124575.2

(51) Int. Cl. C12Q 1/68

(52) U.S. Cl. 435/6

(57) ABSTRACT

The present invention relates to an isolated nucleic acid sequence which encodes a novel protein, where the protein is a new member in a family of enzymes with peptidase activity. Also provided is the use of the nucleic acid sequence and/or protein in medicine and research, a method for diagnosing, or determining predisposition to disease, methods for preventing or treating disease, and kits for use in the methods and the use of the nucleic acid sequence, protein and inhibitors thereof in treating or preventing inflammatory diseases, and in screens for identifying new inhibitors. Also provided are nucleic acid expression vectors, host cells, screens and non-human transgenic animals.

The present invention relates to an isolated nucleic acid sequence which encodes a novel protein, where the protein is a new member in a family of enzymes with peptidase activity. Also provided is the use of the nucleic acid sequence and/or protein in medicine and research, a method for diagnosing, or determining predisposition to disease, methods for preventing or treating disease, and kits for use in the methods and the use of the nucleic acid sequence, protein and inhibitors thereof in treating or preventing inflammatory diseases, and in screens for identifying new inhibitors. Also provided are nucleic acid expression vectors, host cells, screens and non-human transgenic animals.

The diseases of asthma, eczema and hay fever are typified by Immunoglobulin E mediated reactions to common allergens. These diseases are known as “atopic”. They are increasing in prevalence, and are now a major source of disability throughout the developed world. They are the result of complex interactions between genetic and environment mechanisms. The atopic state is characterised by prolonged exuberant Immunoglobulin E (IgE) responses to common inhaled proteins, known as allergens. Atopy is accompanied by elevation of the total serum IgE concentration, and by the presence of IgE specific to allergens. This specific IgE may be measured directly in the serum, by ELISA or other techniques. It may also be detected by prick skin tests, in which minute traces of allergen are introduced through a prick in the skin surface: atopic individuals respond to the test with a visible wheal on the skin surface.

Asthma is a major cause of disease in children and young adults and is becoming more prevalent and is the most common disease of childhood and arises due to the interaction between strong environmental and genetic factors. Asthma may be identified by intermittent mucosal inflammation, wheezing, and shortness of breath.

Asthma is usually recognised epidemiologically by standard symptom questionnaires or by physician diagnosis. Physician-diagnosed asthma ascertained by questionnaire has a heritability of 60-70%. Linkage to asthma and its associated phenotypes has been demonstrated near the IL1 cluster on chromosome 2. Genome-wide scans for linkage to atopy and asthma-associated phenotypes have identified the marker D2S160 on chromosome 2q14 near the IL-1 complex which has been associated with “wheeze” (p-0.001). As the region contains IL-1 and its homologues, it was investigated for association between asthma and known SNPs in IL1 cluster genes. Contrary to expectations there was no association found to these known polymorphisms in a combined panel of 246 families containing 1122 individuals and 381 asthmatics. However a replicated association between asthma and alleles of a D2S308 microsatellite, approximately 1 Mb distant from the IL1 genes, has been found.

The genetic factors which predispose an individual to asthma are thought to be variants of DNA structure (“polymorphisms”) that alter the level of expression or the function of genes. Variants of DNA sequence at a particular site (“locus”) are known as “alleles”.

Disease causing alleles will be in linkage disequilibrium with non-functional polymorphisms from the same chromosomal segment. It is therefore possible to detect allelic association with disease from particular chromosomal segments, without identifying the exact polymorphism and gene underlying the disease state.

The detection of allelic association may therefore give information as to disease susceptibility in a particular individual. Furthermore, allelic association is indicative of exons of a disease-causing gene are usually present within a limited distance (50-500 Kb) of DNA in either direction from the allele.

Identification of genetic polymorphisms in linkage disequilibrium with asthma. or atopy will allow the identification of children at risk of such diseases before the disease has developed (for example immediately after birth), with the potential for prevention of disease. The presence of particular polymorphisms or combinations of polymorphisms may predict the clinical course of disease (e.g. severe as opposed to mild) or the response to particular treatments. This diagnostic information will be of use to the health care, pharmaceutical and insurance industries.

Oligopeptidases are endopeptidases that act only on smaller polypeptides or oligopeptides. These enzymes perform important, specialized biological functions that include the modification or destruction of peptide messenger molecules. Oligopeptidases have few naturally occurring inhibitors, and their distinctive specificity prevents them from interacting with α2-macroglobulin, unlike the great majority of endopeptidases. Members of the prolyl-oligopeptidase family S9 (enzyme nomenclature committee EC 3.4.14.5), subfamily S9B include DPP4, DPP6, DPP8 and DPP9. The S9 family contains serine proteases with a varied range of relatively restricted substrate specificities. S9 peptidases are either soluble, cytosolic proteins or integral type II membrane-bound proteins and do not appear to exist as pro-enzymes, and are synthesised in an active form. The active site triad, Ser, Asp, His has been identified in DPP4, DPP6, DPP8, and DPP9, although the putative catalytic serine. residue is substituted with aspartate in DPP6 and glycine in Drosophila Melanogaster CG9059. In all known members of the family, these residues are within 130 residues of the carboxyl-terminus. All members of the prolyl-oligopeptidase family contain a conserved 7 amino-acid motif DW(V/I/L)YEEE in the predicted β-propeller domain. Two of the glutamate residues within this conserved motif have been shown to be essential for serine protease enzyme activity. The membrane-bound members of the S9 family contain membrane-spanning domains near the amino-terminus. Prolyl-oligopeptidases are responsible for cleaving the C-terminal peptide bond adjacent to a di-peptide sequence Ala-Pro or Gly-Pro. Substrates for these enzymes include chemokines, growth factors, and neuro- and vaso-active peptides.

Certain cell surface peptidases have been identified as specific antigens of immune cells. These include DPP4 (CD26), neutral endopeptidase (CD10), and aminopeptidase N (CD13). These cell surface antigens play an important role in the differentiation of immune cells. It has also been postulated that prolyl-olgopeptidases may play a role in the alteration of the Thl/Th2 (T-helper cell) balance during the inflammatory process. DPP4 (CD26) has been shown to have multifunctional properties in addition to its peptidase activity.

Based on it's proposed structure DPP4 (CD26) is predicted to be a multifunctional protein and has been shown to interact with several proteins outside of it's catalytic domain. DPP4 (CD26) is an adhesion receptor for both collagen and fibronectin and has been shown to mediate the lung colanisation of breast cancer cells in a rat model, an effect mediated primarily through fibronectin binding. DPP4 (CD26) can also bind adenosine deaminase, an enzyme that metabolises adenosine to inosine. The location of the DPP4/ADA complex at the cell surface is an important mechanism which regulates the effective concentration of adenosine in the vicinity of it's receptor. Adenosine suppresses T-cell activation through interaction with the A2a G-protein coupled receptor. Adenosine therefore plays an important role in suppression caused by ADA deficiency.

DPP4 (CD26) also plays an important role in T-cell signaling through interaction with CD45, protein tyrosine phosphatase. While the counter-receptor for DPP4 (CD26) has not yet been identified, cross-linking of DPP4 (CD26) with antibody induces tyrosine phosphorylation of a number of molecules known to be important in T-cell activation including p56Ick, P59fyn and ZAP-70.

Of the S9B prolyl oligopeptidases, DPP6 homologues have been cloned from Bos taurus, Rattus norvegicus and Mus musculus. DPP6 has been reported to exist as 2 different isoforms (DPPX-S and DPP-L) as a result of alternative mRNA splicing and possible differential (tissue-specific) promoter usage. DPPX-S and DPPX-L demonstrate differential tissue expression. Given that the two forms have different cytoplasmic domains, this may form the basis for different transmembrane signalling systems. DPP6 has no detectable catalytic activity, most likely due to the substitution of serine for aspartate in the catalytic triad. In vitro mutagenesis of aspartate to serine failed to restore catalytic activity.

To date, various markers of inflammatory diseases such as asthma and atopy have been identified, and used to identify those people at risk of such disease. Notably, however, there has been no success in linking such markers with a gene whose activity may be at the root of inflammatory disease. The identification of this gene will enable the provision of valuable diagnostic, therapeutic and research tools.

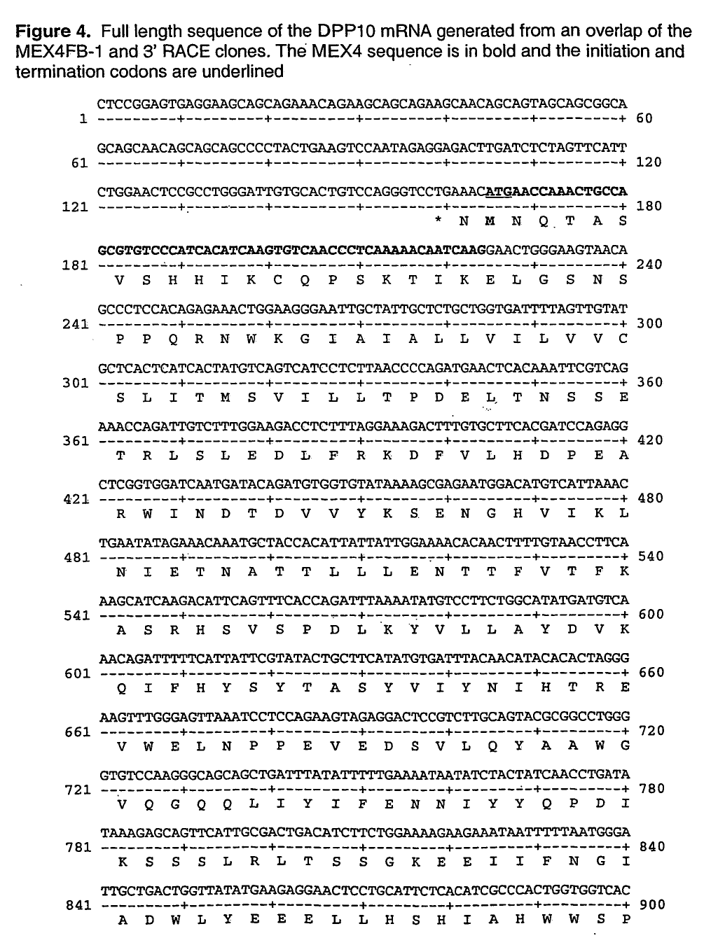

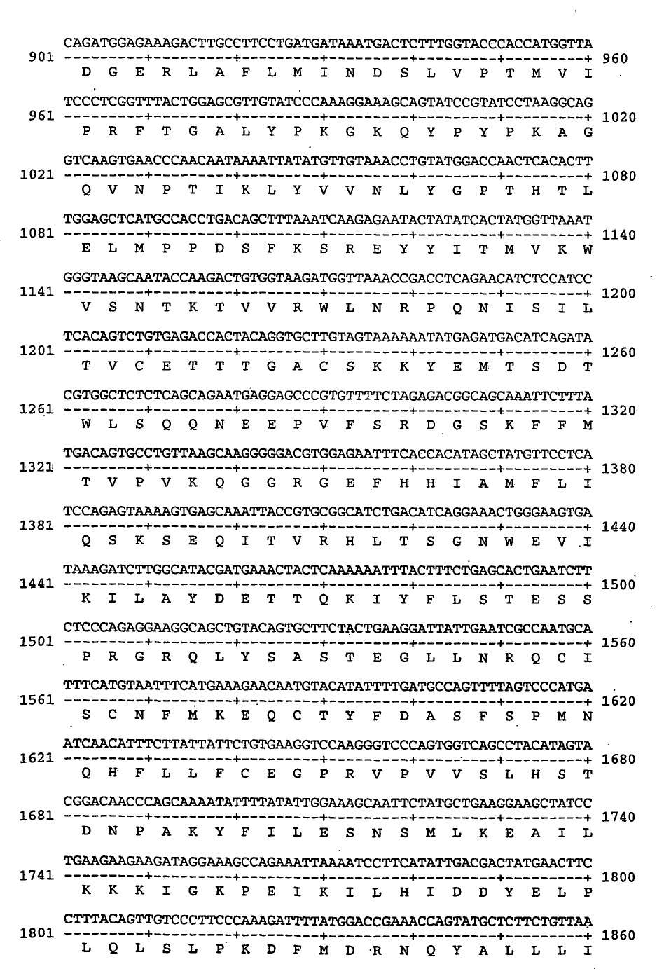

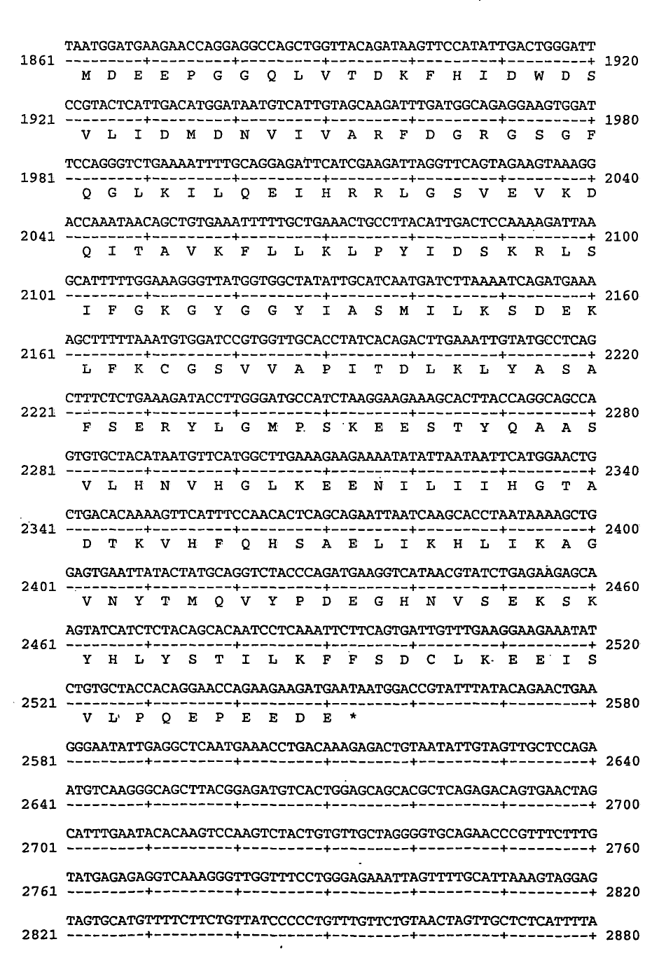

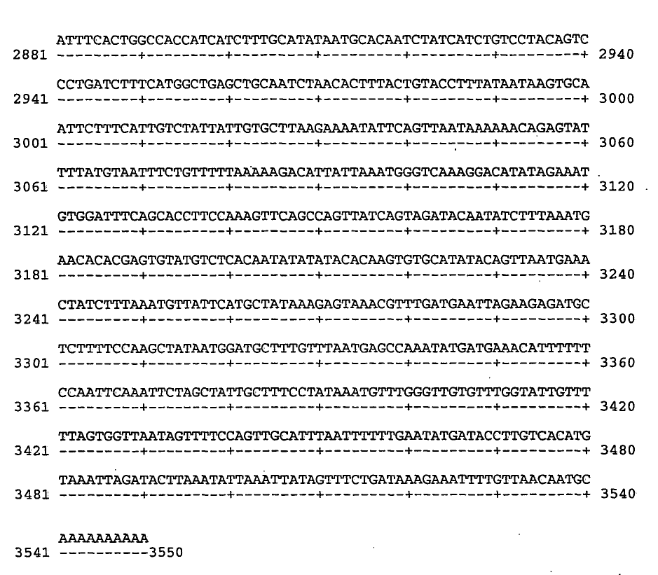

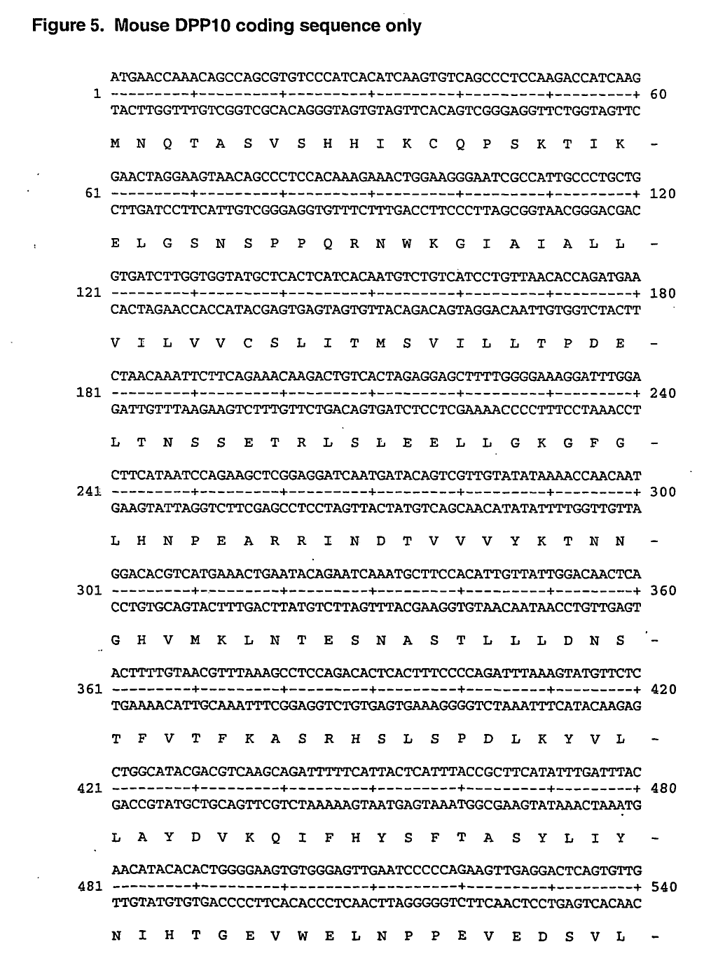

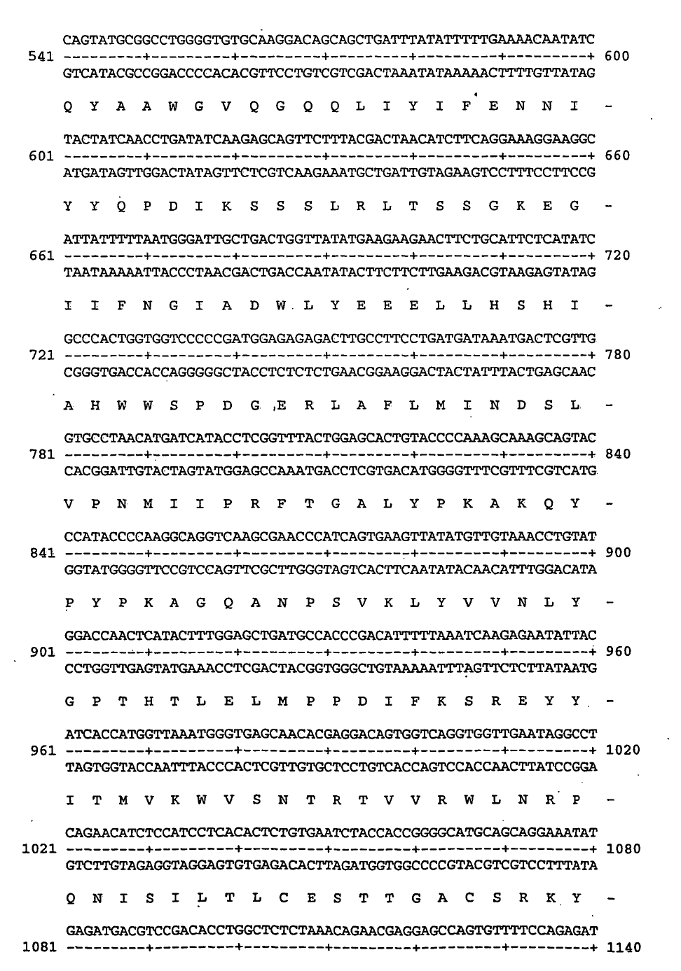

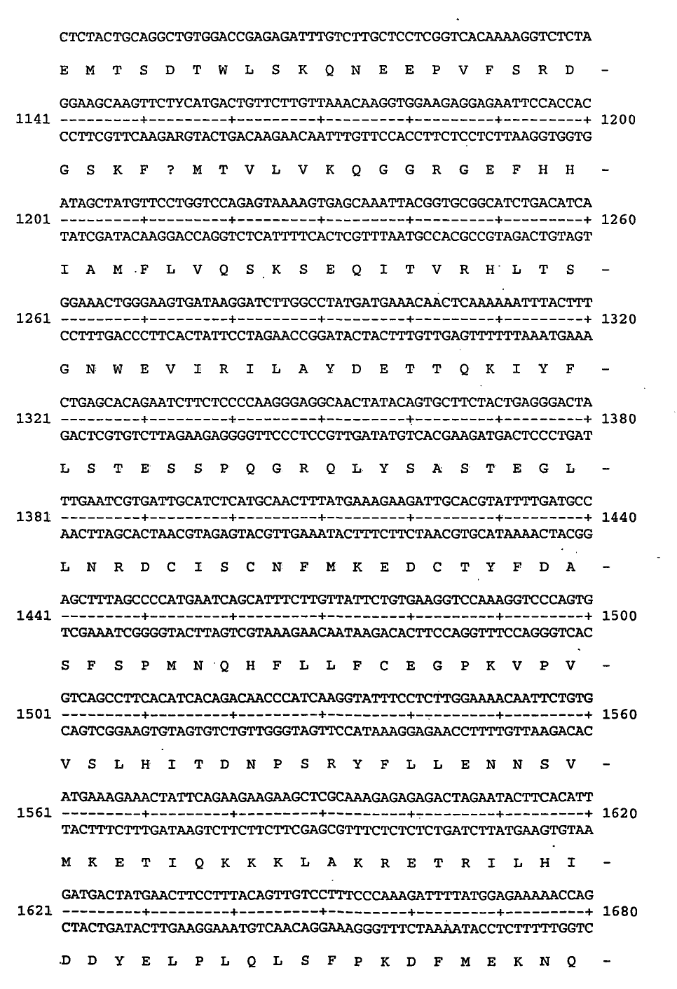

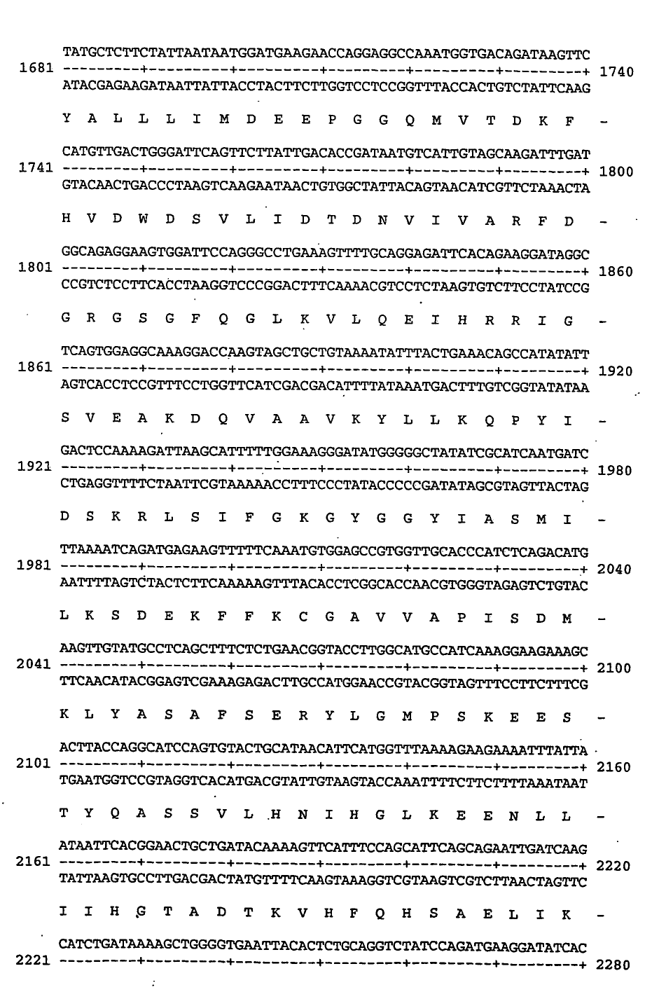

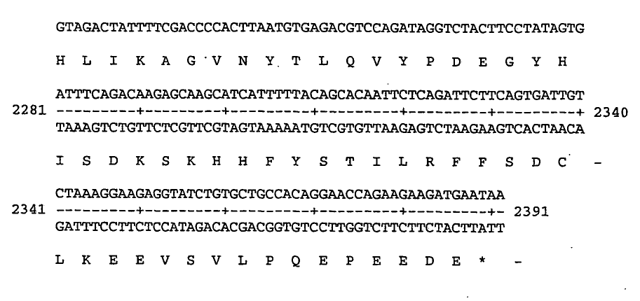

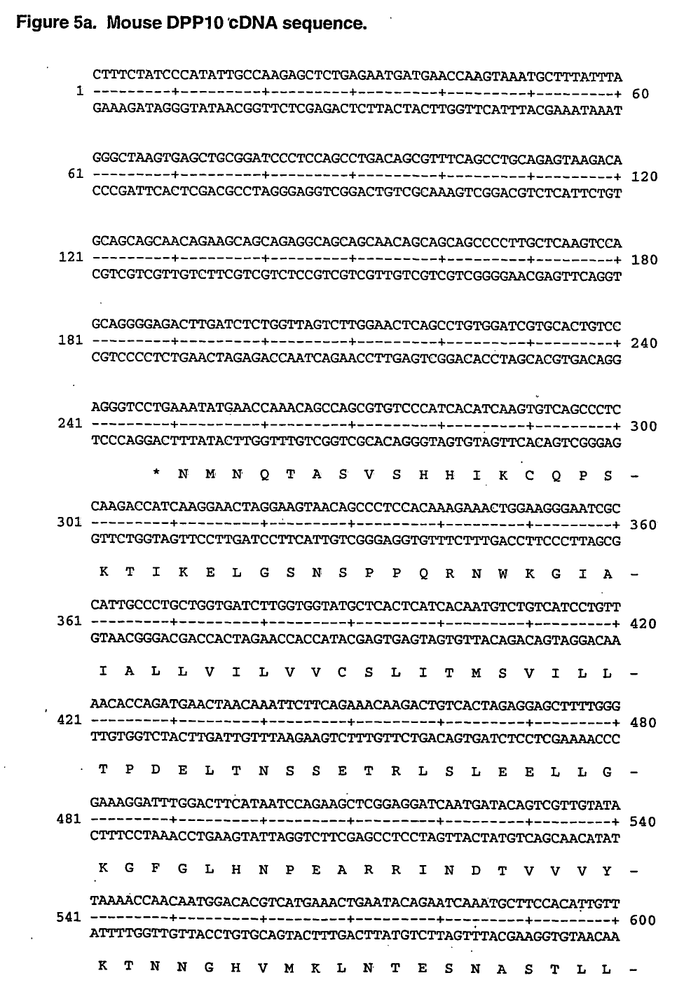

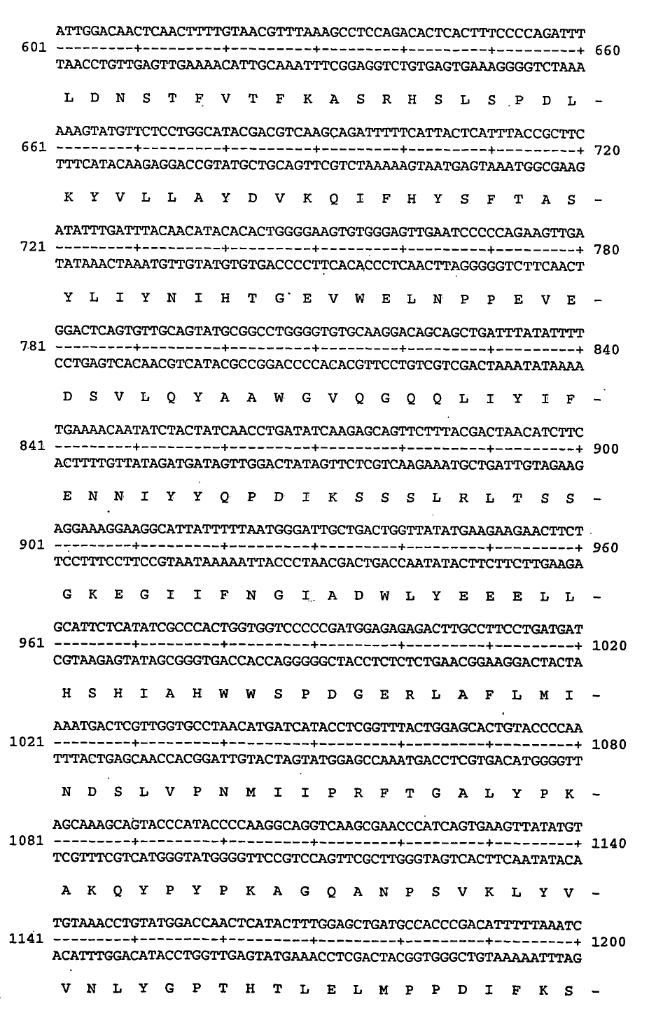

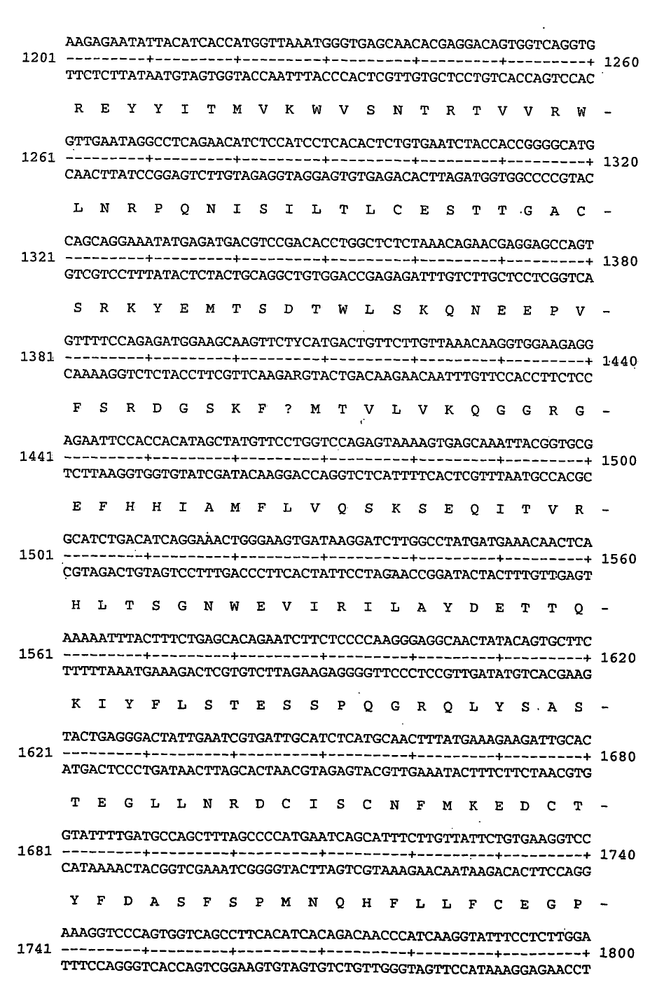

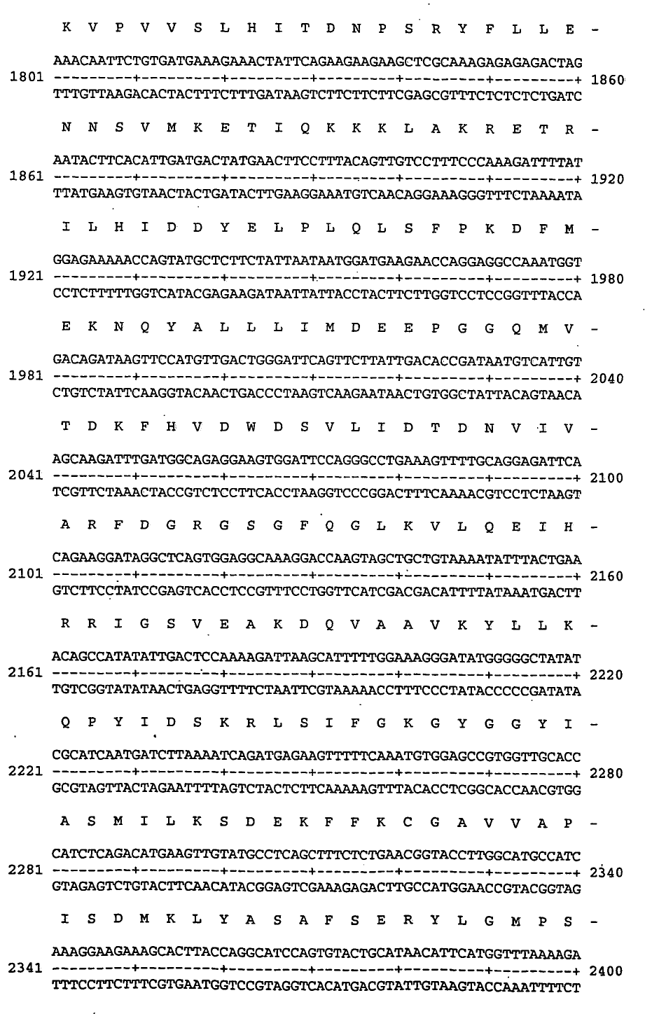

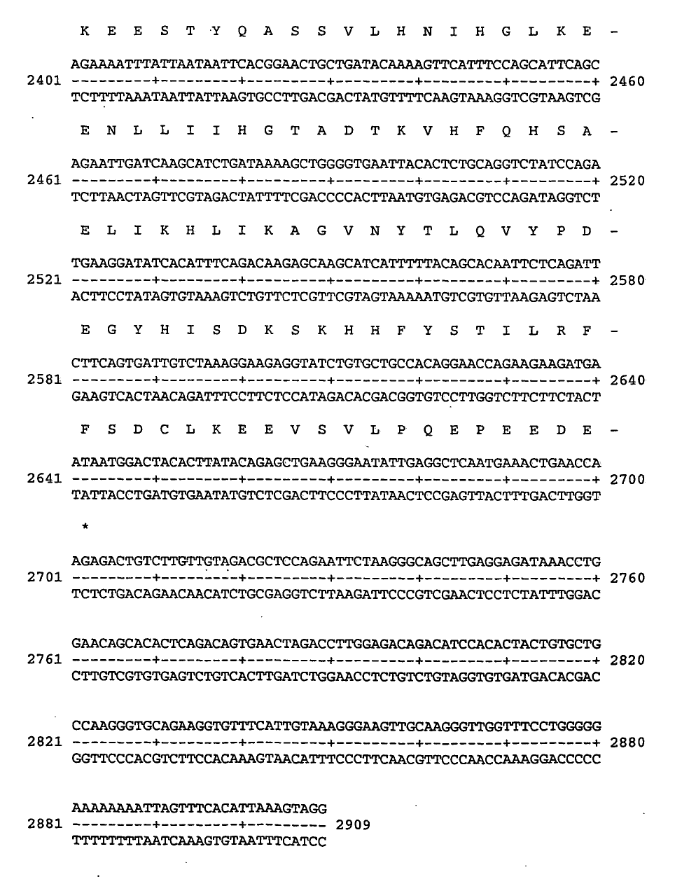

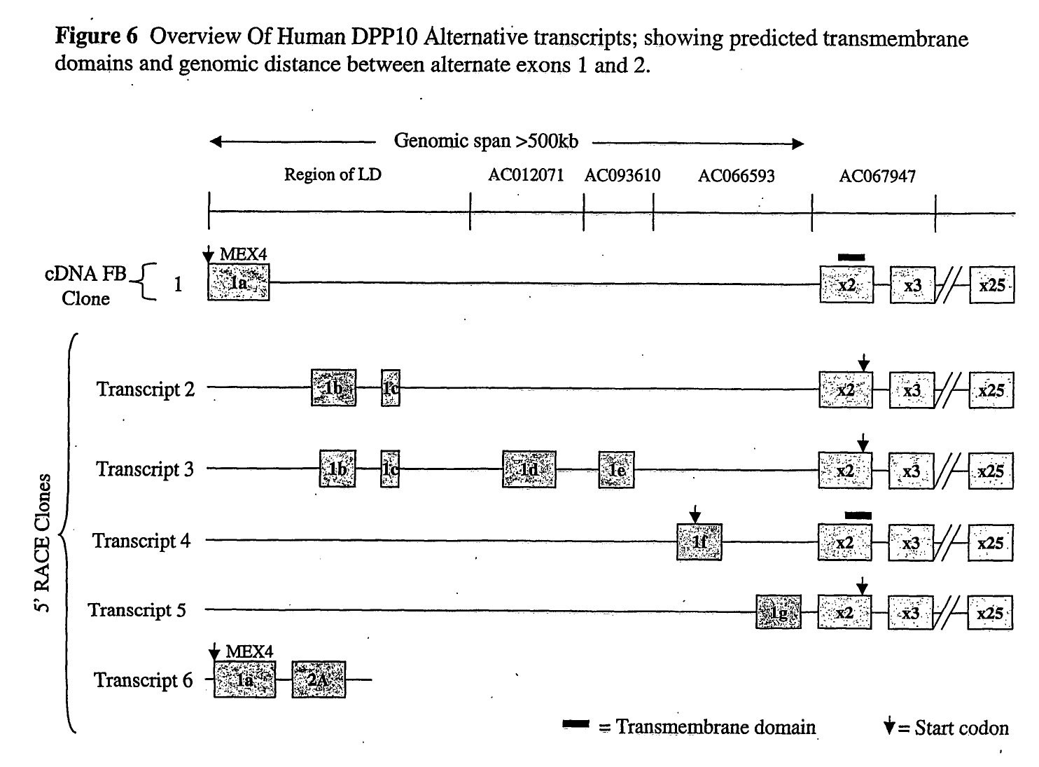

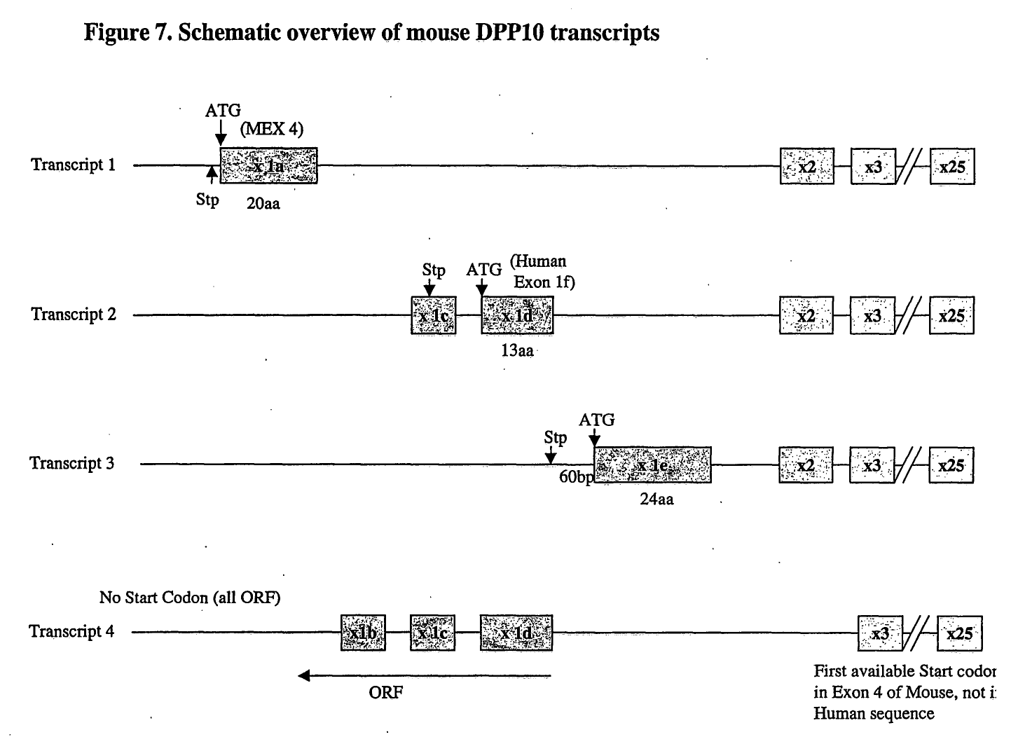

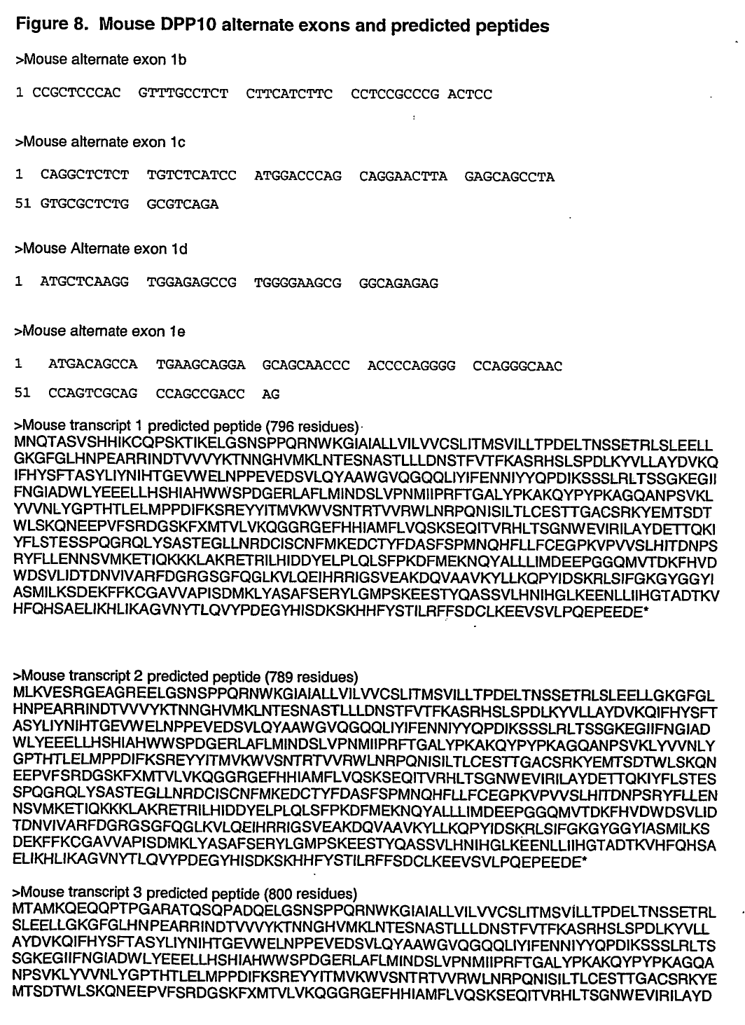

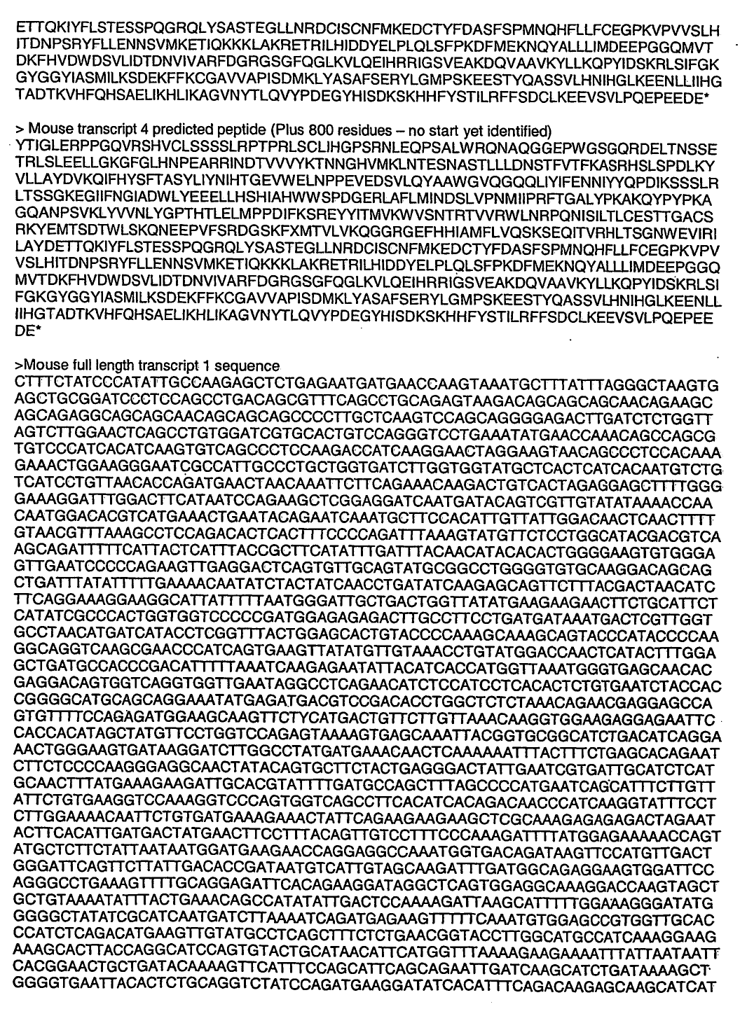

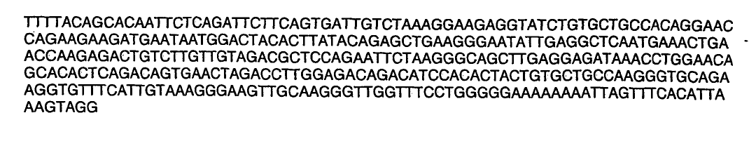

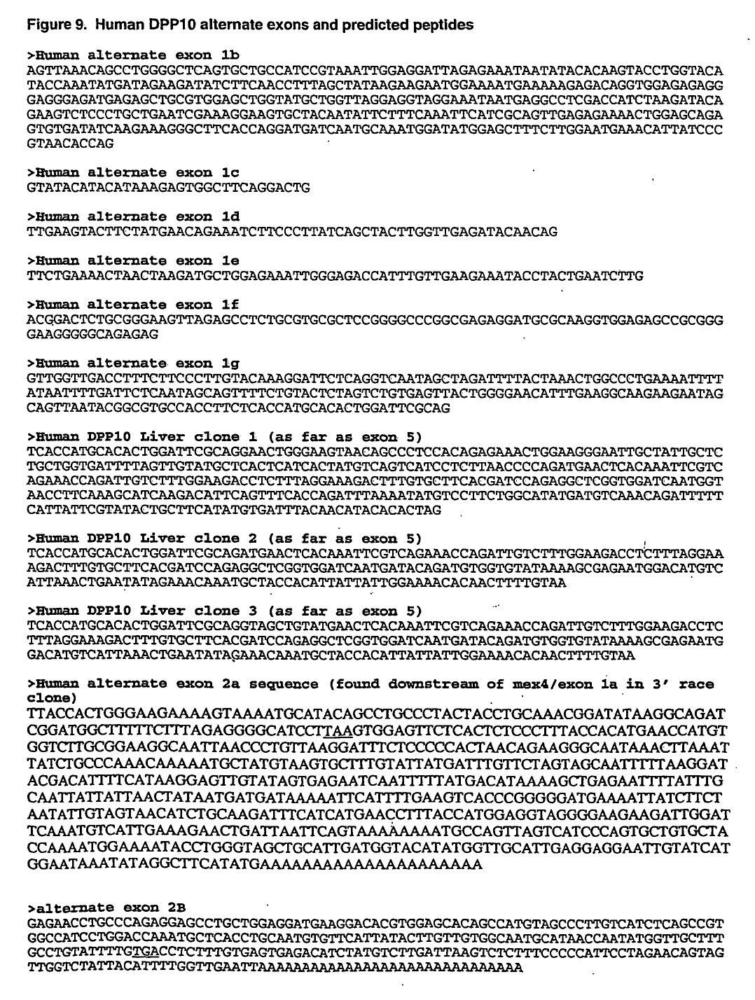

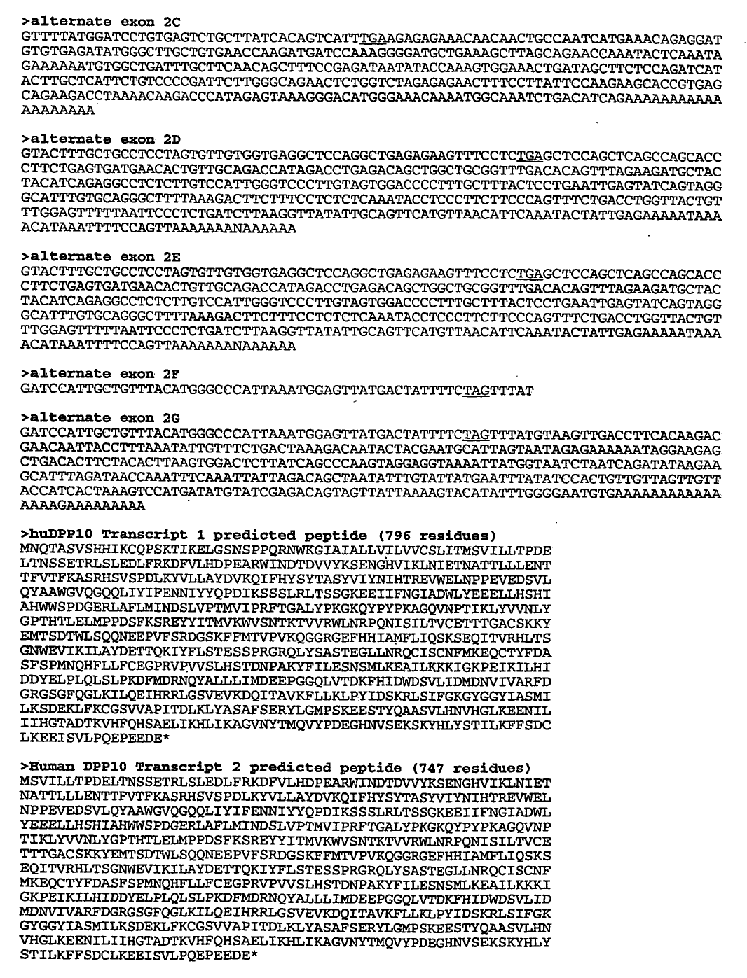

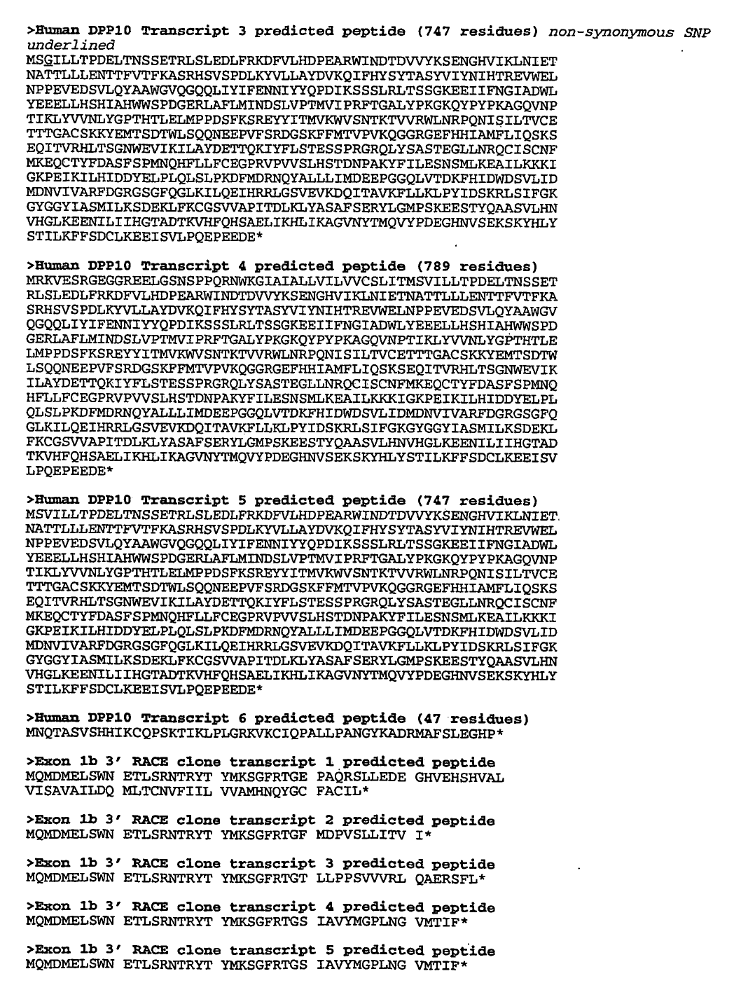

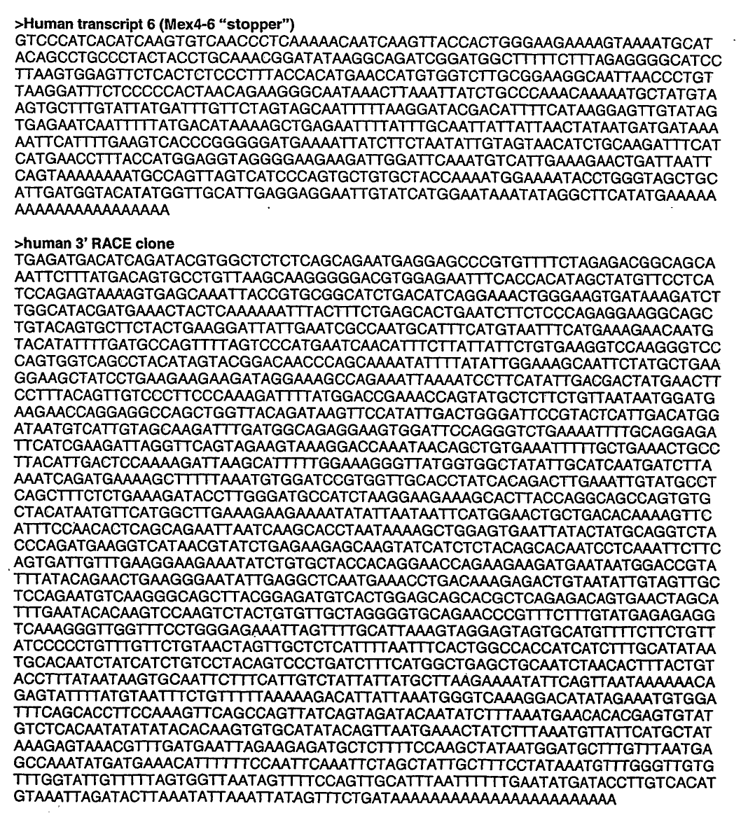

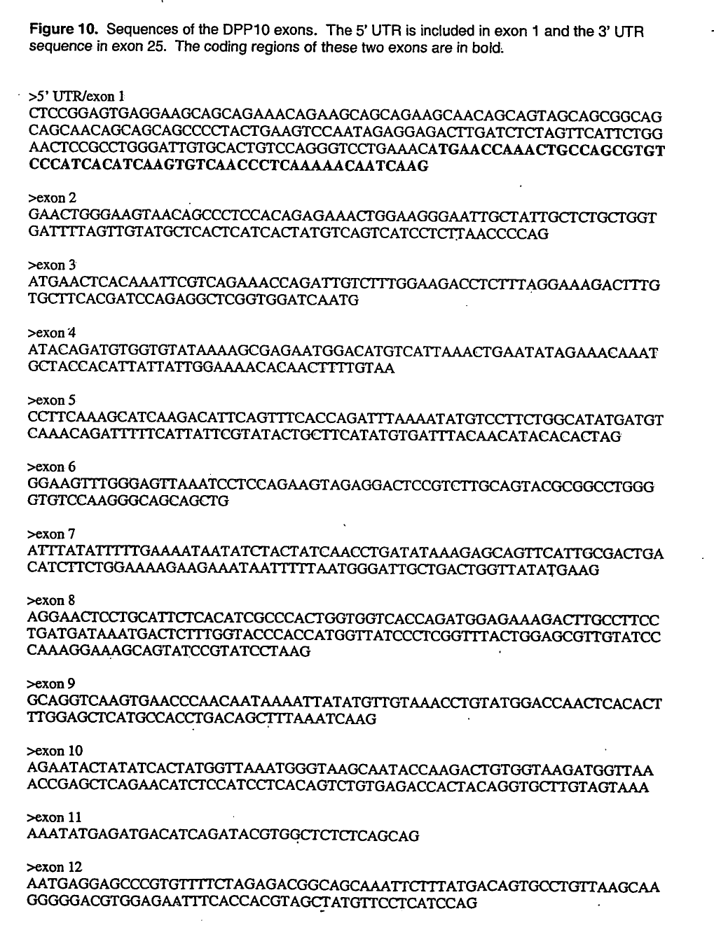

According to a first aspect of the invention there is provided an isolated nucleic acid sequence comprising a sequence as shown in FIG. 4, or a sequence as shown in FIG. 4 which excludes one or more of the exon sequences as set out in FIG. 10 and/or FIG. 4 when one or more of the exon sequences are replaced with one or more alternate exon sequence or one or more liver clone sequence from FIG. 9 or a sequence complementary or substantially homologous thereto, or a fragment thereof. The sequence of FIG. 4 is the human DPP10 mRNA sequence. FIG. 4 is compiled of the exons as shown in FIG. 10. Alternate exons are shown in FIG. 9. Alternative exons are referred to by different letters, for example exons 1a, 1b etc are alternate sequences for exon 1 (likewise for exons 2A, 2B etc). Upper or lower case designation of exons does not have any significance. Alternate transcripts for FIG. 4 can be seen schematically in FIG. 6. In addition, there is provided an isolated nucleic acid sequence comprising the sequence shown in FIG. 5a and/or a sequence as shown in FIG. 5a which excludes one or more exons or a sequence as set out in FIG. 5a when one or more of the exons are replaced with an alternate exon from FIG. 8. FIG. 7 shows a schematic overview of mouse transcripts, or a sequence complementary or substantially homologous thereto, or a fragment thereof. The sequence of FIG. 5a is the mouse DPP10 cDNA sequence. The mouse DPP10 mRNA sequence is shown in FIG. 5. The DPP10 nucleic acid sequence can comprise any combination of one or more exons from 1a, 1b, 1c, 1d, 1e, 1f, 1g, 2a, 2b, 2c, 2d, 2e, 2f, 2g, 3, 4, 5, 6, 7, 8, 9, 10, 11, 12, 13, 14, 15, 16, 17, 18, 19, 20, 21, 22, 23, 24, 25 or the sequences of liver clones 1, 2 or 3 or a sequence substantially homologous thereto, or a fragement thereof. These combinations are for all DPP10 sequences including human and mouse—where the exons of the mouse do not include if or lg.

The following isolated nucleic acid sequences are part of the first aspect.

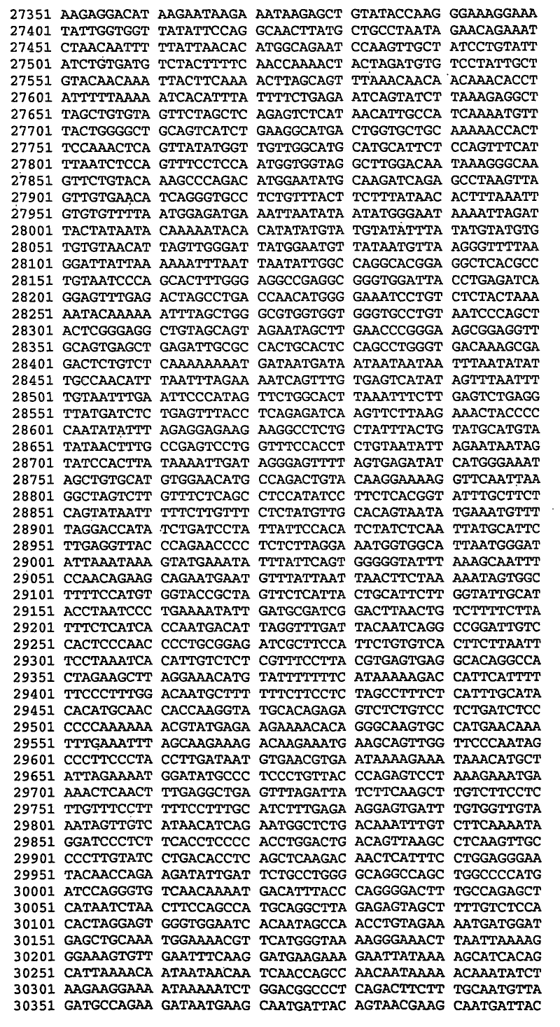

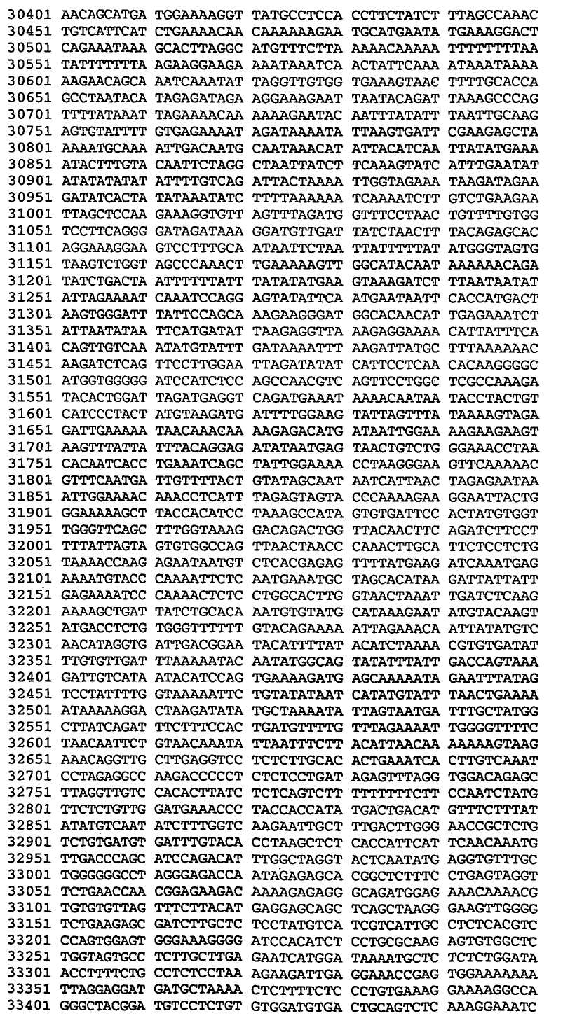

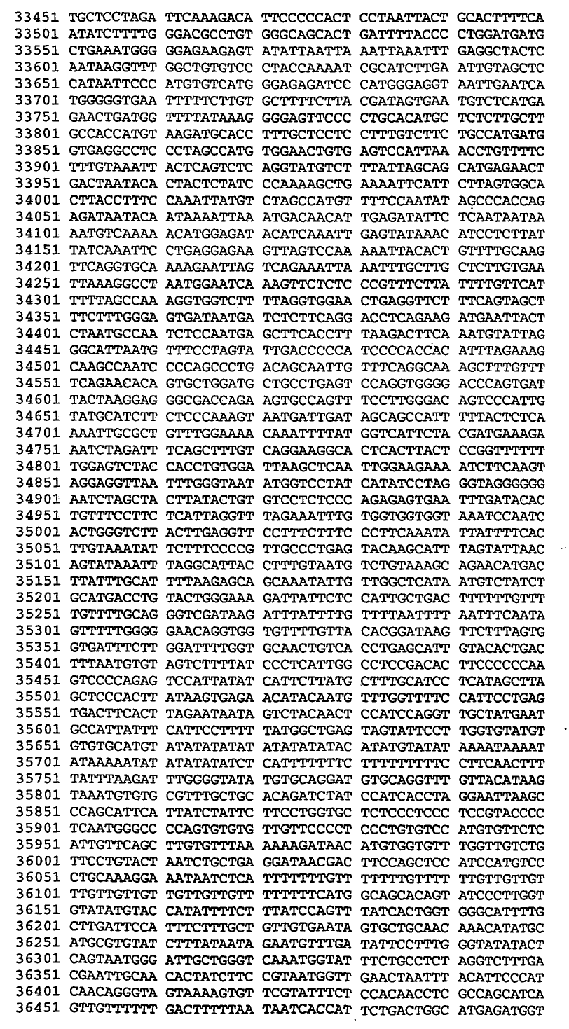

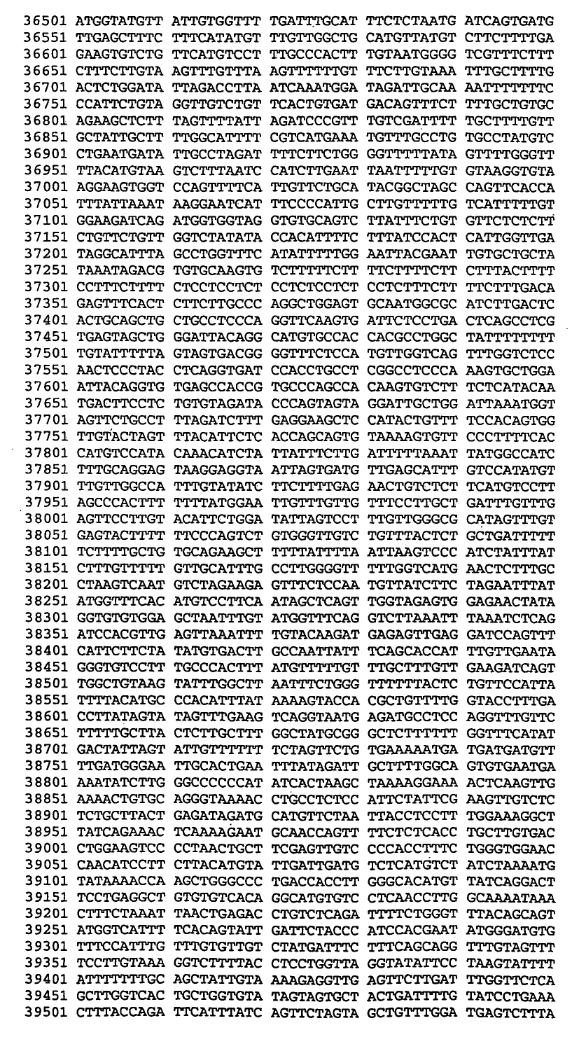

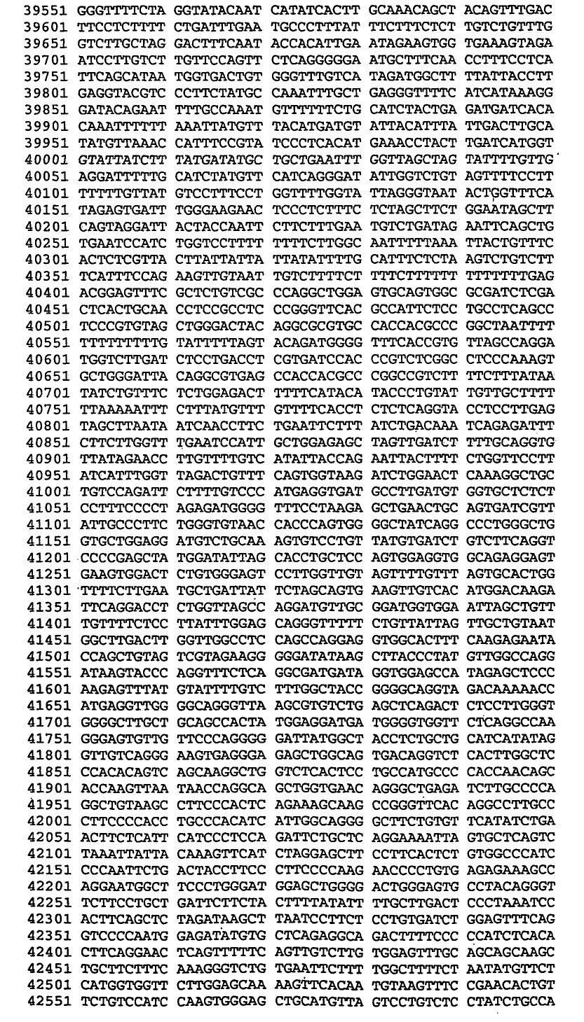

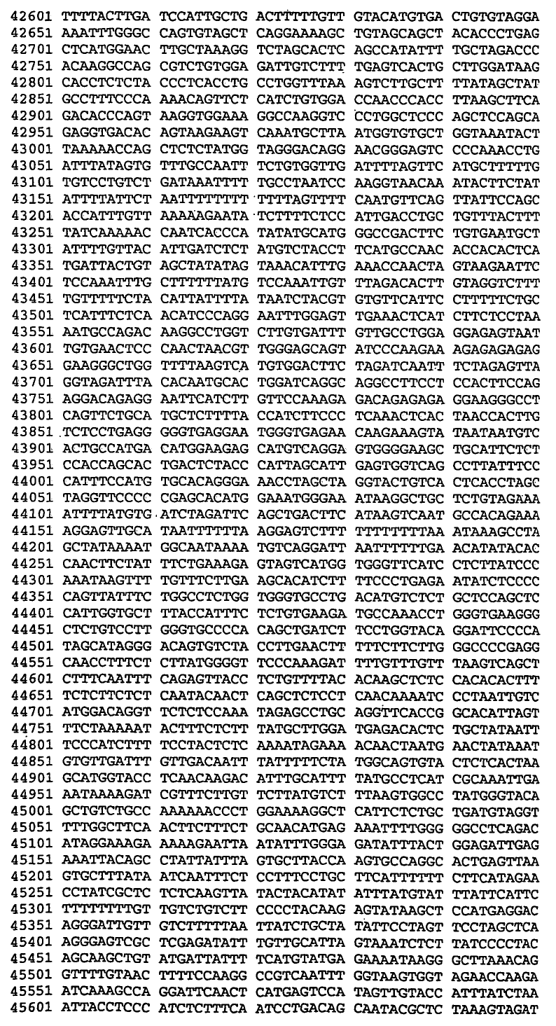

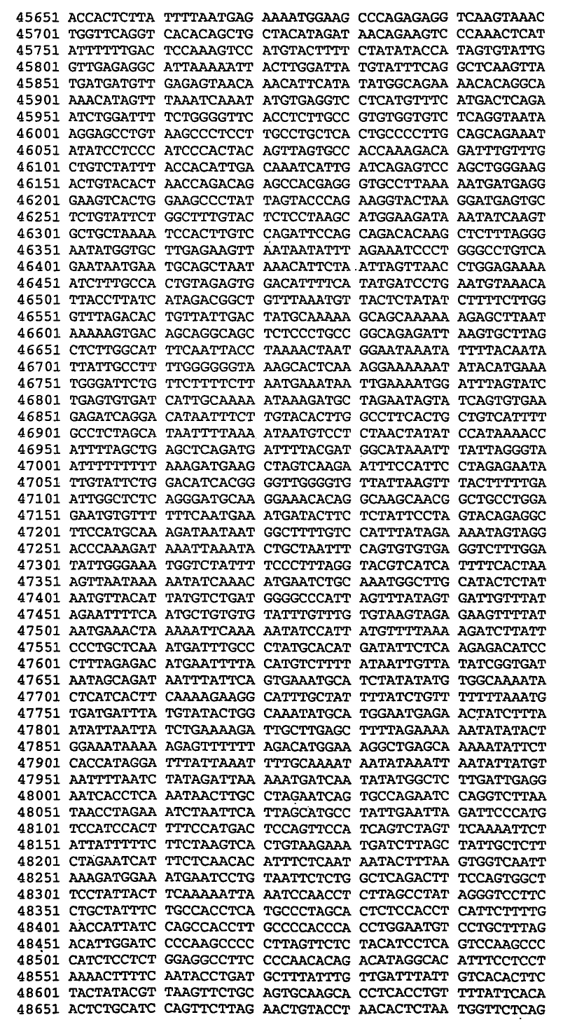

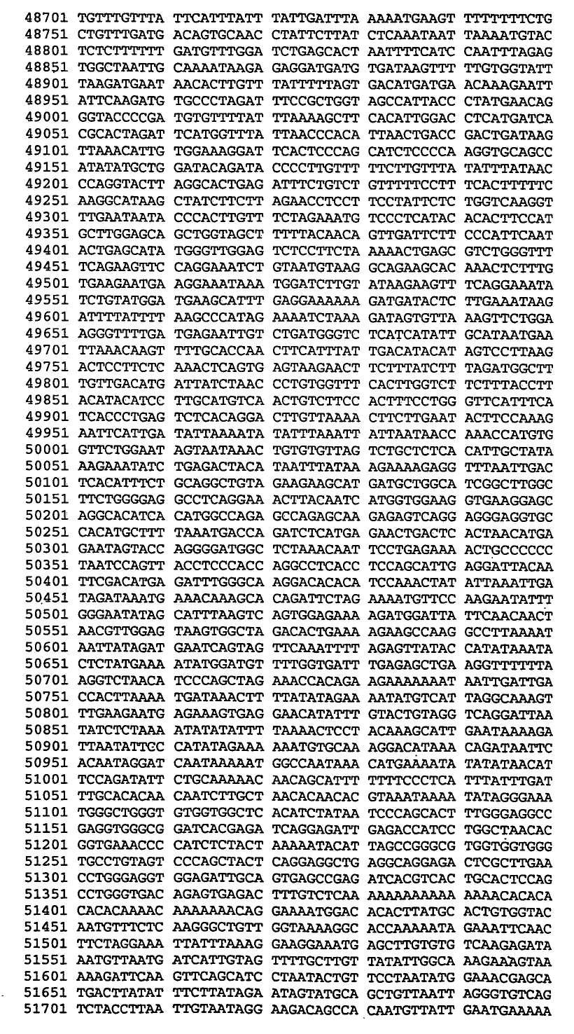

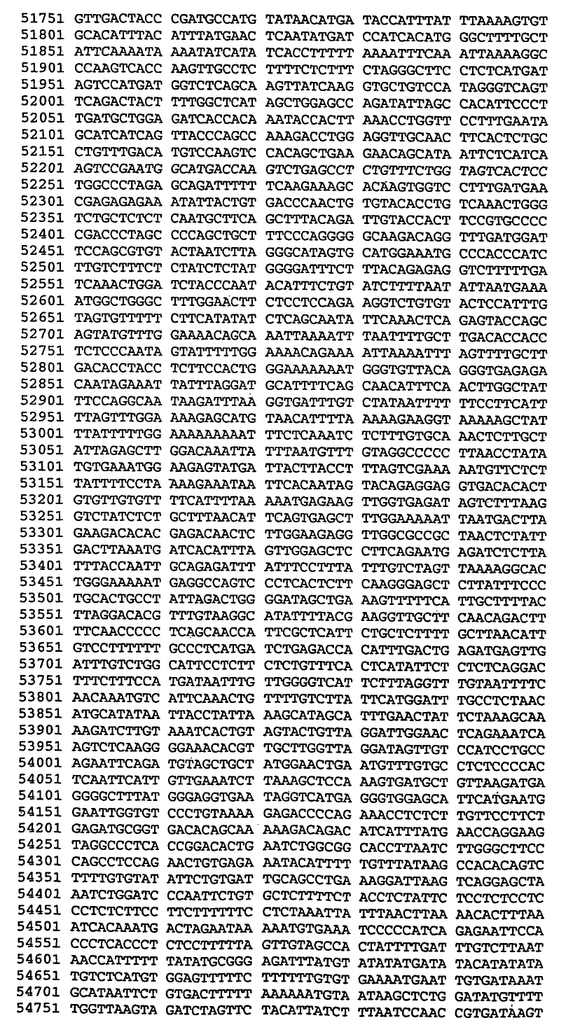

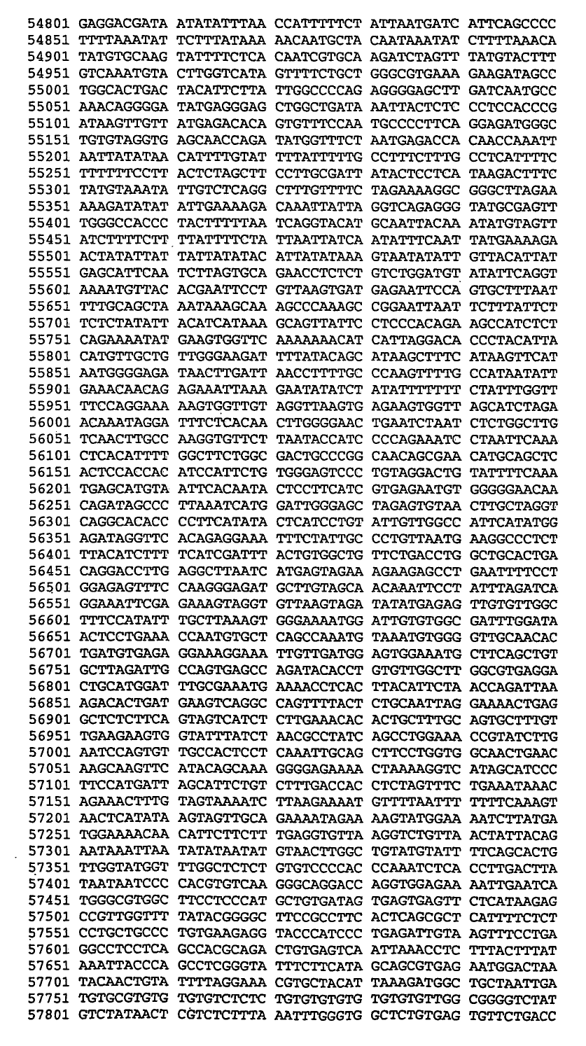

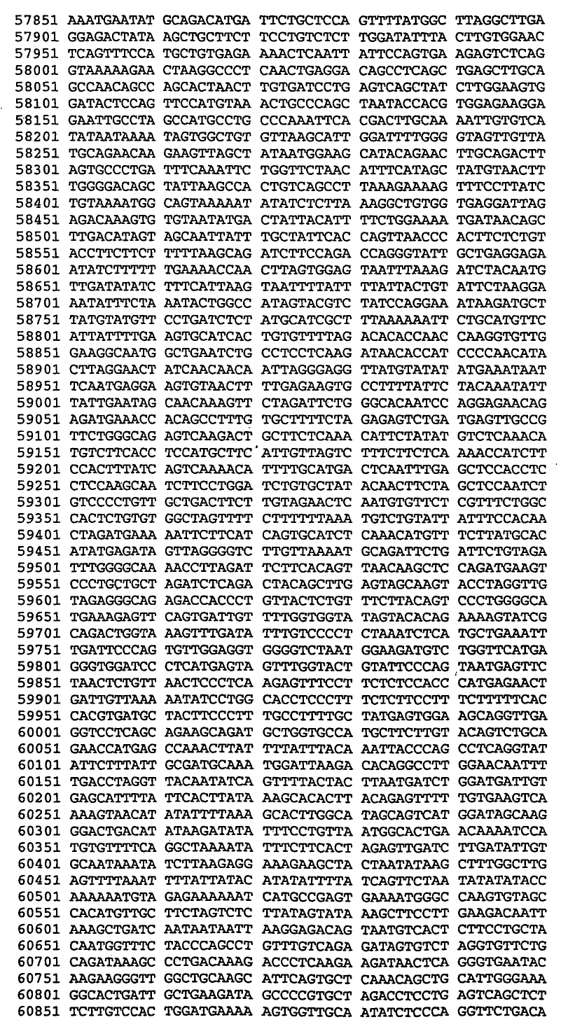

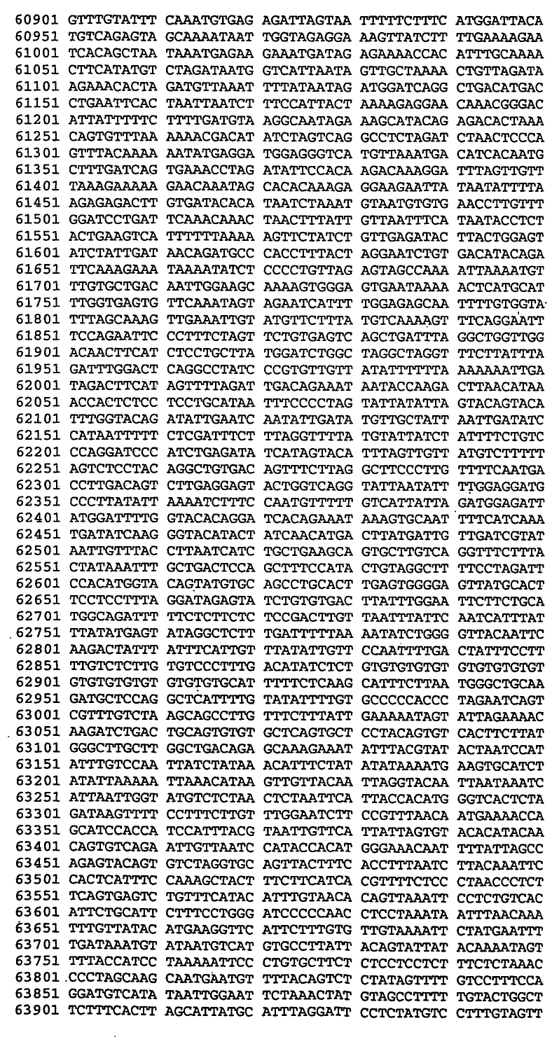

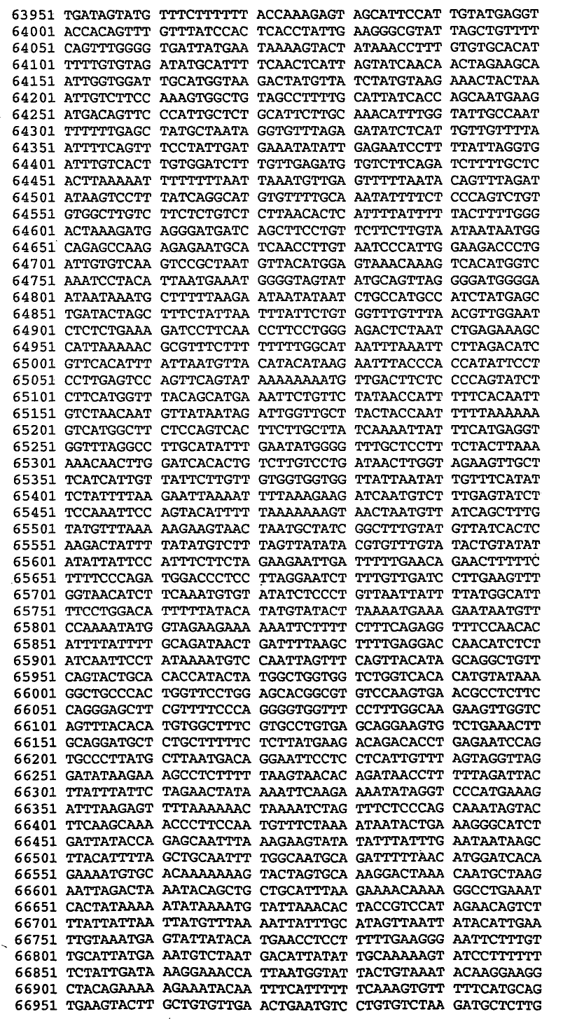

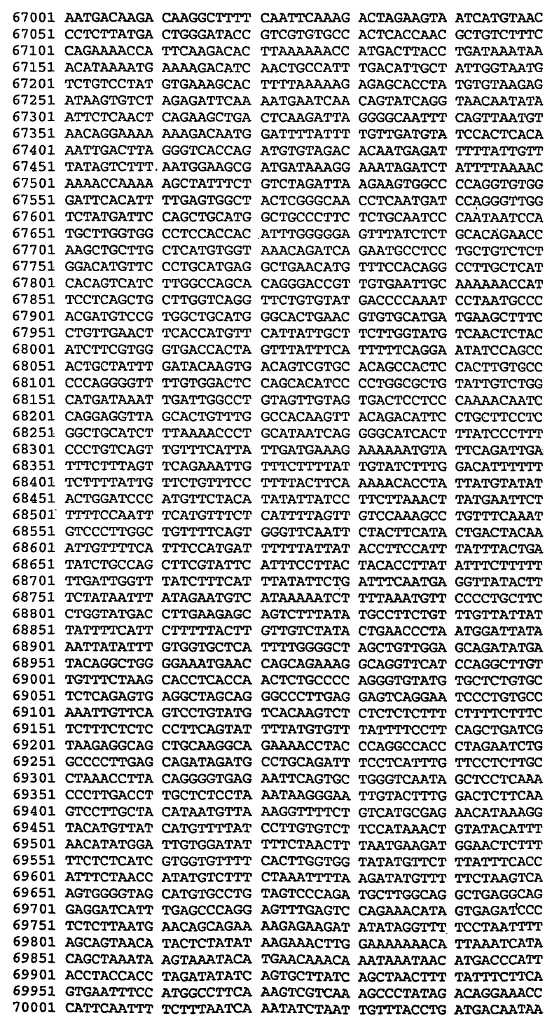

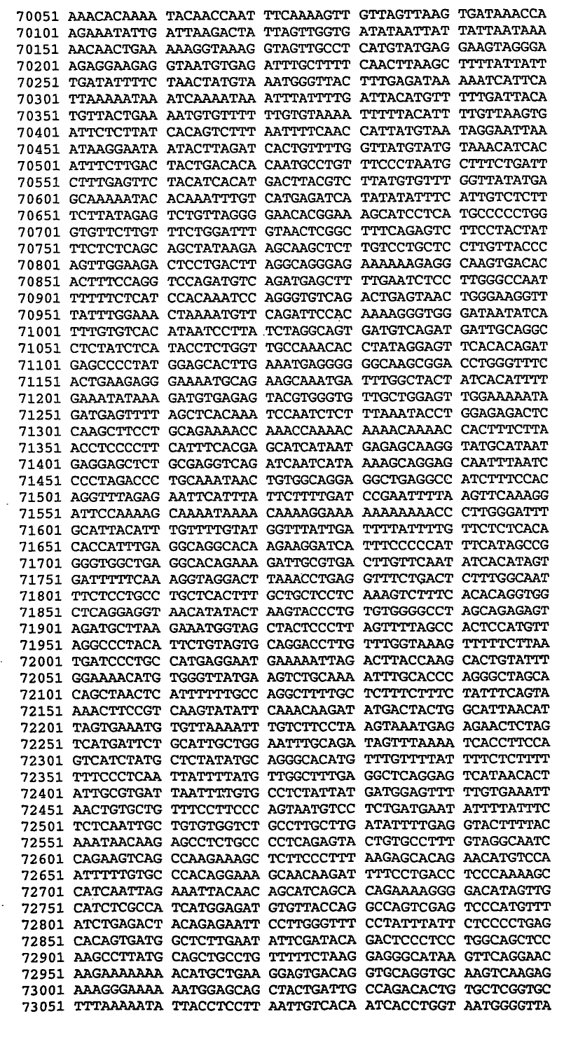

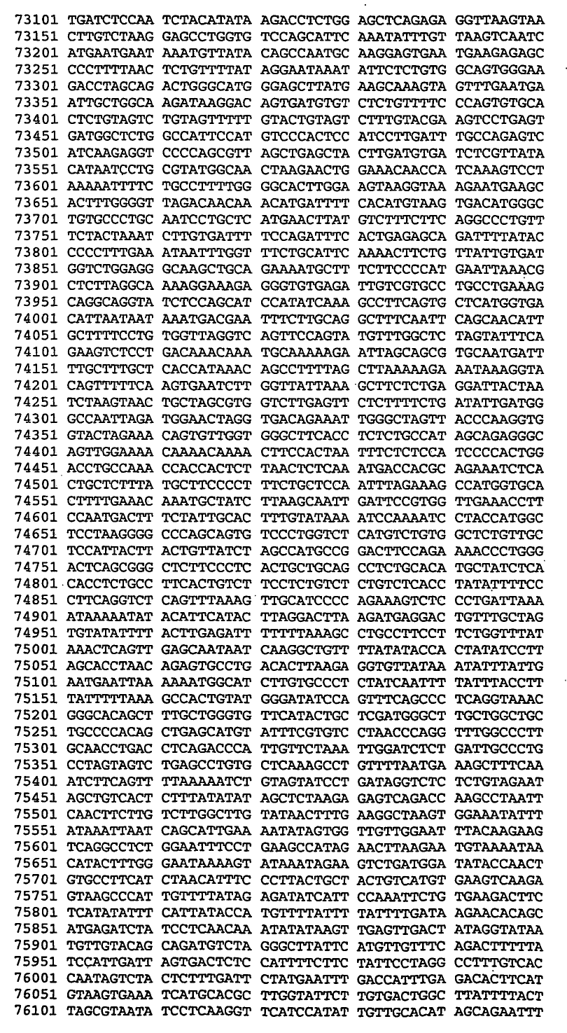

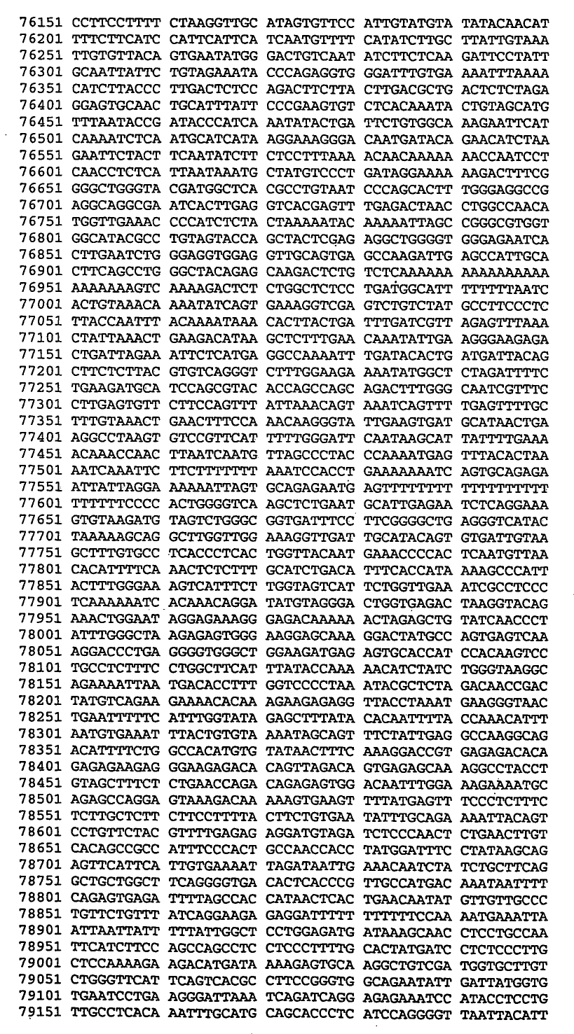

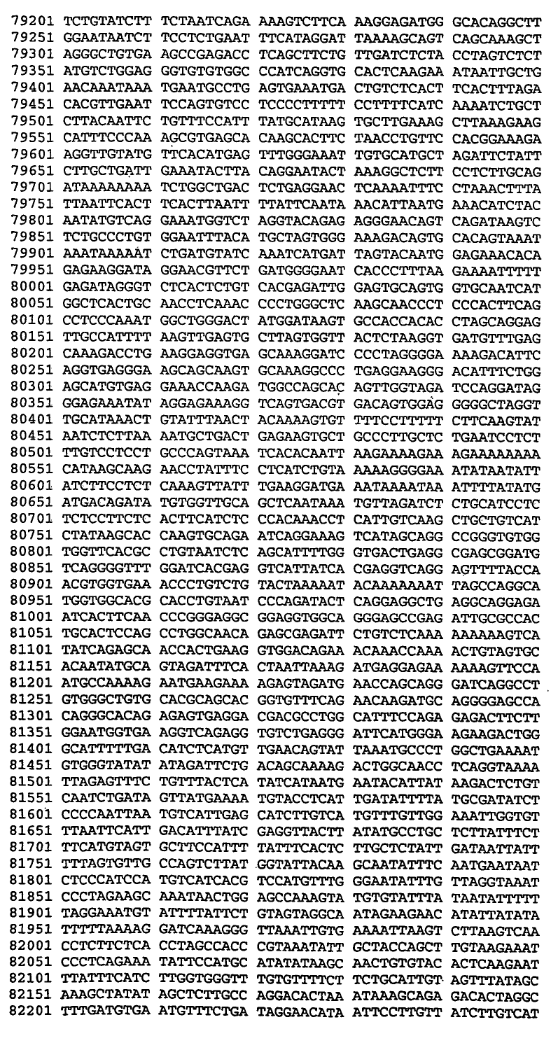

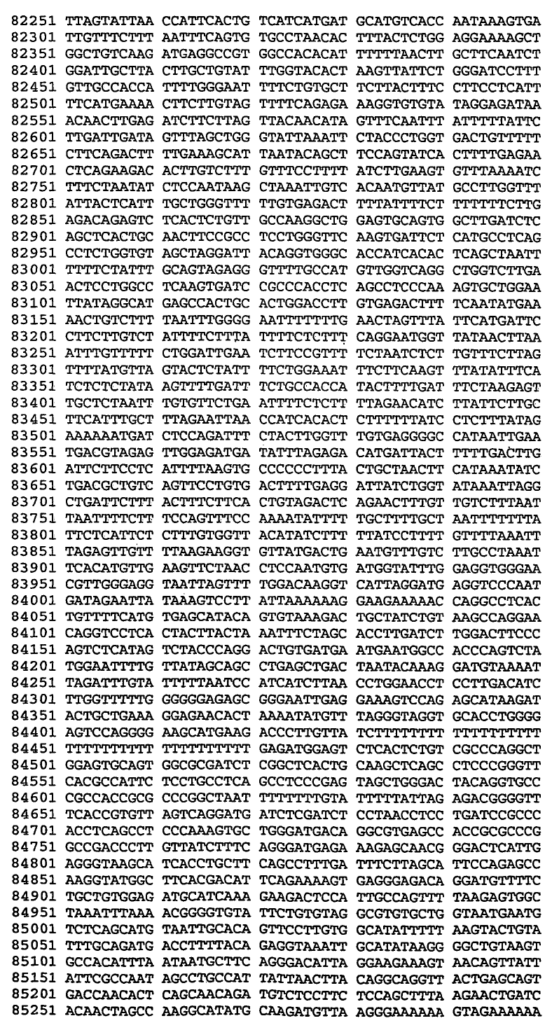

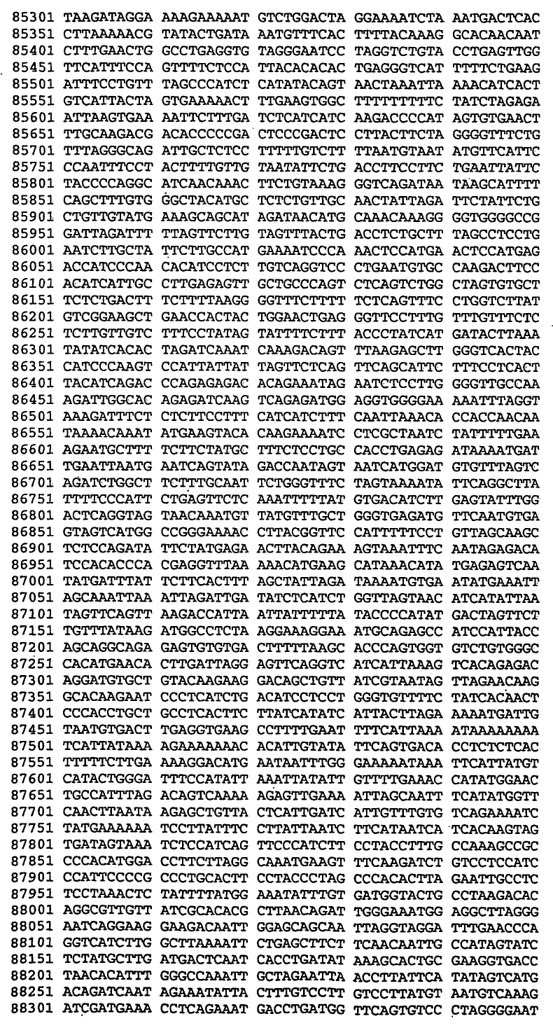

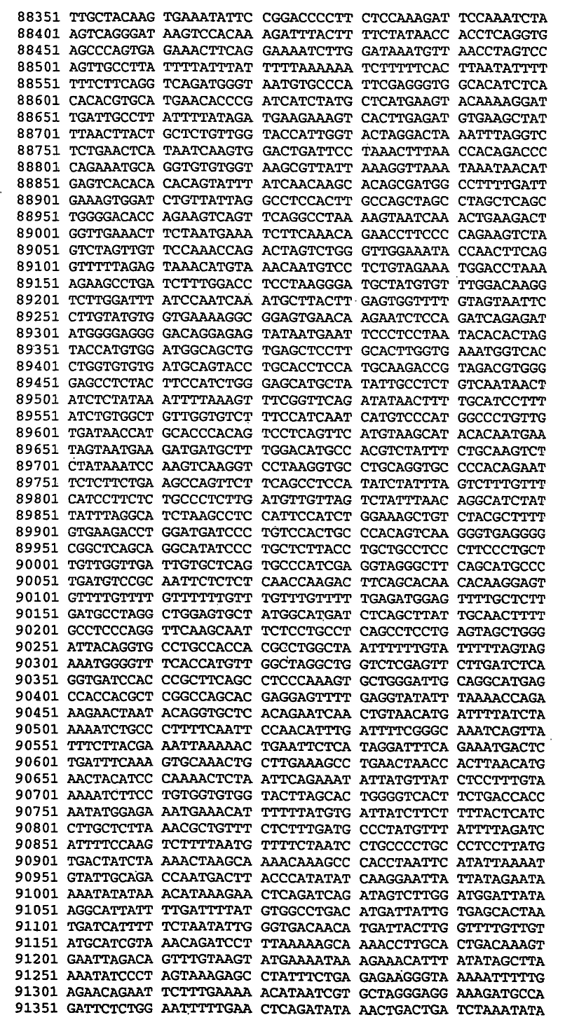

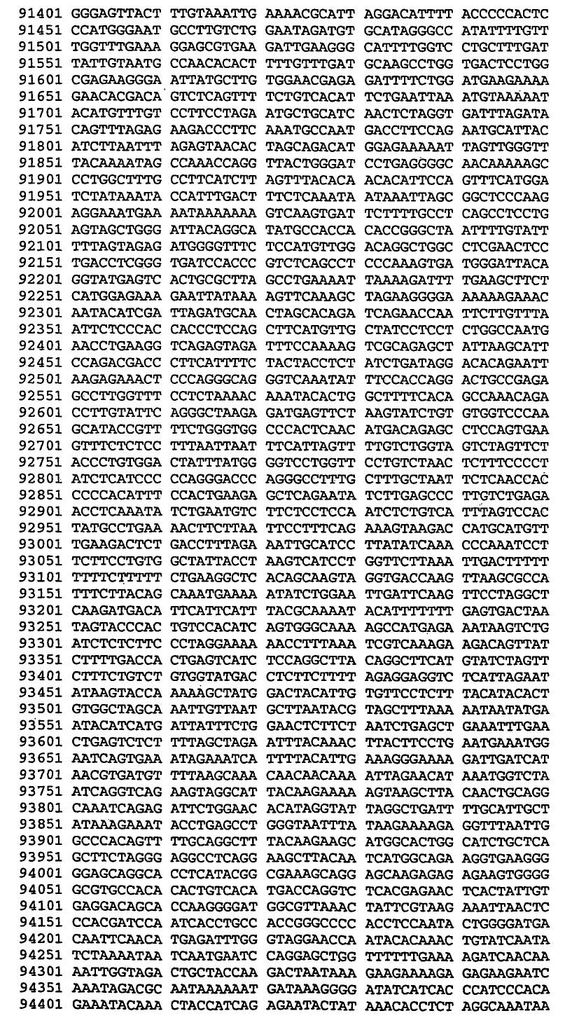

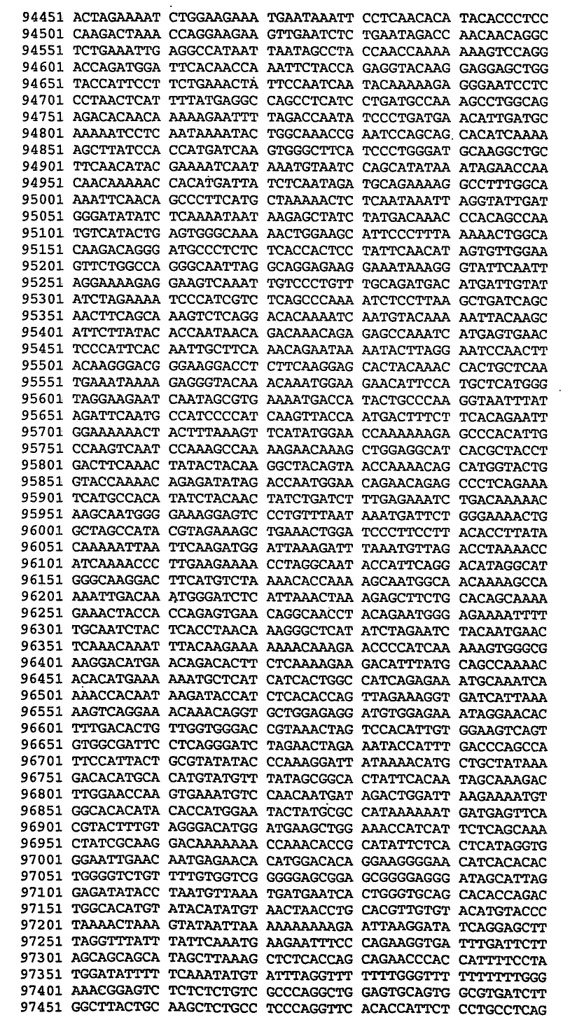

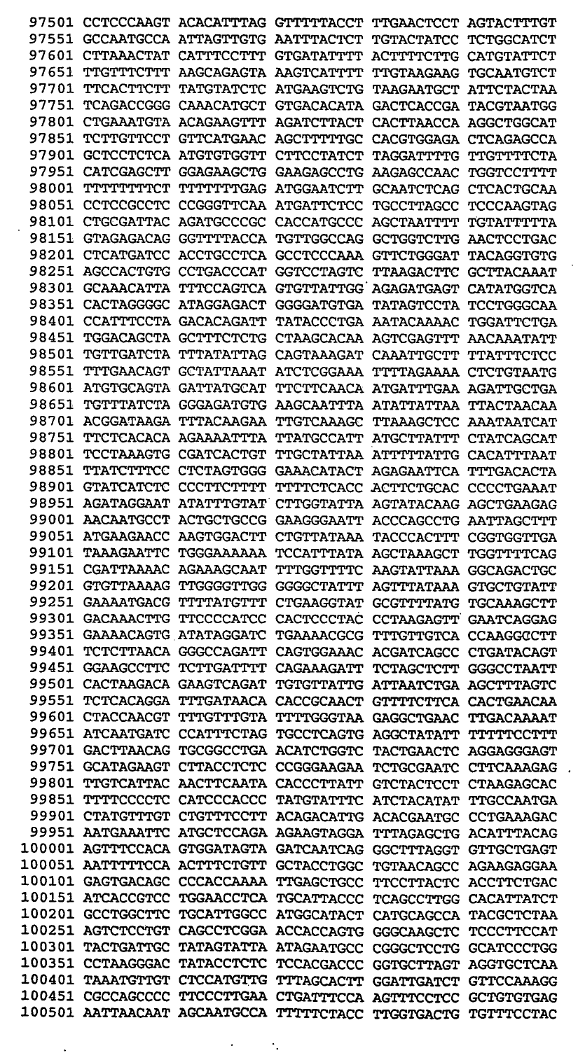

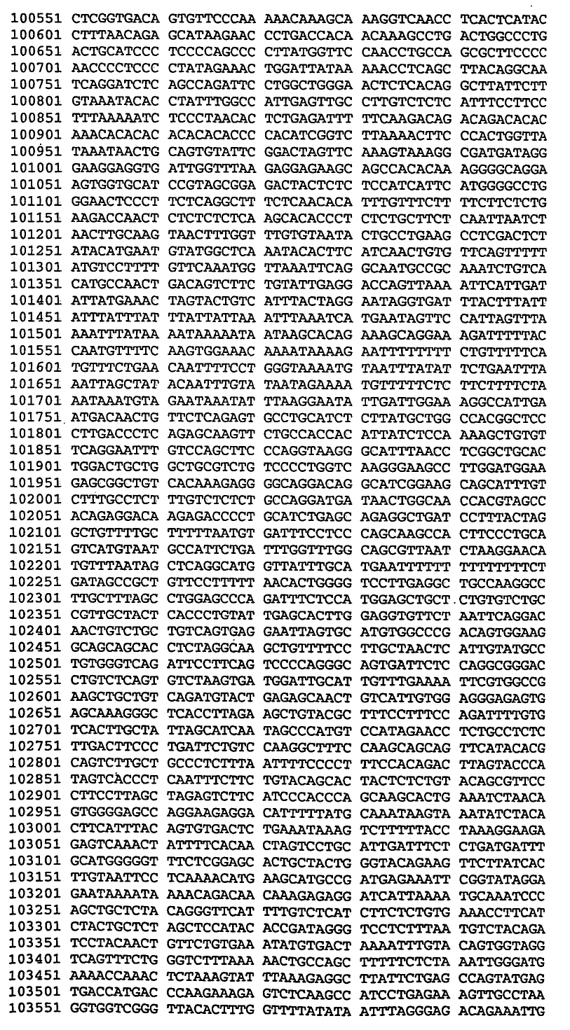

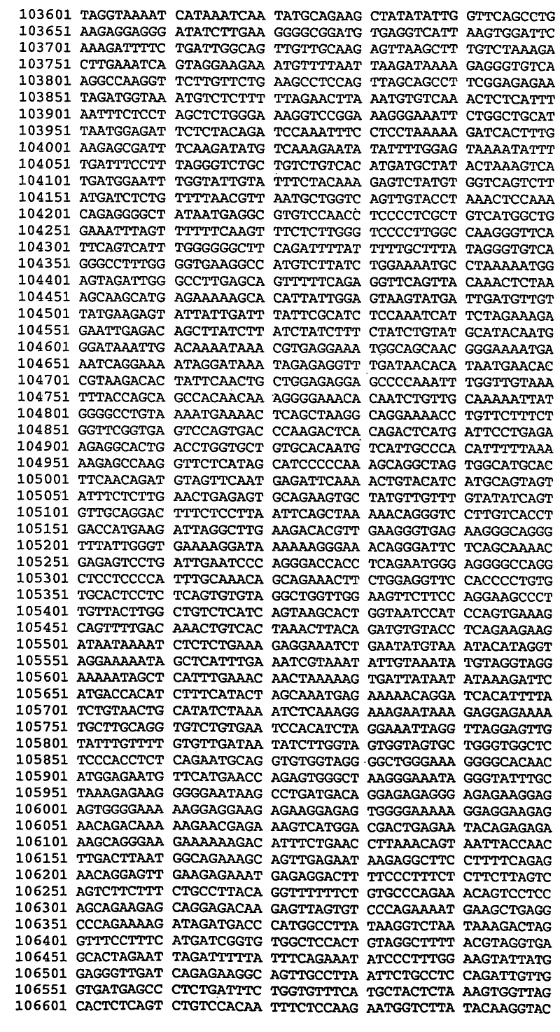

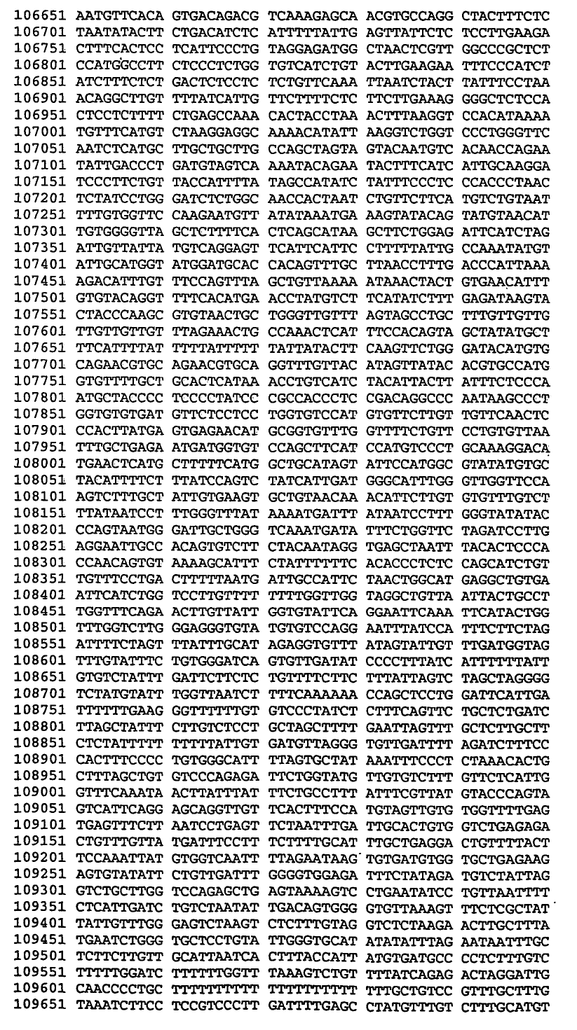

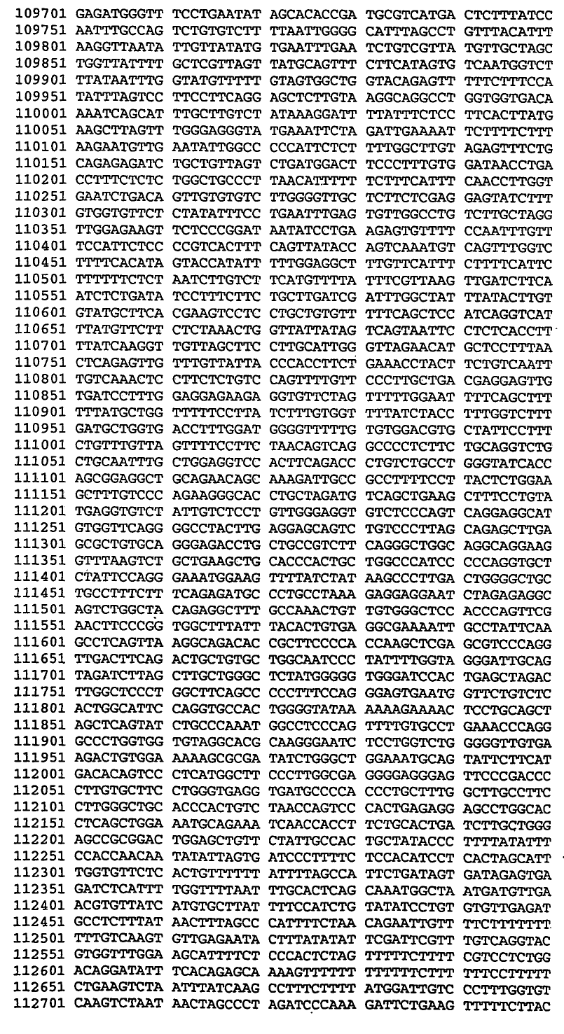

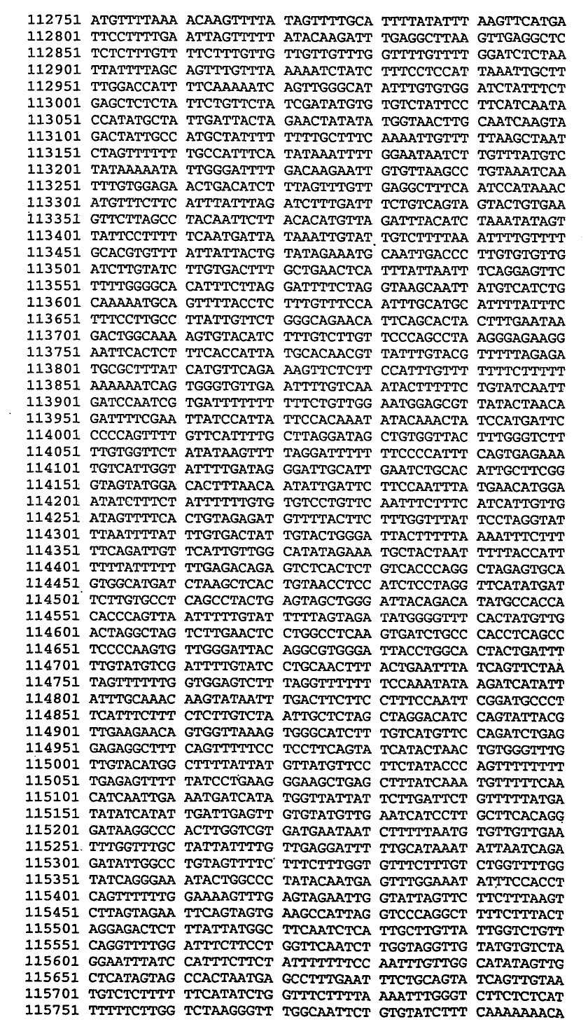

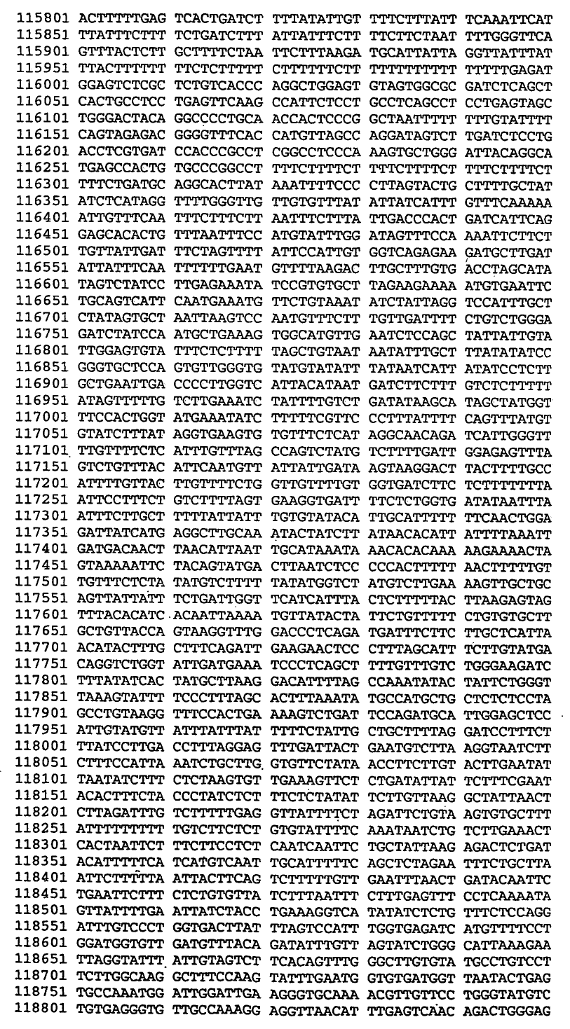

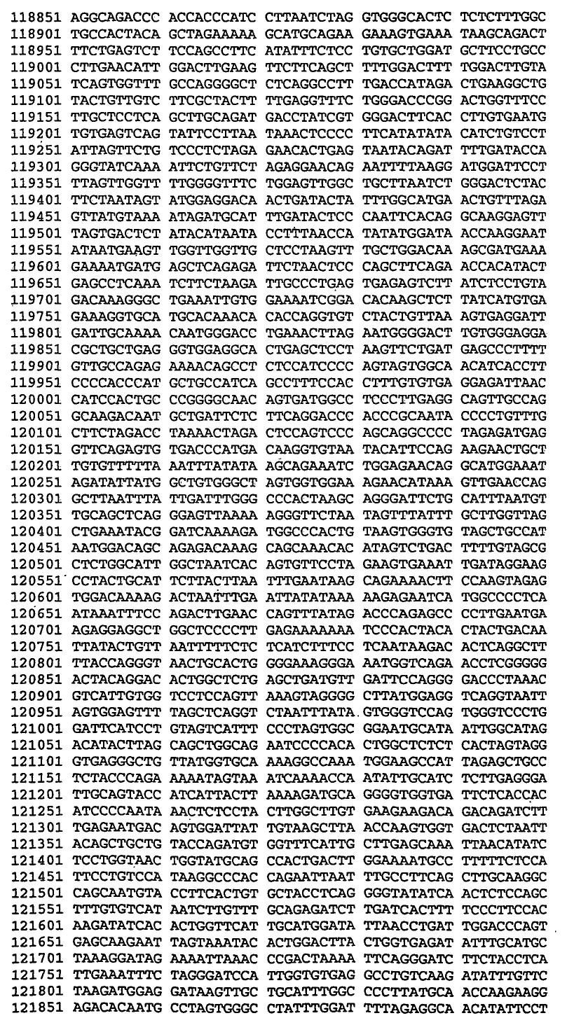

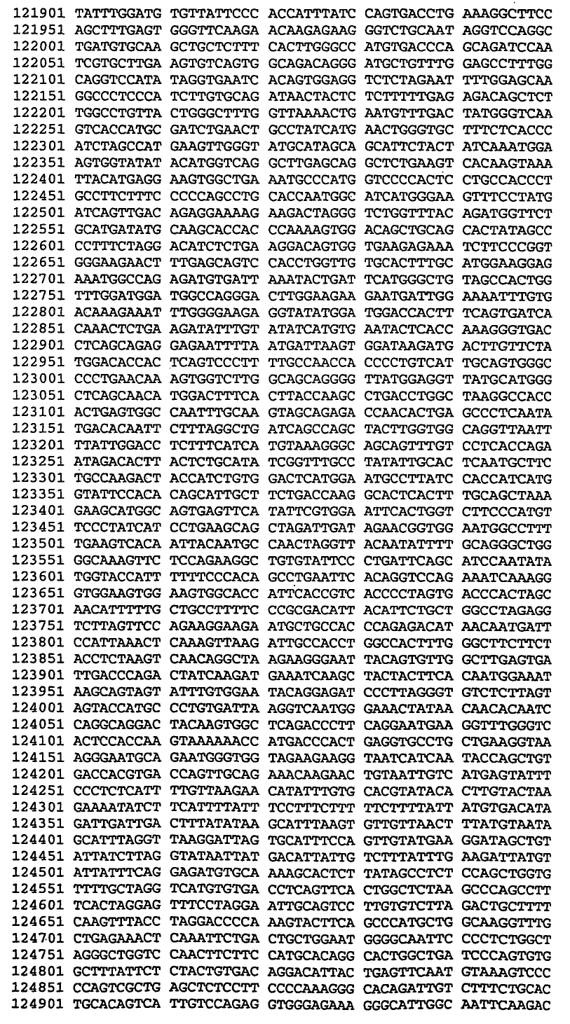

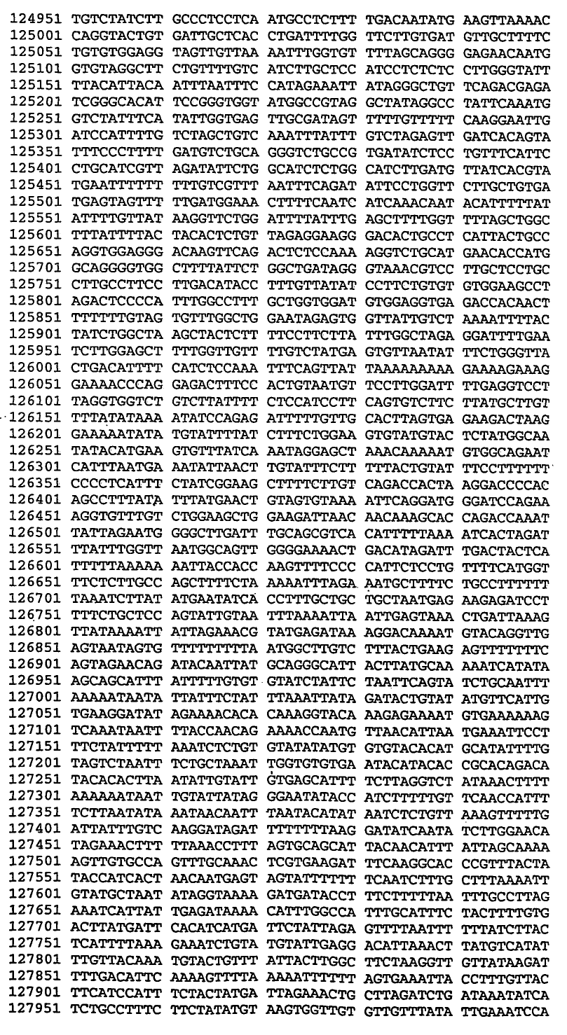

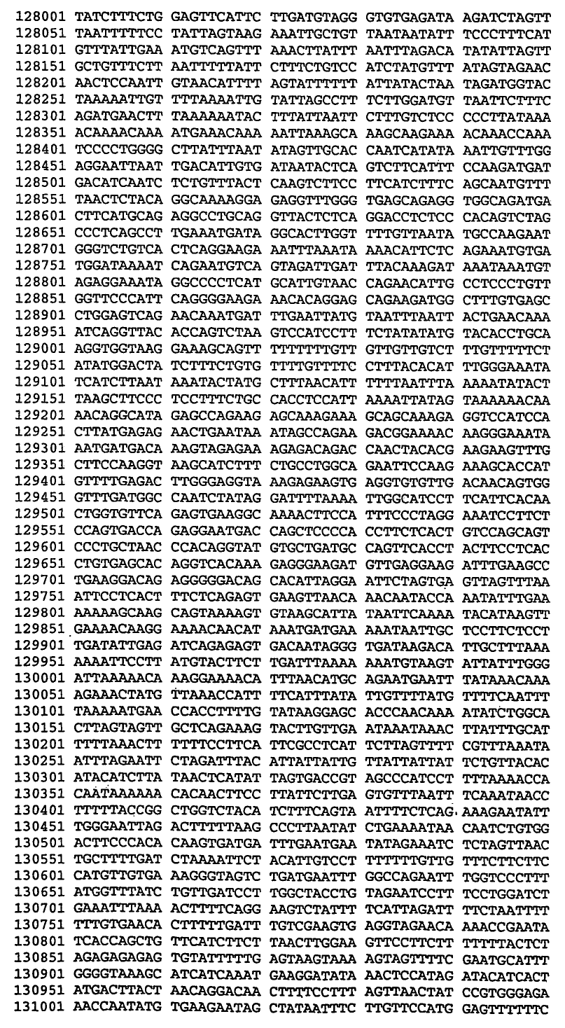

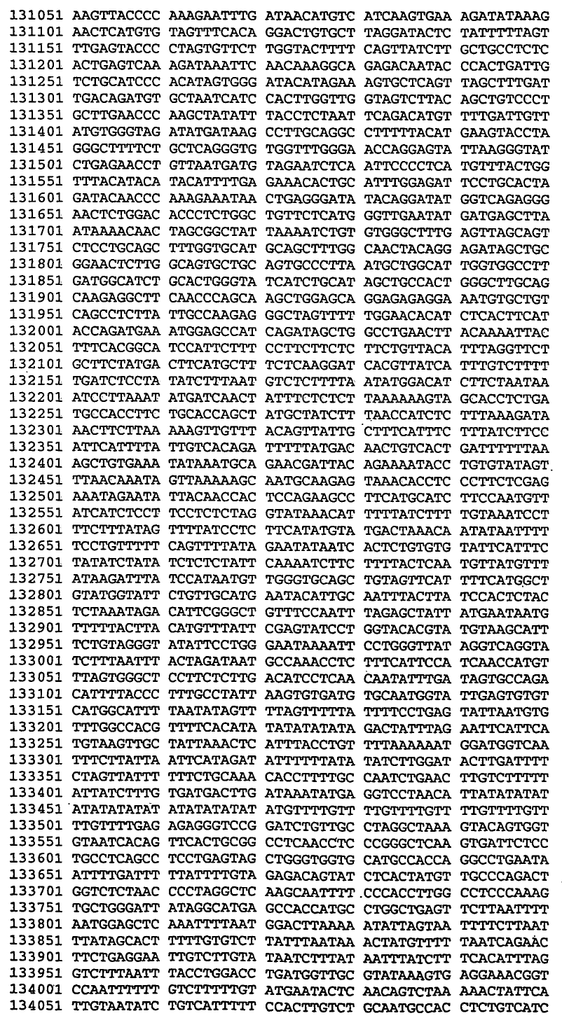

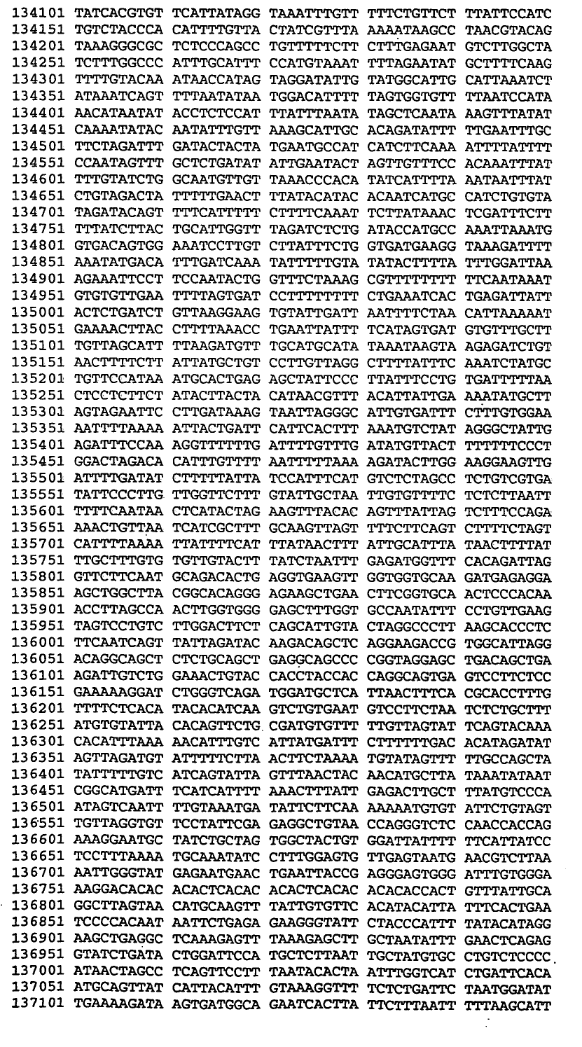

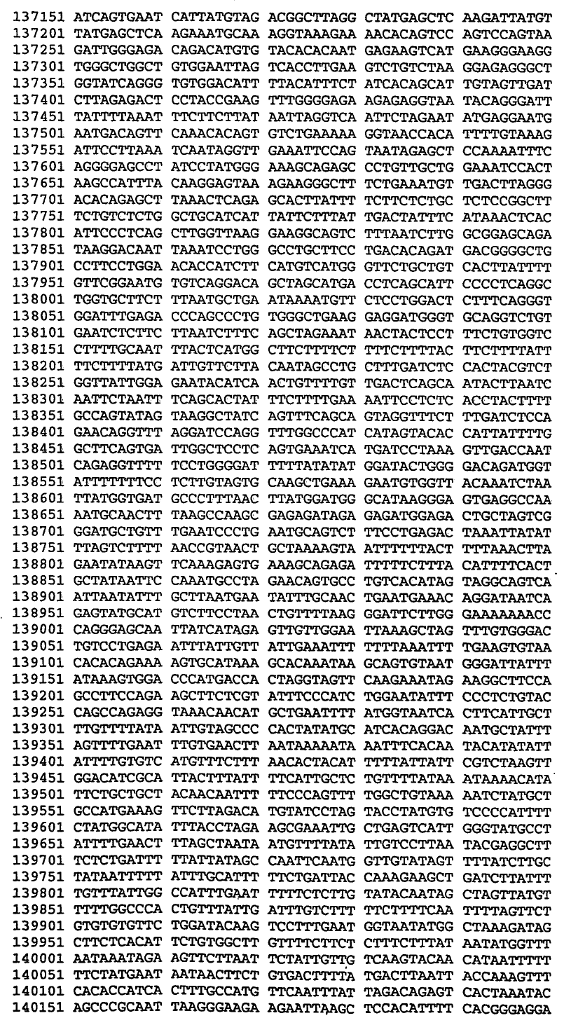

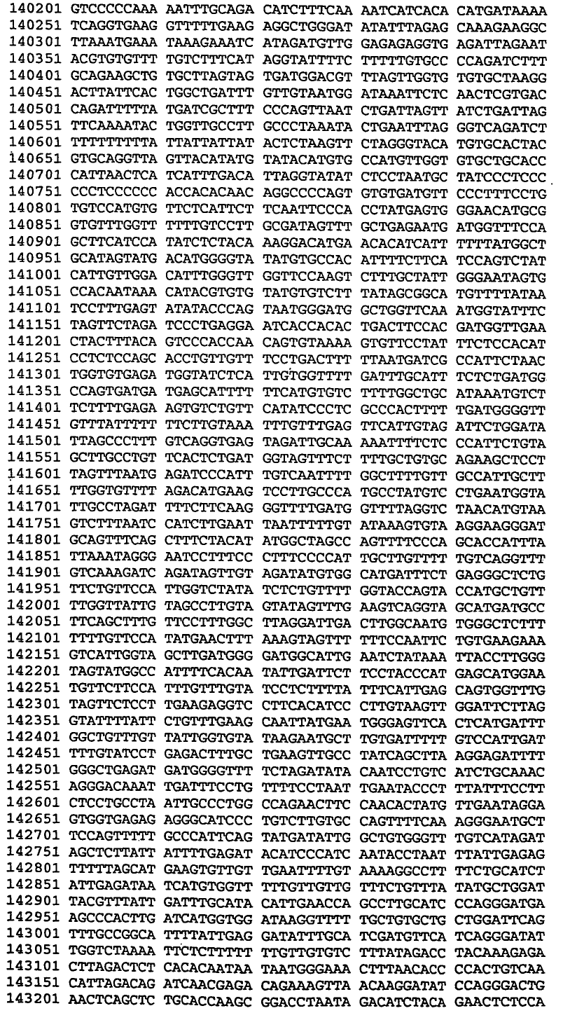

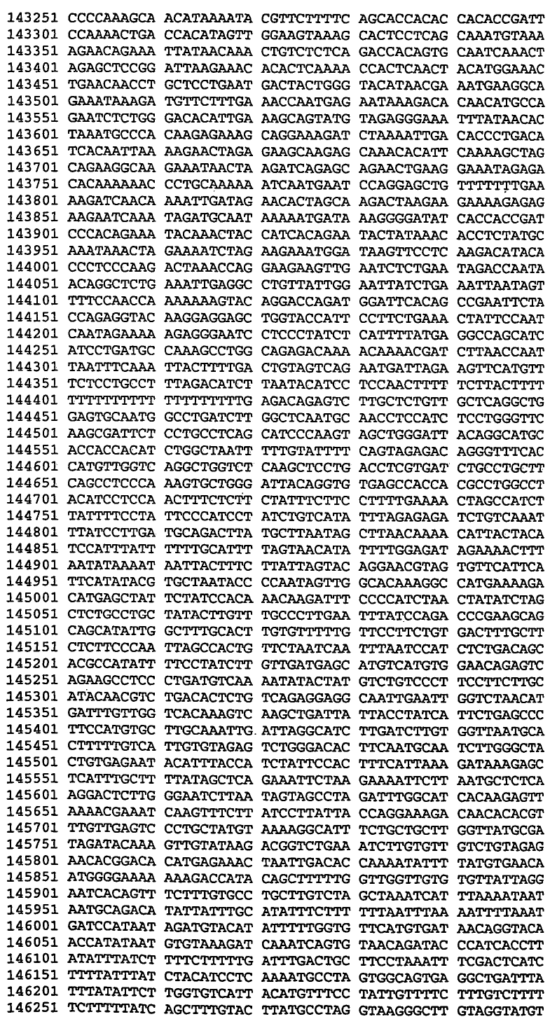

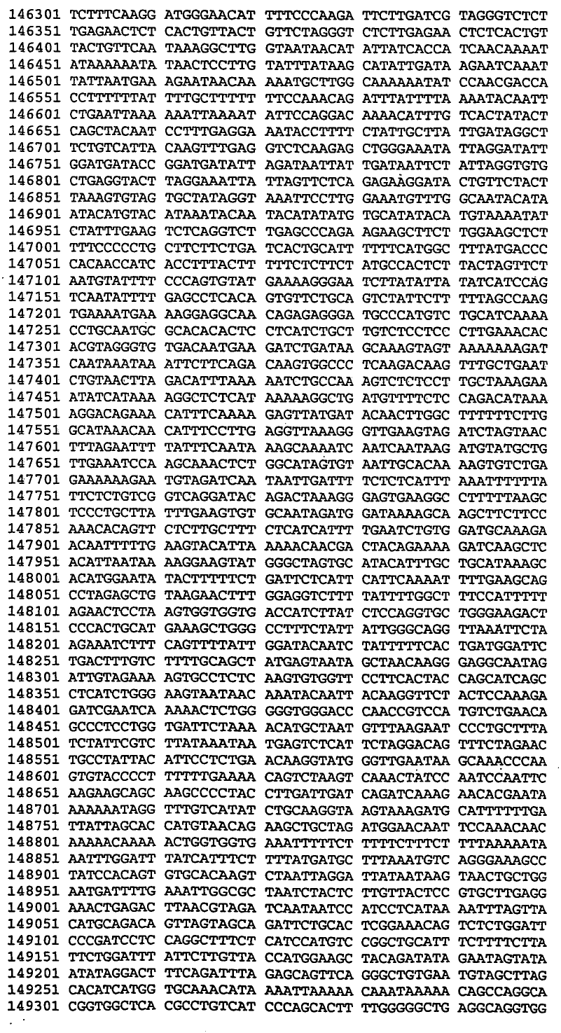

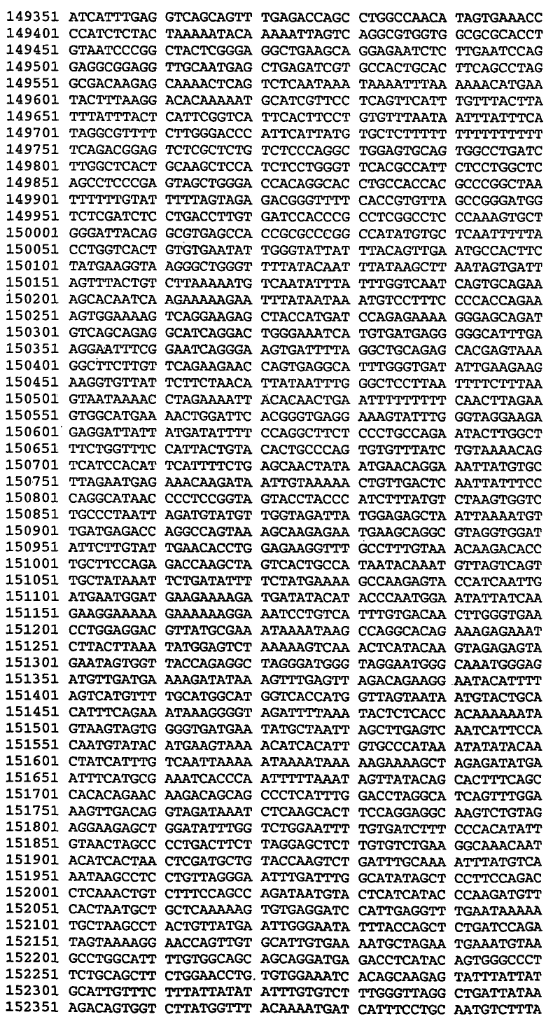

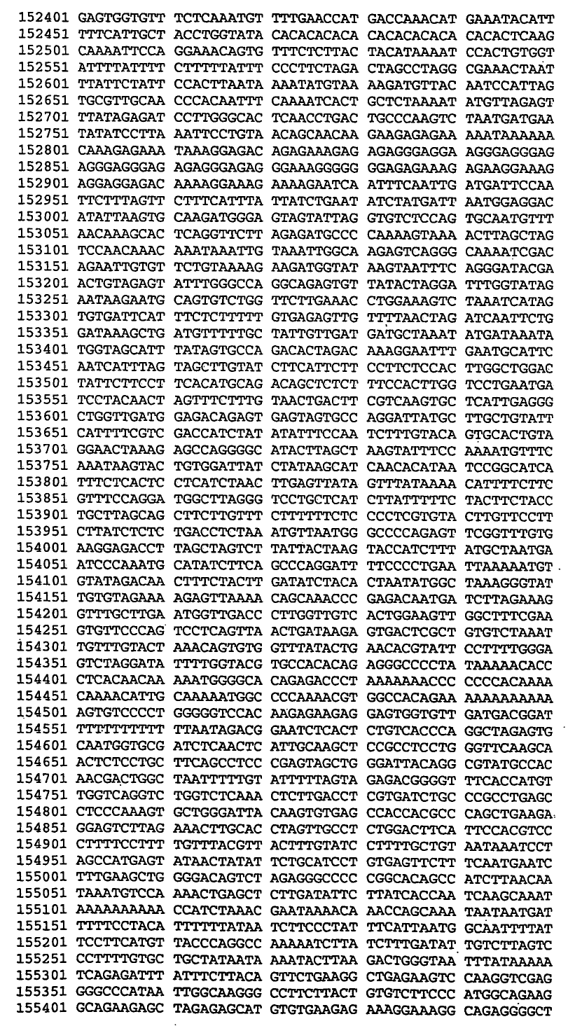

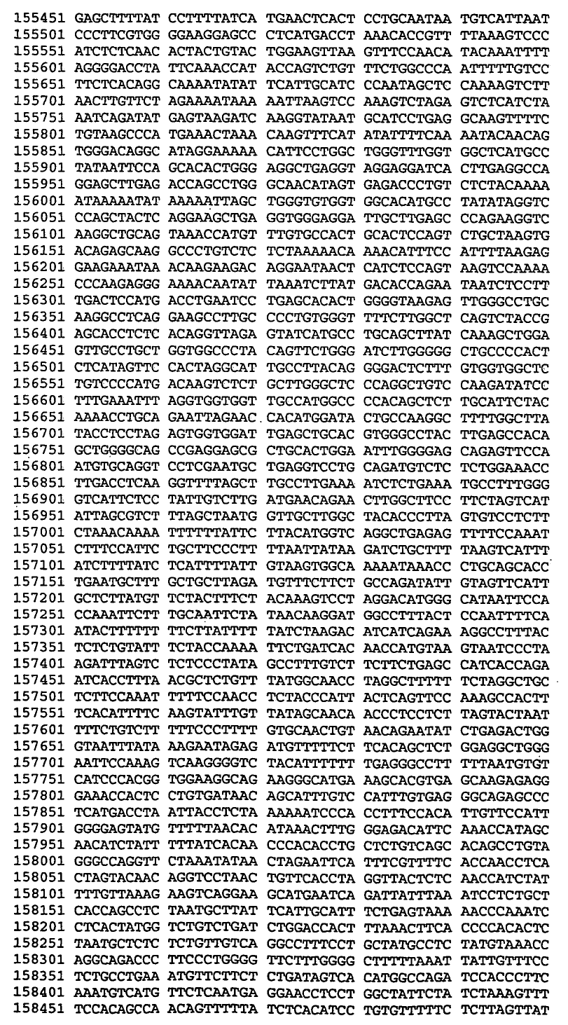

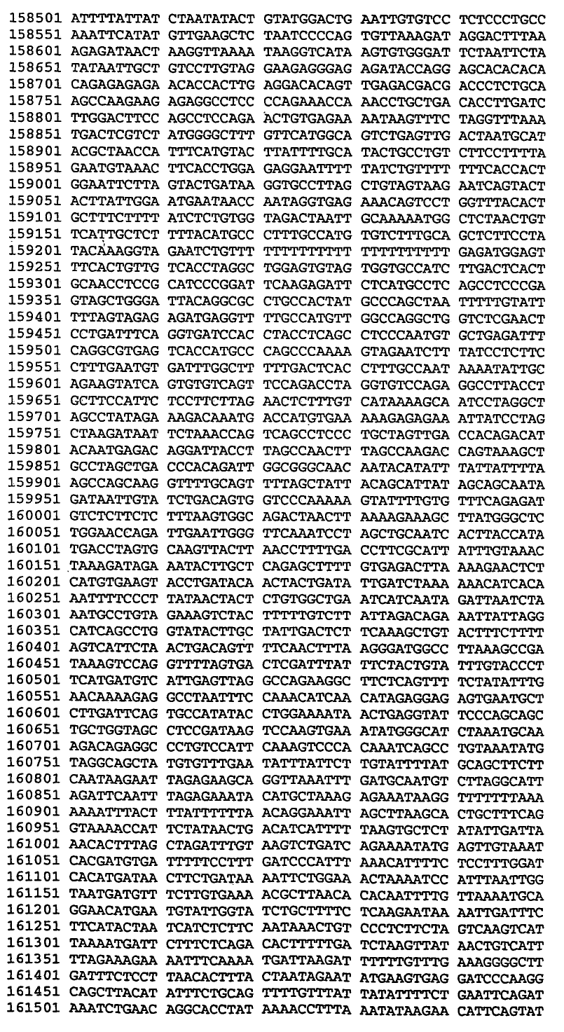

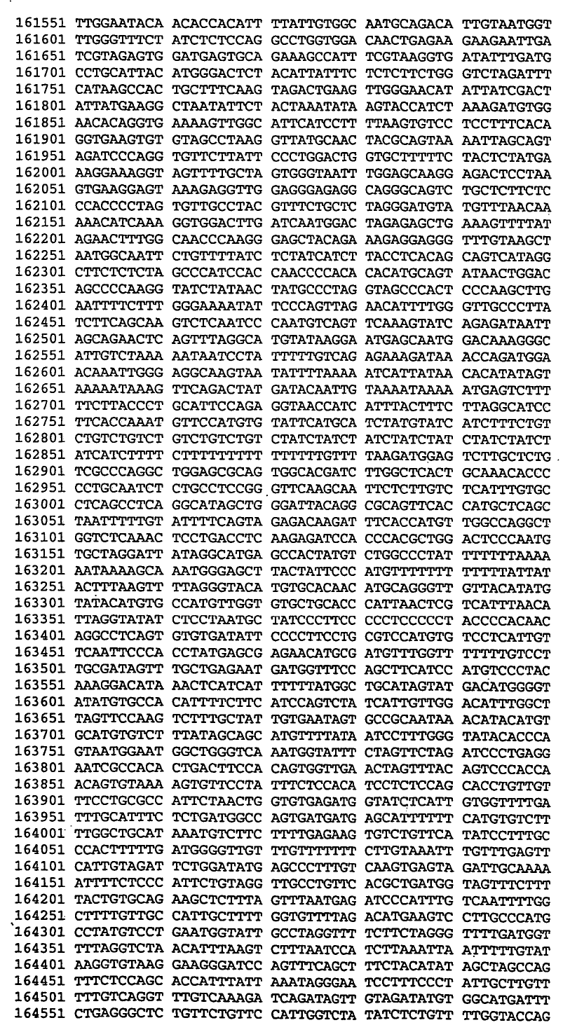

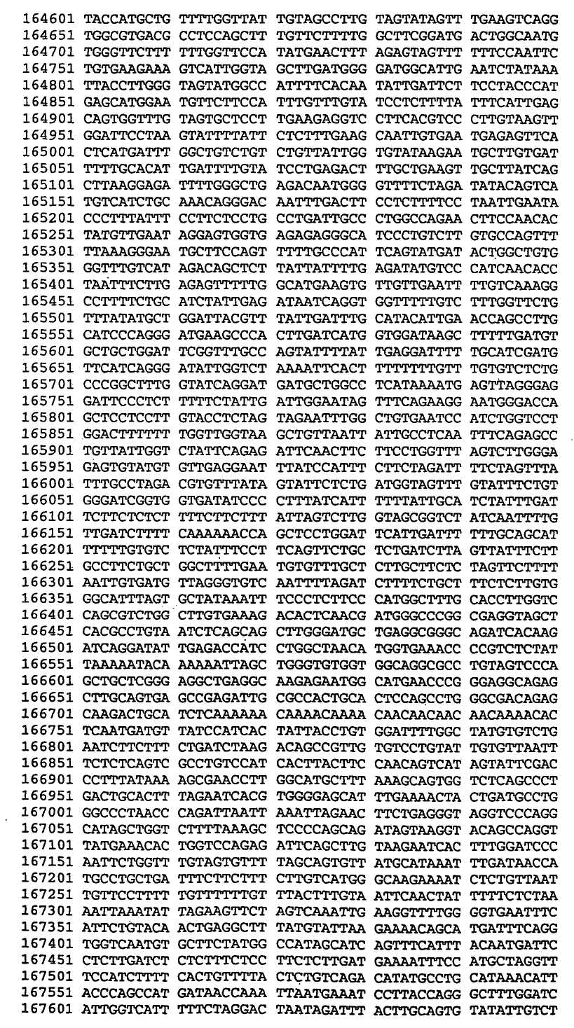

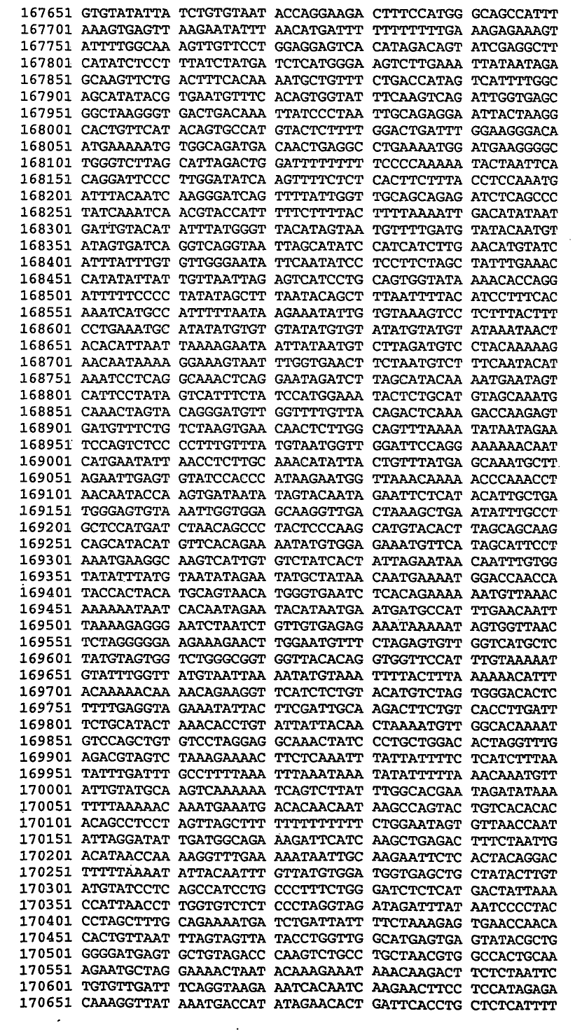

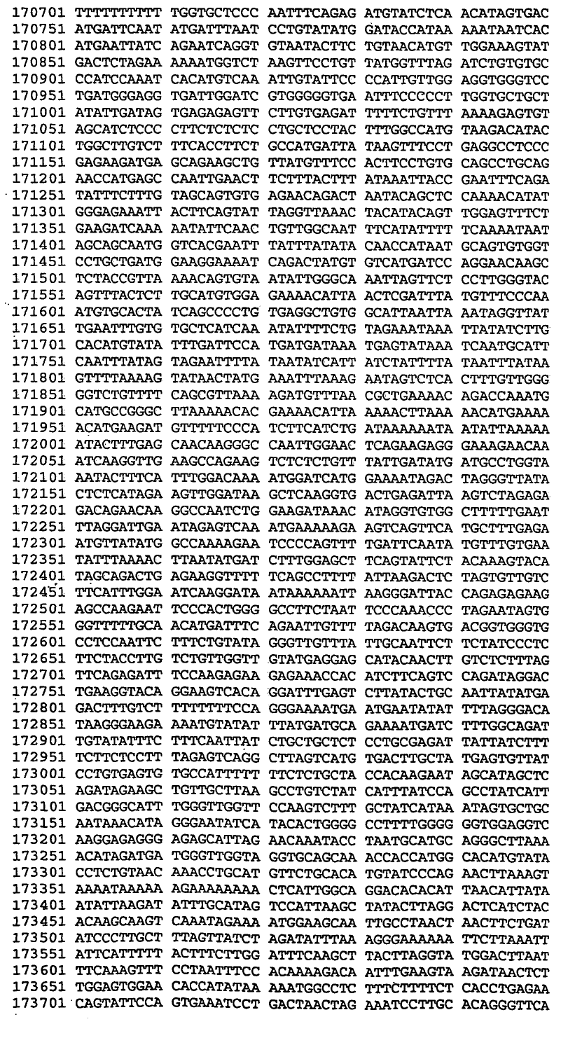

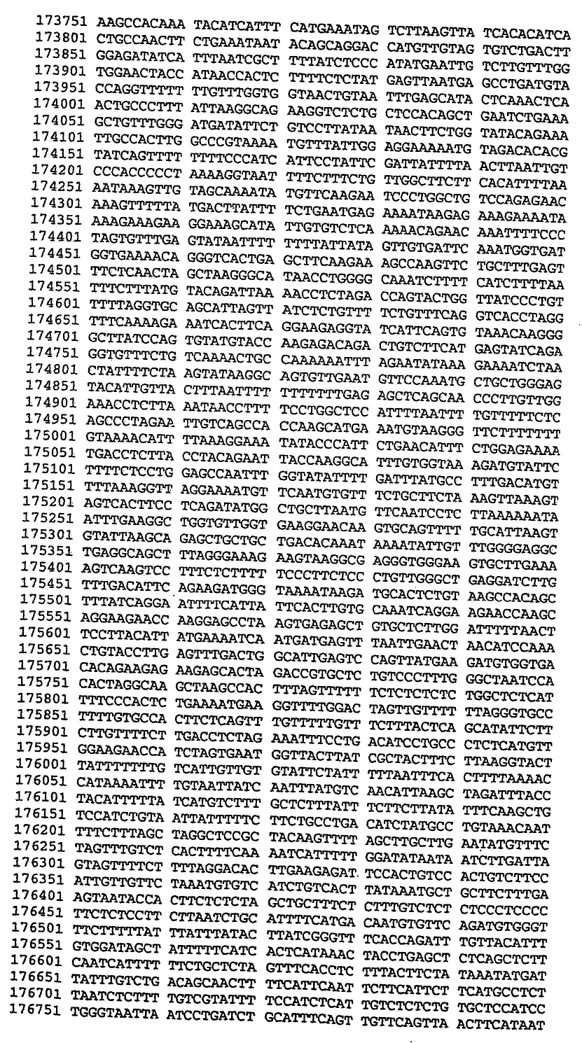

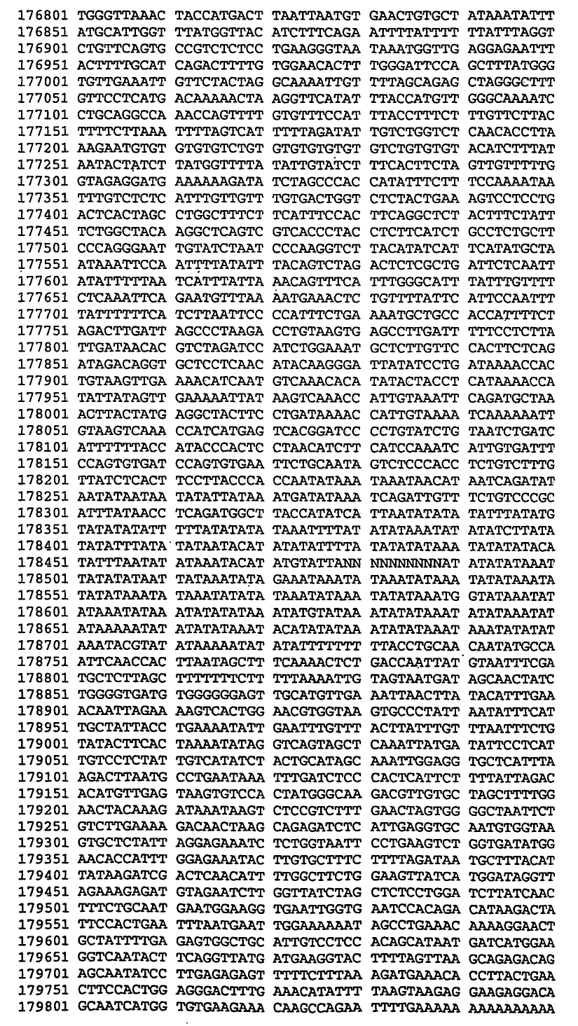

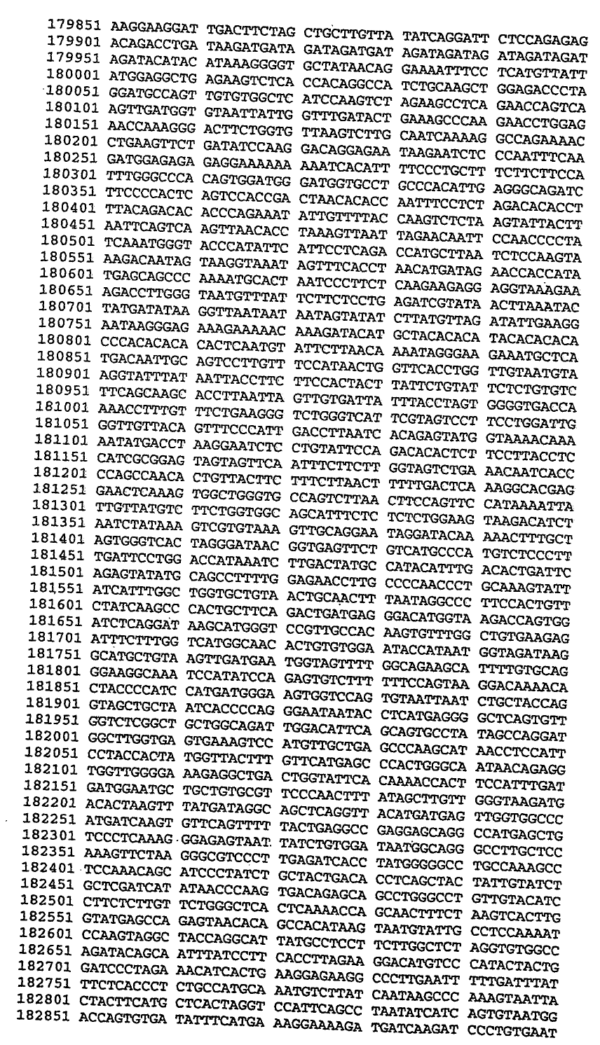

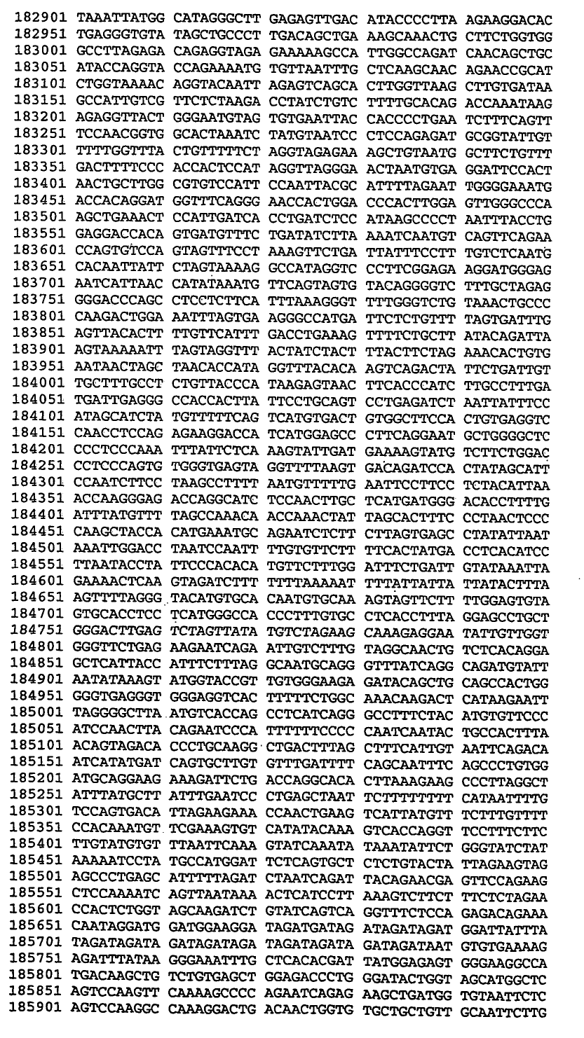

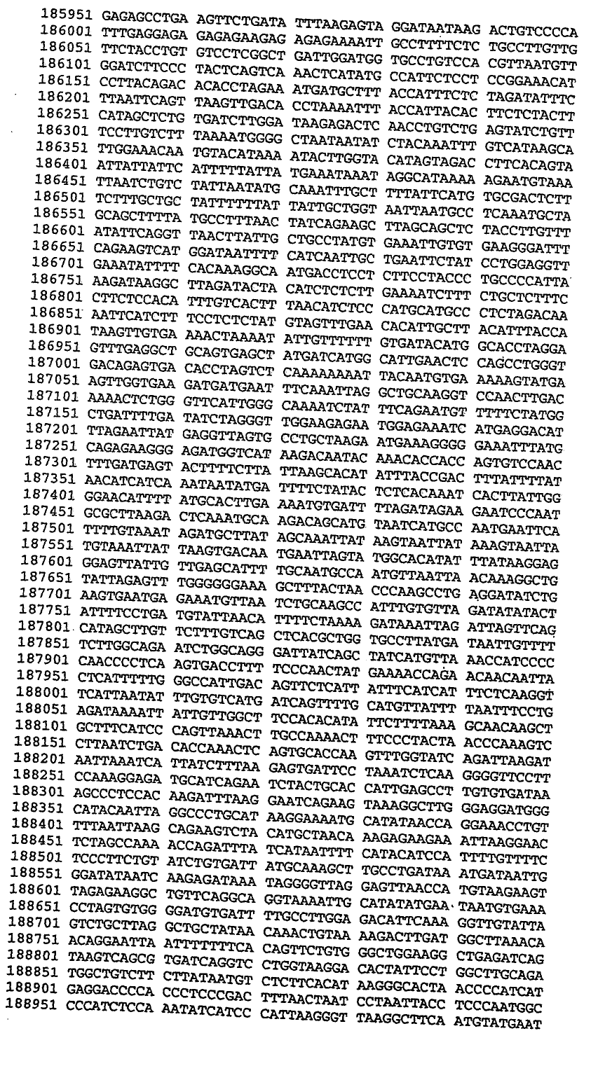

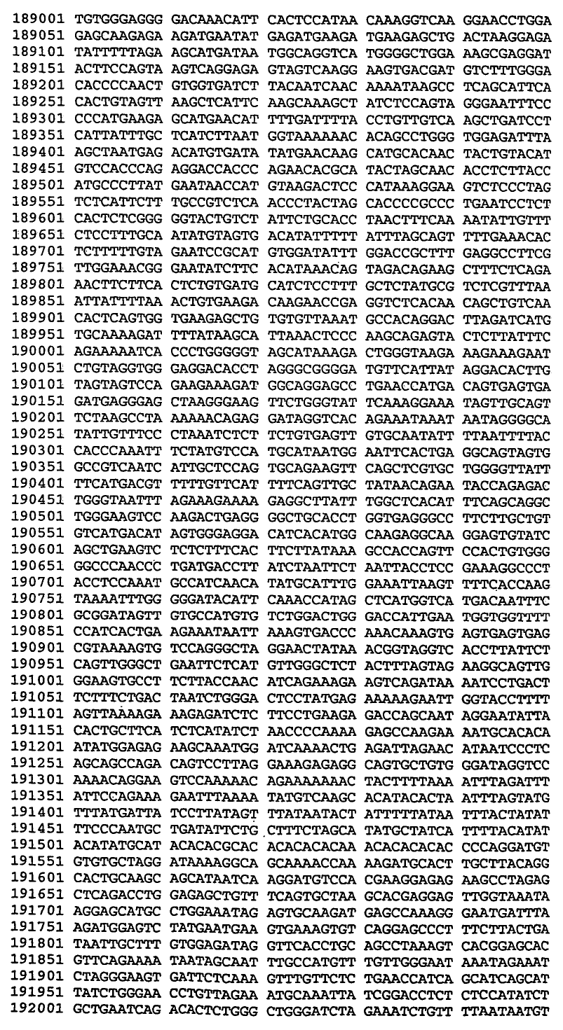

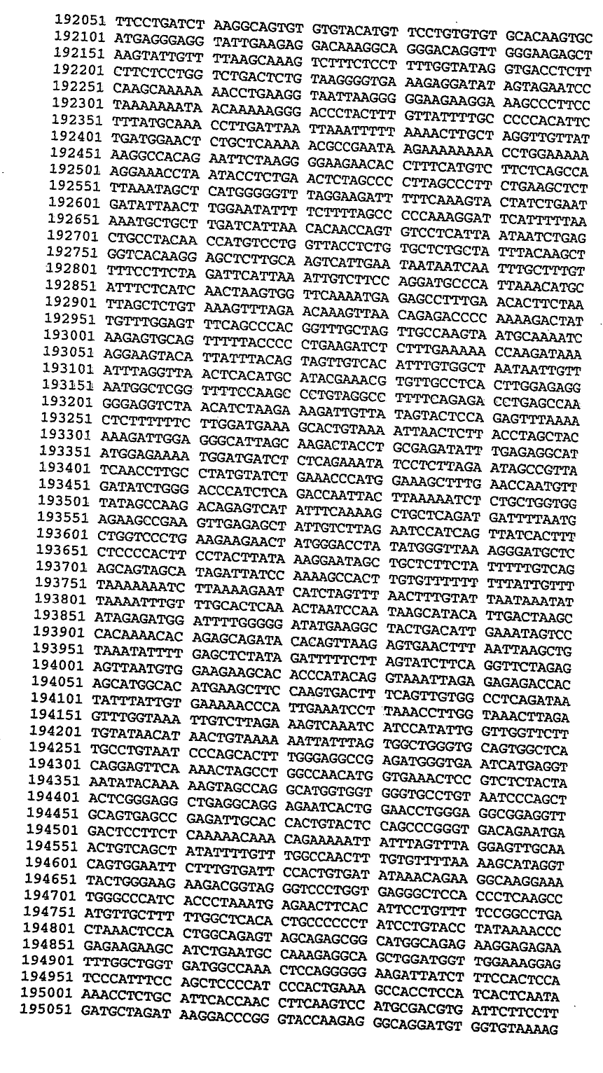

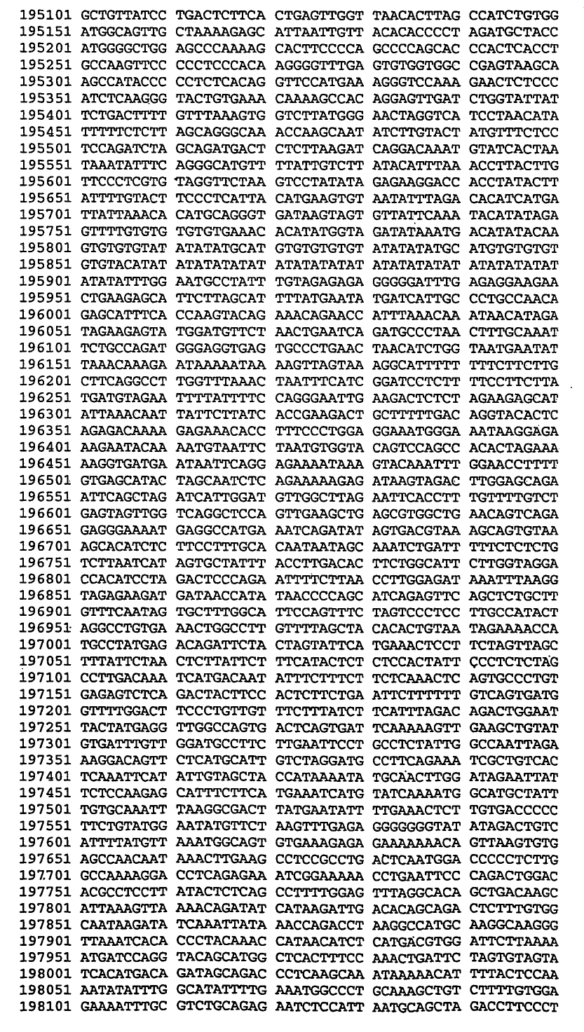

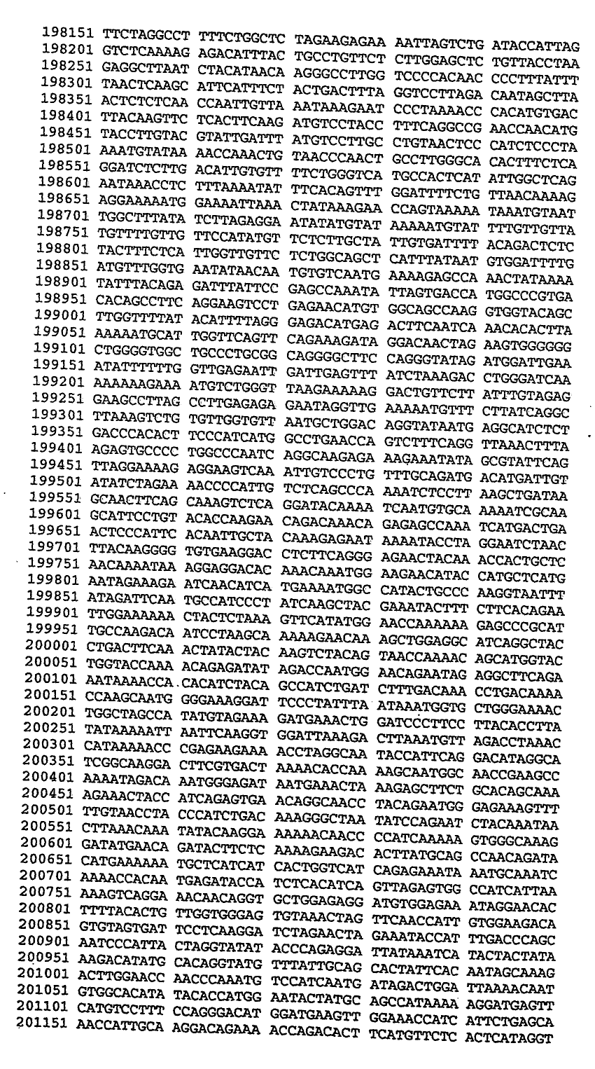

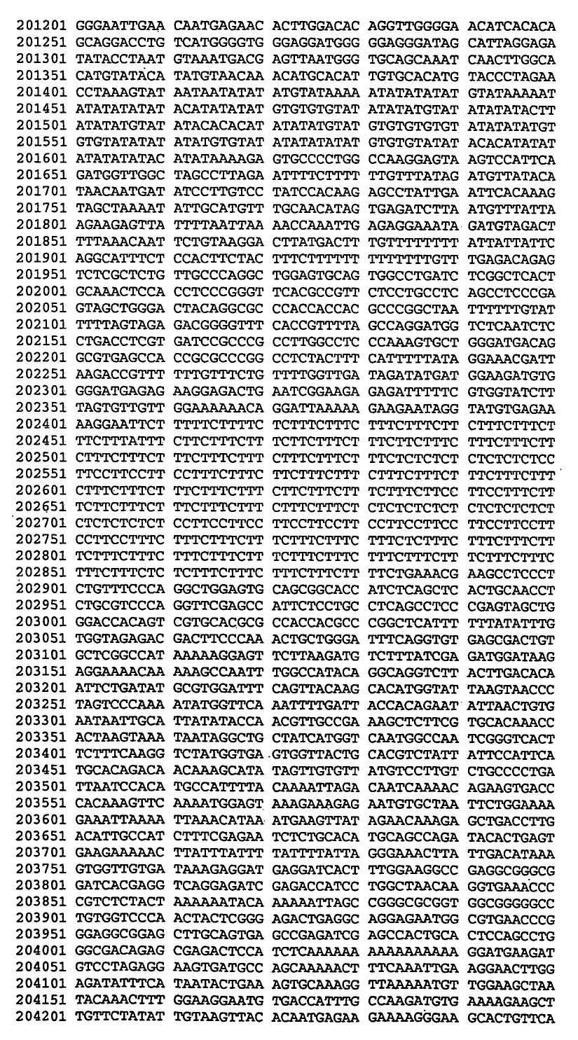

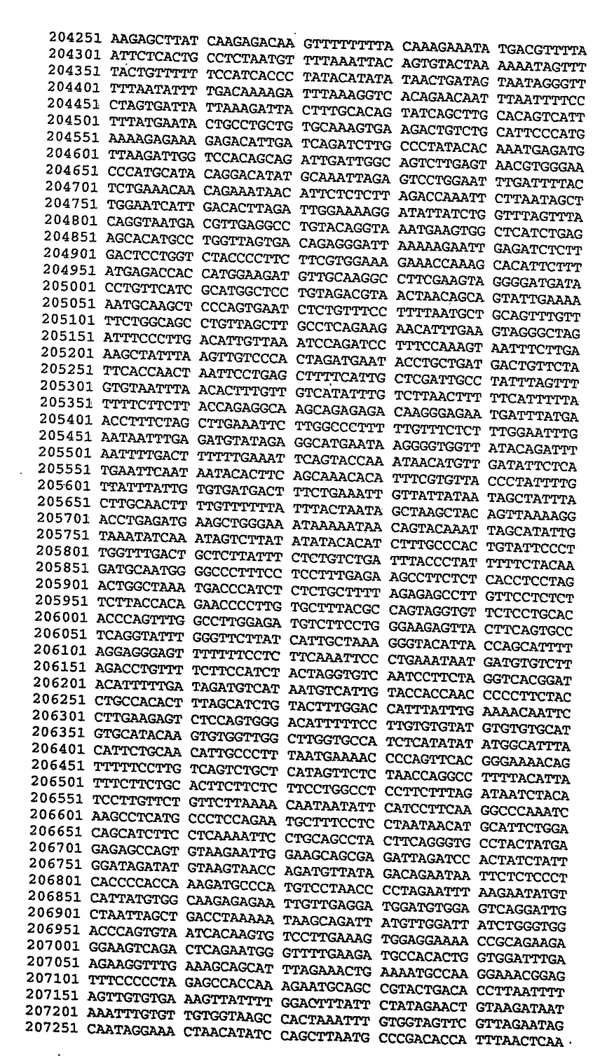

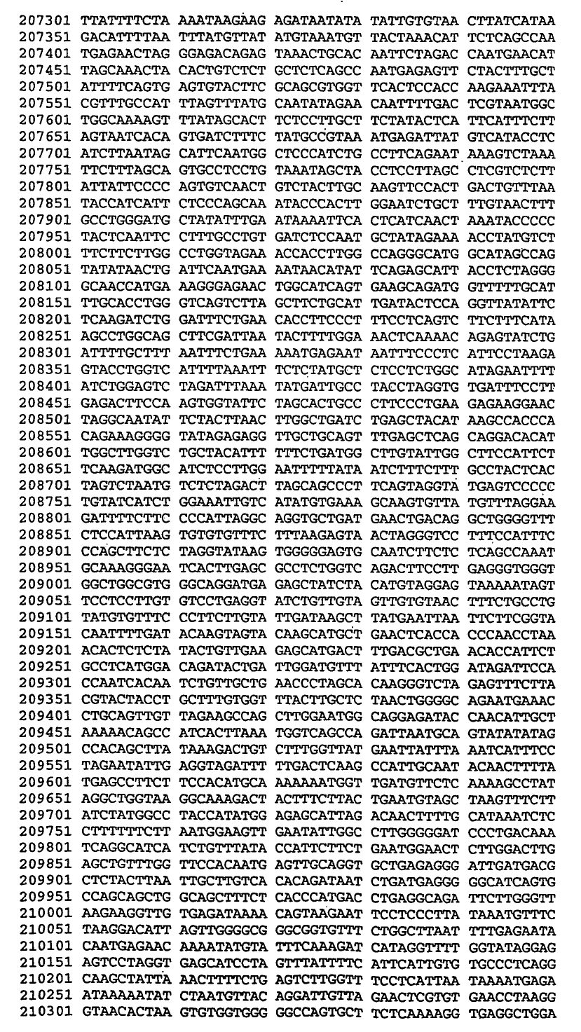

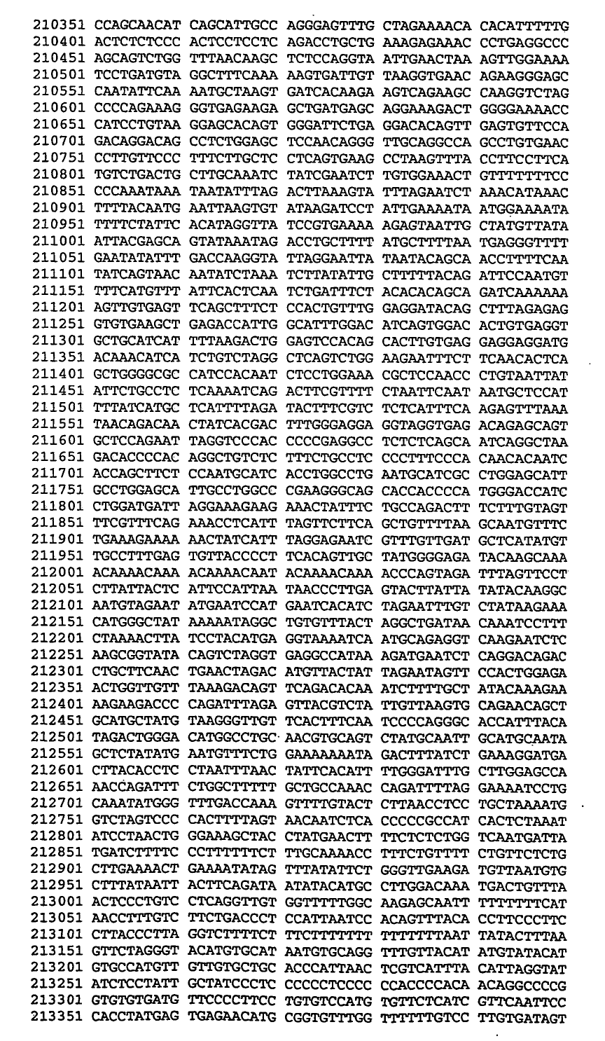

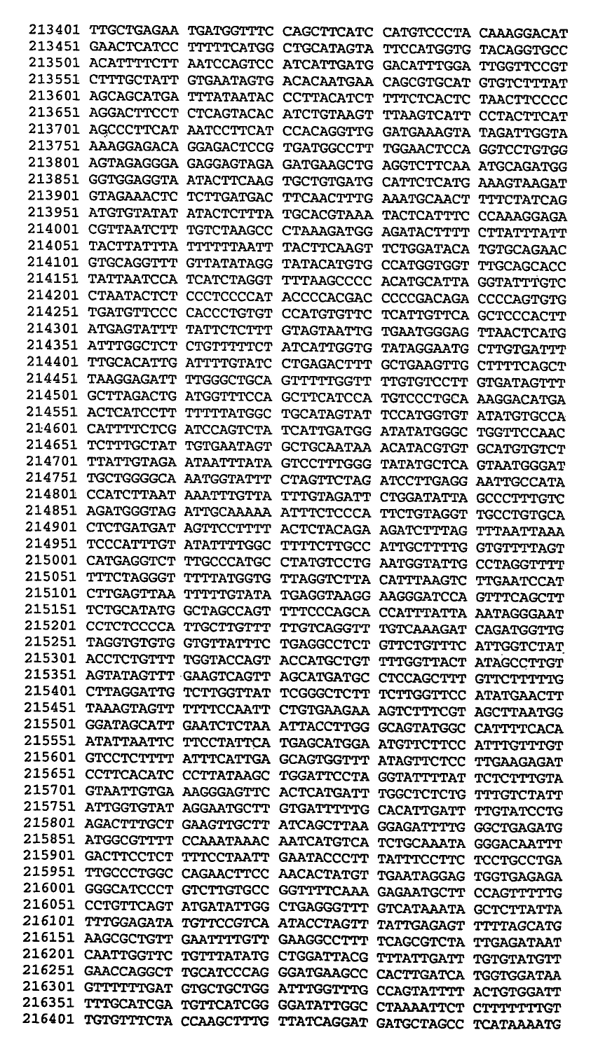

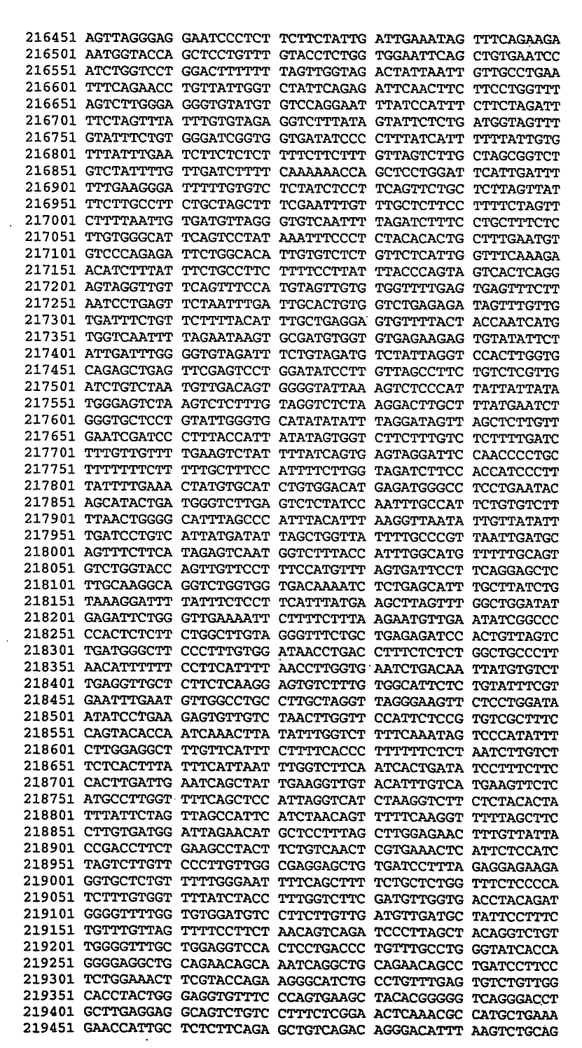

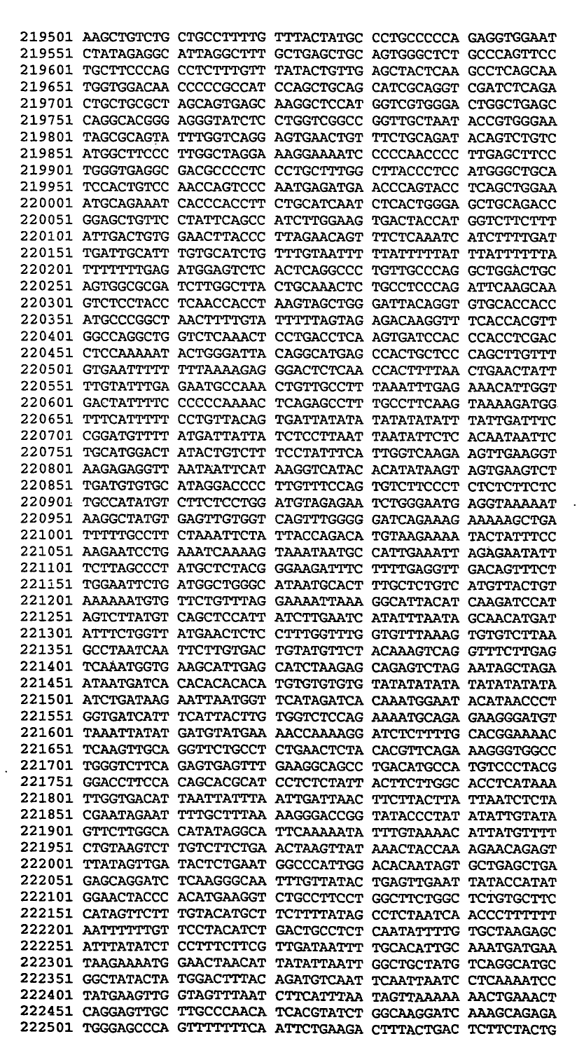

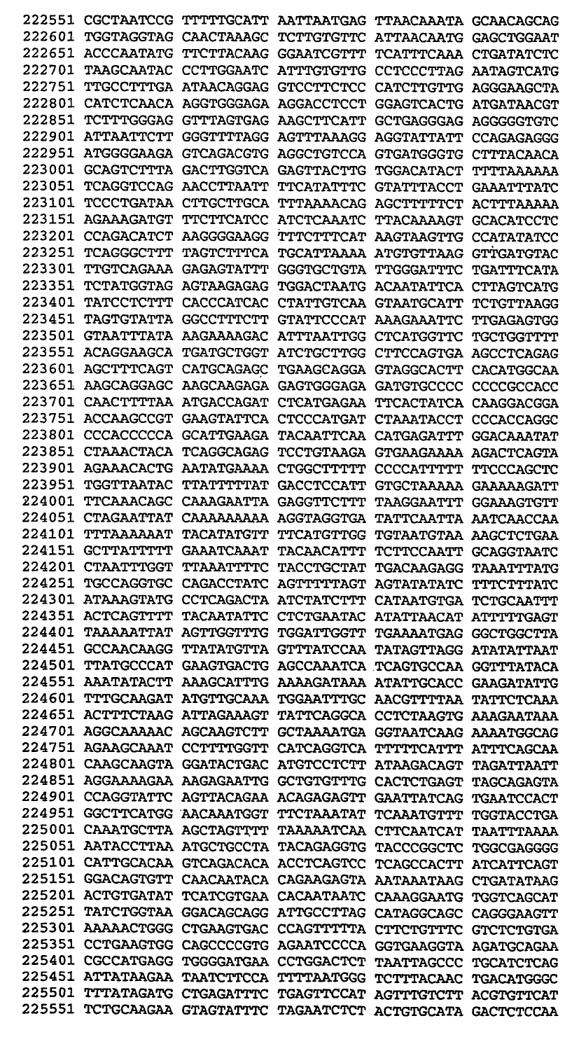

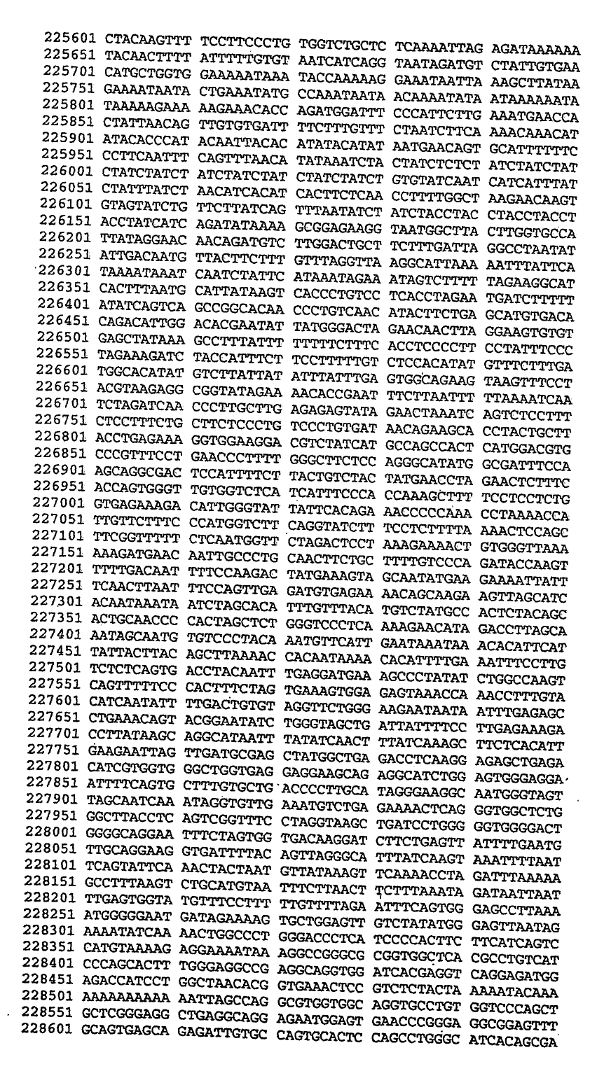

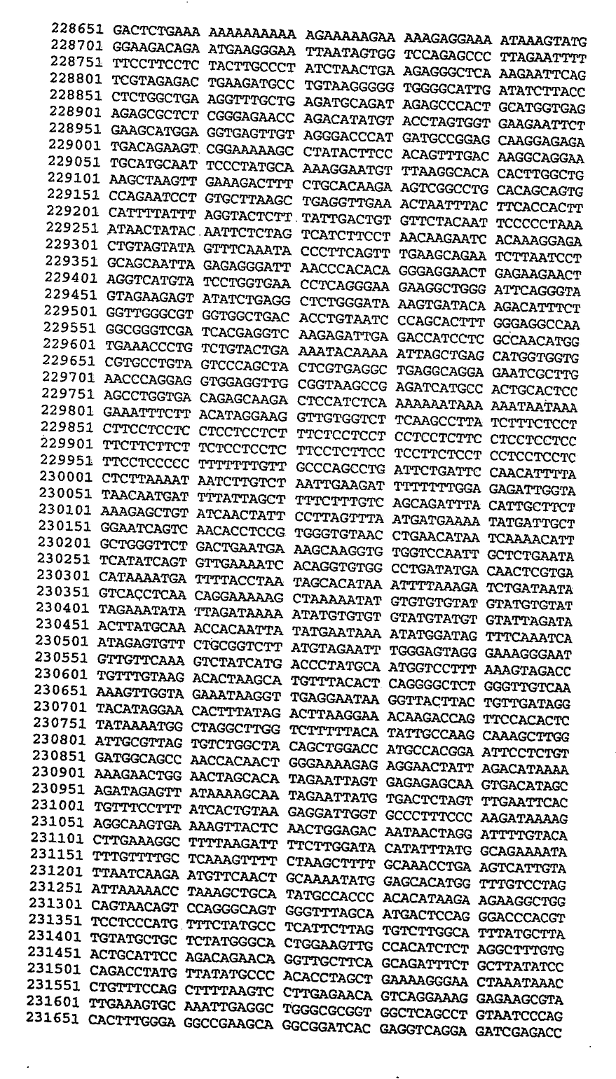

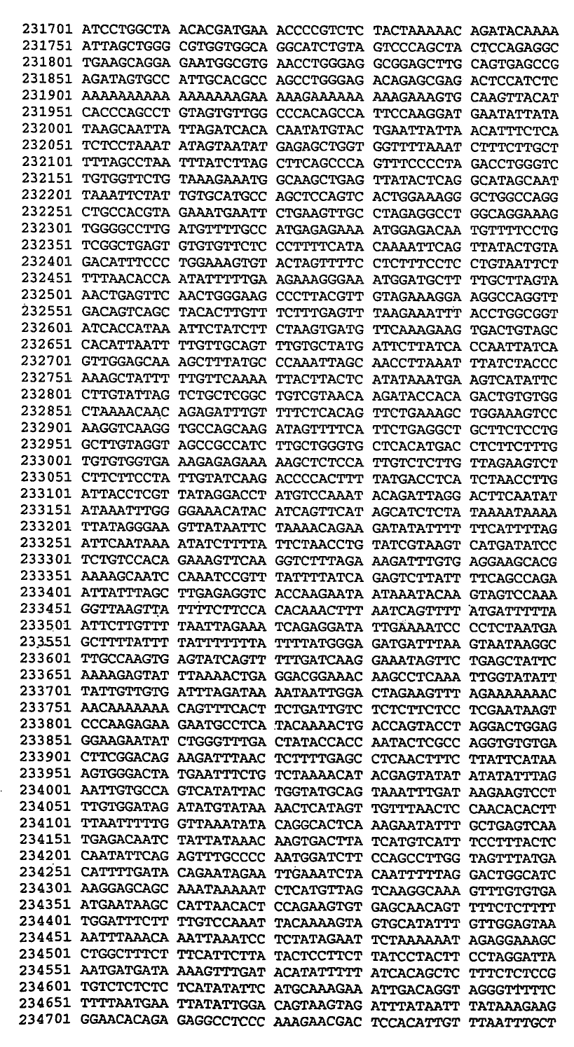

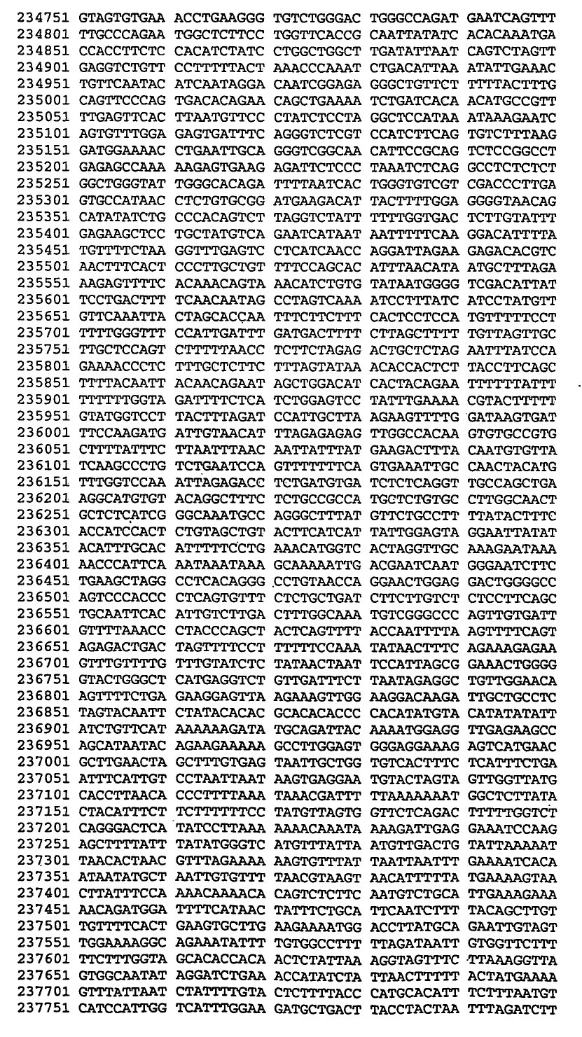

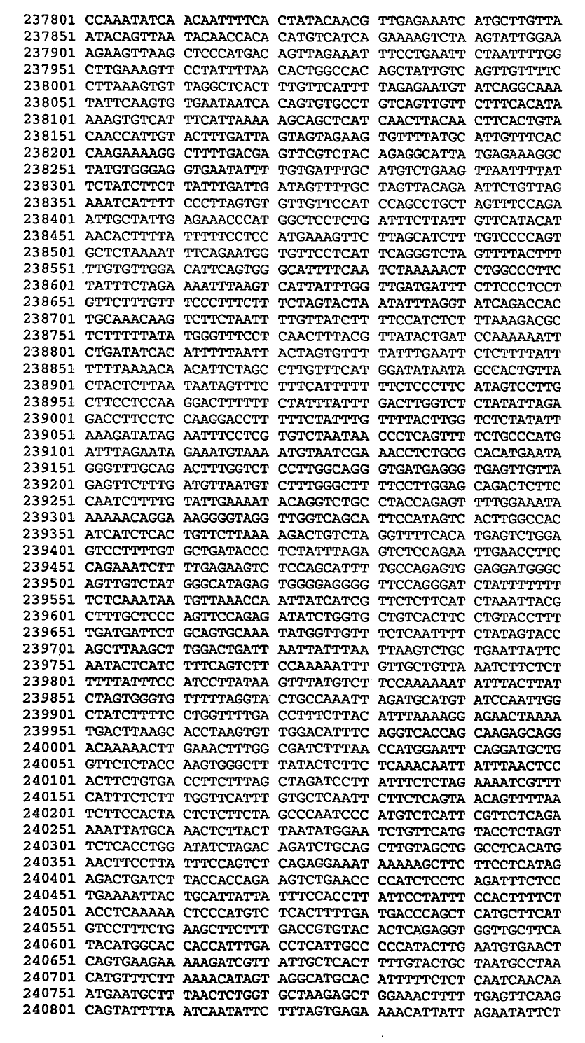

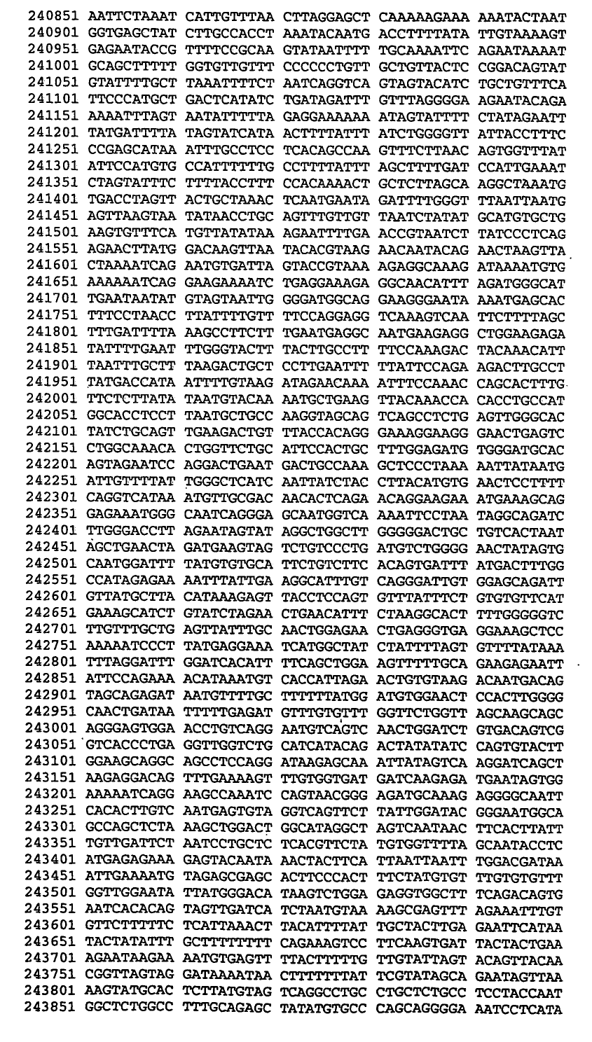

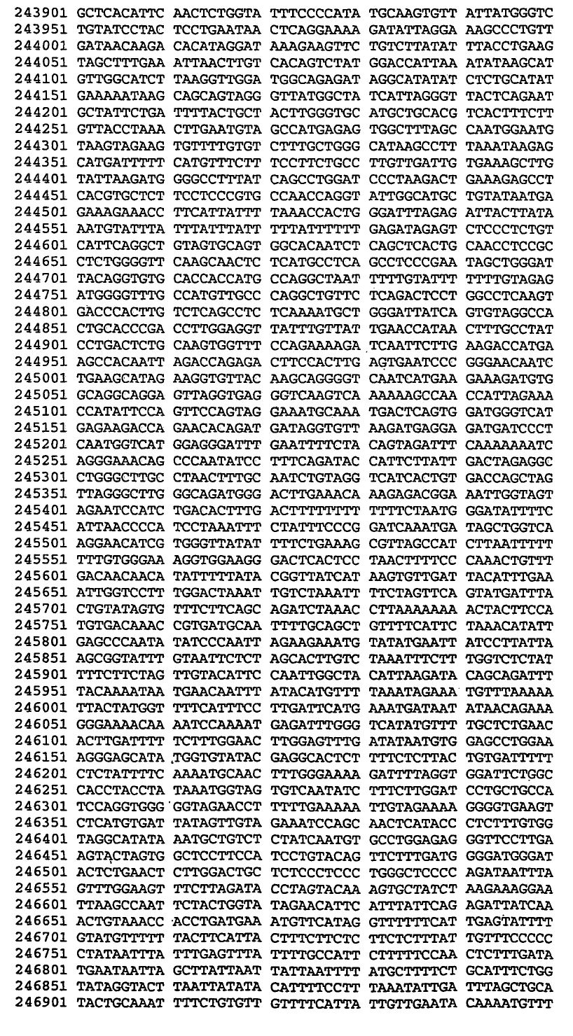

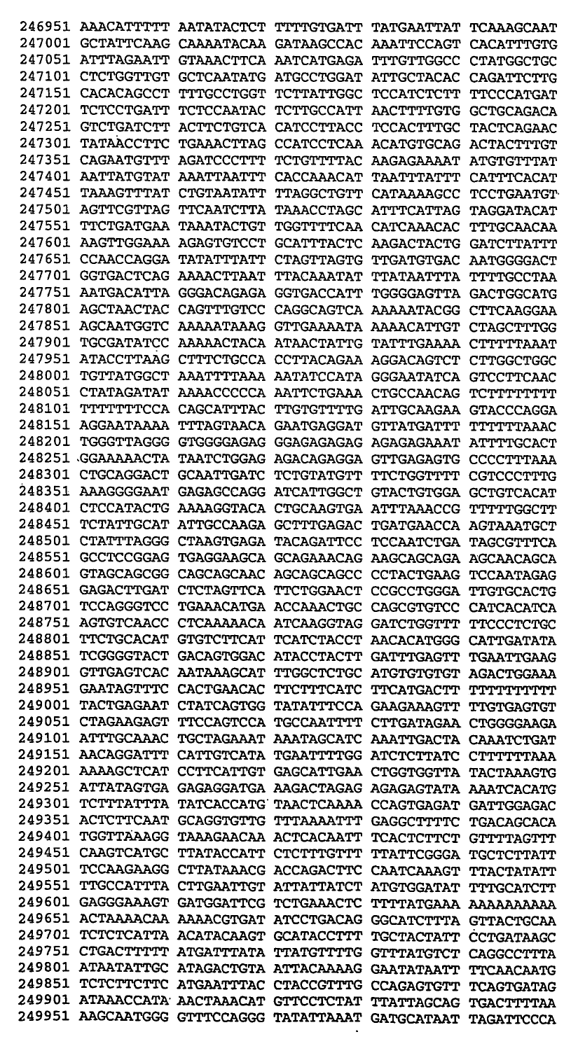

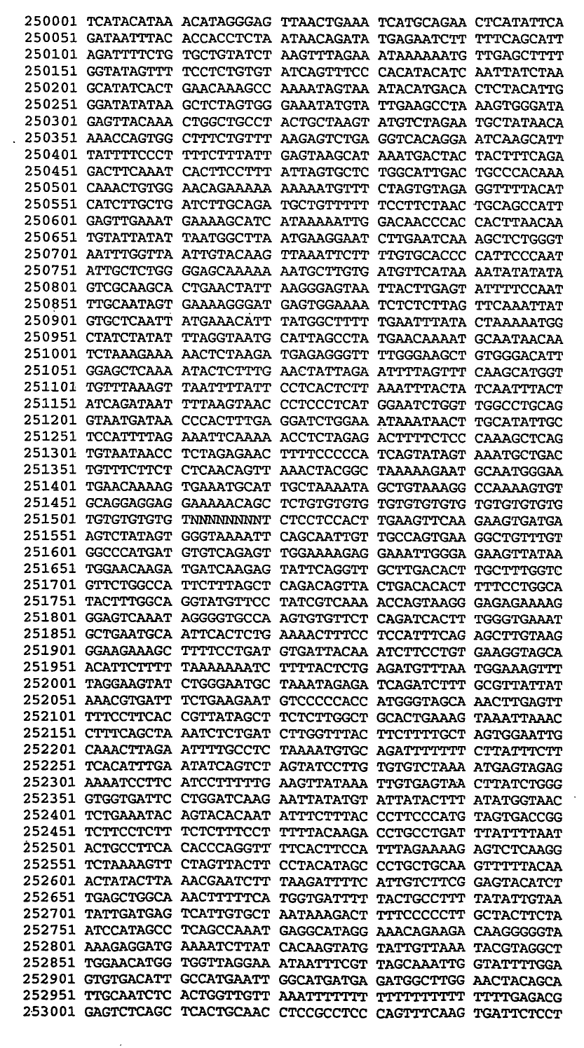

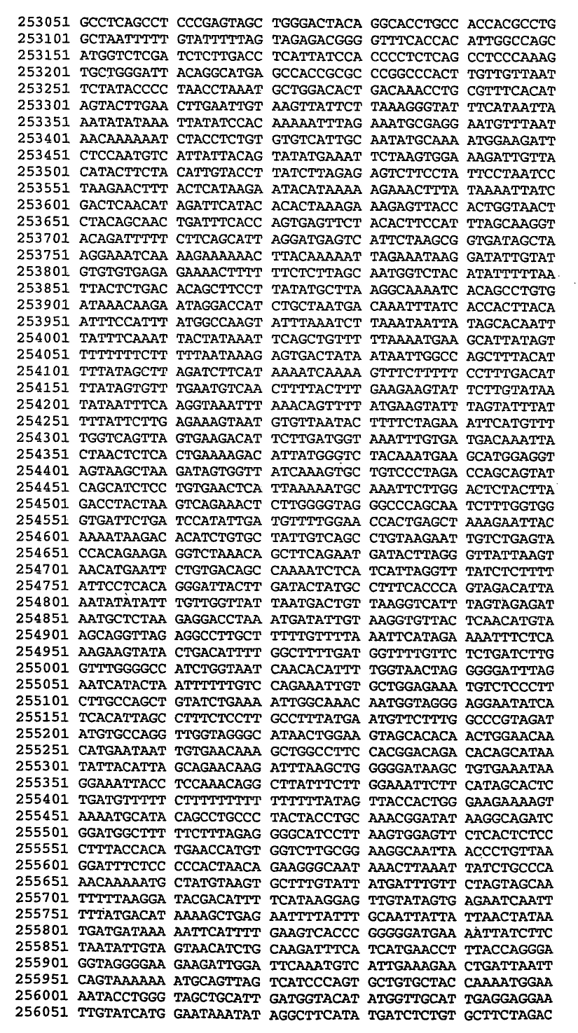

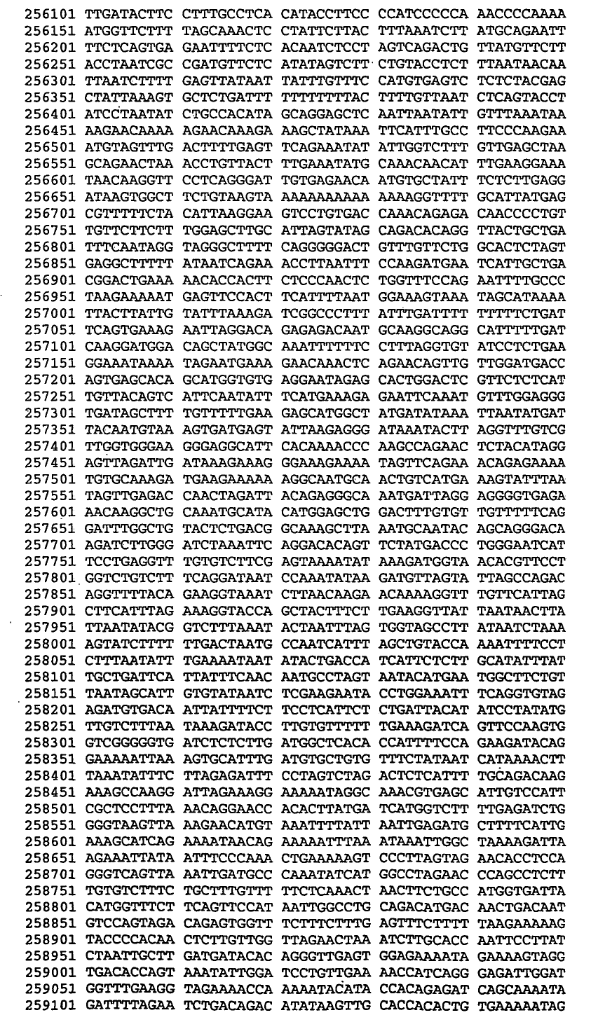

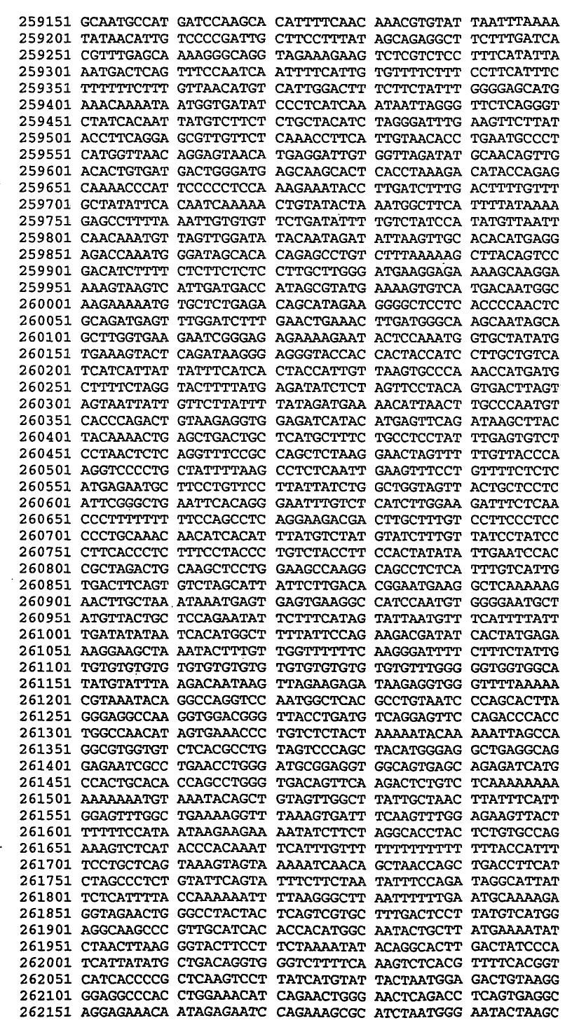

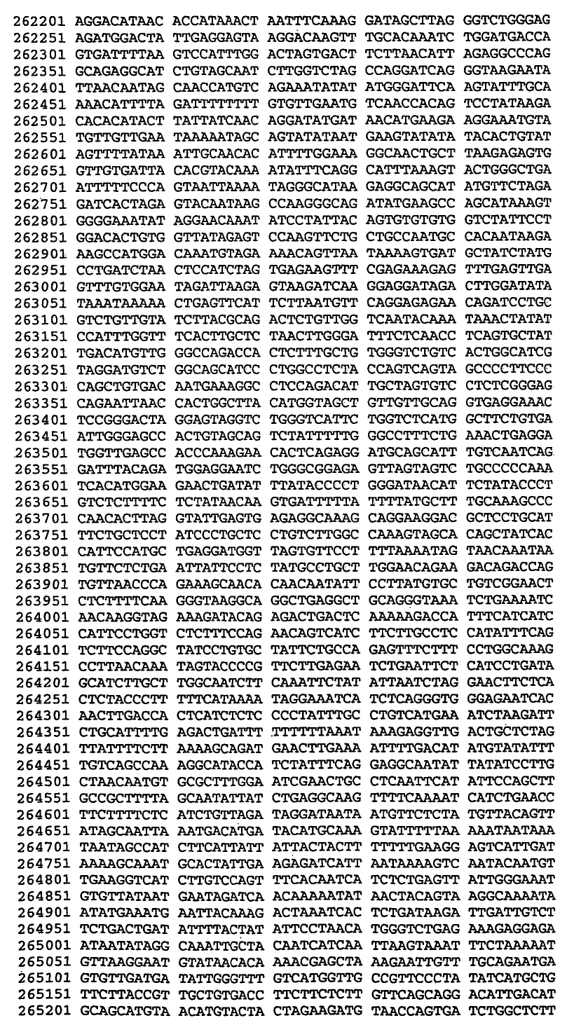

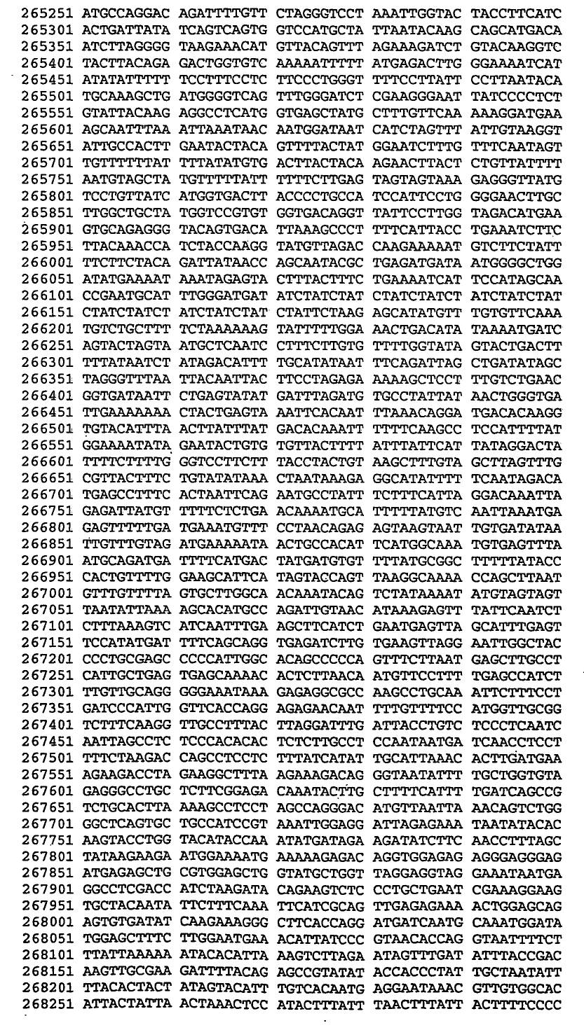

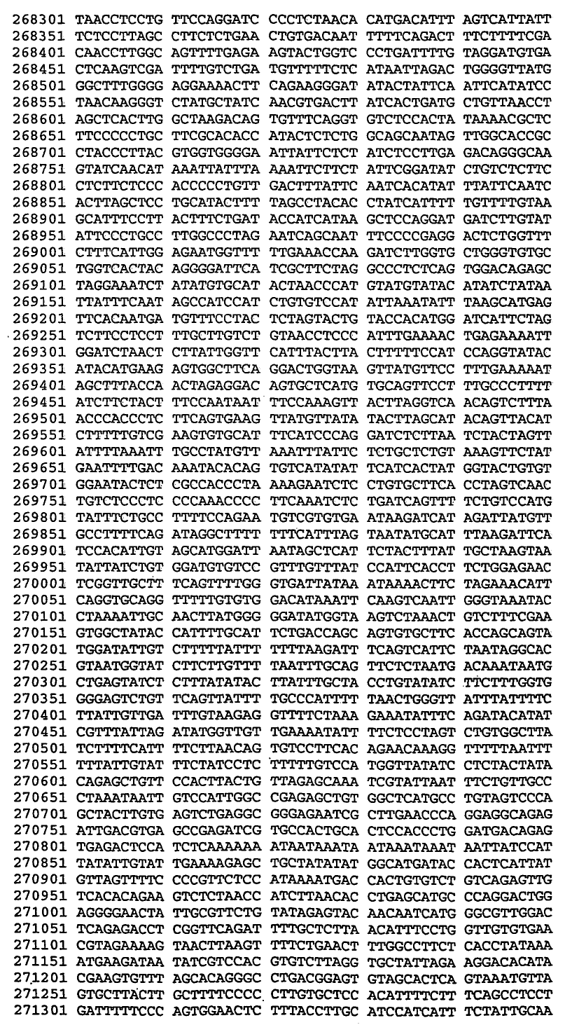

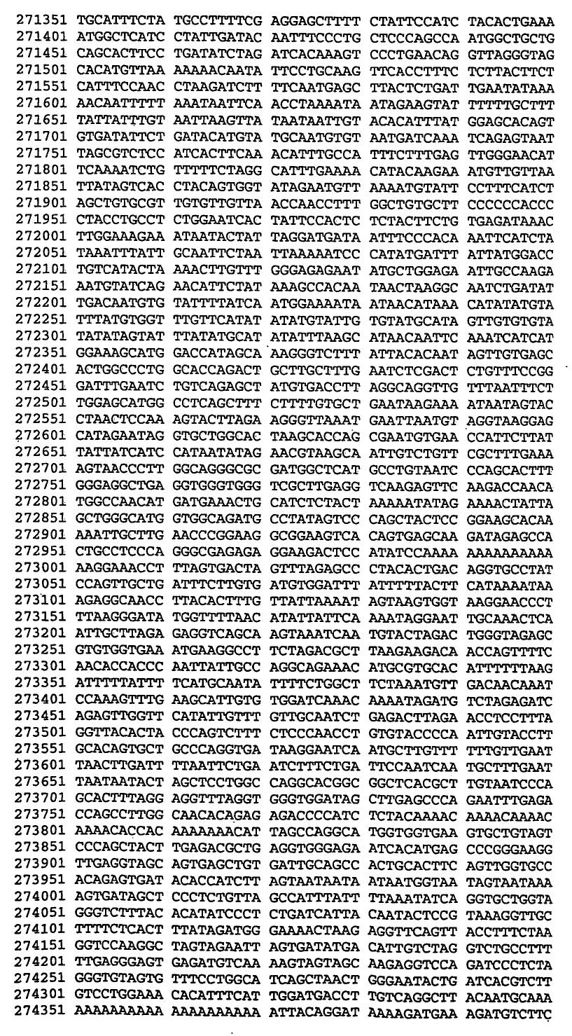

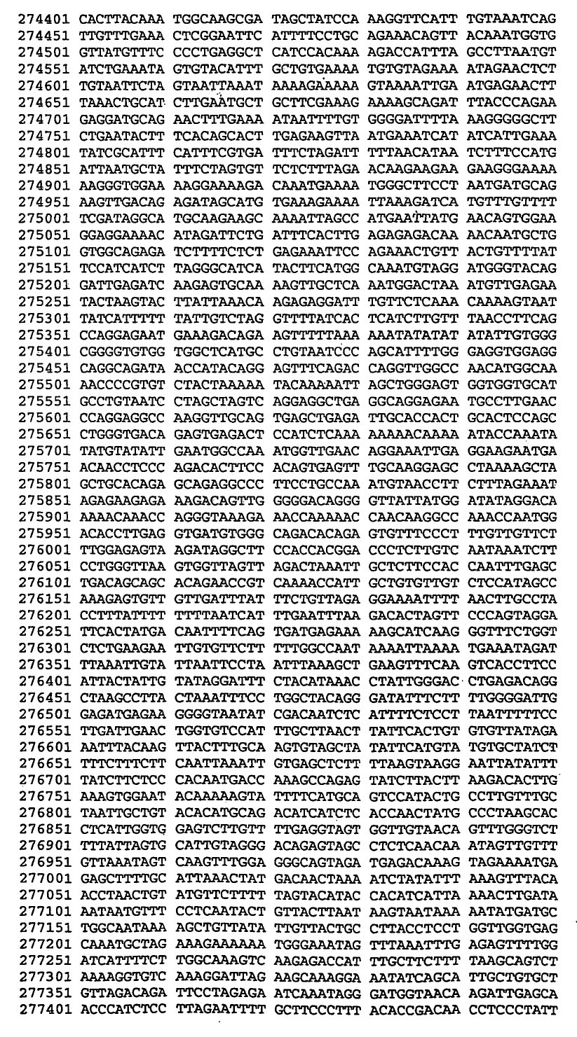

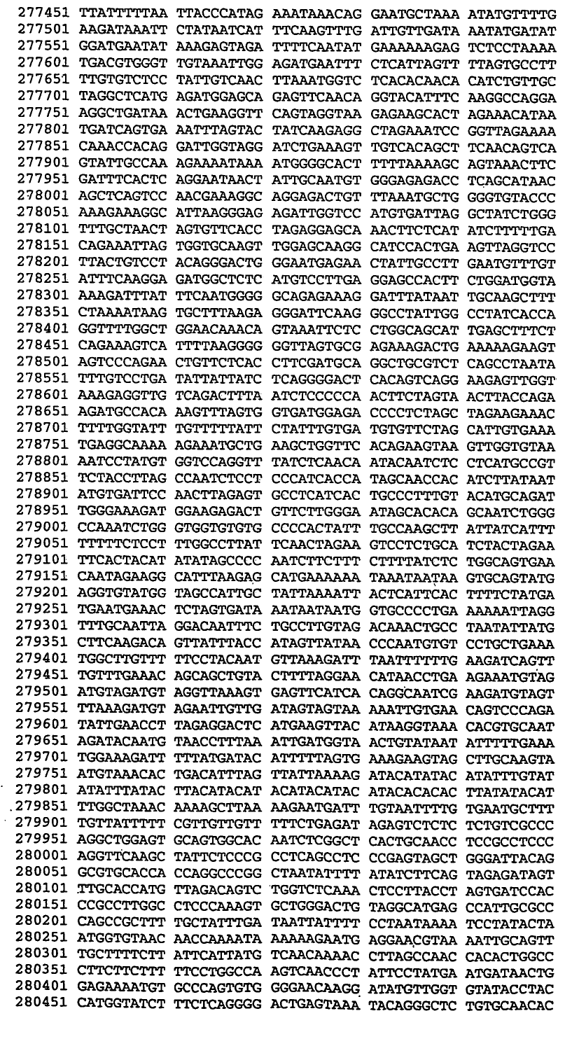

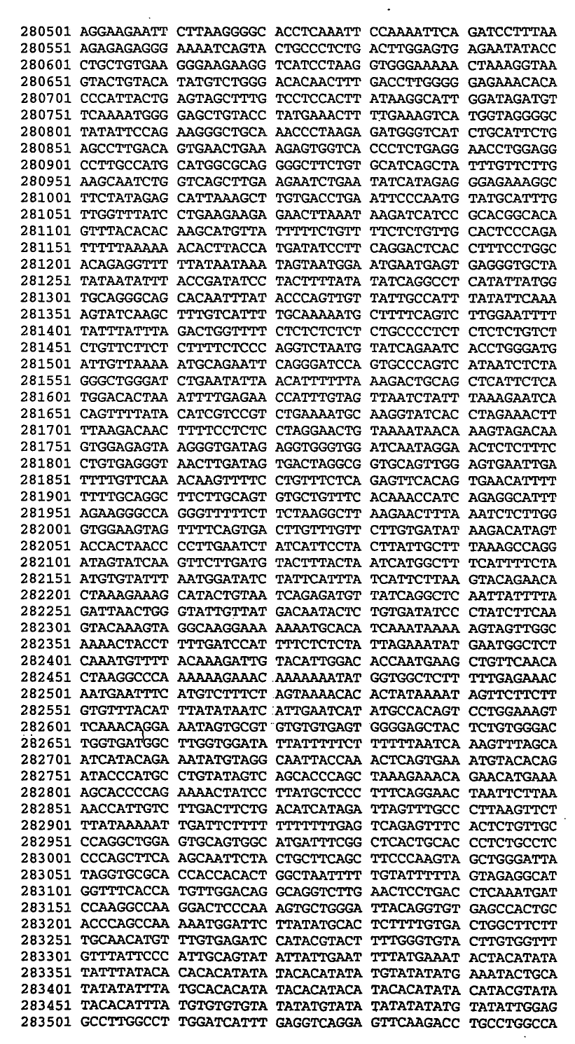

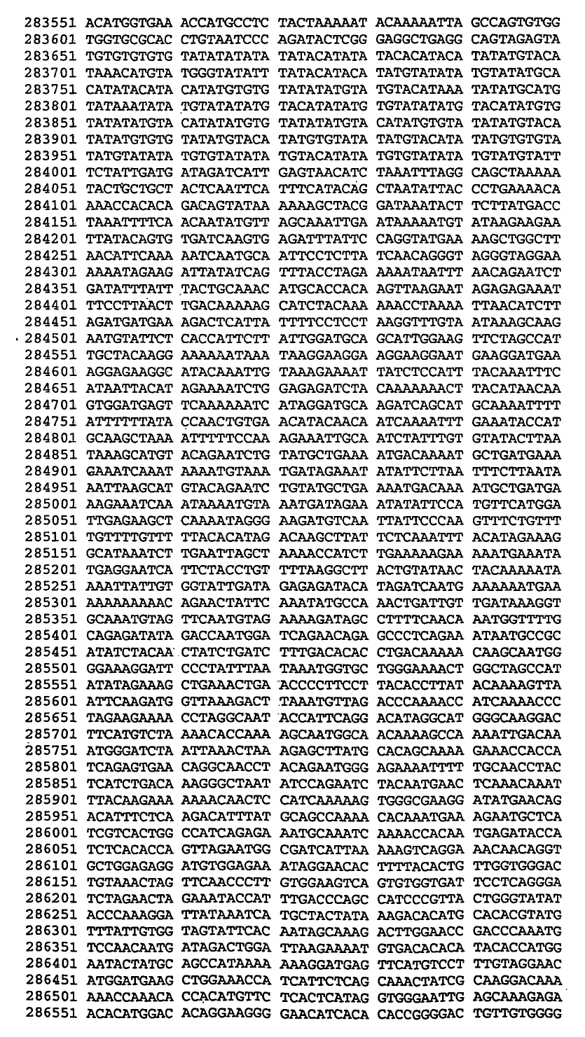

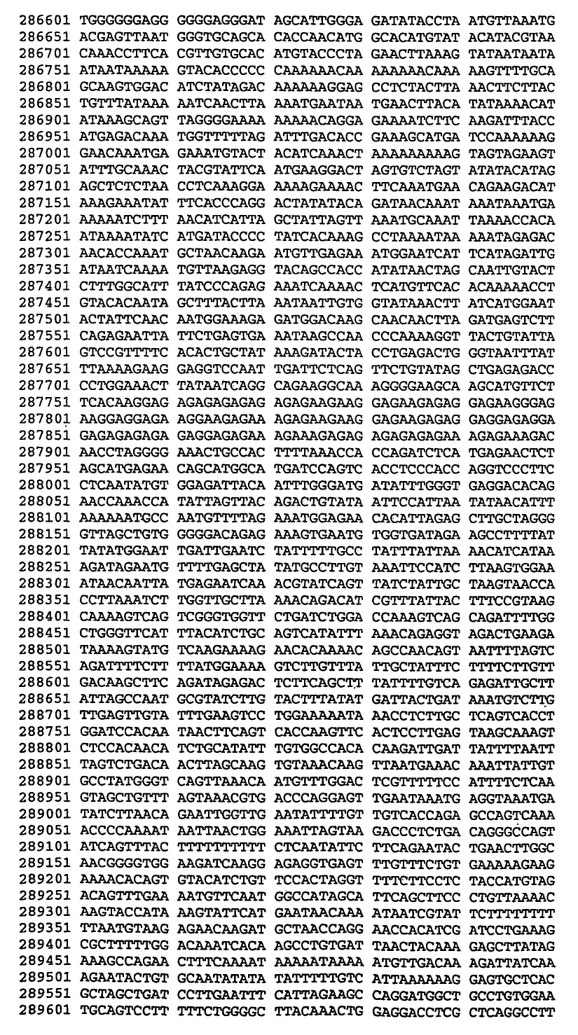

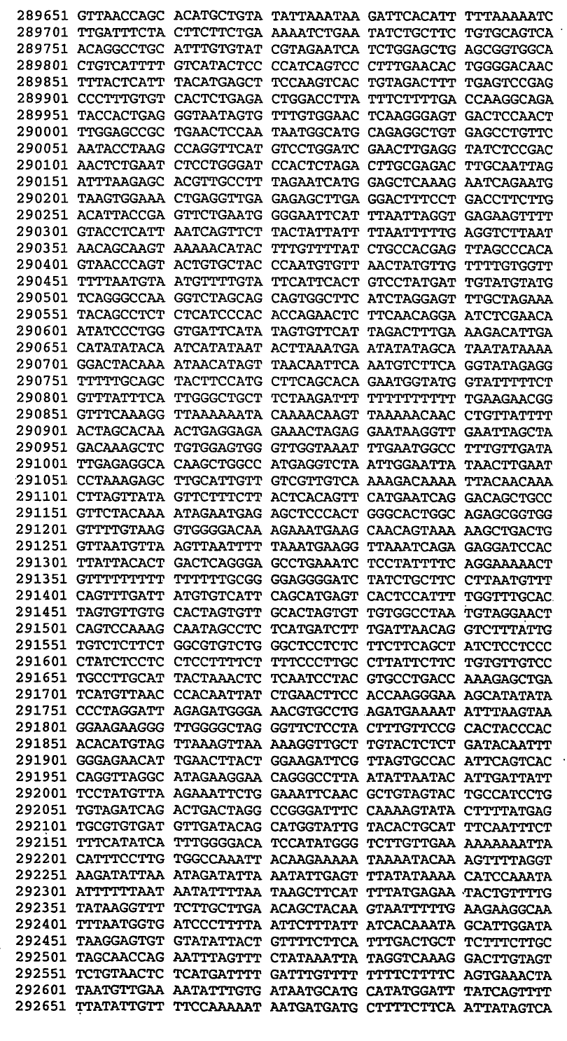

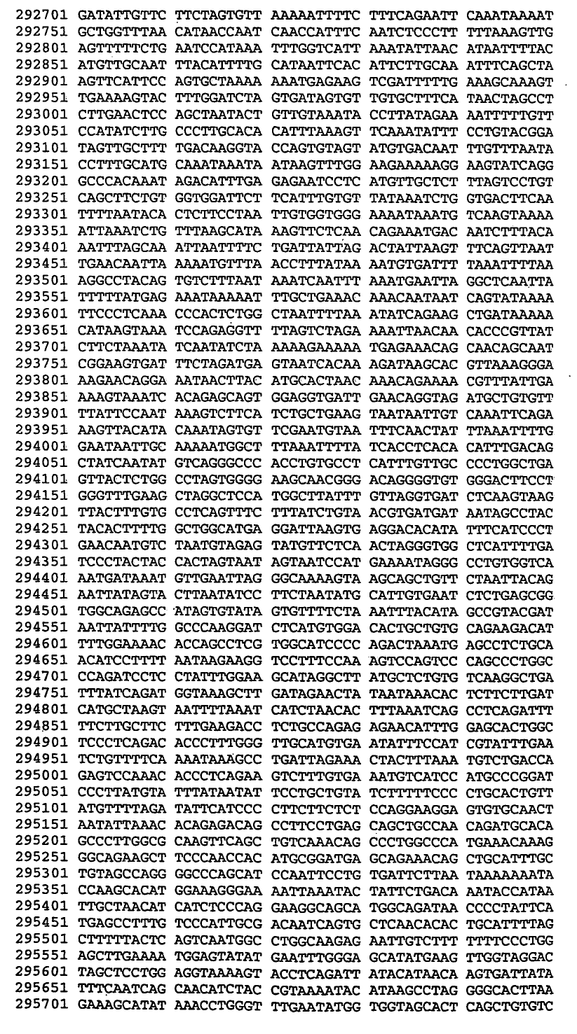

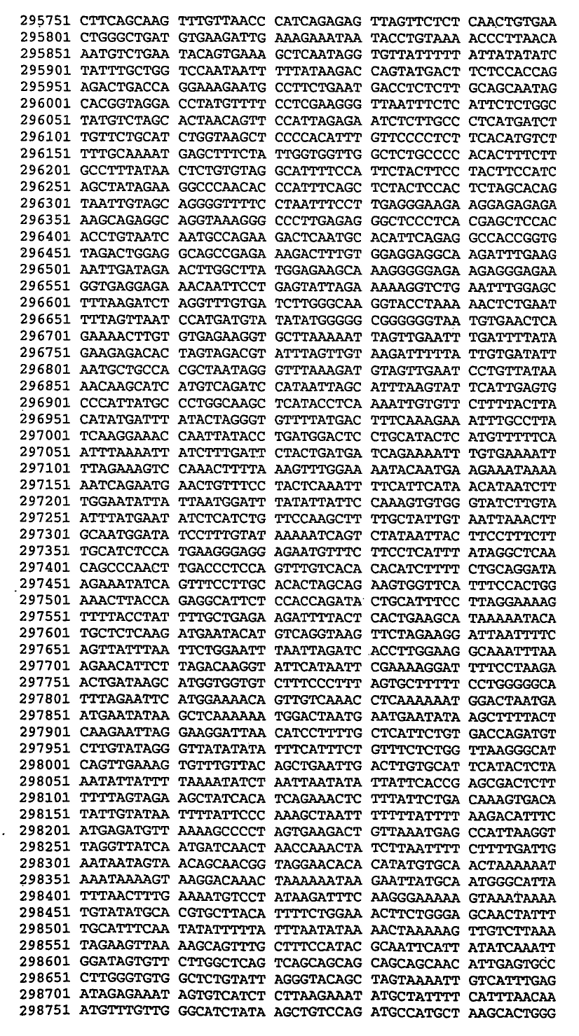

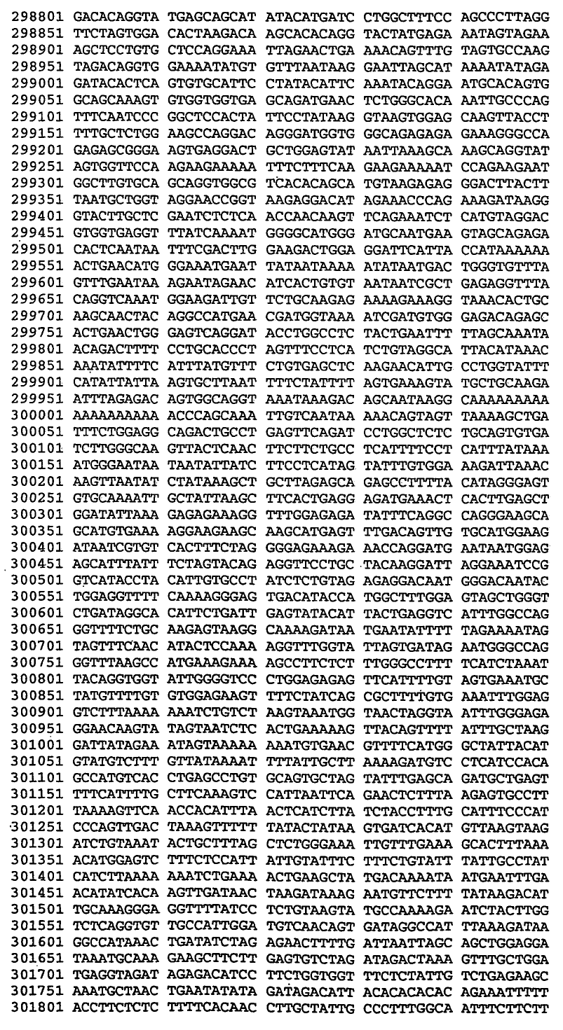

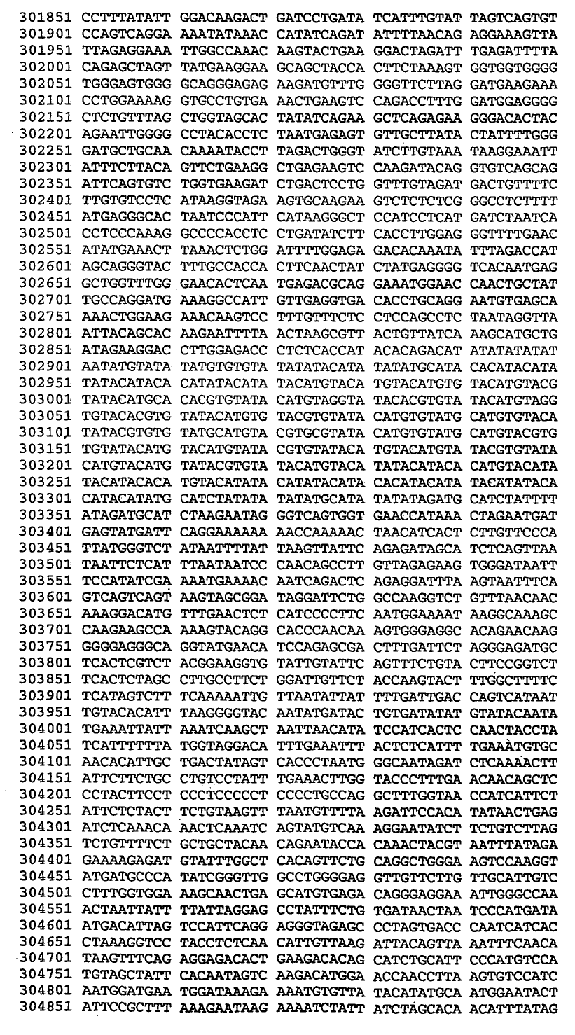

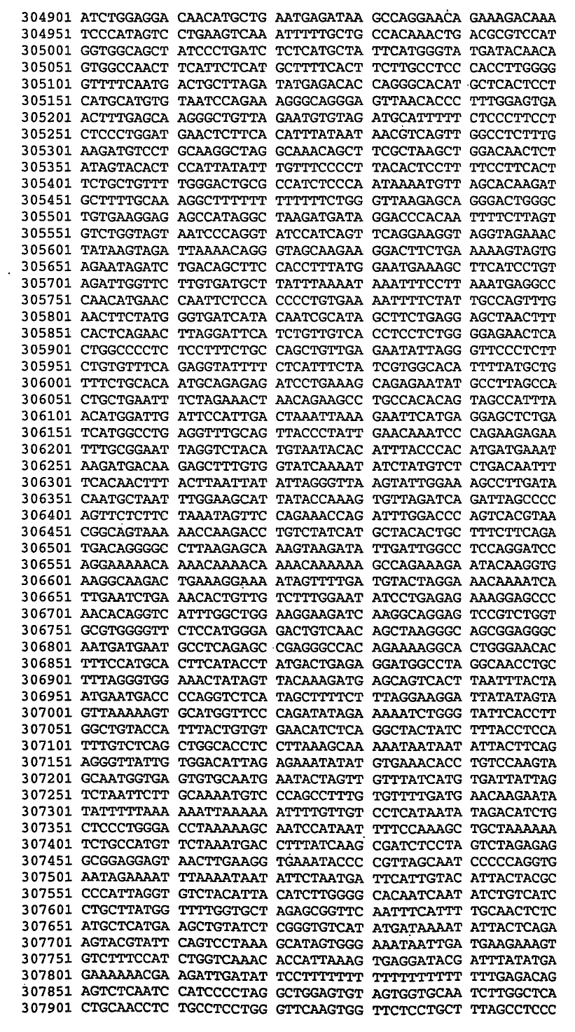

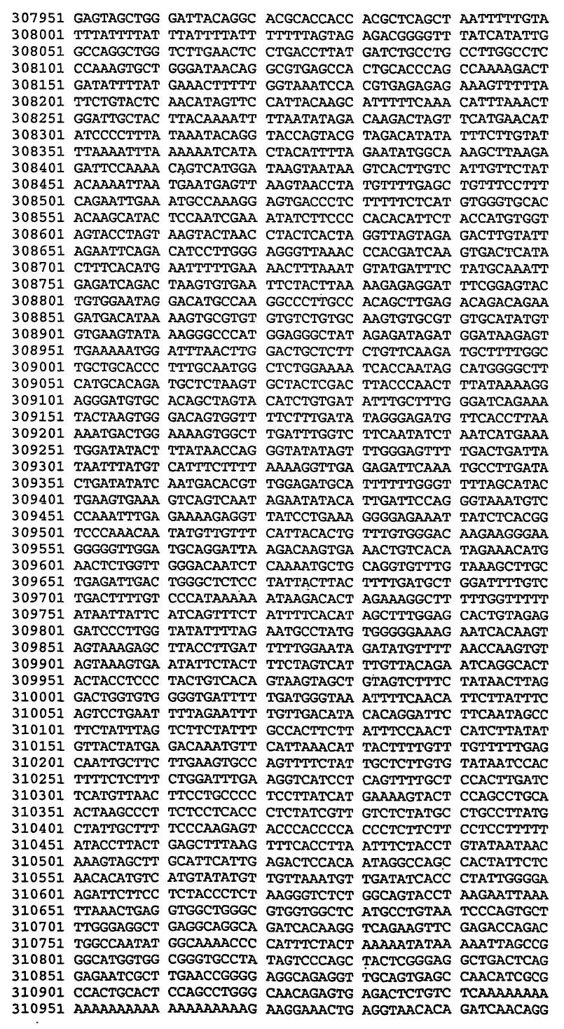

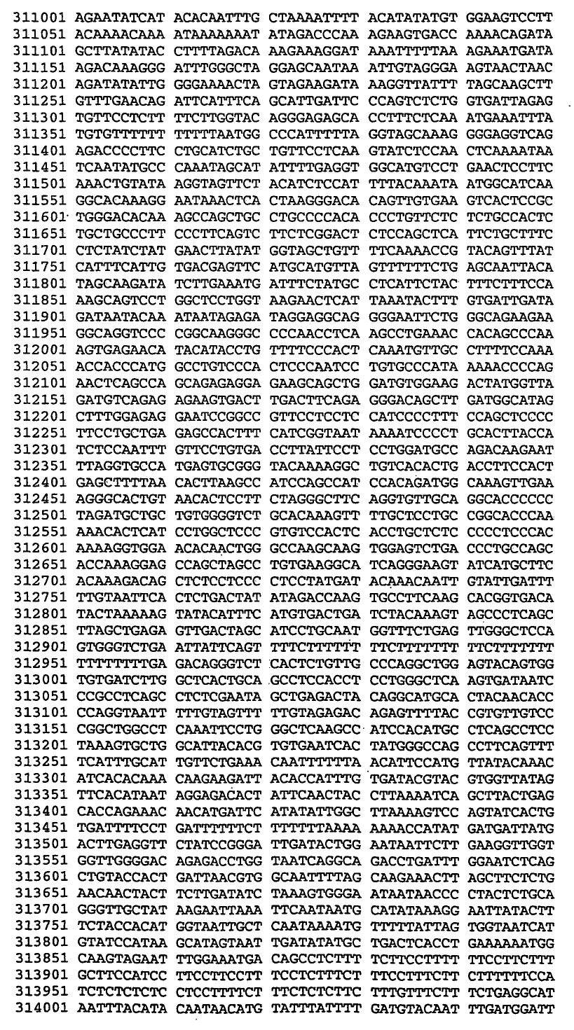

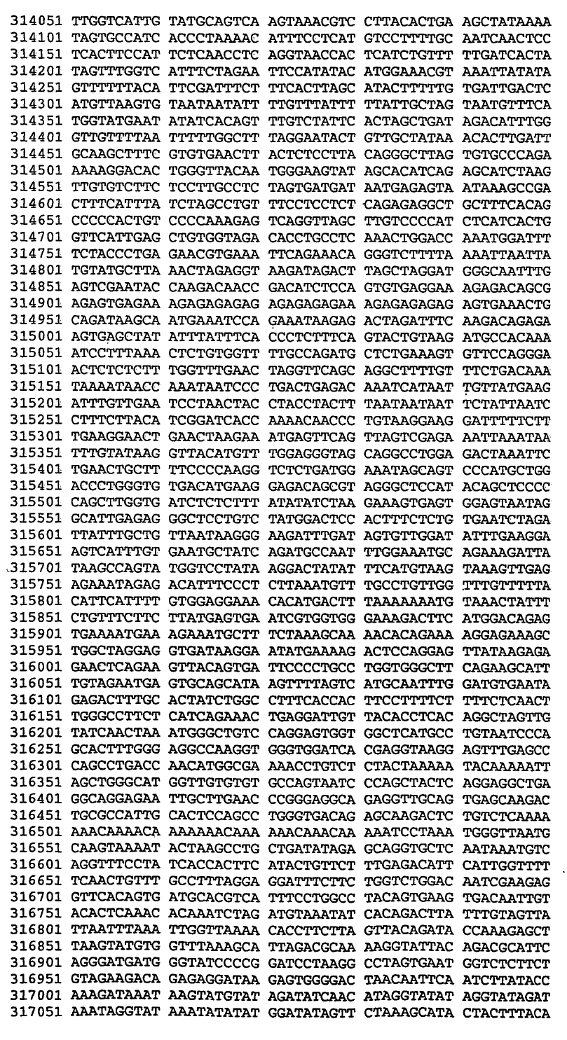

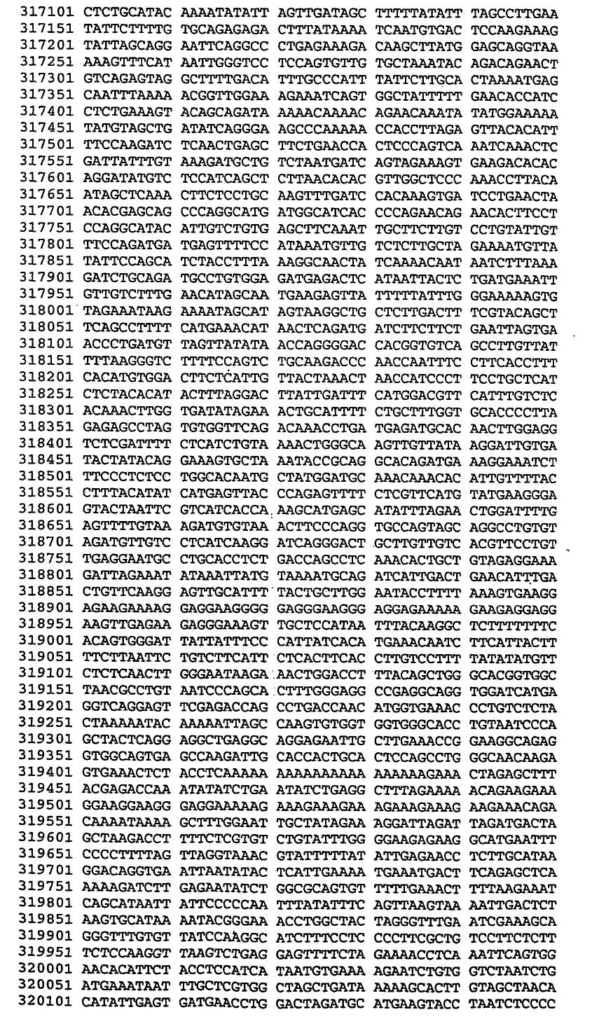

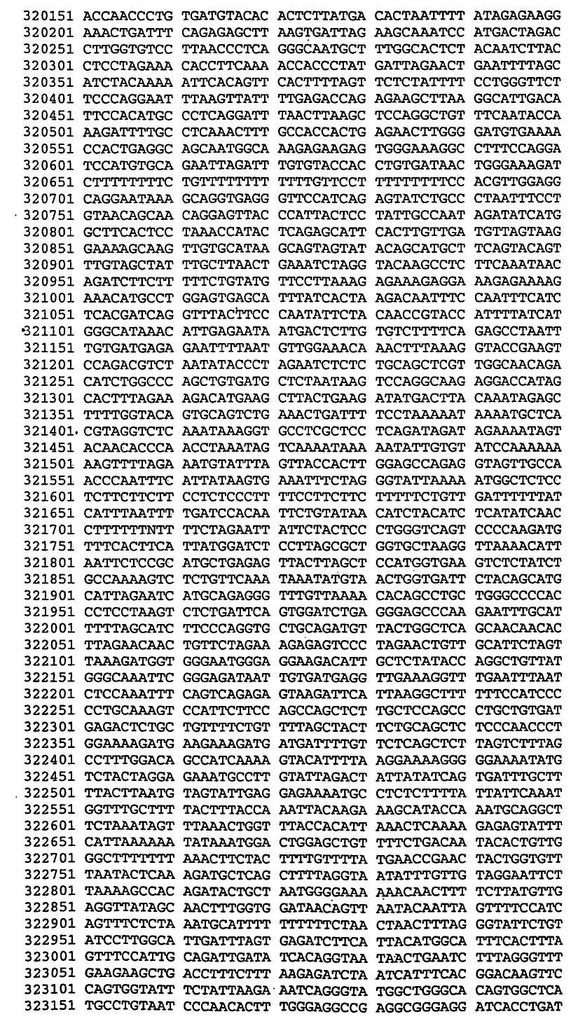

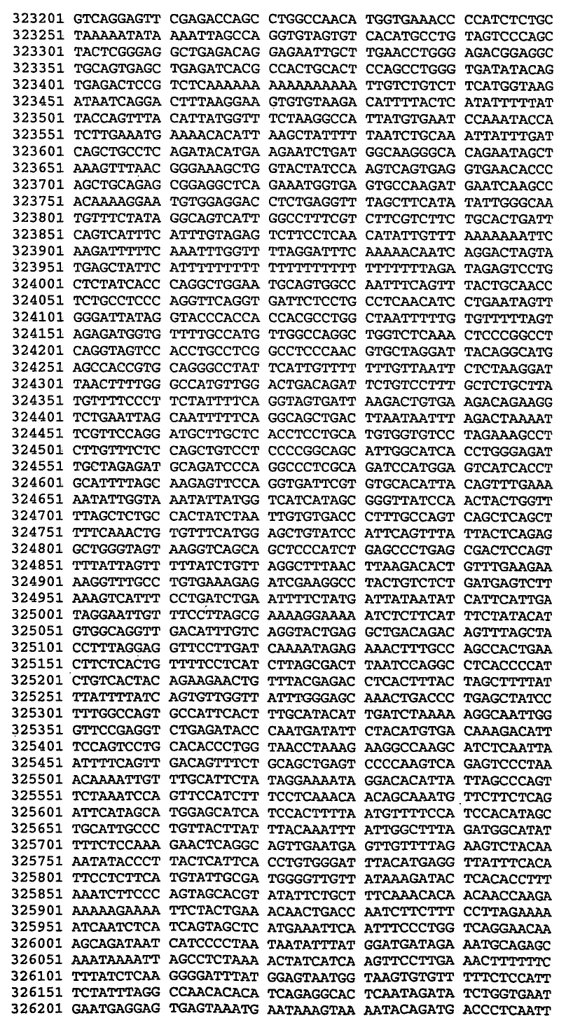

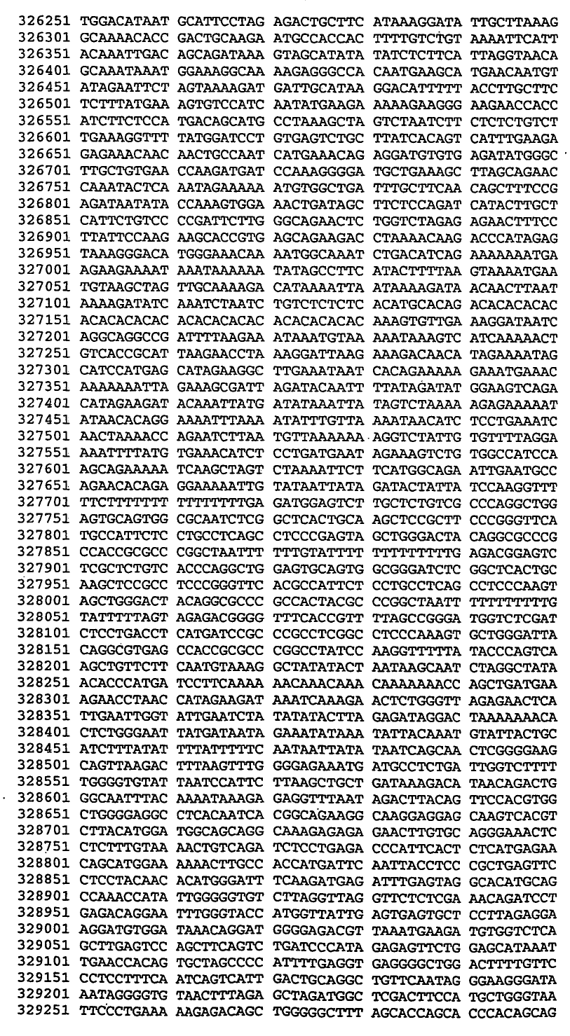

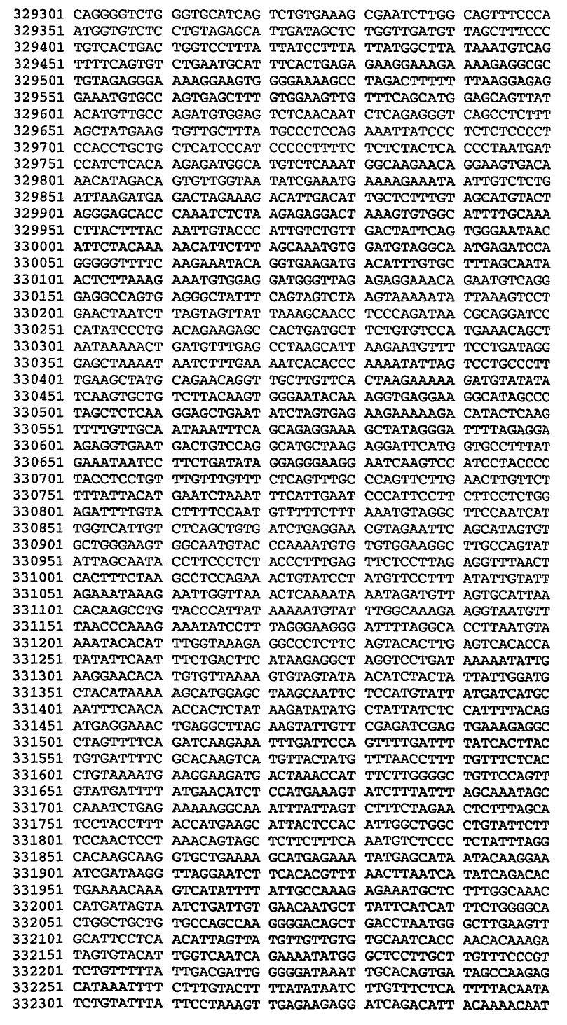

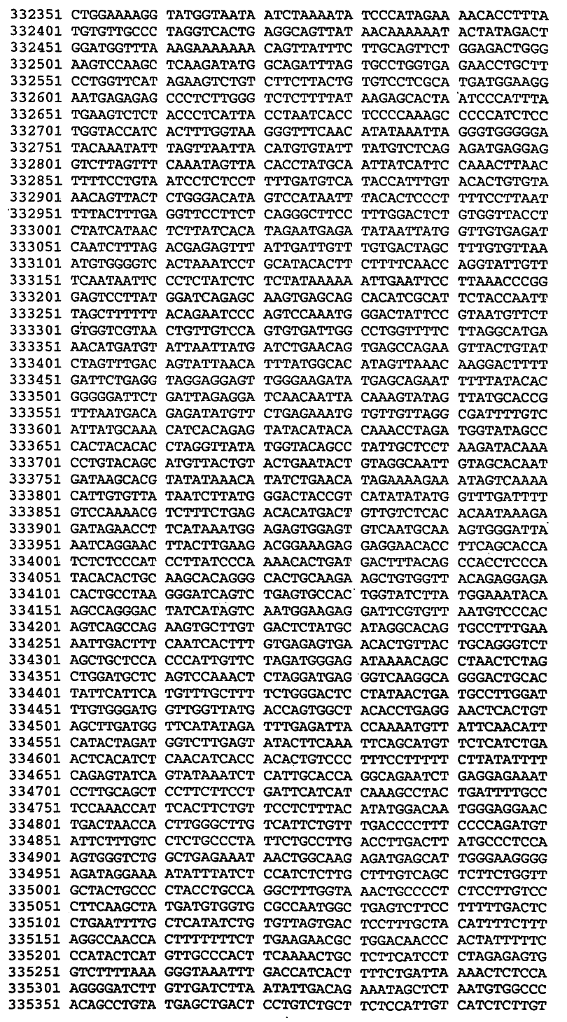

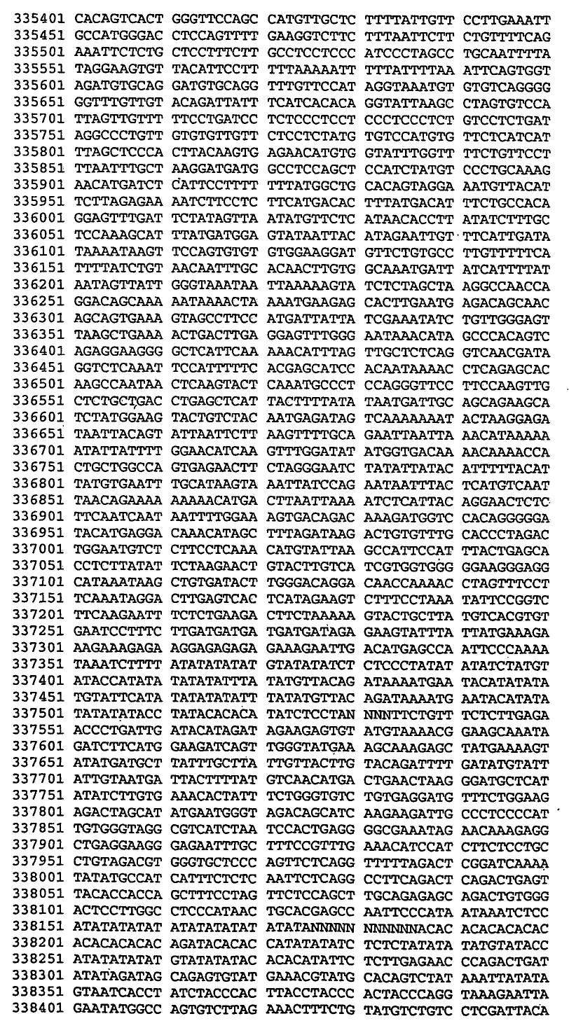

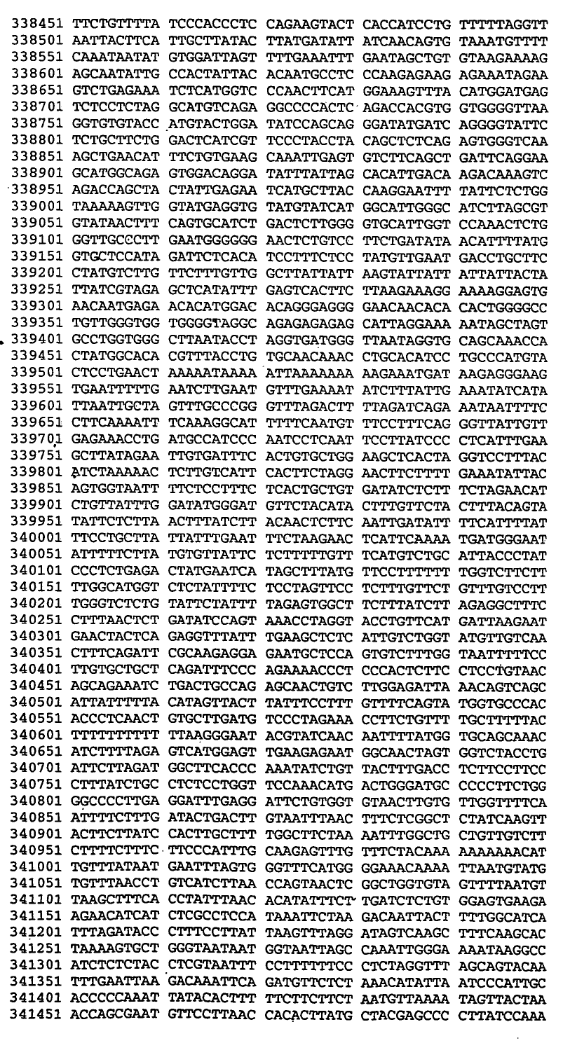

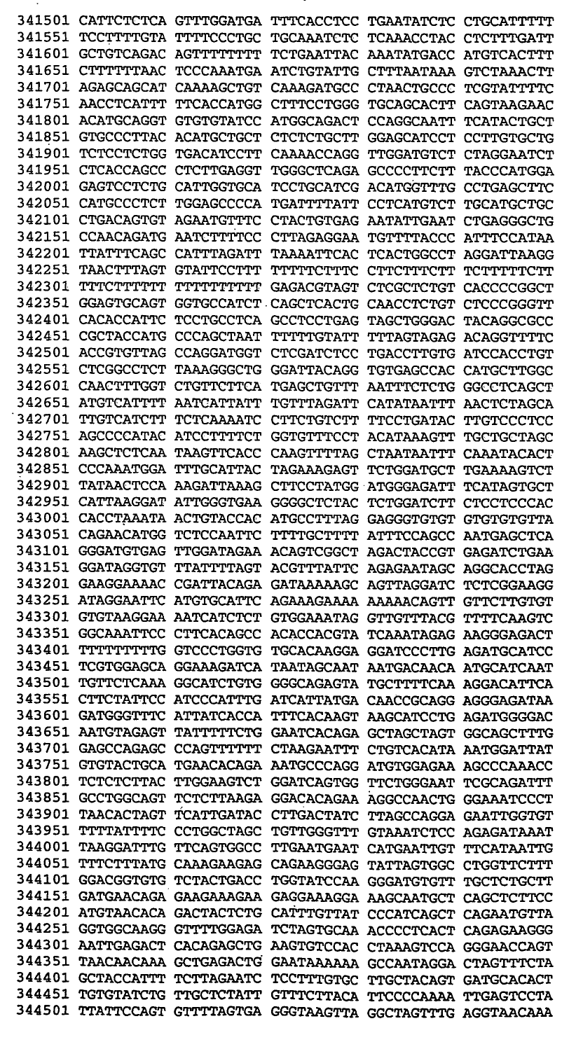

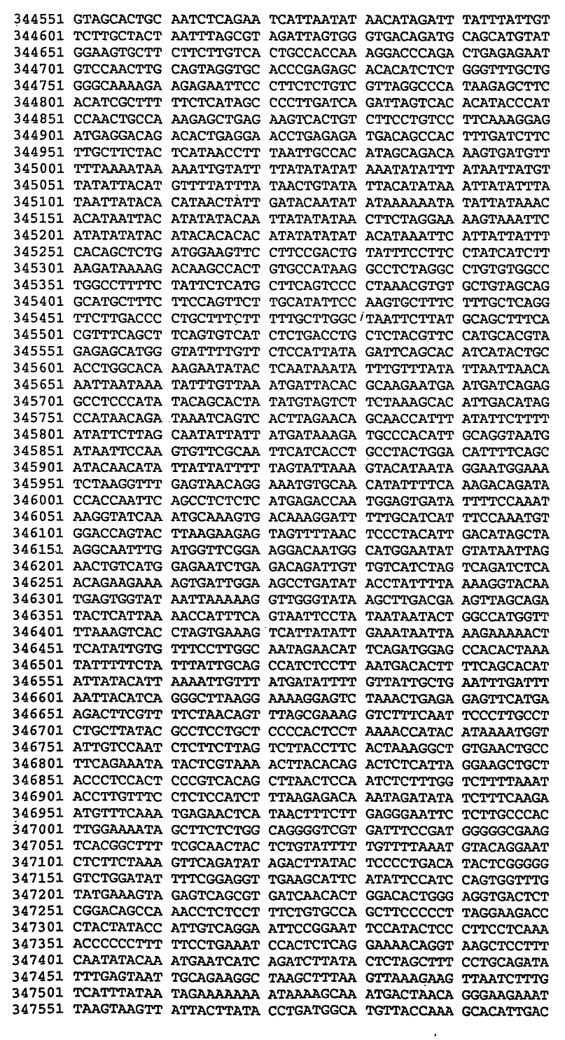

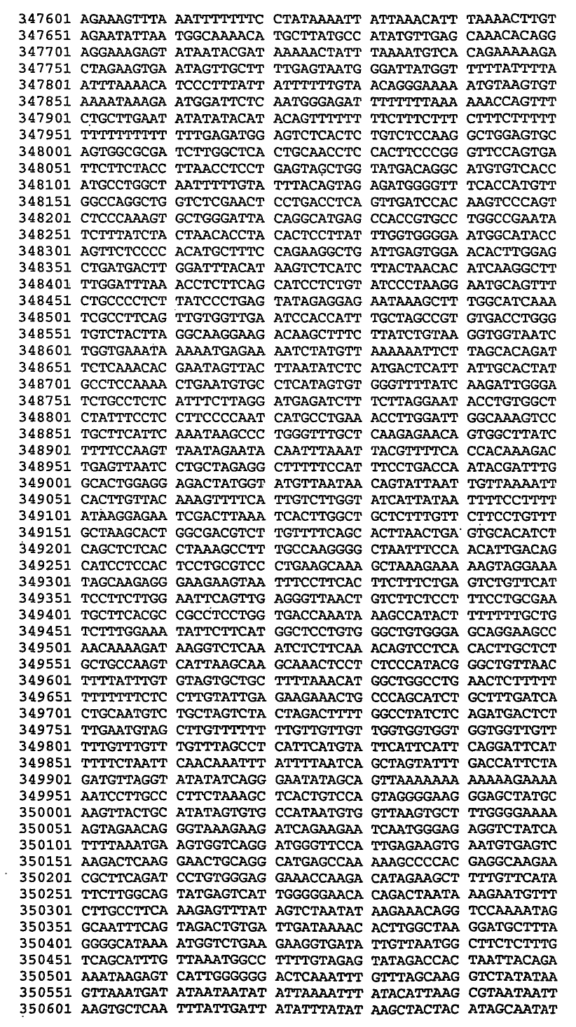

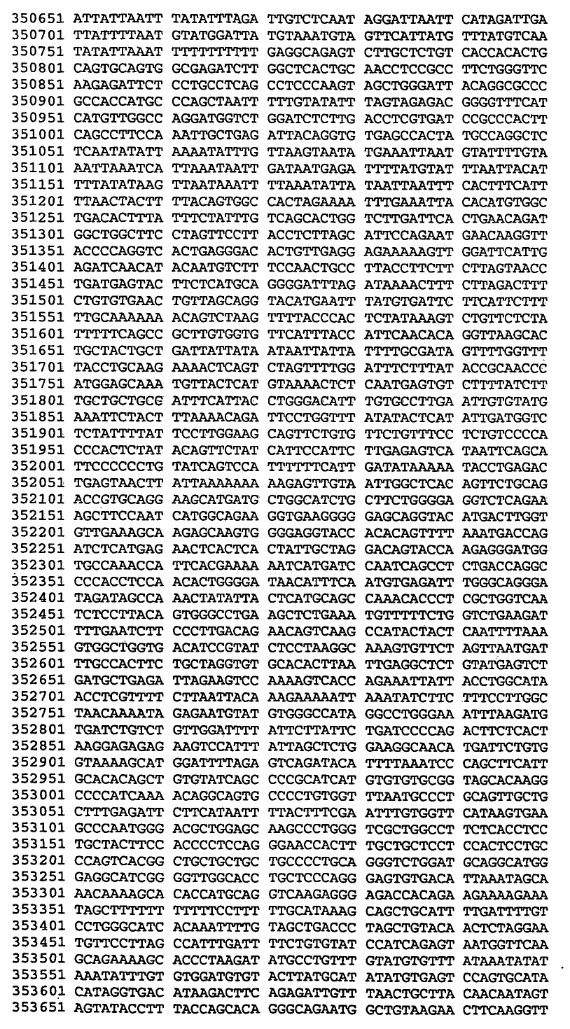

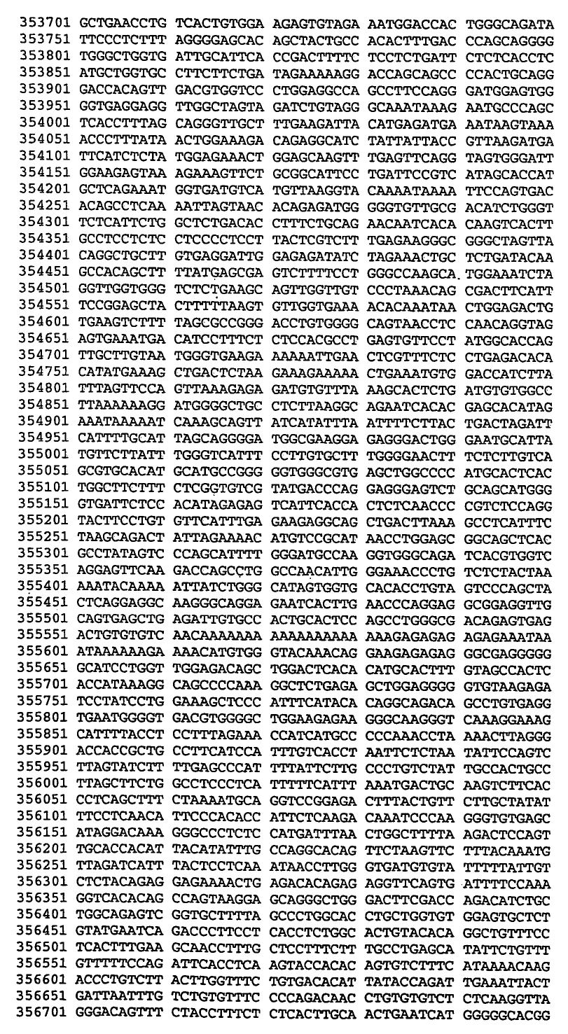

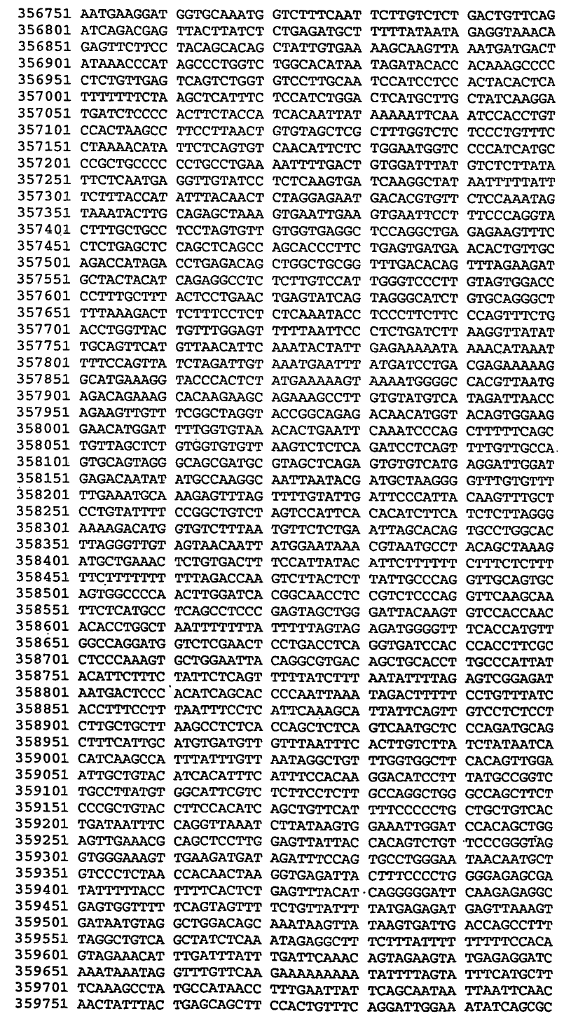

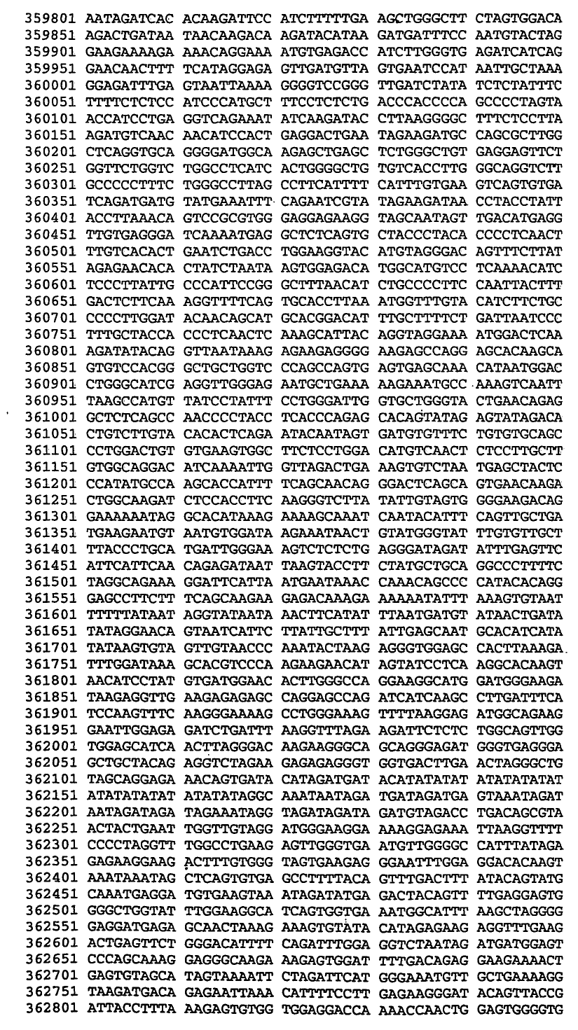

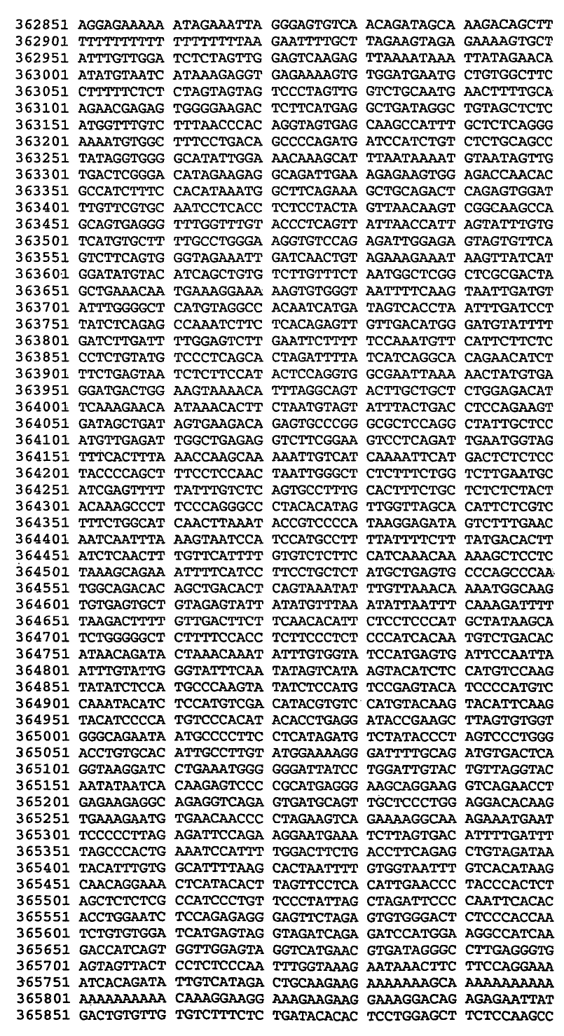

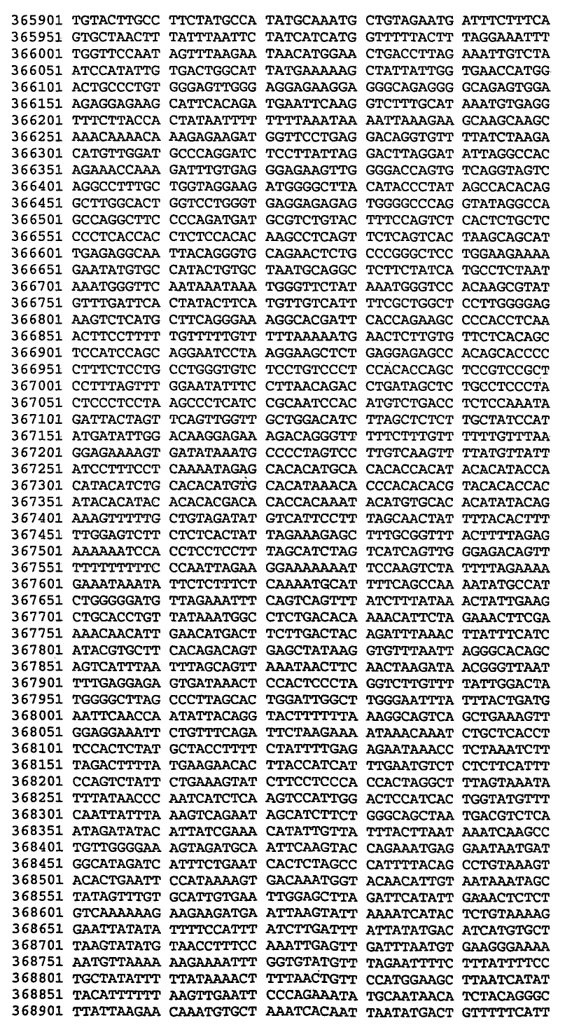

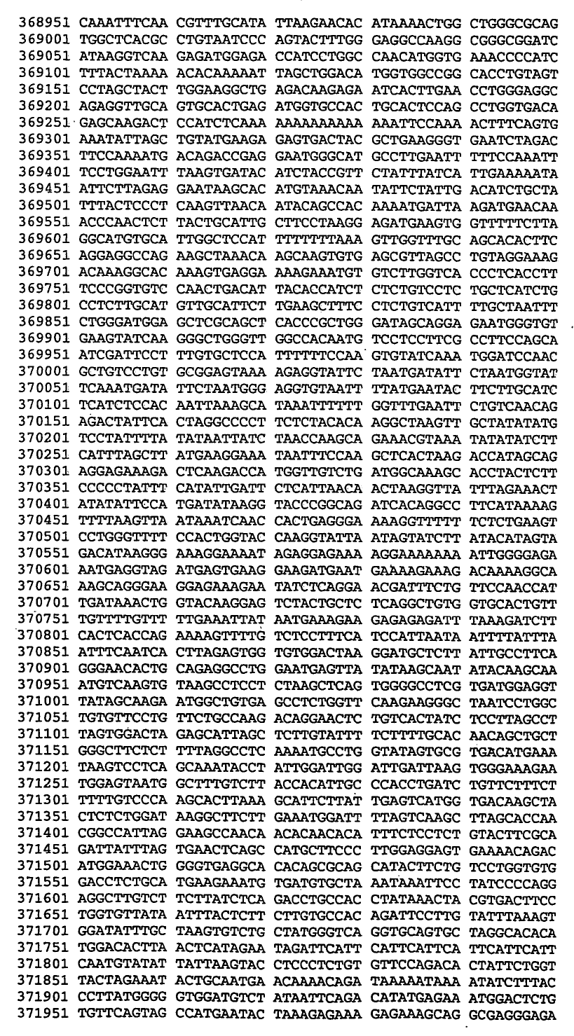

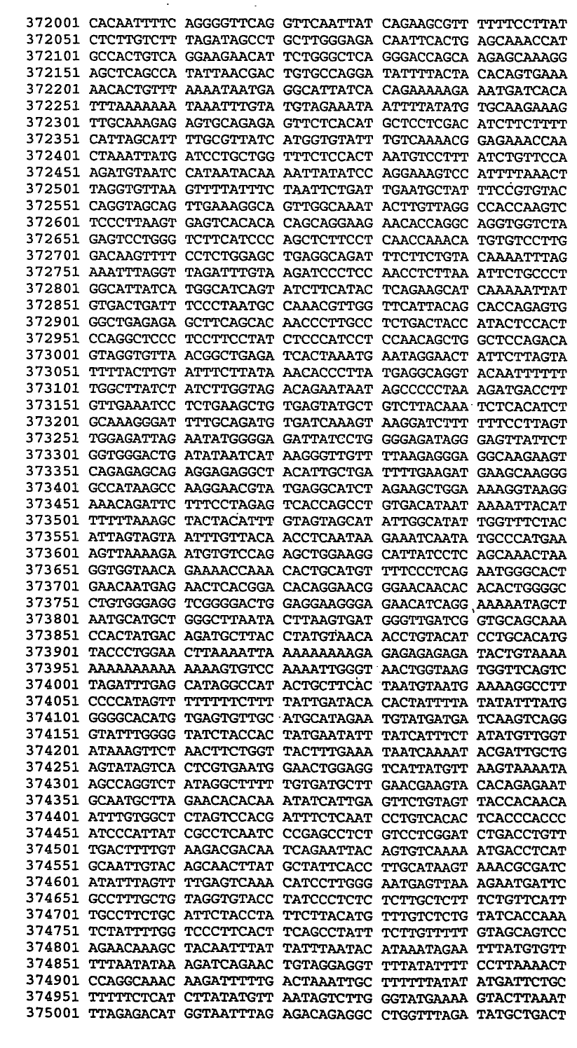

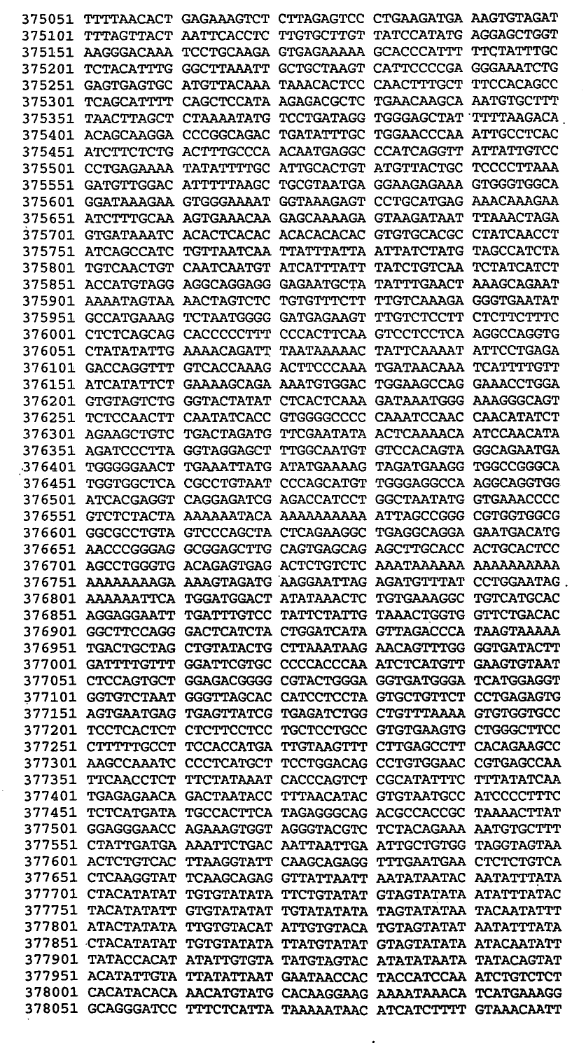

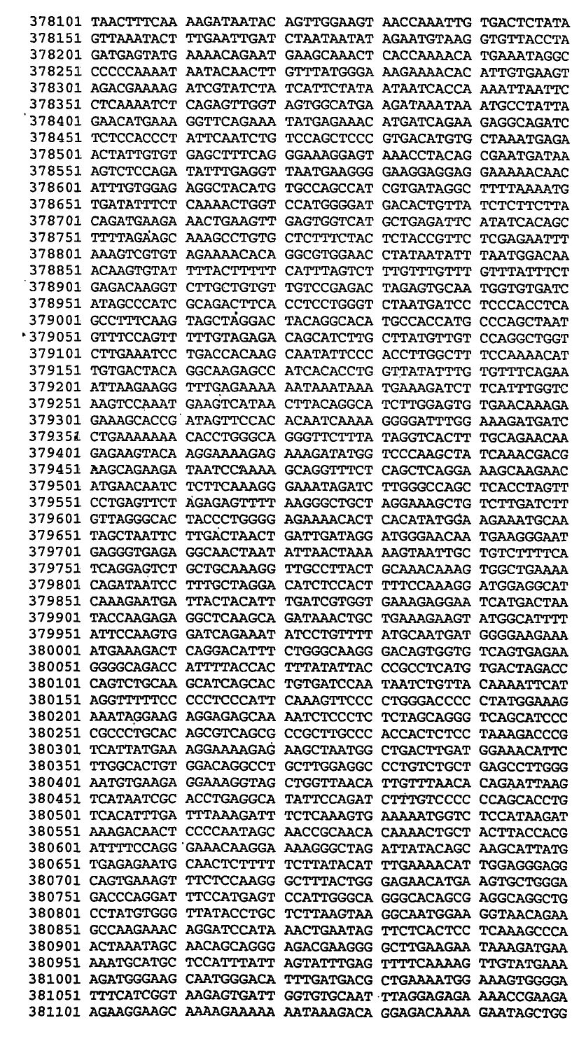

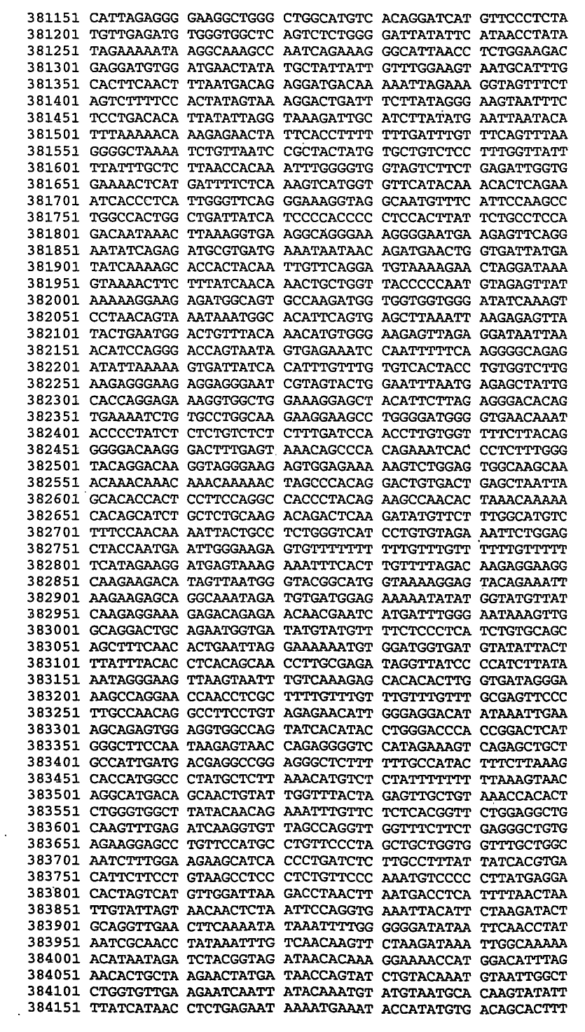

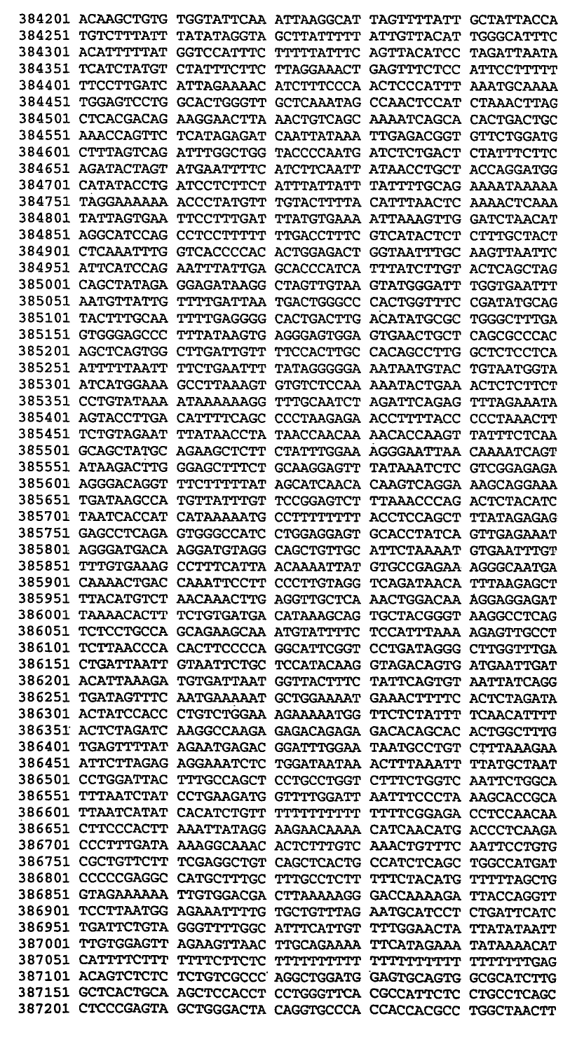

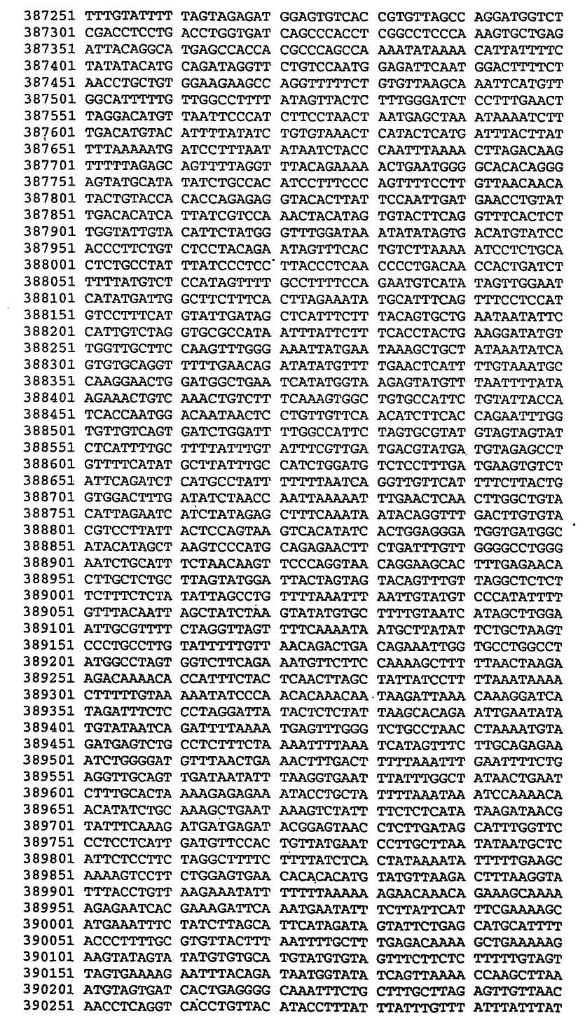

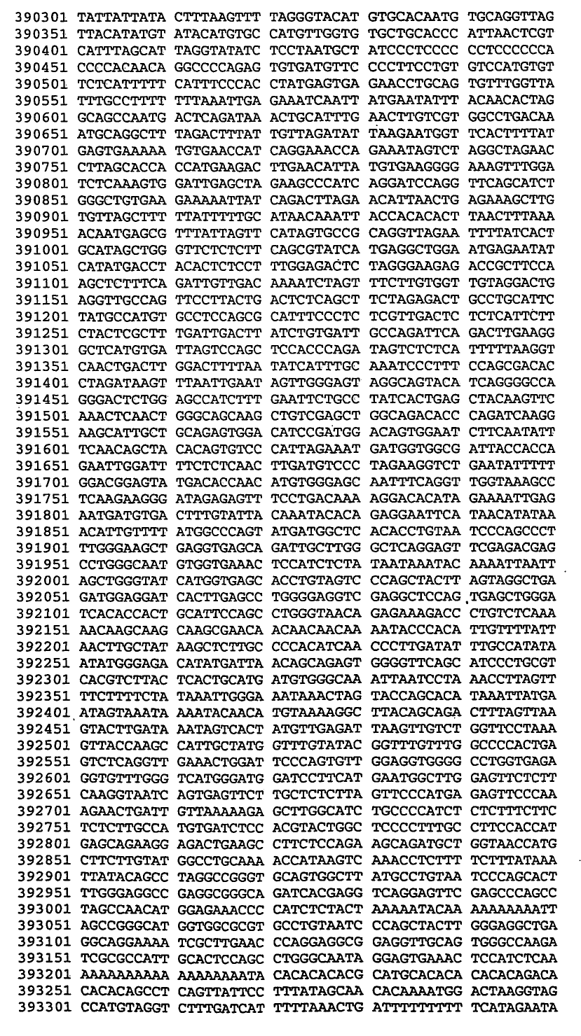

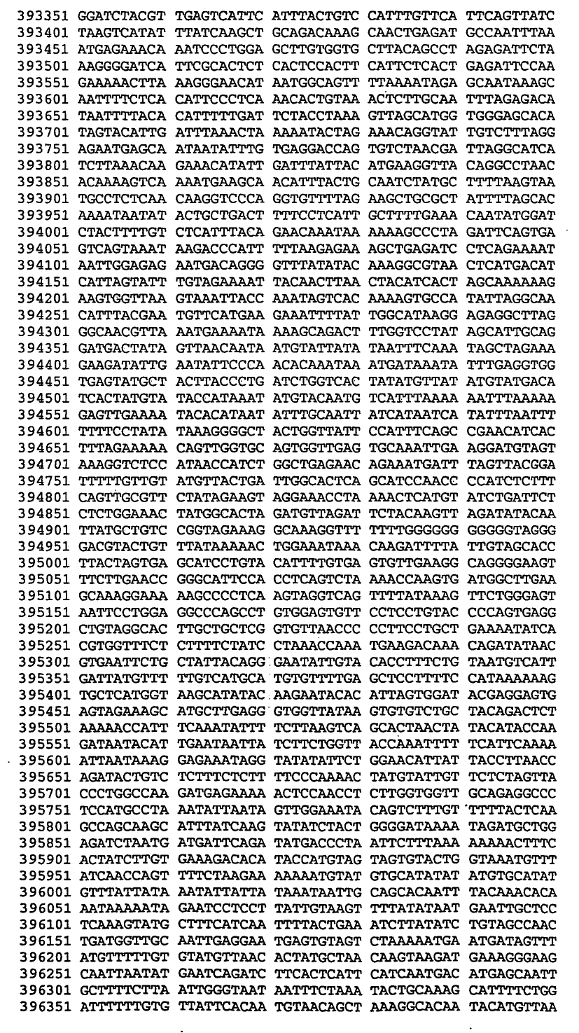

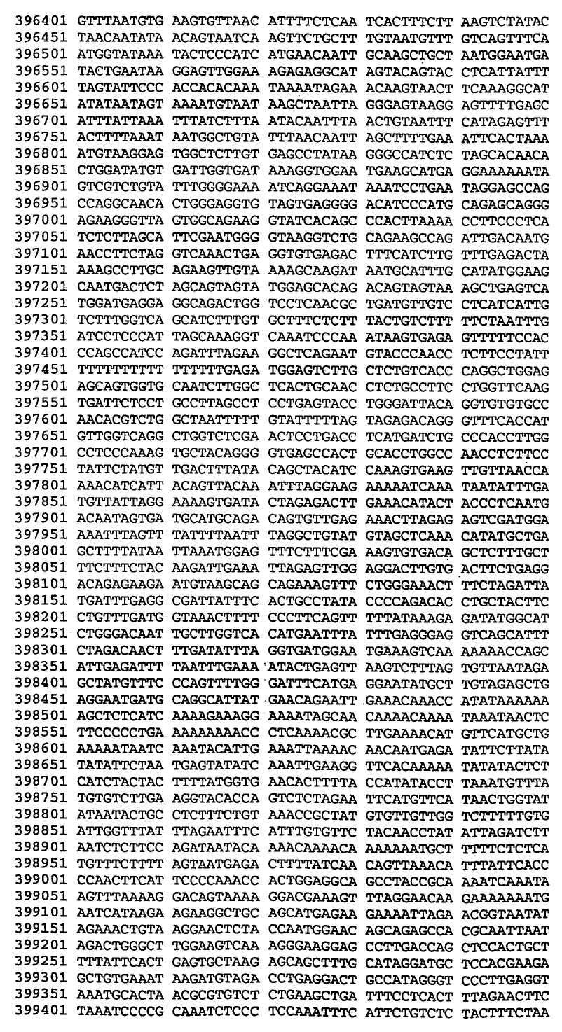

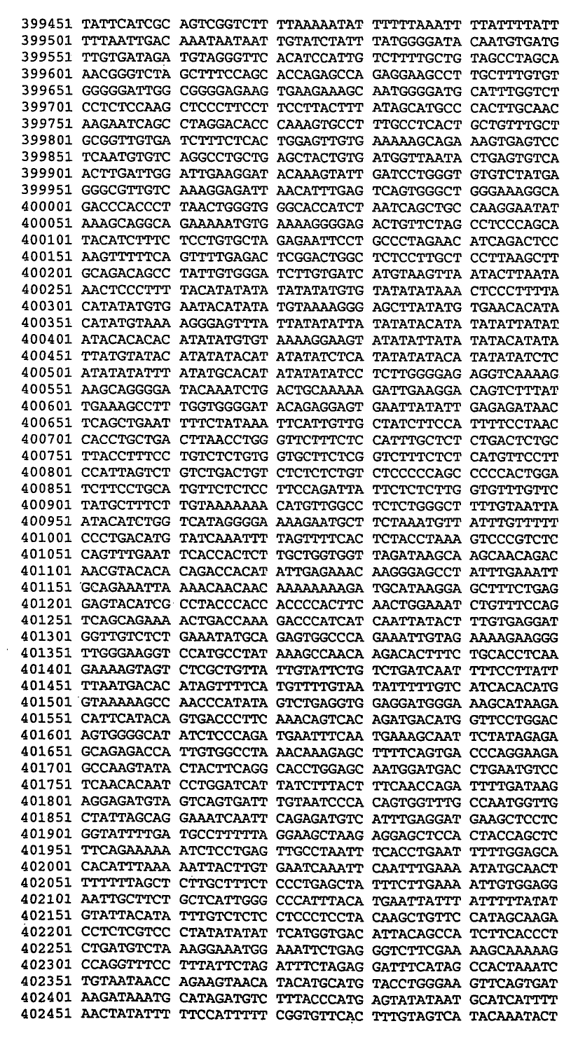

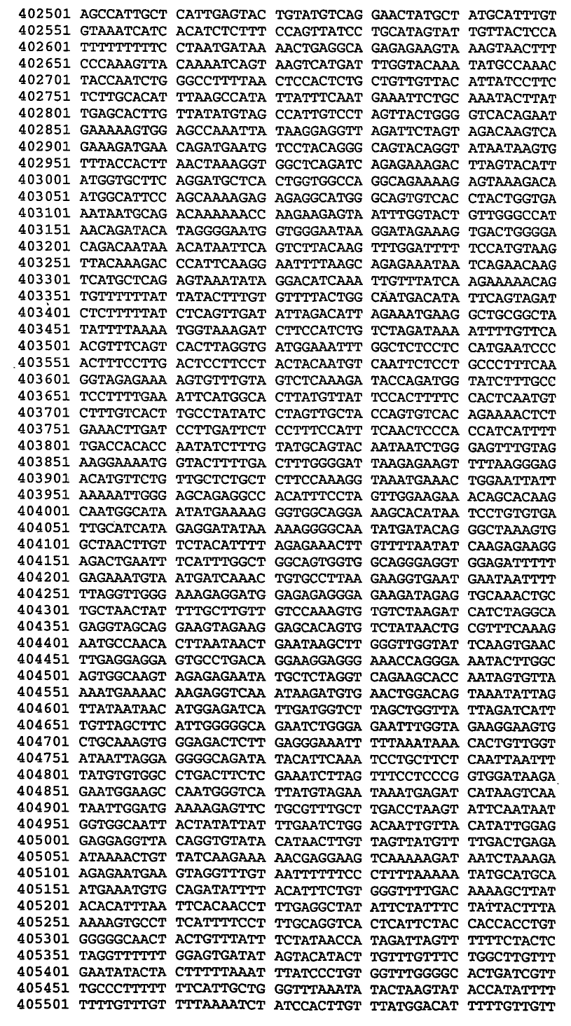

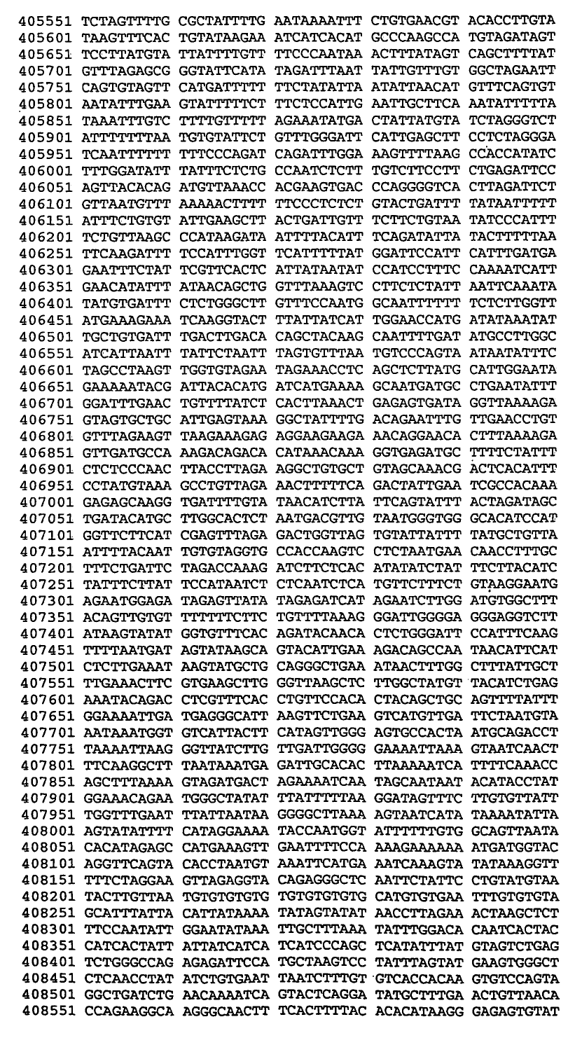

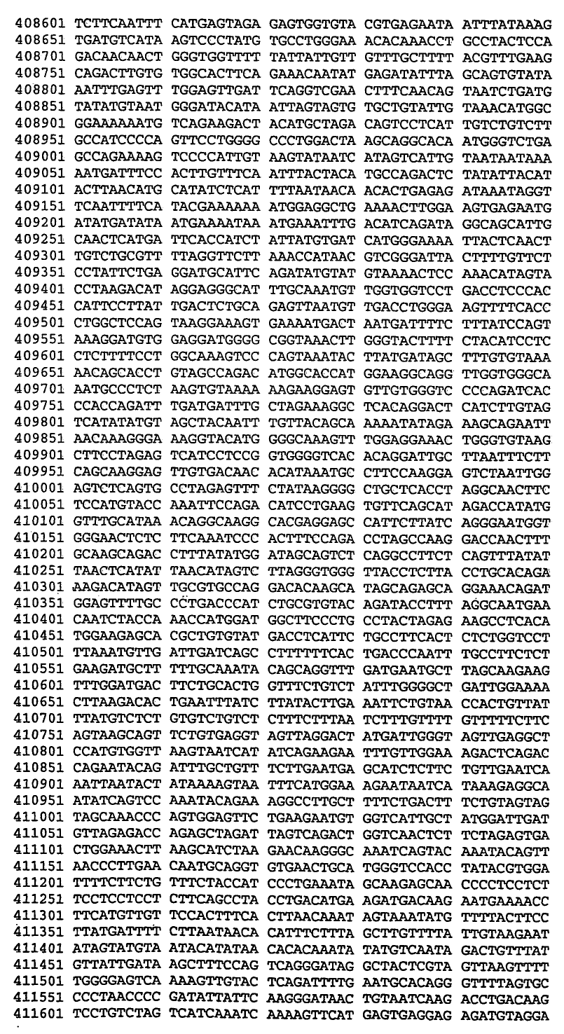

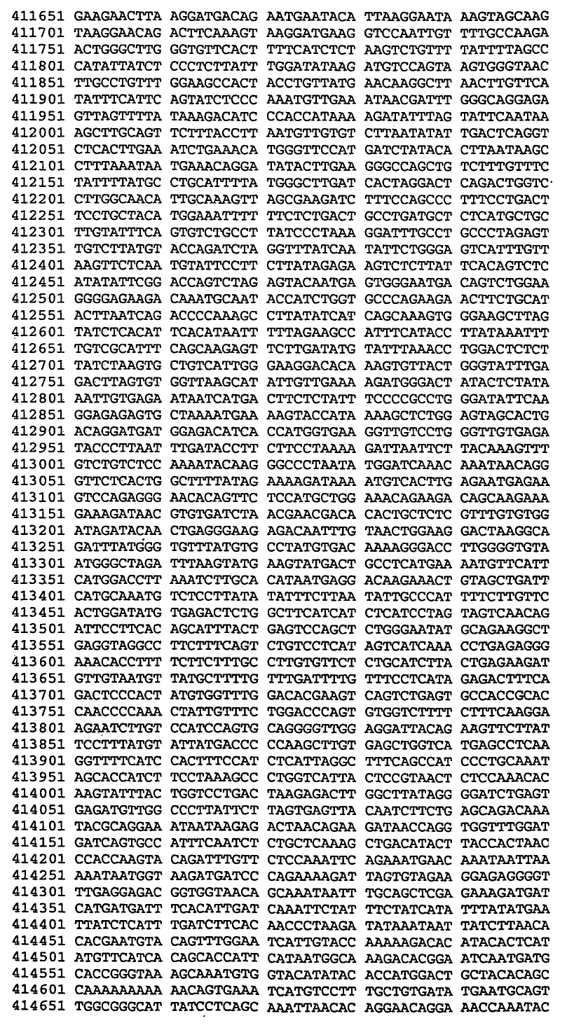

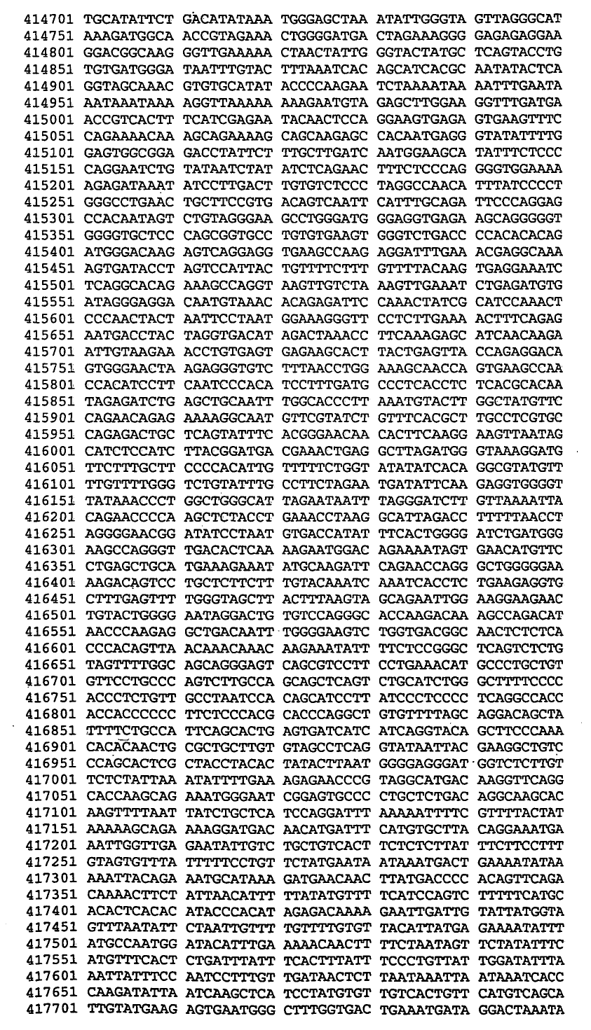

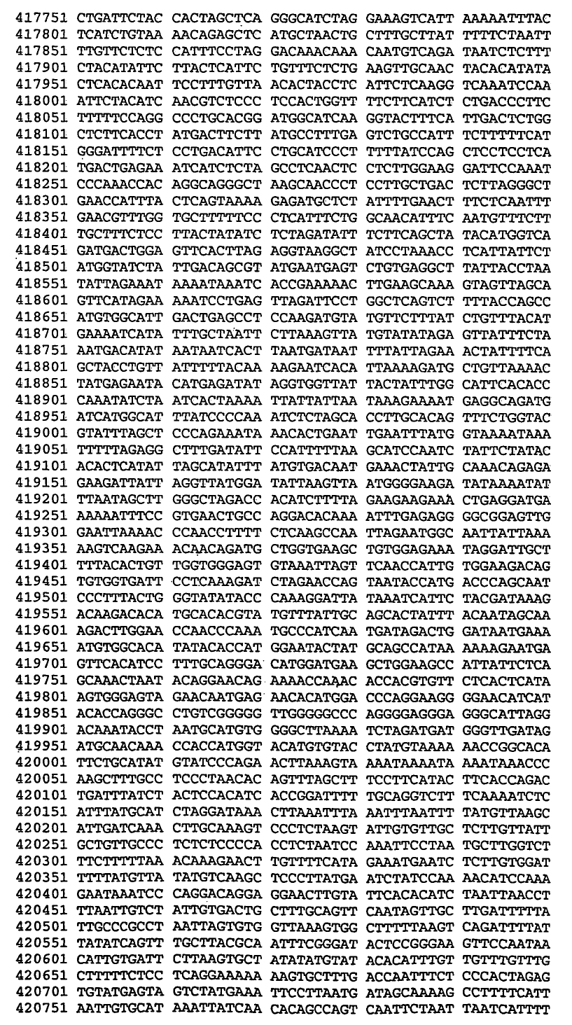

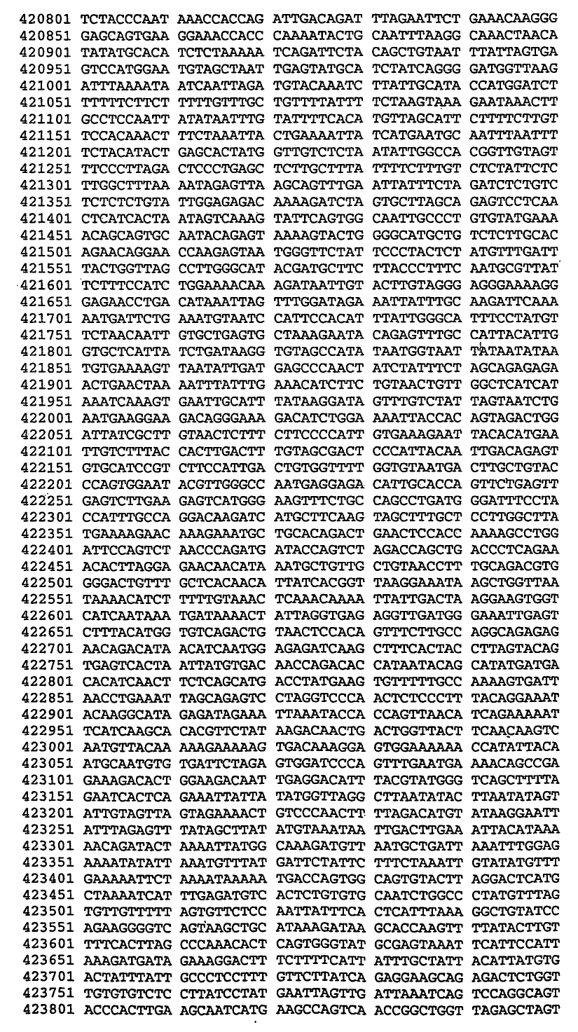

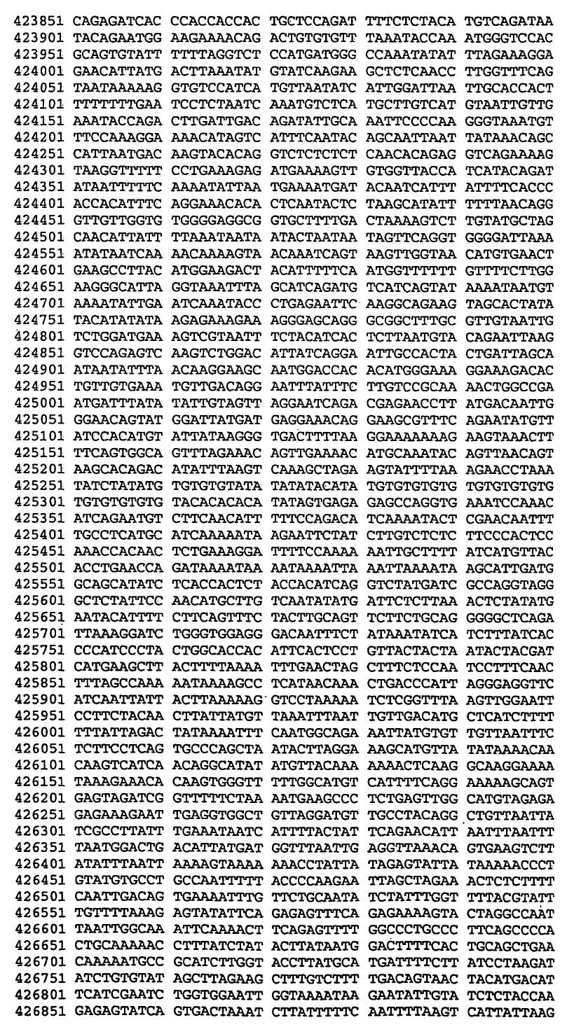

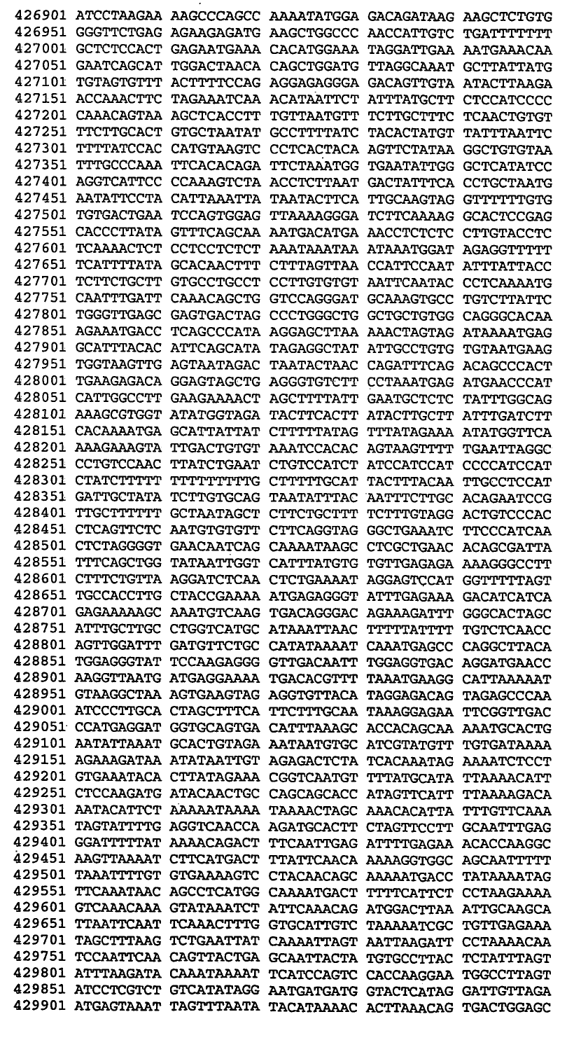

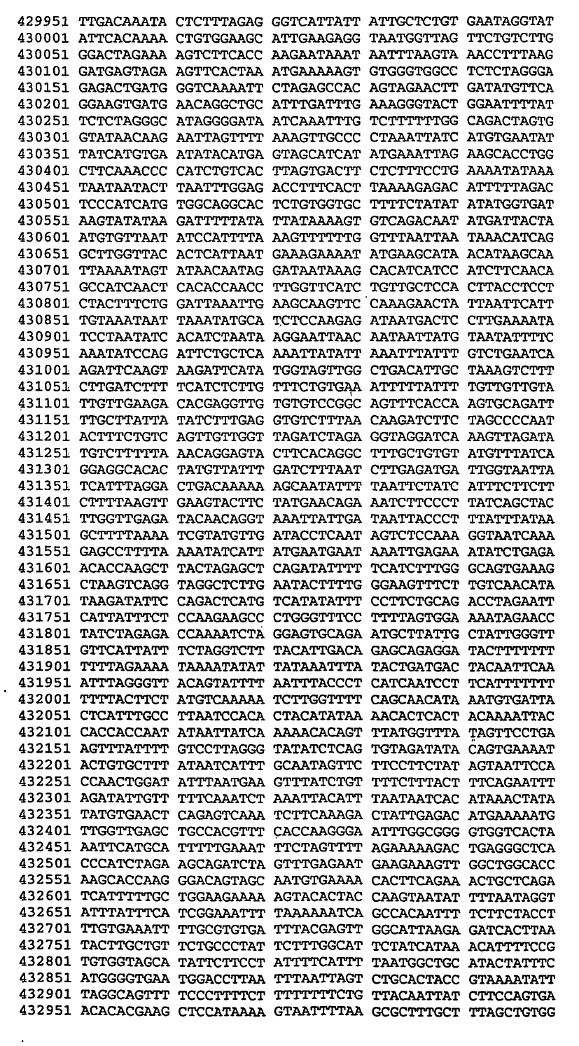

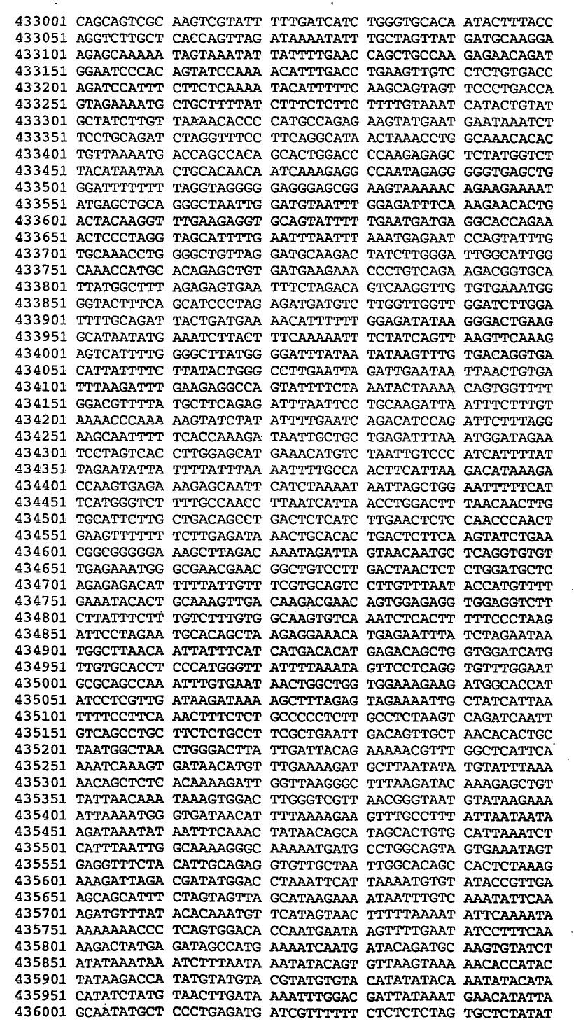

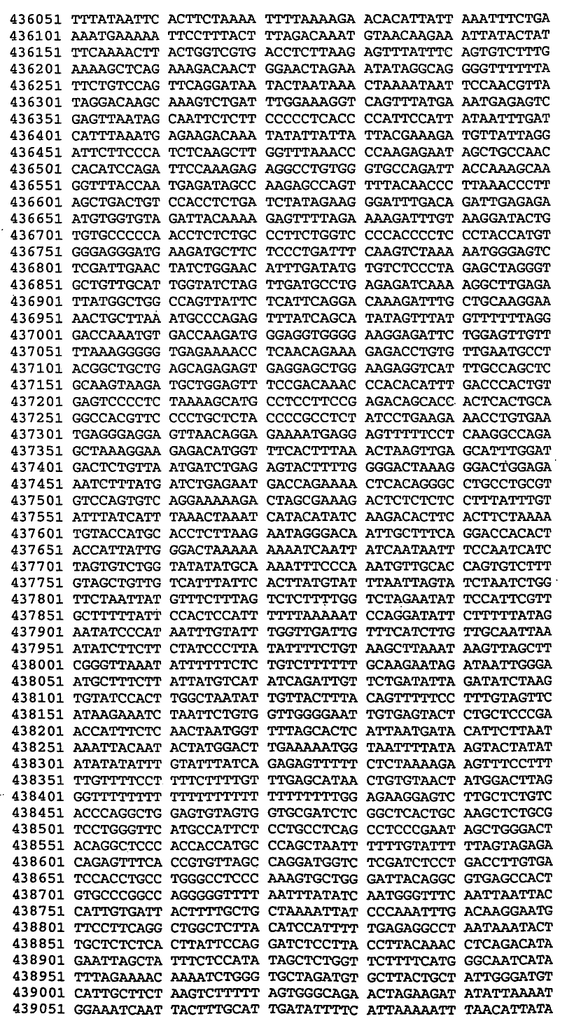

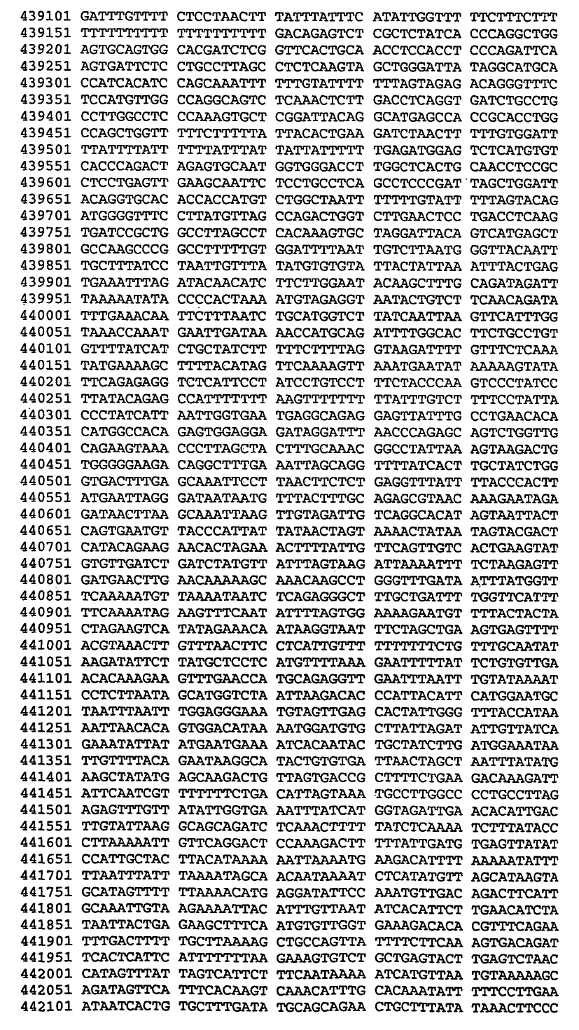

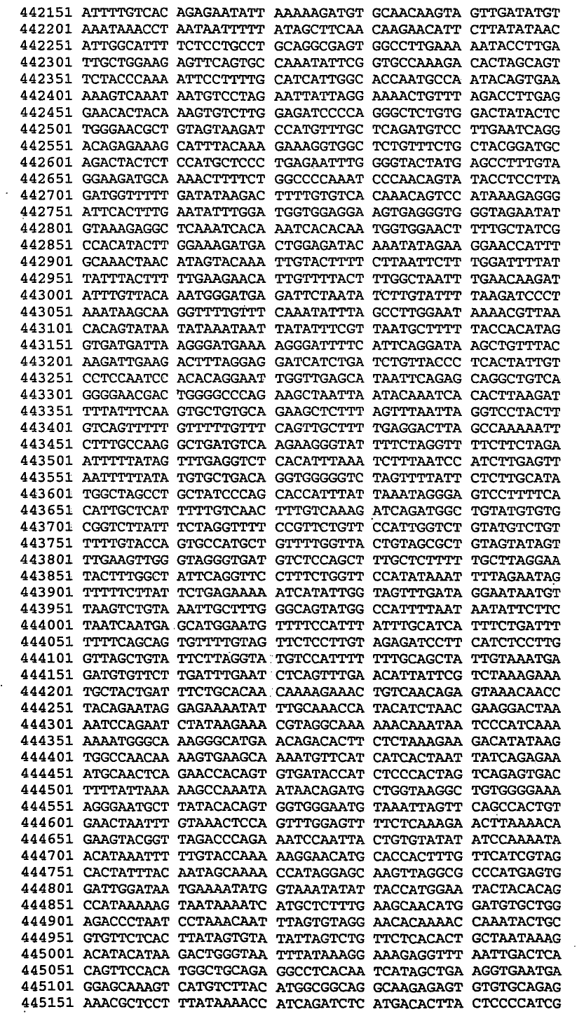

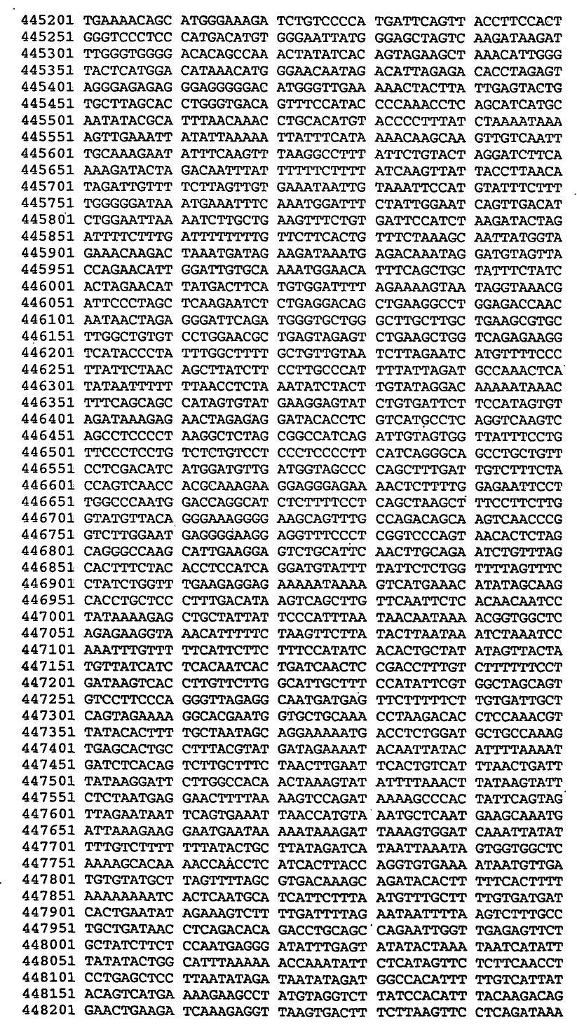

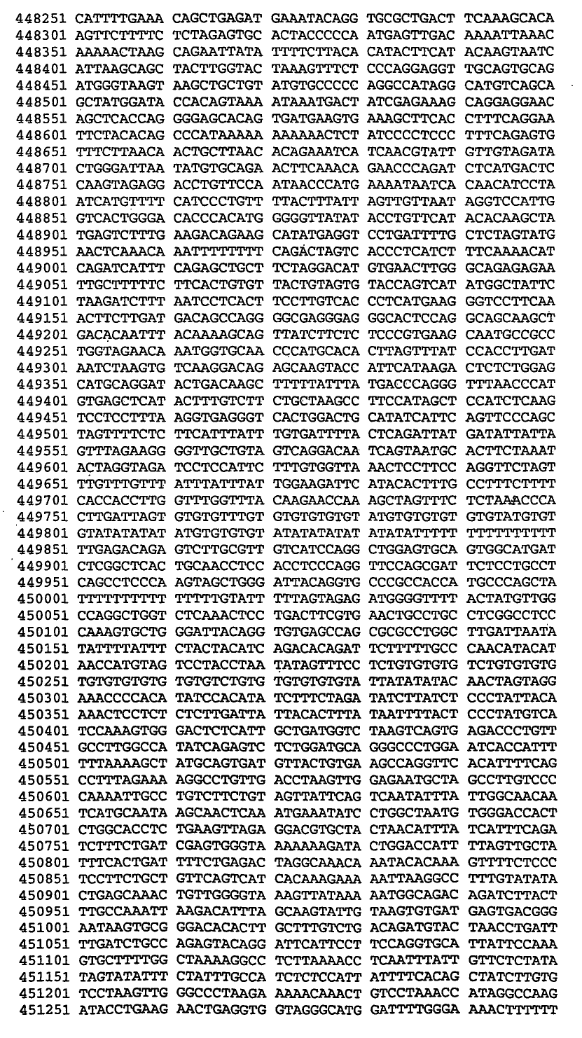

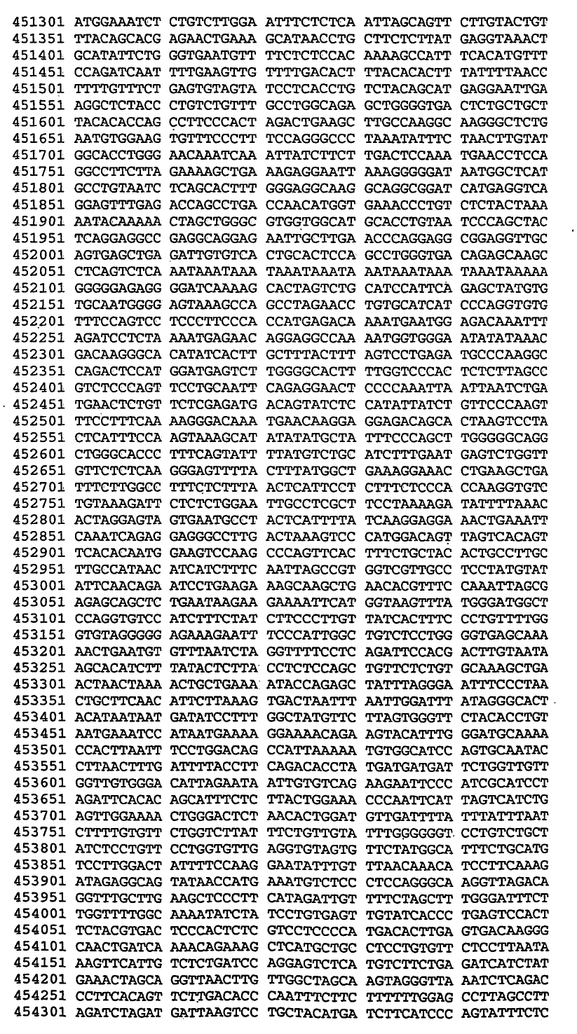

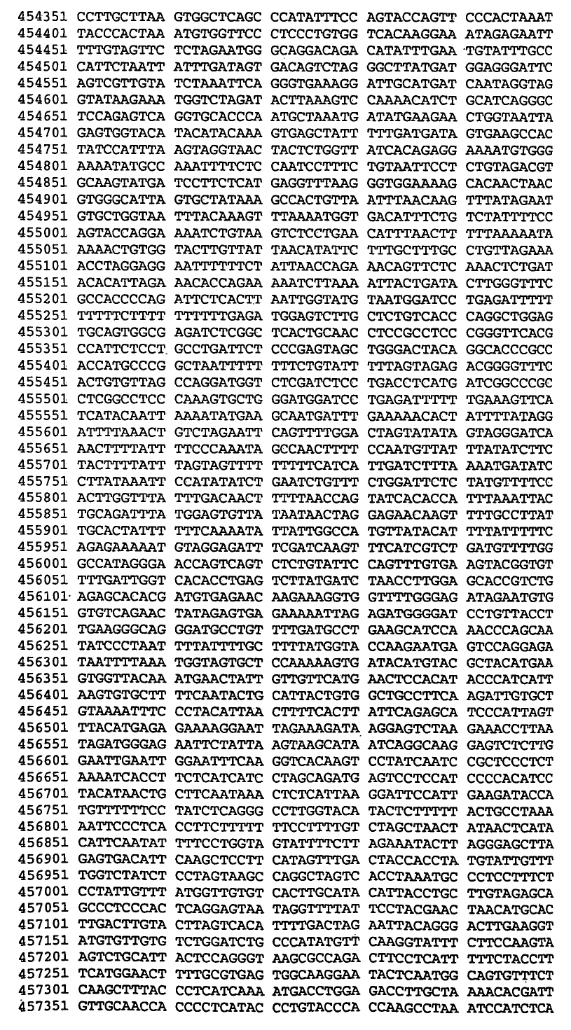

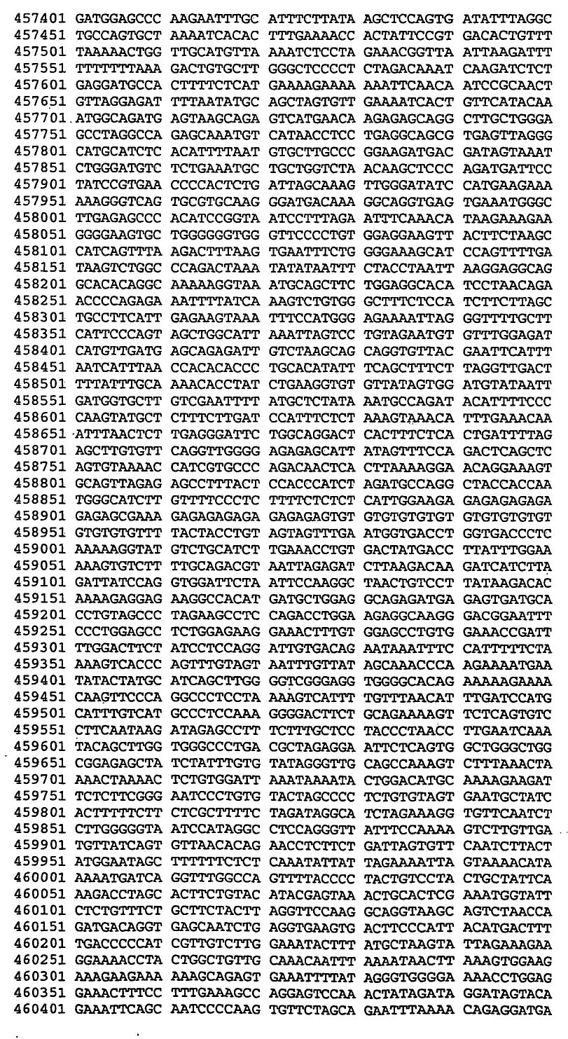

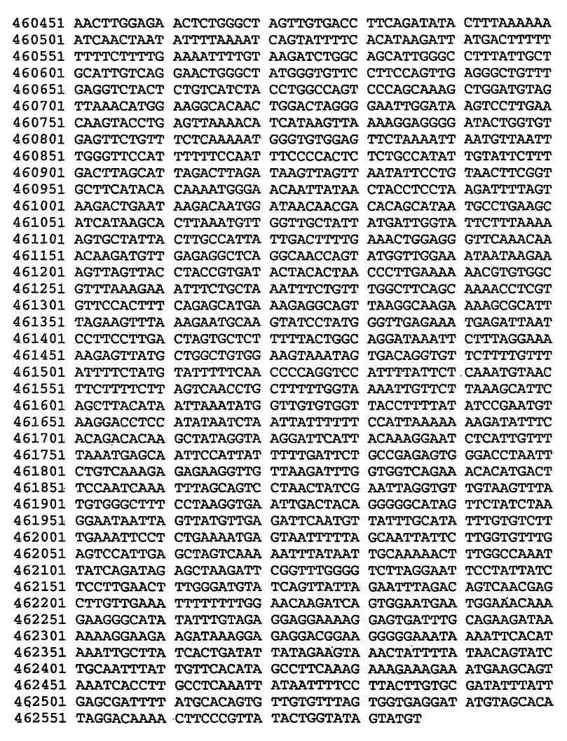

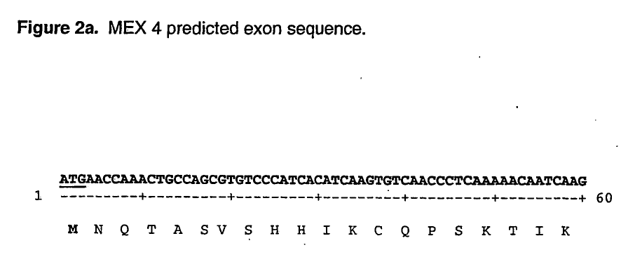

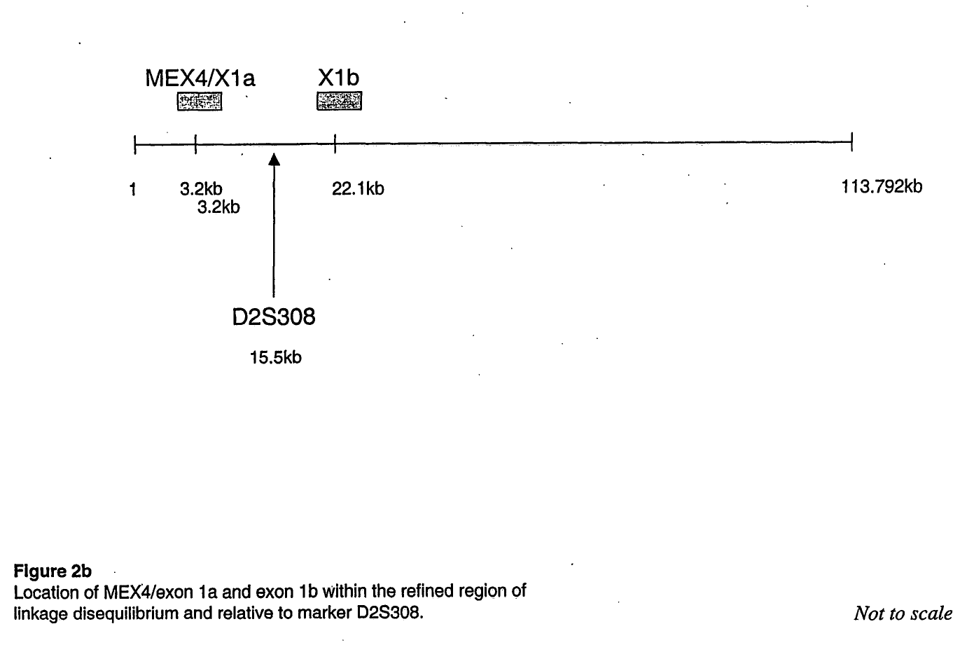

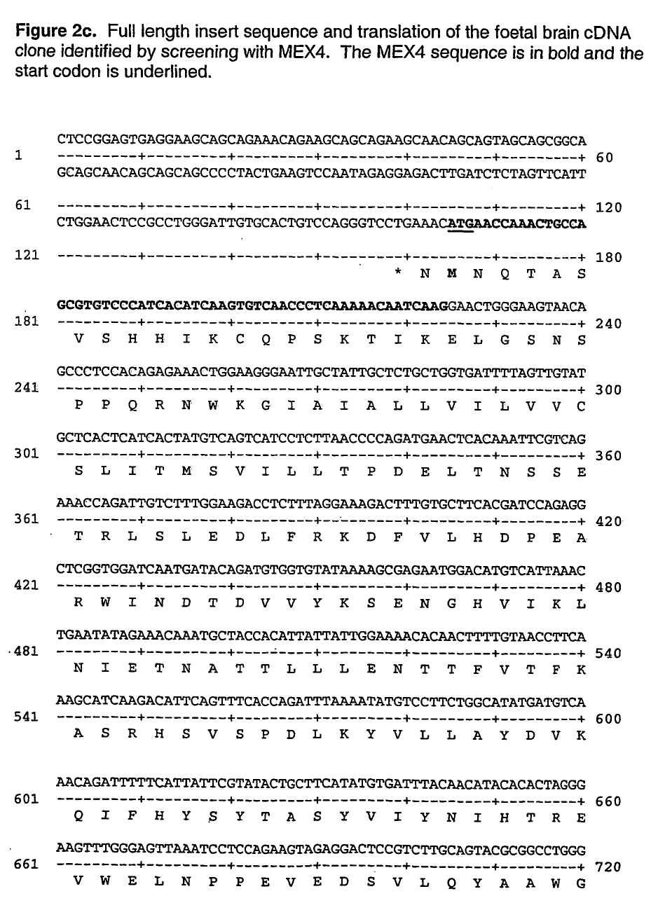

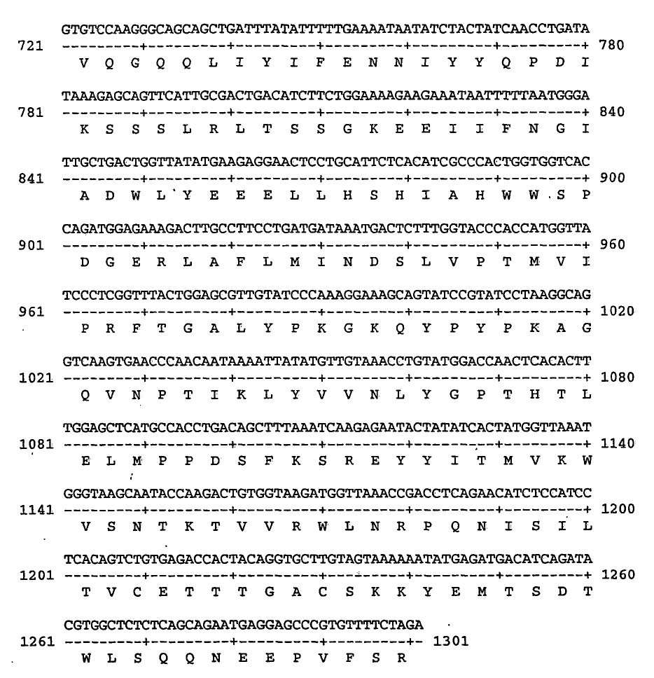

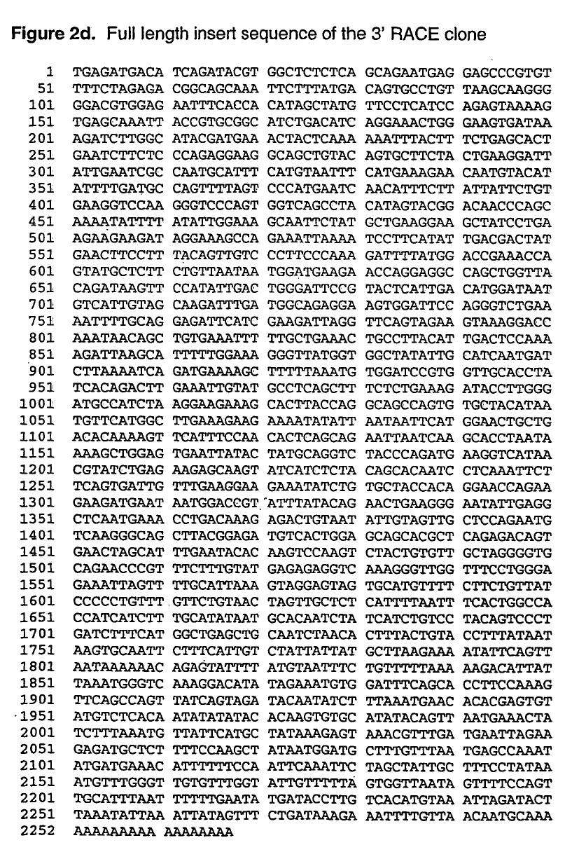

There is provided an isolated nucleic acid sequence comprising one or more of the exons of DPP10, for example those exons shown in FIGS. 6, 7, 8, 9, 10 or 15 or the sequence set out in FIG. 2c, a sequence complementary or substantially homologous thereto, or a fragment thereof.

There is provided an isolated nucleic acid sequence comprising the exon sequence of FIG. 2a, or a sequence which is complementary or substantially homologous thereto.

There is provided an isolated nucleic acid sequence comprising exons 1a and 2 to 25, or a sequence which is complementary or substantially homologous thereto, or a fragment thereof (Transcript 1), FIG. 10.

There is provided an isolated nucleic acid sequence comprising exons 1b, 1c and 2 to 25, or a sequence which is complementary or substantially homologous thereto, or a fragment thereof (Transcript 2), FIGS. 9 and 10.

There is provided an isolated nucleic acid sequence comprising exons 1b, 1c, 1d, 1e and 2 to 25, or a sequence which is complementary or substantially homologous thereto, or a fragment thereof (Transcript 3), FIGS. 9 and 10.

There is provided an isolated nucleic acid sequence comprising exons 1f and 2 to 25, or a sequence which is complementary or substantially homologous thereto, or a fragment thereof (Transcript 4), FIGS. 9 and 10.

There is provided an isolated nucleic acid sequence comprising exons 1g and 2 to 25, or a sequence which is complementary or substantially homologous thereto, or a fragment thereof (Transcript 5), FIGS. 9 and 10.

There is provided an isolated nucleic acid sequence comprising exons 1a and 2A, or a sequence which is complementary or substantially homologous thereto, or a fragment thereof (Transcript 6), FIGS. 9 and 10.

There is provided an isolated nucleic acid sequence comprising exons 1b, 1c and 2B, or a sequence which is complementary or substantially homologous thereto, or a fragment thereof, FIGS. 9 and 15.

There is provided an isolated nucleic acid sequence comprising exons 1b, 1c and 2C, or a sequence which is complementary or substantially homologous thereto, or a fragment thereof, FIGS. 9 and 15.

There is provided an isolated nucleic acid sequence comprising exons 1b, 1c and 2D, or a sequence which is complementary or substantially homologous thereto, or a fragment thereof, FIGS. 9 and 15.

There is provided an isolated nucleic acid sequence comprising exons 1b, 1c and 2E, or a sequence which is complementary or substantially homologous thereto, or a fragment thereof, FIGS. 9 and 15.

There is provided an isolated nucleic acid sequence comprising exons 1b, 1c and 2F, or a sequence which is complementary or substantially homologous thereto, or a fragment thereof, FIGS. 9 and 15.

There is provided an isolated nucleic acid sequence comprising one or more of the mouse exons of DPP10, for example those exons shown in FIG. 5, 5a, 7 or 8, or a sequence complementary or substantially homologous thereto, or a fragment thereof. There is provided an isolated nucleic acid sequence comprising mouse exons 1a and 2 to 25, or a sequence which is complementary or substantially homologous thereto, or a fragment thereof (Mouse Transcript 1), FIGS. 5a, 7 and 8.

There is provided an isolated nucleic acid sequence comprising mouse exons 1c, 1d and 2 to 25 or a sequence which is complementary or substantially homologous thereto, or a fragment thereof (Mouse Transcript 2), FIGS. 7 and 8.

There is provided an isolated nucleic acid sequence comprising mouse exons 1e and 2 to 25 or a sequence which is complementary or substantially homologous thereto, or a fragment thereof (Mouse Transcript 3), FIGS. 7 and 8.

There is provided an isolated nucleic acid sequence comprising mouse exons 1b, 1c, Id and 2 to 25 or a sequence which is complementary or substantially homologous thereto, or a fragment thereof (Mouse Transcript 4), FIGS. 7 and 8. Promoter sequence for DPP10: A consensus palindromic IFN-gamma activation site (GAS) element was identified 18181base pairs upstream from Exon 1b. The GAS site is underlined. Represented in italics are other important promoter motifs: a CATT box and a TATA box.

CCATTCTCTTTGTTTTTATTCGGGATGCTCTTATTTCCAAGAAGGCTTATAAA

This motif is also present in the promoter of the CD26/dipeptidylpeptidase IV (DPP4) gene (a member of the prolyl oligopeptidase S9B subfamily). Interferons (IFNs alpha, beta and gamma) and trans retinoic acid (RA) have the ability to activate genes with GAS sites.

All the sequences of the present invention are isolated, or alternatively may be recombinant. By isolated is meant a nucleic acid or polypeptide sequence which has been purified, and is substantially free of other protein and nucleic acid. Such sequences may be obtained by PCR amplification, cloning techniques, or synthesis on a synthesiser. By recombinant is meant nucleic acid sequences which have been recombined by the hand of man.

The polynucleotide sequences of the invention may be genomic or cDNA, or RNA, preferably mRNA, or PNA. In the present invention, gene products include polynucleotide sequences and protein. References to polypeptide sequences include proteins and peptides.

In the present application, sequences which are complementary or substantially homologous are those sequences which hybridise under stringent conditions to the defined sequence or its gene products. Thus, for example, a nucleic acid sequence substantially homologous to a reference nucleic acid will be capable of hybridising to a gene product (i.e. mRNA) of the reference nucleic acid, under stringent conditions. A complementary sequence is one which is capable of hybridising to the nucleic acid. sequence itself, under stringent conditions. Also provided in the present invention are complements of the substantially homologous sequences. A substantially homologous sequence preferably has at least 75, 76, 77, 78, 79, 80, 81, 82, 83, 84, 85, 86, 87, 88, 89, 90, 91, 92, 93, 94, 95, 96, 97, 98 or 100% sequence identity with the defined sequence. This definition of substantially homologous applies to both nucleic acid and polypeptide sequences. Thus, polypeptide sequences having conservative amino acid substitutions that do not affect structure or function are also included. For any given DNA sequence, references to a complementary sequence include the corresponding mRNA sequence and any cDNA sequence derived on such an RNA sequence. “% identity” is a measure of the relationship between two nucleic acid or polypeptide sequences, as determined by comparing their sequences. In general, the two sequences to be compared are aligned to give a maximum correlation between the sequences. The alignment of the two sequences is examined and the number of positions giving an exact amino acid or nucleotide correspondence is determined, and divided by the total length of the alignment, and the result is multiplied by 100 to give a % identity. The % identity may be determined over the whole length of the sequence to be compared, which is particularly suitable for sequences of the same or similar lengths or for sequences which are highly homologous, or over shorter defined lengths which is more suitable for sequences of unequal lengths and with a lower homology.

Methods for comparing the identity of two or more sequences are known in the art. For example, programs available in the Wisconsin Sequence Analysis Package version 9.1 (Devereux J et al., Nucl Acid Res 12 387-395 (1984), available from Genetics Computer Group, Madison, Wis., USA), such as BESTFIT and GAP may be used.

BESTFIT uses the “local homology” algorithm of Smith and Waterman (Advances in Applied Mathematics, 2:482489, 1981) and finds the best single region of similarity between two sequences. BESTFIT is more suited to comparing two polynucleotide or two polypeptide sequences which are dissimilar in length, the program assuming that the shorter sequence represents a portion of the longer. In comparison, GAP aligns two sequences finding a “maximum similarity” according to the algorithm of Neddleman and Wunsch (J. Mol. Biol. 48:443-354, 1970). GAP is more suited to comparing sequences which are approximately the same length and an alignment is expected over the entire length. Preferably, the parameters “Gap Weight” and “length Weight” used in each program are 50 and 3 for polynucleotide sequences and 12 and 4 for polypeptide sequences, respectively. Preferably, % identities and similarities are determined when the two sequences being compared are optimally aligned.

Other programs for determining identity and/or similarity between sequences are also known in the art, for instance the BLAST family of programs (Altschul et al, J. Mol. Biol., 215:403410, (1990) and Altschul et al, Nuc Acids Res., 25:289-3402 (1997), available from the National Center for Biotechnology Information (NCB), Bethesda, Md., USA and accessible through the home page of the NCBI at www.ncbi.nlm.nih.gov) and FASTA (Pearson W. R. and Lipman D. J., Proc. Nat. Acac. Sci., USA, 85:2444-2448 (1988), available as part of the Wisconsin Sequence Analysis Package). Preferably, the BLOSUM62 amino acid substitution matrix (Henikoff S. and Henikoff J. G., Proc. Nat. Acad Sci., USA, 89:10915-10919, (1992)) is used in polypeptide sequence comparisons including where nucleotide sequences are first translated into amino acid sequences before comparison.

Preferably, the program BESTFIT is used to determine the % identity of a query polynucleotide or a polypeptide sequence with respect to a polynucleotide or a polypeptide sequence of the present invention, the query and the reference sequence being optimally aligned and the parameters of the program set at the default value.

In relation to the present invention, “stringent conditions” refers to the washing conditions used in a hybridisation protocol. In general, the washing conditions should be a combination of temperature and salt concentration so that the denaturation temperature is approximately 5 to 20° C. below the calculated Tₘ of the nucleic acid under study. The Tₘ of a nucleic acid probe of 20 bases or less is calculated under standard conditions (1M NaCi) as [4° C.×(G+C)+2° C.×(A+T)], according to Wallace rules for short oligonucleotides. For longer DNA fragments, the nearest neighbor method, which combines solid thermodynamics and experimental data may be used, according to the principles set out in Breslauer et al., PNAS 83: 3746-3750 (1986). The optimum salt and temperature conditions for hybridisation may be readily determined in preliminary experiments in which DNA samples immobilised on filters are hybridised to the probe of interest and then washed under conditions of different stringencies. While the conditions for PCR may differ from the standard conditions, the Tm may be used as a guide for the expected relative stability of the primers. For short primers of approximately 14 nucleotides, low annealing temperatures of around 44° C. to 50° C. are used. The temperature may be higher depending upon the base composition of the primer sequence used. Suitably stringent conditions are those under which non-specific hybridisation (e.g. to non-DPP10 encoding sequences) are avoided. Suitable stringent conditions are 0.5×SSC/1%SDS/58° C./30mins for a 21 mer oligonucleotide probe.

The complementary sequences of the invention (which may also be referred to herein as “antisense”) may be useful as probes or primers, or in the regulation of DPP10 expression. Preferably, the primer sequences are capable of amplifying all or a portion of a DPP10 gene. Preferred primer sequences are disclosed in the Examples. Pairs of primers for amplification of all or part of the gene, or alleles, or variants thereof, form another aspect of the invention. Similarly, DPP10 probes will be useful in detecting the presence or expression levels of DPP10, or variant forms thereof, in a sample from a subject. The probes may also be useful in analysing the expression pattern of DPP10 in a subject.

In the present application, fragments are any contiguous 10 residue sequence, or greater, such as 20, 30, 40, or 50 residue sequence. Preferably, fragments of nucleic acid or polypeptide sequences share one or more functional characteristics with DPP10 or its gene, or are capable of modulating (i.e. inhibiting or enhancing) such a functional characteristic. The novelty of a fragment according to the present embodiment may be easily ascertained by comparing the nucleotide or polypeptide sequence of the fragment with sequences catalogued in databases such as Genebank at the priority date, or by using computer programs such as DNASIS (Hitachi Engineering Inc) or Word Search or FASTA of the Genetic Computer Group (Madison, USA).

The fragments may be used in a variety of diagnostic, prognostic or therapeutic methods or may be useful as research tools for example in screening. Fragments of the sequences of the first aspect or their complements may be used as primer sequences as described above.

In a second aspect of the invention, the isolated nucleic acid sequences of the invention may be provided in the form of a vector to enable the in vitro or in vivo expression of DPP10. Vectors include plasmids, chromosomes, artificial chromosomes and viruses and may be expression vectors, which are capable of expressing nucleic acid sequences in vitro or in vivo, or transformation vectors which are capable of transferring the nucleic acid sequence from one environment to another. The nucleic acid molecules of the invention may be operably linked to one or more regulatory elements including a promoter.

The term regulatory elements includes response elements, consensus sites, methylation sites, locus control regions, post-transcriptional modifications, splice variants, homeoboxes, inducible factors, DNA binding domains, enhancer sequences, initiation codons, secretion signals and, polyA sequences. Regions upstream or downstream of a promoter such as enhancers, which regulate the activity of the promoter are also regulatory elements.

The vector may also comprise an origin of replication; appropriate restriction sites to enable cloning of inserts adjacent to the polynucleotide molecule; markers, for example antibiotic resistance genes; ribosome binding sites: RNA splice sites and transcription termination regions; polymerisation sites; or any other element, such a secretion signals, which may facilitate the cloning and or expression of the polynucleotide molecule.

Within a vector the gene may be expressed upstream or downstream of an expressed protein tag such as a histidine tag, V5 epitope tag, green fluorescent protein tag, MHC tag or other such tag known to those skilled in the art. Use of such a tag allows easy localisation, affinity purification and detection of the fusion protein with an antibody to the tag moiety.

Where two or more nucleic acid molecules of the invention are introduced into the same vector, each may be controlled by its own regulatory sequences, or all molecules may be controlled by the same regulatory sequence. In the same manner, each molecule may comprise a 3′ polyadenylation site. Examples of suitable vectors will be known to persons skilled in the art and include pBluescript II, lambdazap, and pCMV-Script (Stratagene Cloning Systems, La Jolla, USA).

Appropriate regulatory elements, in particular, promoters will usually depend upon the host cell into which the expression vector is to be inserted. Where microbial host cells are used, promoters such as lactose promoter system, tryptophan (Trp) promoter system, β-lactamase promoter system or phage lambda promoter system are suitable. Where yeast cells are used, preferred promoters include alcohol dehydrogenase I or glycolytic promoters. In mammalian host cells, preferred promoters are those derived from immunoglobulin genes, SV40, Adenovirus, Bovine Papilloma virus etc. Suitable promoters for use in various host cells would be readily apparent to a person skilled in the art (See, for example, Current Protocols in Molecular Biology Edited by Ausubel et al, published by Wiley). In addition, the regulatory elements may be modified, for example by the addition of further regulatory elements, to achieve a desired expression pattern.

By operably linked is meant that the components of the vector or sequence are in a relationship which allows them to function as intended.

These vectors may be used to transform host cells, for example, prokaryotic or eukaryotic cells. These cells may be used in the production of recombinant DPP10 gene products, or in the regulation or analysis of DPP10. The transformed host cells form part of the invention. Preferred cells include E. coli, yeast, filamentous fungi, insect cells, mammalian cells, preferably immortalised, such as mouse, CHO, HeLa, Myeloma or Jurkat cell lines, human and monkey cell lines and derivatives thereof

According to a third aspect of the invention, there is provided a polypeptide sequence comprising a polypeptide sequence encoded by a nucleic acid sequence of the first aspect of the invention. Preferably the polypeptide sequences are encoded by a nucleic acid sequence of FIGS. 2a, 4, 5, 5a, 6, 9 or 10.

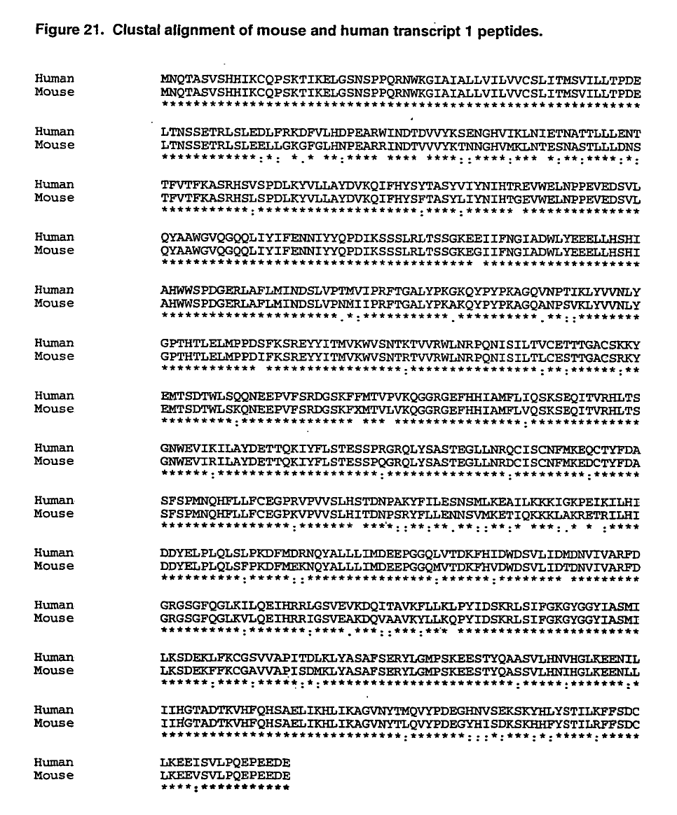

The third aspect of the invention includes a polypeptide sequence comprising a polypeptide sequence as shown in any one of FIGS. 2a, 2c, 4, 5, 5a, 8, 9, 11, 12 or 21 or a sequence homologous thereto, or a fragment thereof. The sequences of FIGS. 2c, 4, 9, 11 and 12 are the predicted human DPP10 polypeptide sequences. The sequences of FIGS. 5, 5a and 8 are the predicted mouse DPP10 polypeptide sequences. FIG. 21 shows both mouse and human polypeptide sequences.

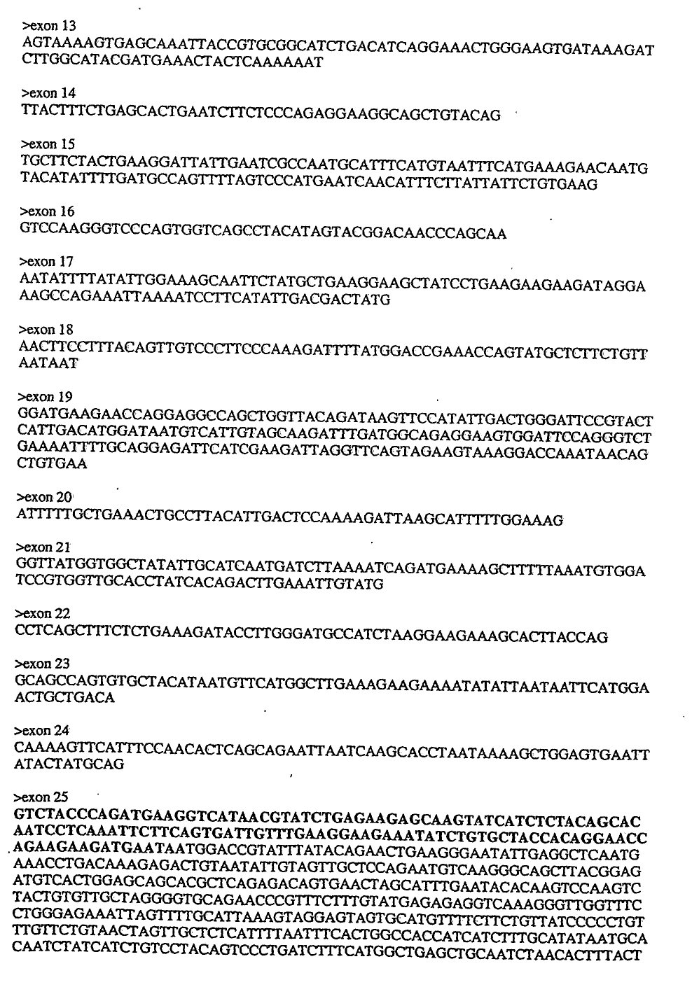

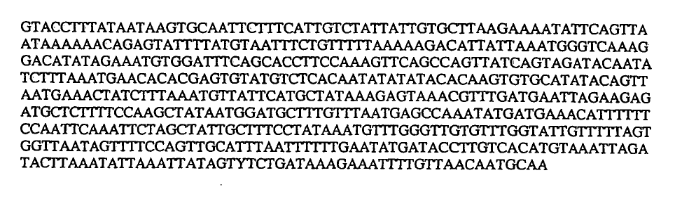

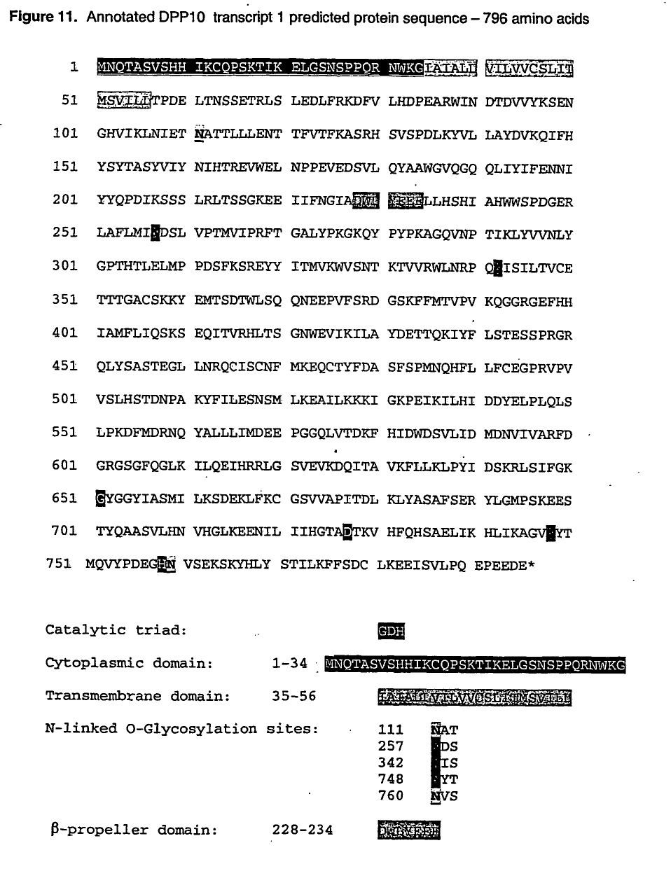

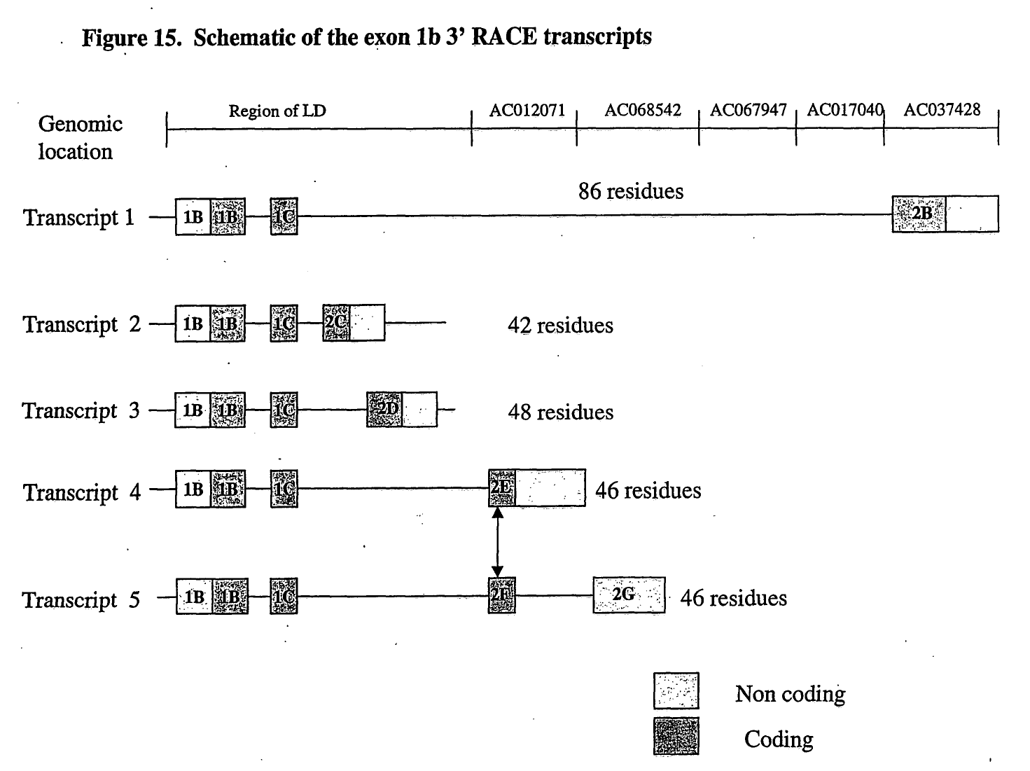

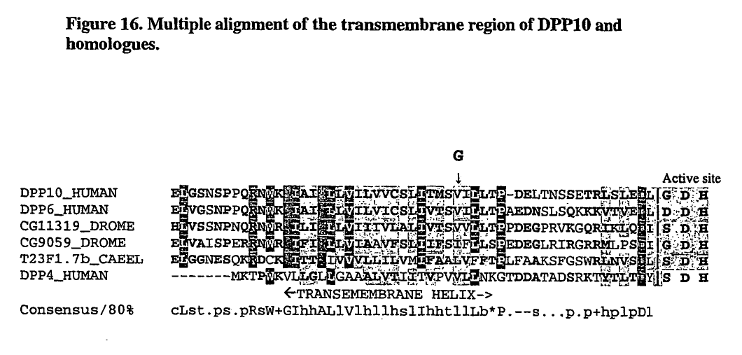

In a preferred embodiment of the third aspect there is provided a membrane bound form of DPP10 protein (FIG. 6, transcript 4; FIG. 4, FIG. 11). In particular the membrane bound form of DPP10 includes amino acids 35 to 56 of FIG. 11 which form the transmembrane domain (also FIG. 16). The mouse equivalents are transcripts 1, 2 and 3 (FIG. 7).

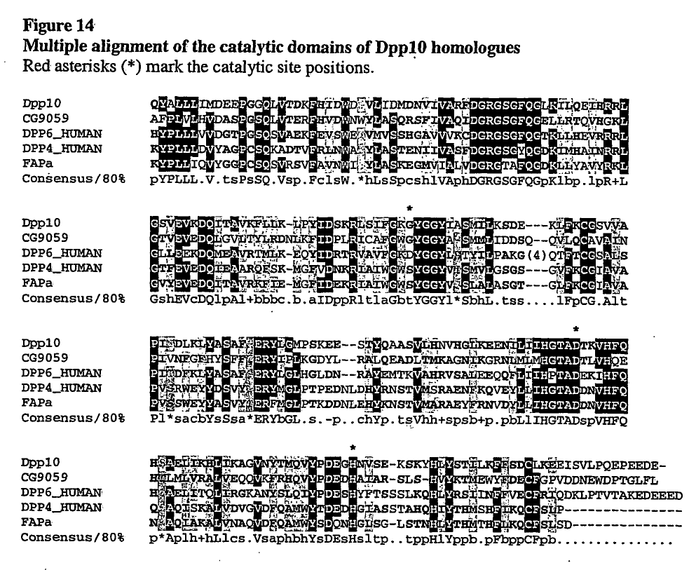

In a preferred embodiment of the third aspect, there is provided a soluble form of the DPP10 protein (Transcripts 2, 3, 5 and 6 of FIG. 6), which lacks a transmembrane domain or the catalytic domain (FIG. 14), or the beta-propeller domain (FIG. 17). In particular, the soluble DPP10 protein lacks amino acids 35 to 56 of FIG. 11, which form the transmembrane domain. In a most preferred embodiment, the soluble DPP10 protein comprises amino acids 57 to 751 or 796 of FIG. 11. The mouse equivalent is transcript 4 (FIG. 7).

The soluble DPP10 protein may be operably linked to a secretion signal, to assist its secretion from the golgi apparatus to another part of the cell. Suitable secretion signals can be provided by recombinant vectors such as pSecTag2 (Invitrogen Corporation, Carlsbad, Calif.). Proteins expressed from such vectors are fused at the N-terminus to the murine Ig kappa chain leader sequence. The secretion signal may be linked to the soluble DPP10 polypeptide sequence using techniques available in the art, including recombinant DNA technology.

The DPP10 protein or a sequence substantially homologous thereto of a fragment thereof may be subject to post-translational modification. Post-translational modification (PTM) is defined herein as including modification of a protein following translation by proteolytic cleavage e.g. cleavage of a preprotein, a proprotein or a preproprotein by removal of a signal sequence or activation of a zymogen. PTM also includes the attachment of a carbohydrate to a protein, the predominant sugars attached include glucose, galactose, mannose, fucose, GalNAC, GlcNAC and NANA. The carbohydrates may be linked to the protein either by β-glycosidic or N-glycosidic bonds. Also included are acylation; methylation; phosphorylation; sulfation and prenylation. Vitamin C-dependent modifications such as proline and lysive hydroxylation and carboxy terminal amidation and vitamin K-dependent modifications such as carboxylation of glutamine residues are also included as is the addition of selenium as selenocysterine in a protein.

The polypeptide sequences of the third aspect are preferably functional, and may be useful in drug screening, diagnosis or therapy. Functional. fragments of DPP10 are those which share immunological or functional characteristics with the full length, membrane bound or soluble form of DPP10. Fragments may be at least 10, preferably 15, 20, 25, 30, 35, 40 or 50 amino acids in length. Preferably, the polypeptide sequences are isolated.

In a fourth aspect of the present invention, there are provided antibodies which are specific for an antigen of a polypeptide sequence of the third aspect or an antigen of the isolated nucleic acid of the first aspect, or fragment of either aspect or which react with an antigen of a polypeptide sequence of the third aspect or the isolated nucleic acid of the first aspect, or fragment of either aspect. Herein the term “react” has the meaning that the antibody is able to interact with the polypeptide or isolated nucleic acid. The term “specific for” has the meaning that the antibody specifically reacts with the polypeptide or isolated nucleic acid.

Antibodies can be made by the procedure set forth by standard procedures (Harlow and Lane, “Antibodies; A Laboratory manual” Cold Spring Harbour Laboratory, Cold Spring Harbour, N.Y., 1998). Briefly, purified antigen can be injected into an animal in an amount and in intervals sufficient to elicit an immune response. Antibodies can either be purified directly, or spleen cells can be obtained from the animal. The cells are then fused with an immortal cell line and screened for antibody secretion. The antibodies can be used to screen DNA clone libraries for cells secreting the antigen. Those positive clones can then be sequenced as described in, for example, Kelly et al., Bio/Technology 10: 163-167 (1992) and Bebbington et al., Bio/Technology 10:169-175 (1992). Preferably, the antigen being detected and/or used to generate a particular antibody will include polypeptide sequences according to the third aspect or isolated nucleic acid sequences according to the first aspect. The antibody may be a polyclonal or monoclonal antibody, a chimeric antibody, a humanised antibody or a bifunctional antibody or a fragment of any of the above. A bifunctional antibody is an antibody that can bind to two different antigens, these antigens may be different antigens present in the DPP10 polypeptide or isolated nucleic acid or may be an antigen of DPP10 combined with e.g. a cellular antigen.

In particular, the antibody may be raised against a particular domain of DPP10, such as the cytosolic soluble form; the β-propeller domain, or the external domain. Such antibodies will be useful in diagnostic and therapeutic aspects of the invention. In particular, the antibodies will be useful in the development of assays for detecting or measuring DPP10 in a sample from a subject.

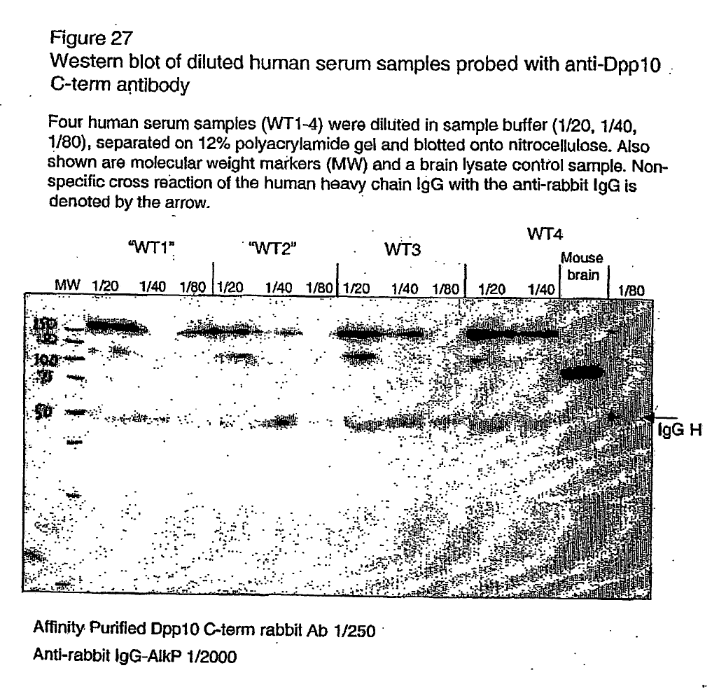

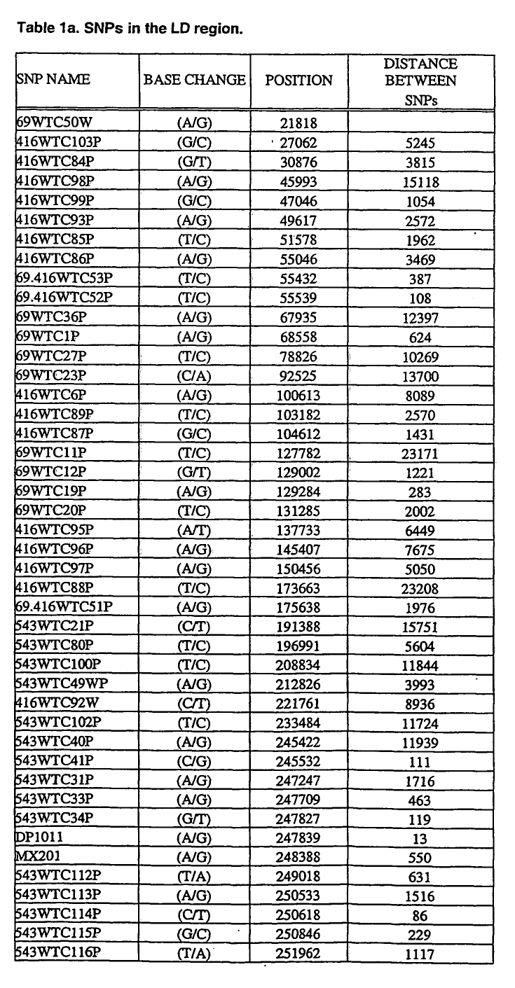

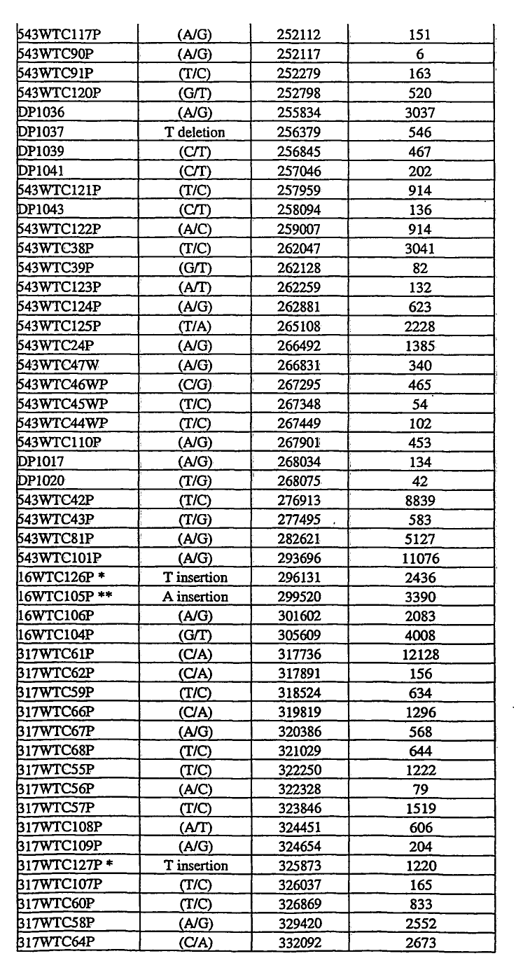

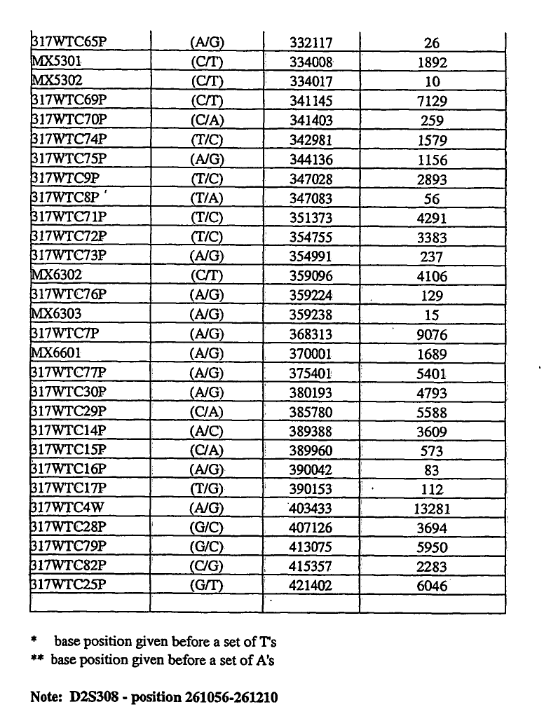

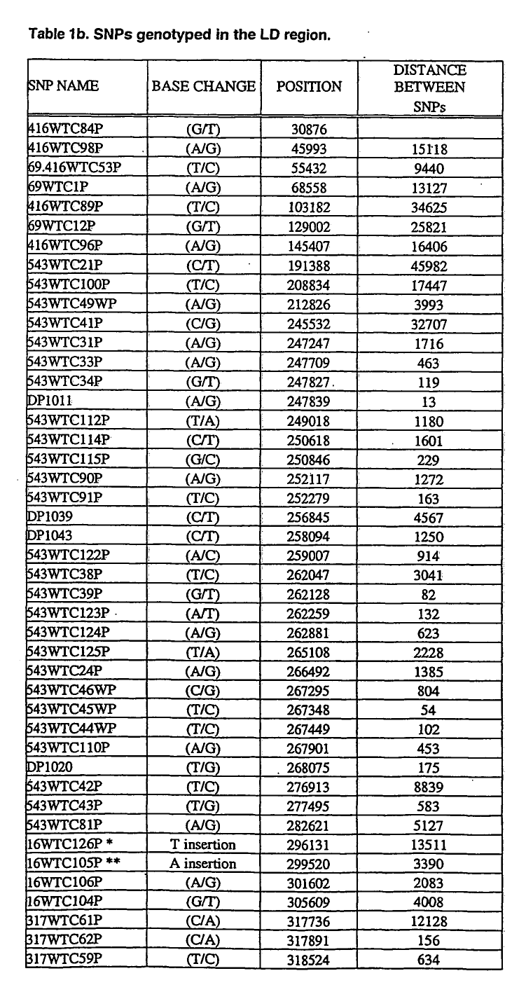

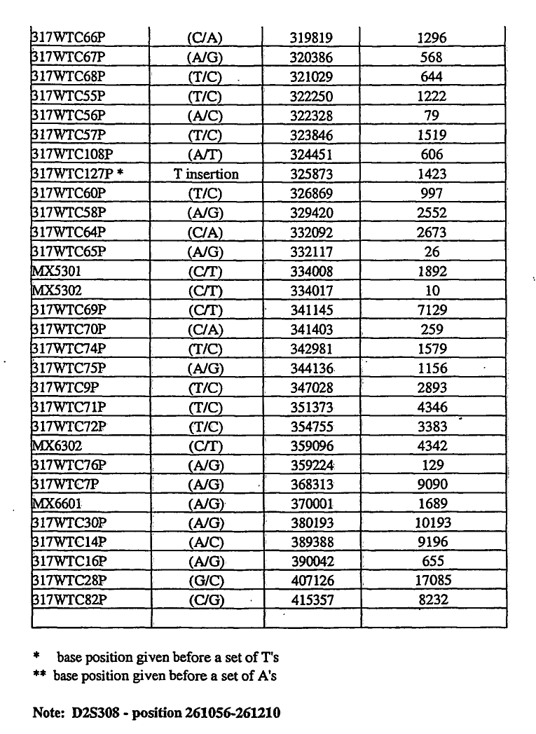

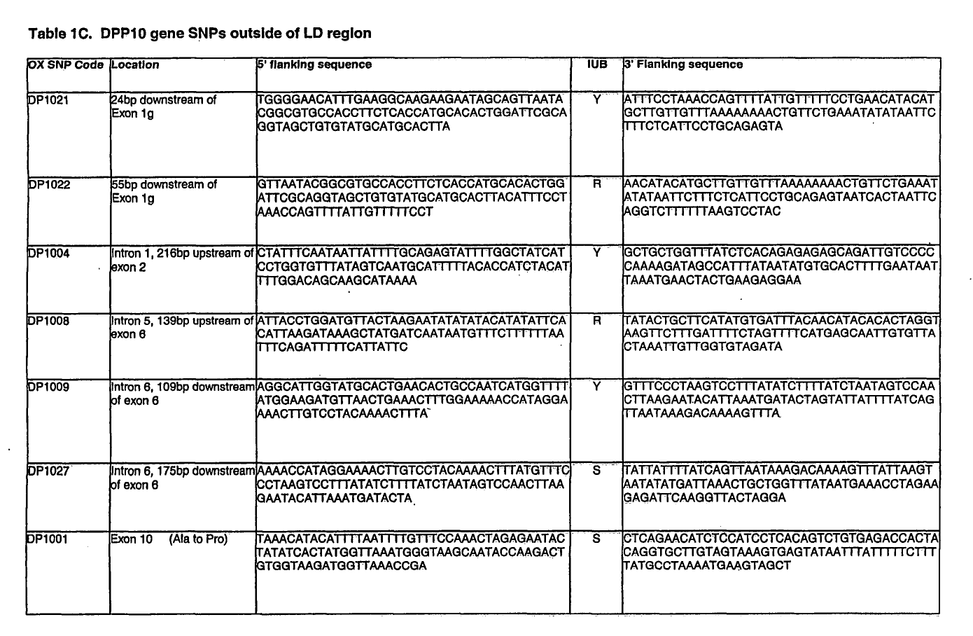

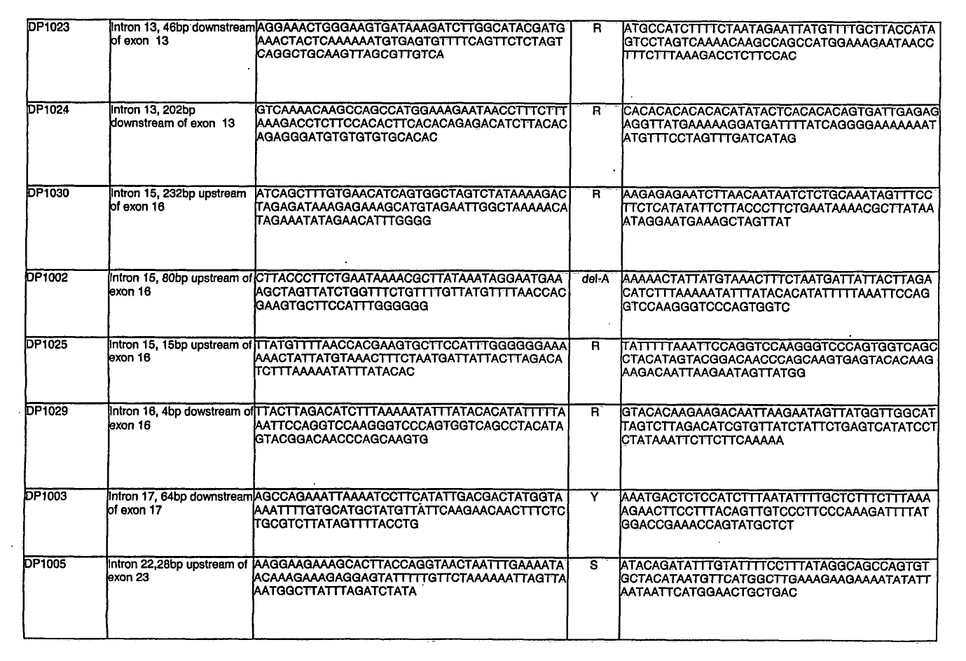

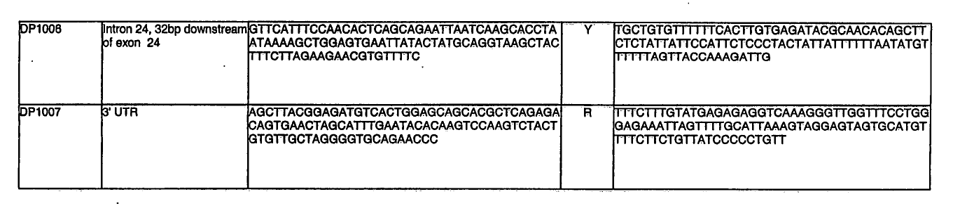

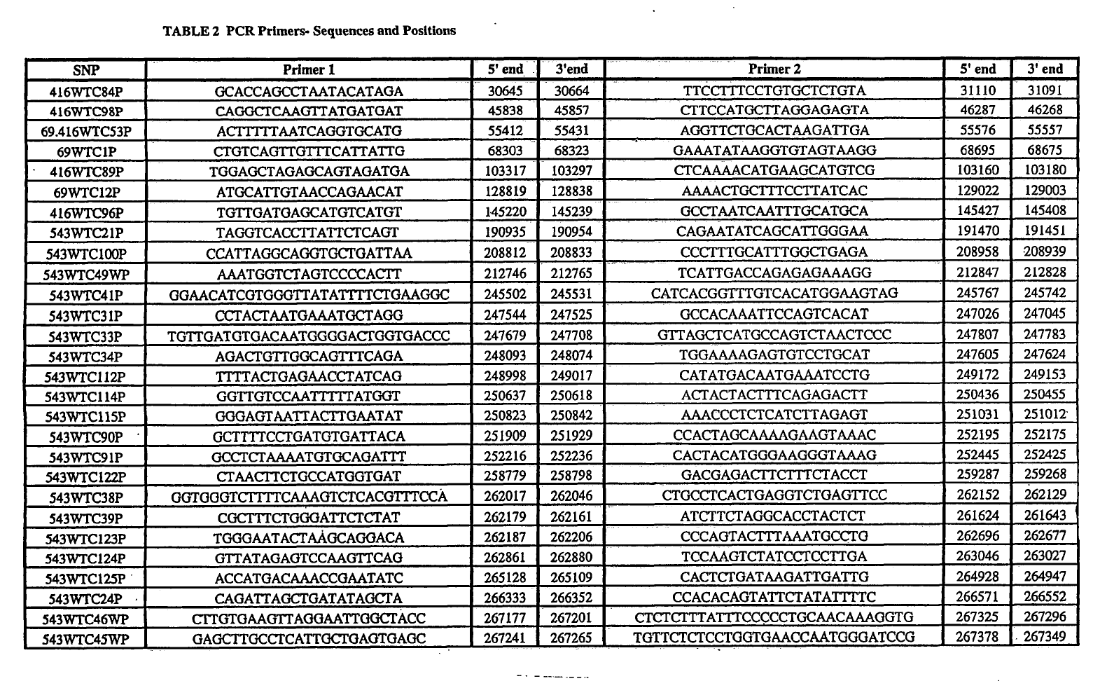

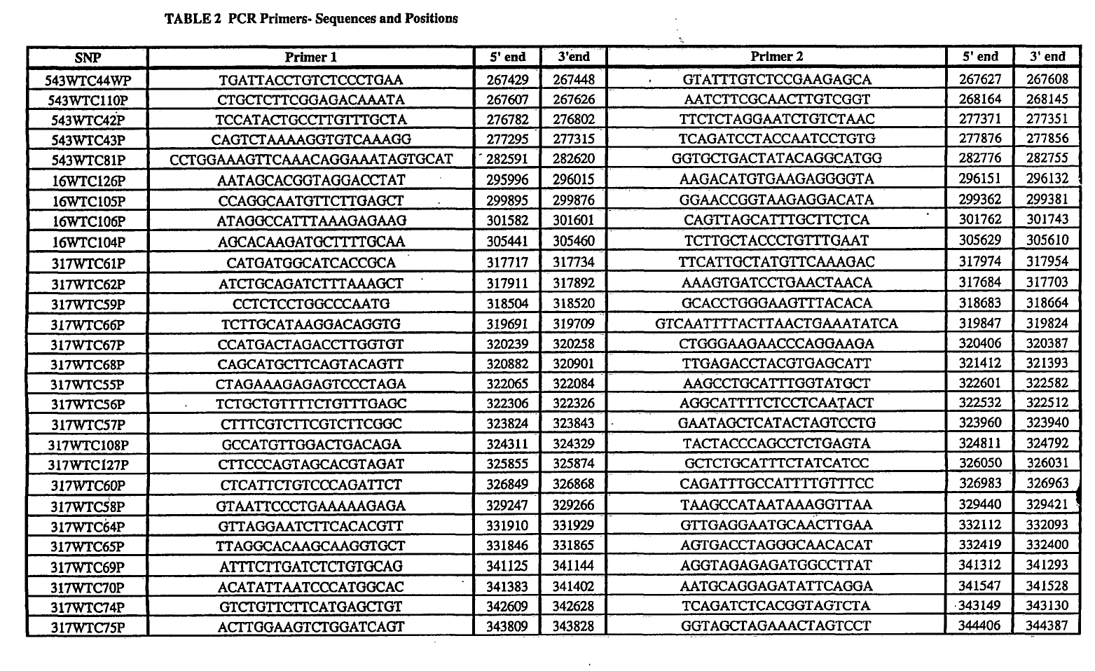

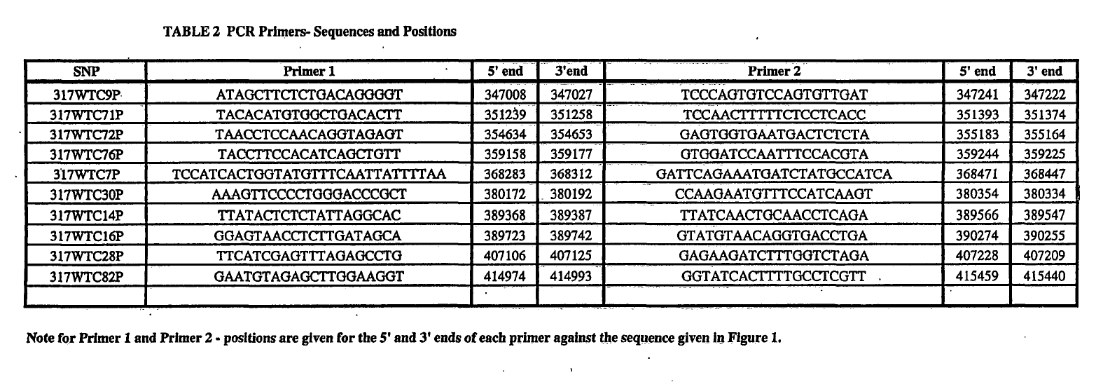

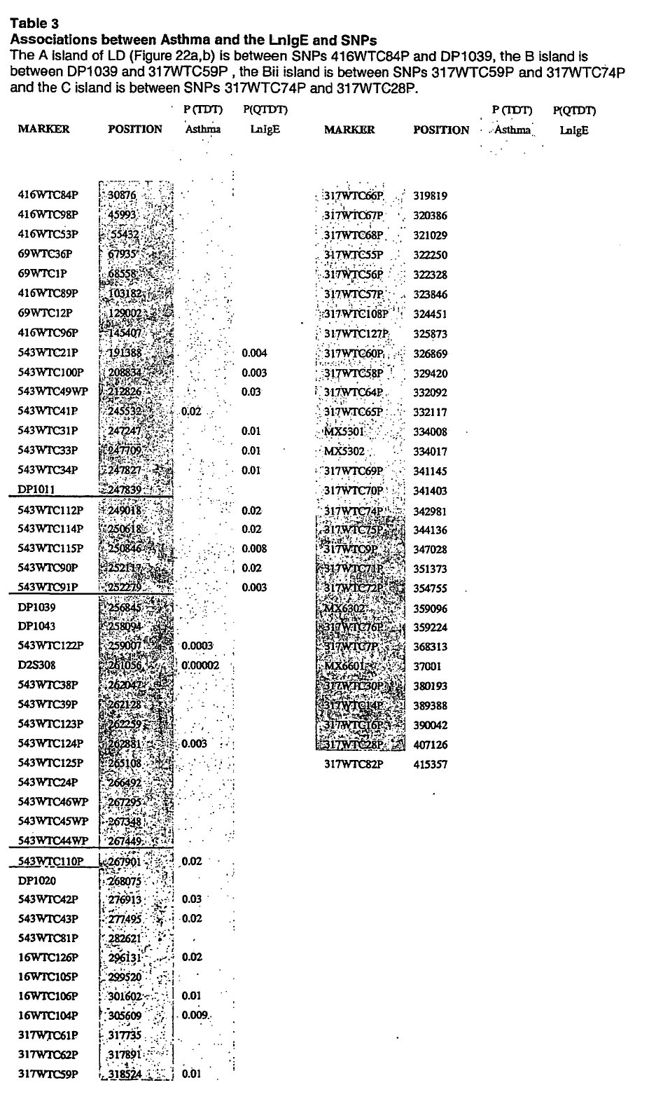

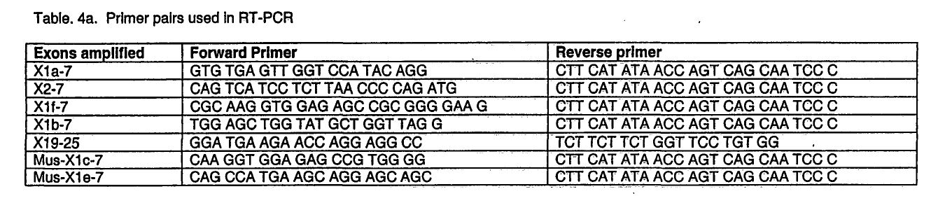

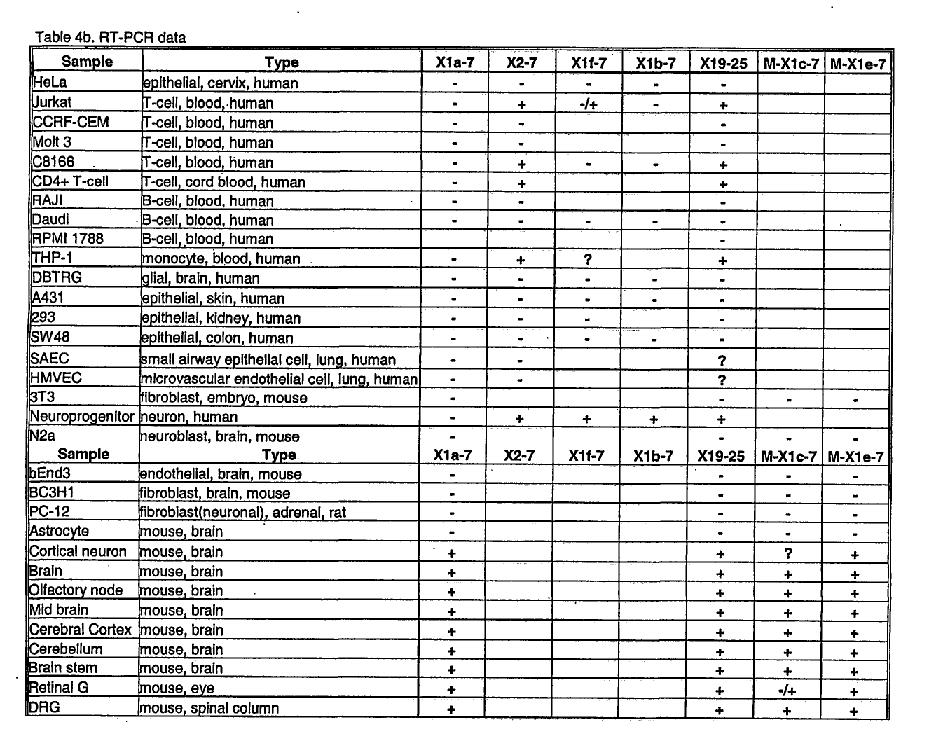

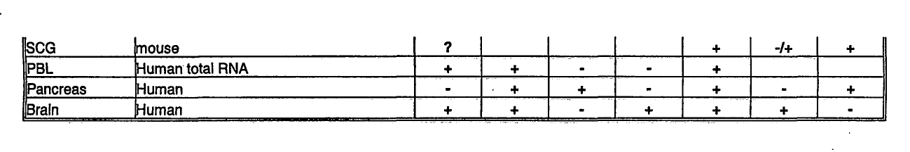

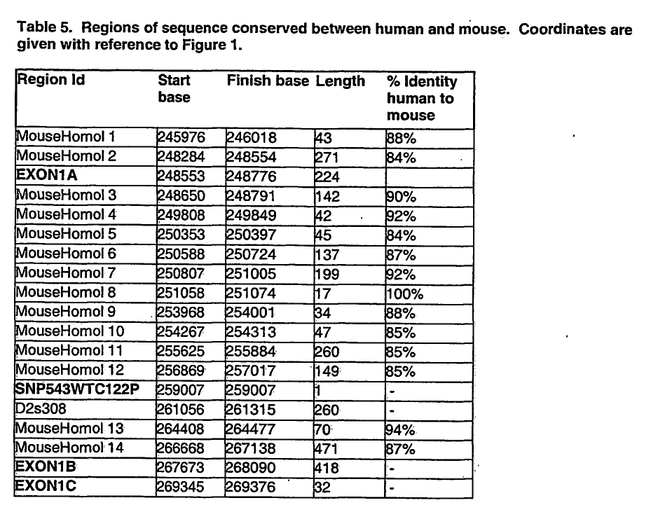

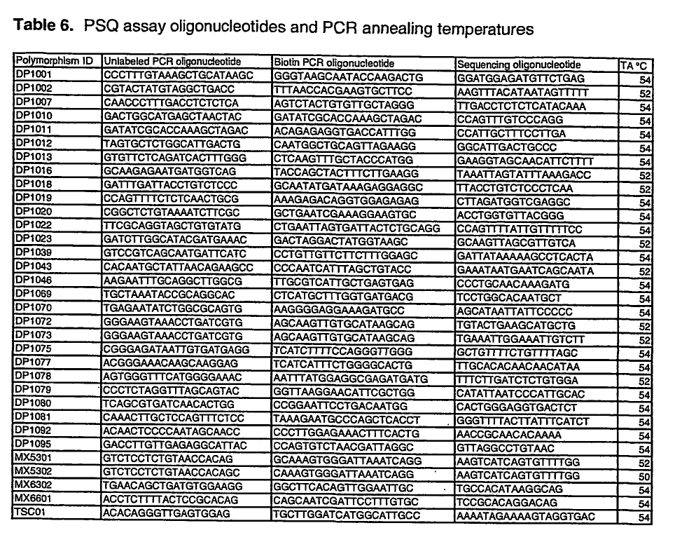

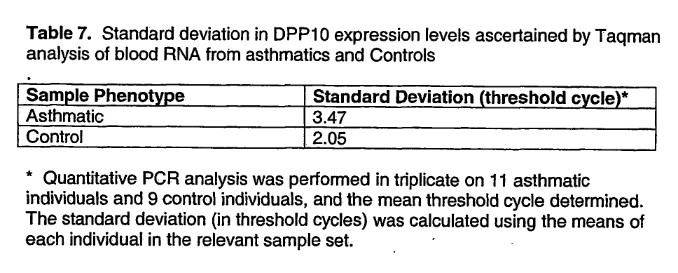

In a preferred embodiment, the antibody may be used to assay the level of cytosolic, soluble DPP10 protein in a serum sample obtained from a subject (FIG. 27). According to a fifth aspect of the invention, there is provided a process for the preparation of a nucleic acid sequence as defined above, the process comprising ligating together successive nucleotide and/or oligonucleotide residues together. Such a process may be carried out using chemical synthesis methods or by using enzymic catalysis. Alternatively, a suitable host cell may be transfected with an appropriate DNA or RNA sequence so as to cause production of the desired sequence in a host cell.

In a sixth aspect of the invention, there is provided a process for the preparation of a polypeptide as defined above, the process comprising ligating together successive amino acids and/or oligonucleotides together. Such a process may be carried out using chemical synthesis methods or by using enzymic catalysis. Alternatively, a suitable host cell may be transfected with an appropriate DNA or RNA sequence so as to cause production of the desired sequence in a host cell.

In the context of the present invention, references to DPP10 include the soluble, membrane bound or insoluble forms of the protein, which comprise at least the polypeptide sequence encoded by exons 2 to 25. Thus, references to DPP10 include proteins encoded by transcripts having one or more of exons 1a, 1b, 1c, 1d, 1e, 1f or 1g.

In a seventh aspect of the present invention, there is provided the following; a cell comprising a nucleic acid sequence according to an aforementioned aspect of the invention; or a transgenic non-human animal comprising a nucleic acid sequence according to an aforementioned aspect of the invention. Such cells (either alone, in suspension, in culture or as part of a group of cells representing an organ) and transgenic non-human animals are useful for the analysis of a single nucleotide polymorphisms and their phenotypic effect and so for the analysis of DPP10 and its phenotypic effect. Expression of a polynucleotide sequence of the invention in a transgenic non-human animal is usually achieved by operably linking the polynucleotide to a promoter and/or enhancer sequence, preferably to produce a vector of the above aspect, and introducing this into an embryonic stem cell of a host animal by microinjection techniques (Hogan et al., A Laboratory Manual., Cold Spring harbour and Capecchi Science (1989) 244: 1288-1292). The transgene construct should then undergo homologous recombination with the endogenous gene of the host. Those embryonic stem cells comprising the desired nucleic acid sequence may be selected, usually by monitoring expression of a marker gene, and used to generate a non-human transgenic animal. Preferred host animals include mice, rabbits and other rodents.

The nucleic acid sequence introduced may not be native to the host animal, i.e. it may be foreign. Such transgenic animals may be distinguished from native, non-transgenic animals using methods known in the art, for example a nucleic acid sample from the transgenic animal may be compared with that from a native animal—the transgenic animal will have a nucleic acid sequence such as a foreign promoter, marker genes etc. Alternatively, the phenotypes of the animals can be compared.

Where it is desirable to use the transgenic non-human animal of the seventh aspect to study disease, it may be desirable for the nucleic acid introduced into the animal to encode a variant of DPPIO which results in allergy, atopy, asthma or inflammatory disease including rheumatoid arthritis, ankylosing spondylitis, inflammatory bowel disease, Crohn's disease, multiple sclerosis or type II diabetes. In such embodiments where the disease has been artificially introduced, the transgenic non-human animal will be modulated such that it no longer expresses the native DPP10 gene. These animals may be referred to as “knock-out” (Manipulating The Mouse Embryo—A Laboratory Manual, Hogan et al 1986). In some cases, it may be desirable to modulate the expression of the foreign nucleic acid and/or the native gene in a temporal or spatial manner. This approach removes viability problems if the expression of the native gene is abolished in all tissues.

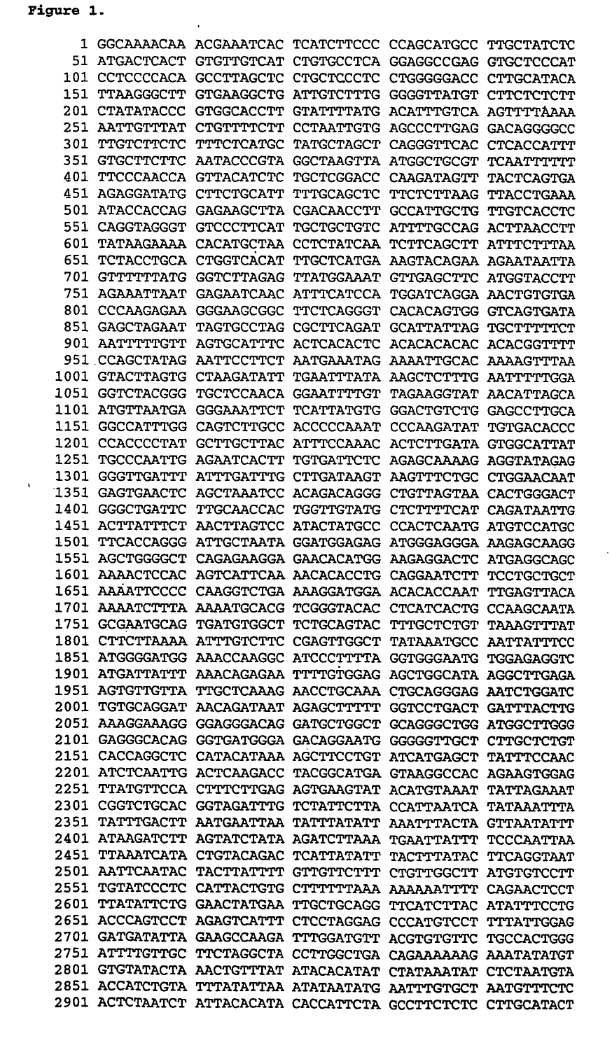

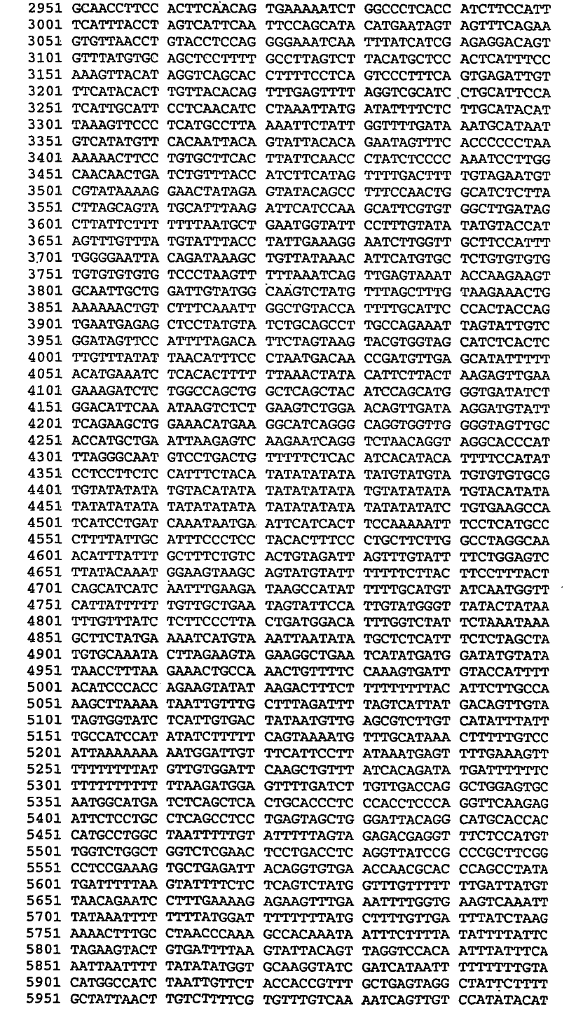

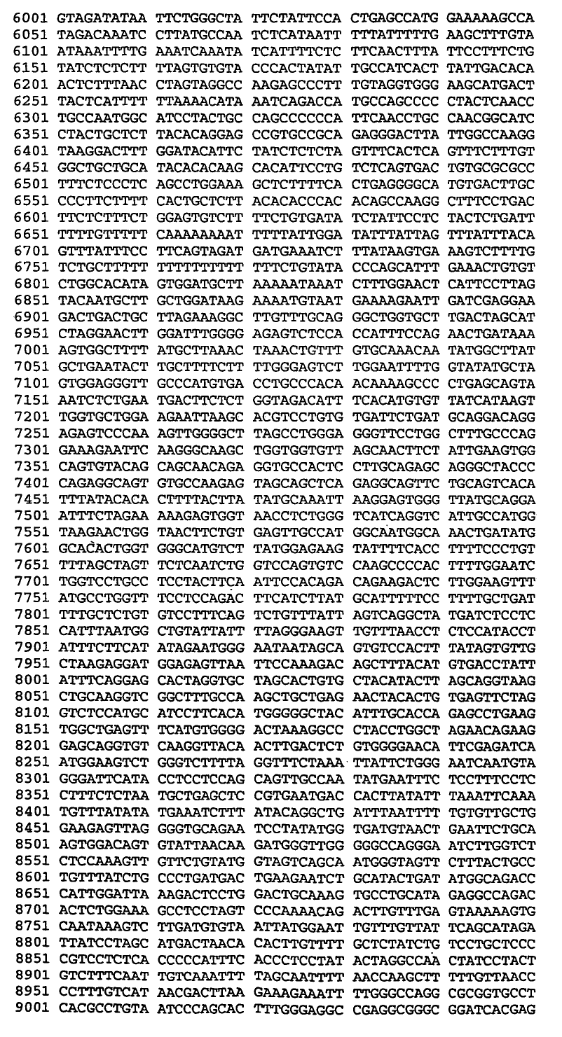

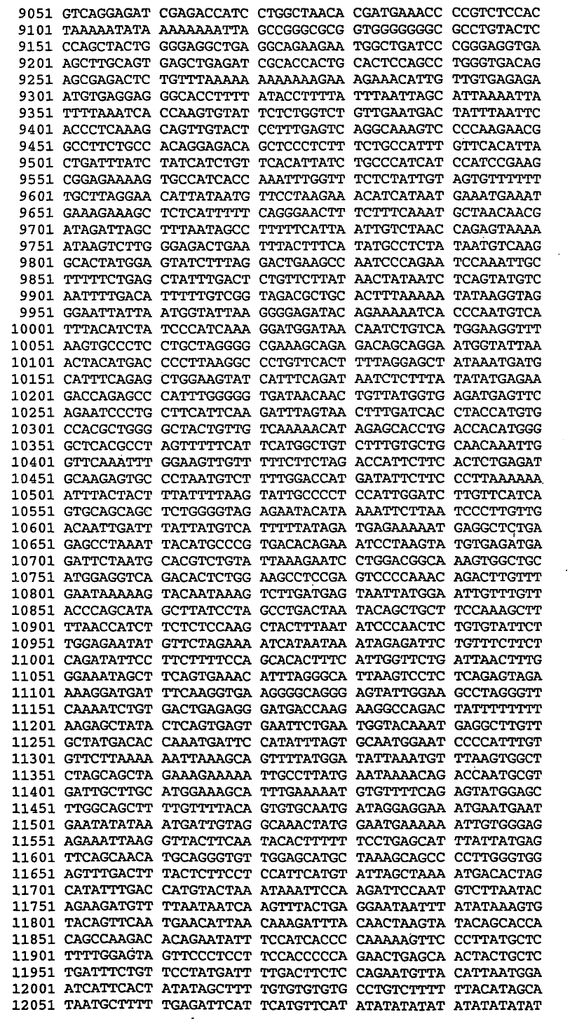

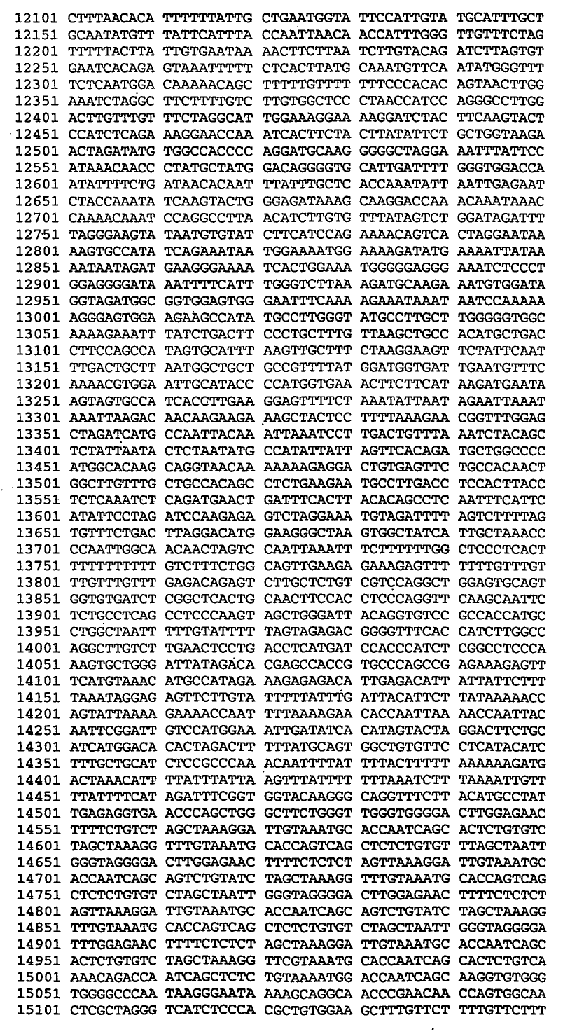

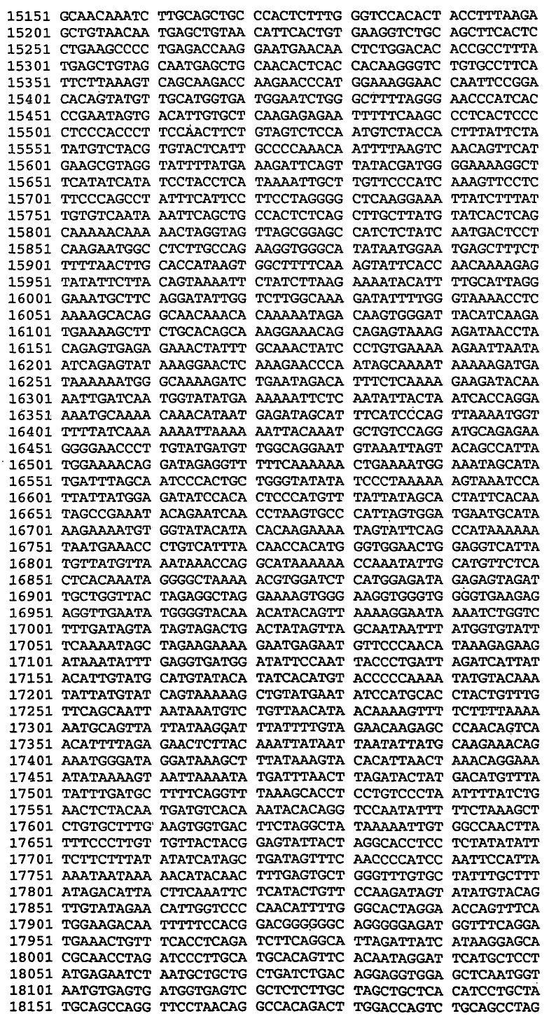

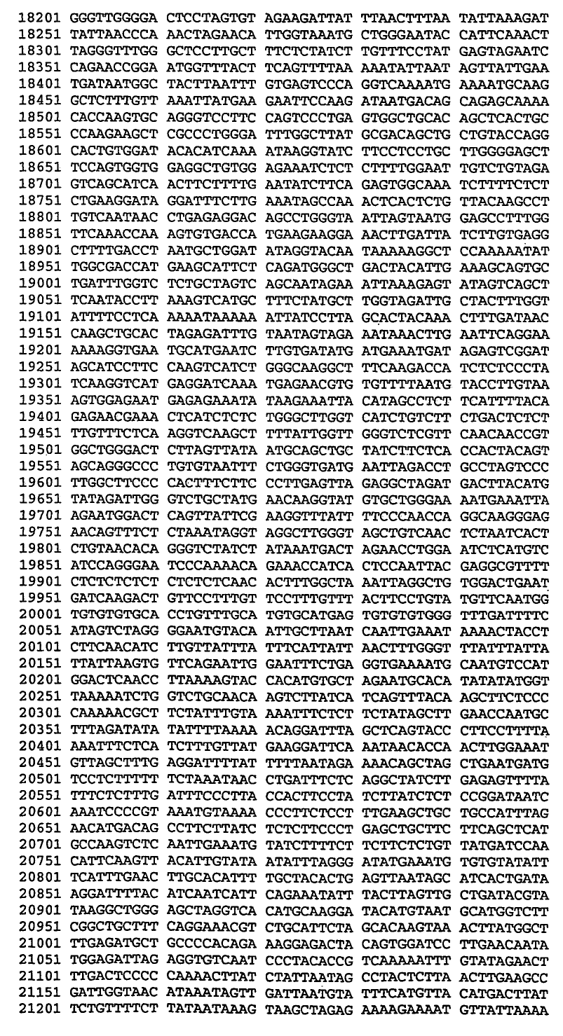

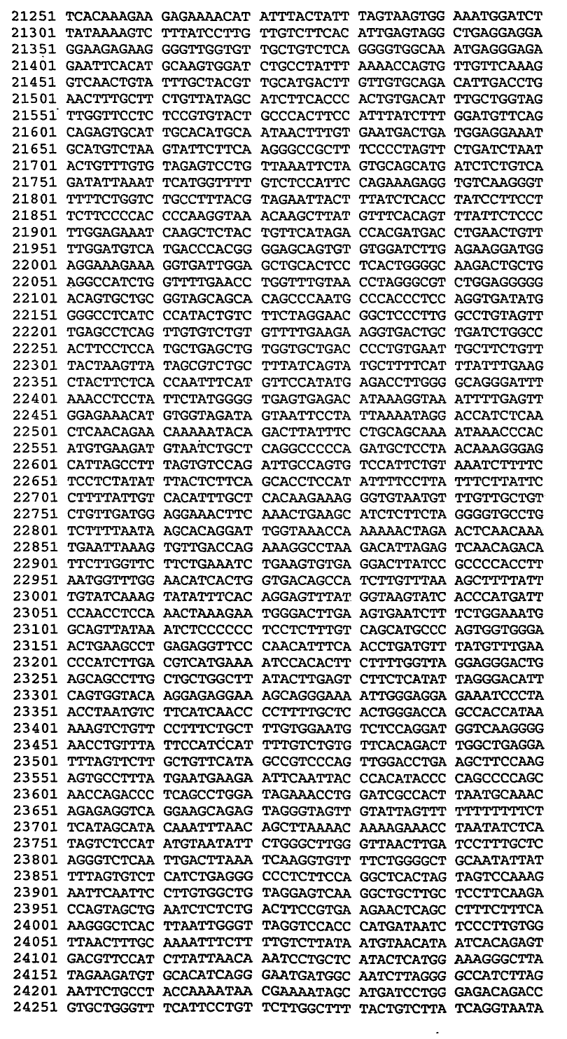

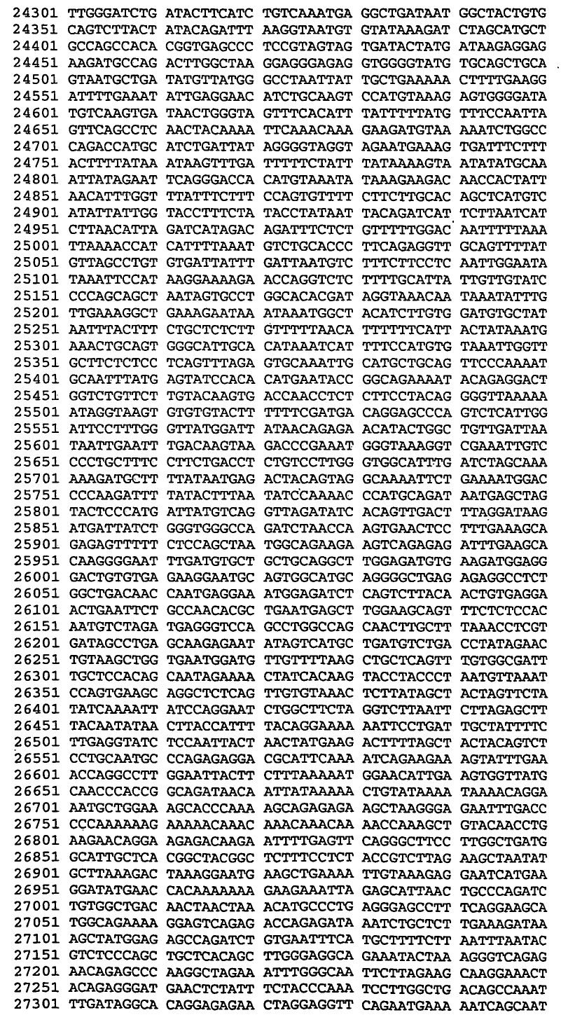

In a most preferred embodiment, there is provided a transgenic mouse comprising a nucleic acid encoding a variant form of DPP10 which causes allergy, atopy, asthma or inflammatory disease including rheumatoid artitis, ankylosing spondylitis, inflammatory bowel disease, Crohn's disease, multiple sclerosis or type II diabetes. Most preferably, the nucleic acid molecule comprises a SNP at the position which corresponds to Position 259007 (where the base adenine is changed to cytosine), Position 267901 (where the base adenine is changed to guanine) and/or Position 318524 (where the base thymine is changed to cytosine) of FIG. 1. Preferably, the mouse is modulated so that it no longer expresses DPP10 in a temporally and/or spatially appropriate manner using homologous recombination techniques or alternatively to over express DPPlO protein as a result of transgenic manipulation.

If a functional, polymorphism is identified (i.e. a “mutation”) a DPP10 construct containing this polymorphism can be introduced into the mouse germ line (i.e. a knock-in) to produce a pathological variant of the DPP10 protein rather than knocking it out. Alternatively a pathological variant of the DPP10 gene may be overexpressed.

In the context of the present invention, inflammatory diseases include those resulting from overexpression of DPP10, or the presence of a variant form of DPP10. Specifically, such diseases include allergies, and atopic diseases such as asthma, or inflammatory bowel disease e.g. ulcerative colitis and Crohn's disease, and inflammatory joint disease such as rheumatoid arthritis and ankylosing spondylitis, or psoriasis, multiple sclerosis or type II diabetes.

In an eighth aspect of the present invention, there is provided a method of diagnosing, or determining susceptibility of a subject to inflammatory disease. The method may comprise determining the presence of a variant form of DPP10, which is known to be associated with a disease state, or measuring the levels of DPP10. A variant form of DPP10 includes nucleic acid and amino acid variants. A variant includes any SNP from the wild-type (e.g. for humans) (FIG. 1) or other mutation or alteration from the wild-type.

For example, probes or primers as described above may be useful in detecting nucleic acid encoding DPP10 or a variant thereof Information regarding the expression pattern or forms of DPP10 present will be useful in determining whether the individual is susceptible to inflammatory diseases, resulting from altered expression of DPP10; possibly by influencing the ratio of membrane bound to cytosolic forms of the DPP10 protein.

In a preferred embodiment, the method may additionally, or alternatively, comprise determining the presence or absence of a risk allele of one or more of the SNPs of Tables 1a, 1b and 1c or Table 3, where presence of a risk allele is indicative of disease or predisposition to disease. The method may also comprise genotyping one or more known polymorphisms. Any combination of such polymorphisms may be genotyped.

The SNPs of the invention are listed in Tables 1a, 1b and 1c or Table 3, where the nature of the polymorphism is described in the format wild type allele/variant allele. For example, the SNP at position 21818 (denoted 69WTC50W) has an adenosine residue in the wild type sequence, which is substituted for a guanine residue in the variant sequence. The SNPs are positioned with respect to FIG. 1, where nucleotide position 1 is the 1^{st} nucleotide in the FIG. 1.

Preferably, the allele of the SNP polymorphisms are as follows: a nucleotide residue other than adenine at position 259007; a nucleotide residue other than adenine at position 267901; and a nucleotide residue other than thymine at position 318524 of FIG. 1. More specifically, the risk allele are a cytosine residue at position 259007; a guanine residue at position 267901; and a cytosine residue at position 318524 of FIG. 1.

The alleles for the remaining SNPs identified in the present invention are described in Tables 1a, 1b and 1c or Table 3.

Any technique, including those known to persons skilled in the art, may be used in the above method. These may include the use of probes or primers as described above, or antibodies of the fourth aspect, for example in ELISA assays or in immuno-localisation. Preferably, the method comprises first removing a sample from a subject. More preferably, the method comprises isolating from a sample a nucleic acid or a polypeptide sequence.

In particular, methods for use in this aspect include those known to persons skilled in the art for identifying differences between nucleic acid sequences, for example direct probing, allele specific hybridisation, PCR methodology including Pyrosequencing (Ahmadian A, Gharizadeh B, Gustafsson AC, Sterky F, Nyren P, Uhlen M, Lundeberg J. Single-nucleotide polymorphism analysis by pyrosequencing, Anal Biochem. 2000 Apr 10;280(l):103-10; Nordstrom T, Ronaghi M, Forsberg L, de Faire U, Morgenstern R, Nyren P. Direct analysis of single-nucleotide polymorphism on double-stranded DNA by pyrosequencing. Biotechnol Appl Biochem. 2000 Apr,31 (Pt 2):107-12) Allele Specific Amplification (ASA) (W093/22456), Allele Specific Hybridisation, single base extension (US patent No. 4,656,127), ARMS-PCR, Taqman TM (US 4683202; 4683195; and 4965188), oligo ligation assays, single-strand conformational analysis ((SSCP) Orita et al PNAS 86 2766-2770 (1989)), Genetic Bit Analysis (WO 92115712) and RFLP direct sequencing, mass-spectrometry (MALDI-TOF) and DNA arrays. The appropriate restriction enzyme, will, of course, be dependent upon the polymorphism and restriction site, and will include those known to persons skilled in the art. Analysis of the digested fragments may be performed using any method in the art, for example gel analysis, or southern blots.

There is provided a method of diagnosing, or determining predisposition to disease, comprising determining the presence or absence of a risk allele of a SNP at position 259007 of FIG. 1, wherein presence of the risk allele is diagnostic of disease or predisposition of disease.

In addition, there is provided a method for diagnosing, or determining predisposition to, disease comprising determining the presence or absence of risk alleles of a SNP at positions 267901 and/or 318524 of FIG. 1, wherein presence of a risk allele is diagnostic of disease or predisposition to disease.

The present invention is advantageous in that it facilitates the accurate diagnosis of disease, or the determination of predisposition to disease. Thus, by genotyping, an individual may be identified as having or being predisposed to disease. This helps to identify those individuals who are likely to respond positively to particular treatments or preventative measures. Thus, more effective therapies or preventative measures can be administered.

The diseases which are associated with the polymorphisms of the invention include inflammatory diseases, such as inflammatory bowel disease, e.g. ulcerative colitis and Crohn's disease, and inflammatory joint disease, such as rheumatoid arthritis and ankylosing spondylitis, or multiple sclerosis or type II diabetes. Predisposition to disease in the context of the present invention means that. these individuals are at higher risk of developing the disease, or a more severe form of the disease, or a particular form of the disease.

In the context of the present invention, a risk allele is the allele of a polymorphism which is associated with disease or predisposition to disease. The risk allele may be the wild type or the variant allele, as defined below.

The term “polymorphism” refers to the coexistence of multiple forms of a sequence. Thus, a polymorphic site is the location at which sequence divergence occurs. The different forms of the sequence which exist as a result of the presence of a polymorphism are referred to as “alleles”. The region comprising a polymorphic site may be referred to as a polymorphic region.

Examples of the ways in which polymorphisms are manifested include restriction fragment length polymorphisms (13otstein et al Am J Hum Genet 32 314331 (1980)), variable number of tandem repeats, hypervariable regions, minisatellites, di- or multi-nucleotide repeats, insertion elements and nucleotide or amino acid deletions, additions or substitutions. A polymorphic site may be as small as one base pair, which may alter a codon thus resulting in a change in the encoded amino acid sequence.

Single nucleotide polymorphisms arise due to the substitution, deletion or insertion of a nucleotide residue at a polymorphic site. Such variations are referred to as SNPs. SNPs may occur in protein coding regions, in which case different polymorphic forms of the sequence may give rise to variant protein sequences. Other SNPs may occur in non-coding regions. In either case, SNPs may result in defective proteins or regulation of genes, thus resulting in disease. Other SNPs may have no phenotypic effects, but may show linkage to disease states, thus serving as markers for disease. SNPs typically occur more frequently throughout the genome than other forms of polymorphism discussed above, and there is therefore a greater probability of finding a SNP associated with a particular disease state.

Linkage disequilibrium is the coinheritance of two alleles at greater frequencies than would be expected from the separate frequencies of each allele. Conversely, alleles are in linkage equilibrium if they occur together. The expected frequency of two alleles inherited together is the product of the frequency of each allele.

Where two or more polymorphisms are genotyped, the method preferably defines determining the presence or absence of a haplotype which is indicative of disease or predisposition to disease. A haplotype is defined herein as a collection of polymorphic sites in a particular sequence that are inherited in a group, i.e. are in linkage disequilibrium with each other. The identification of haplotypes in the diagnosis of disease helps to reduce the possibility of false positives. The haplotype may be any particular combination of polymorphisms of Tables 1a, 1b and 1c or Table 3, optionally in combination with one or more known polymorphisms. A preferred haplotype is the combination of SNPs at positions 259007, 267901 and 318524 of FIG. 1.

The methods of the eighth aspect are preferably carried out on a sample removed from a subject. Any biological sample comprising cells containing nucleic acid, preferably that of FIG. 1, is suitable for this purpose. Examples of suitable samples include whole blood, leukocytes, semen, saliva, tears, buccal, skin or hair. For analysis of CDNA, mRNA or protein, the sample must come from a tissue in which the sequence of interest is expressed. Blood is a readily accessible sample. Thus, the method of the eighth aspect preferably includes the steps of obtaining a sample from a subject, and preparing nucleic acid from the sample.

The subject is preferably a mammal, and more preferably a human. The subject may be an infant, a child or an adult. Alternatively, the sample may be obtained from the subject prepartum e.g. by amniocentesis.

A subject's risk factor for disease may be determined with reference also to other known genetic factors, and/or clinical, physiological or dietary factors. The above described methods may require amplification of the DNA sample from the subject, and this can be done by techniques known in the art, such as PCR (see PCR Technology: Principles and Applications for DNA Amplification (ed. H. A. Erlich, Freeman Press, N.Y. 1992; PCR Protocols: A Guide to methods and Applications (eds. Innis et al., Academic press, San Diego, Calif. 1990); Mattila et al., Nucleic Acids Res. 19 4967 (1991); Eckert et al., PCR Metlods and Applications 117 (1991) and U.S. Pat. No 4,683,202. Other suitable amplification methods include ligase chain reaction (LCR) (Wu et al., Genomics 4 560 (1989); Landegran et al., Science 241 1077 (1988)), transcription amplification (Kwoh et al., Proc Natl Acad Sci USA 86 1173 (1989)), self sustained sequence replication (Guatelli et al., Proc Natl Acad Sci USA 87 1874 (1990)) and nucleic acid based sequence amplification (NASBA). The latter two methods both involve isothermal reactions based on isothermal transcription which produce both single stranded RNA and double stranded DNA as the amplification products, in a ratio of 30 or 100 to 1, respectively.

Where it is desirable to analyse multiple samples simultaneously, it may be preferable to use arrays as described in WO95/11995. The array may contain a number of probes, each designed to identify variants of DPP10 from a sample.

Where a restriction enzyme is required, it can be selected according to the nature of the polymorphism and restriction site. Suitable enzymes will be known to persons skilled in the art. Analysis of the digested fragments may be performed using any method in the art, for example gel analysis, or southern blots.

Determination of an allele of a polymorphism using the above methods typically involves the use of anti-sense sequences i.e. sequences which are complementary to the nucleic acid sequences of interest, which may include part of the sequence of FIG. 1. Such sequence are described in the third aspect of the invention.

Where it is desirable to identify the presence of multiple single nucleotide polymorphisms, or haplotypes, in a sample from a subject, it may be preferable to use arrays as described in WO95/11995. The array may contain a number of probes, each designed to identify one or more of the above single nucleotide polymorphisms of the invention.

An antibody to DPP10 as previously described may be used in the method of the eighth aspect. The detection of binding of the antibody to the antigen in a sample may be assisted by methods known in the art, such as the use of a secondary antibody which binds to the first antibody, or a ligand. Immunoassays including immunofluorescence assays (IFA) and enzyme linked immunosorbent assays (ELISA) and immunoblotting may be used to detect the presence of the antigen. For example, where ELISA is used, the method may comprise binding the antibody to a substrate, contacting the bound antibody with the sample containing the antigen, contacting the above with a second antibody bound to a detectable moiety (typically an enzyme such as horse radish peroxidase or alkaline phosphatase), contacting the above with a substrate for the enzyme, and finally observing the colour change which is indicative of the presence of the antigen in the sample.

Any biological sample comprising cells containing nucleic acid or protein is suitable for this purpose. Examples of suitable samples include whole blood, semen, saliva, tears, buccal, skin or hair. For analysis of cDNA, mRNA or protein, the sample must come from a tissue in which DPP10 is expressed. Peripheral blood leukocytes are a readily accessible sample.

According to a ninth aspect of the invention, there is provided a method of preventing or treating disease in a subject, wherein the method comprises modulating the activity, expression, half life or post translational modification of DPP10 in the subject. Preferably, the method is carried out in a subject who has been diagnosed as suffering from, or is susceptible to allergies, atopic diseases such as asthma or inflammatory bowel disease e.g. ulcerative colitis and Crohn's disease, and inflammatory joint disease such as rheumatoid arthritis and ankylosing spondylitis, or psoriasis, multiple sclerosis or type II diabetes

Preferably, the method comprise determining the presence or absence of a risk allele of a SNP such as one which has an association with allergy, atopy, asthma or inflammatory disease e.g. at position 259007, 267901 and/or 318524 of FIG. 1; and if the risk allele is present, administering treatment in order to prevent, delay or reduce the disease.

Preferably, the step of comprising determining the presence or absence of a risk allele is carried out in accordance with the eighth aspect, and therefore also comprises determining the presence or absence of risk alleles of SNPs of Tables 1a, 1b and 1c or Table 3, or any combination thereof, for example as described above.

The prevention or treatment of disease according to the ninth aspect may include the administration of any agent capable of modulating the effects of DPP10 or of the disease-causing allele. Preferably, the agent is one which is capable of ameliorating the deleterious effects of the risk allele. The methods include, but are not limited to, gene therapy techniques. Gene therapy techniques typically involve replacing the nucleic acid sequence comprising the risk allele, or otherwise down regulating the effects of the risk allele. The nucleic acid sequences of the second aspect, or sequences anti-sense thereto, will be useful in gene therapy.

By modulating is meant inhibiting or increasing the activity of the enzyme. Preferably, the activity is inhibited. The activity of the enzyme includes any aspect of its production or function, including transcription and translation of nucleic acid sequences, and assembly of the protein, and downstream interactions with other factors.

The DPP10 activity can be modulated in a number of ways. For example, the expression of the gene may be inhibited through the use of antisense sequences, such as those of the first aspect of the invention or by the production of anti sense RNA sequences. Such sequences when introduced into a subject by gene therapy will hybridise to the DPP10 gene or RNA, and inhibit its transcription or translation. This method may be particularly useful where it is desirable to modulate the function or expression of certain splice variants of DPP10 whilst not affecting others.

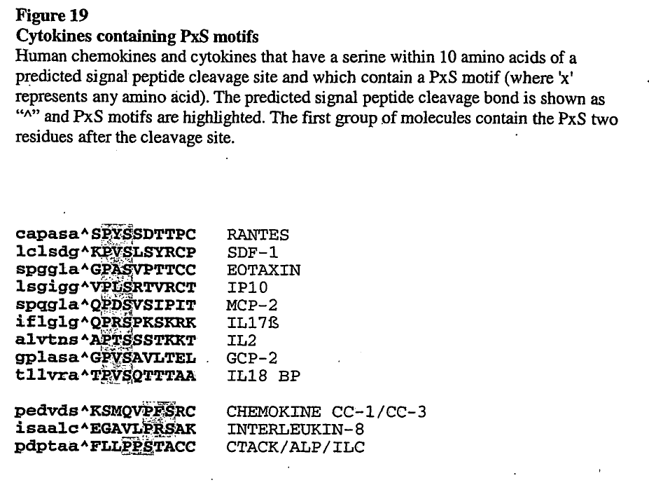

Introduction of a nucleic acid sequence may use gene therapy methods including those known in the art. In general, a nucleic acid sequence will be introduced into the target cells of a subject, usually in the form of a vector and preferably in the form of a pharmaceutically acceptable carrier. Any suitable delivery vehicle may be used, including viral vectors, such as retroviral vector systems which can package a recombinant genome. The retrovirus could then be used to infect and deliver the polynucleotide to the target cells. Other delivery techniques are also widely available, including the use of adenoviral vectors, adeno-associated vectors, lentiviral vectors, pseudotyped retroviral vectors and pox or vaccinia virus vectors. liposomes may also be used, including commercially available liposome preparations such as lipofectin ®, Lipofectamine ®, (GIBCO-BRL, Inc. Gaitherburg, Md.), Superfect ® (Qiagen Inc, Hilden, Germany) and Transfectam ® (Promega Biotec Inc, Madison Wis.). Other means to modulate a biological activity of DPP10 include agents which may affect interaction of DPP10 with downstream factors with which it interacts. For example, the activity of DPP10 may be affected. by inhibiting its interaction, with molecules containing the XPXS motif such as chemokines and cytokines, e.g. those shown in FIG. 19. In particular, the activity of DPP1O may be inhibited by the use of competitive or non-competitive inhibitors of any one or more of these chemokines or cytokines, or by small molecule inhibitors, which may function by inhibiting the active triad of DPP10. Other methods of inhibition will be known to persons skilled in the art.

Also provided is an agent, for use in the prevention or treatment of inflammatory disease in a subject, as defined above. Agents include nucleic acid sequences of the first or second aspects, polypeptide sequences of the third aspect, antibodies of the fourth aspect, and any other agent defined herein, preferably those which are capable of modulating the activity of DPP10.

The subject may be any animal, preferably a mammal, and more preferably human.

Also provided is the use of an agent as defined above in the manufacture of a medicament for use in the prevention or treatment of inflammatory disease, as defined above, in a subject.

In a tenth aspect of the invention there is provided isolated nucleic acid molecules comprising part of the sequence of FIG. 1, and comprising one or more SNPs at positions which correspond to the positions of FIG. 1 listed in any one or more Tables 1a, 1b, 1c or 3.

Particular isolated nucleic acid molecules include those:
comprising a SNP at the position corresponding to position 259007 of FIG. 1 where the adenine is changed to cytosine.
comprising a SNP at the position corresponding to position 267901 of FIG. 1 where the adenine is changed to guanine.
comprising a SNP at the position corresponding to position 318524 of FIG. 1 where the thymine is changed to cytosine.

The nucleic acid molecules of the invention may be DNA, RNA, and single or double stranded sequences. All the molecules of the present invention are isolated, or alternatively may be recombinant. By isolated is meant a nucleic acid molecule which has been purified, and is substantially free of protein and other nucleic acid. Such molecules may be obtained by PCR amplification, cloning techniques, or synthesis on a synthesiser. By recombinant is meant nucleic acid molecules which have been recombined by the hand of man.
The isolated nucleic acid molecules of the present invention are different to the “wild type” or “reference” sequence of FIG. 1. The sequence of FIG. 1, a 465 Kb region surrounding the D2S308 marker, described in WO99/50451 as being associated with asthma, is derived from a BAC/PAC contig which is not part of the invention. The BACs in the contig are 416L5, 543L9 and 317L18. The PAC is 69CE4 is not part of the invention. The nucleic acid sequences of the invention which differ from the sequence of FIG. 1 at any one or more of the positions detailed in Tables 1a, 1b, 1c or 3 are referred to as polymorphic variants of the sequence of FIG. 1 and form part of the invention.

This aspect of the invention also provides antisense sequences. Such sequences are typically single stranded and are capable of hybridizing to the above mentioned nucleic acid sequences of the invention, or to the sequence of FIG. 1, under stringent conditions. Preferred antisense sequences are those which are capable of hybridising to an allele of a polymorphism of the invention, and most preferably is capable of distinguishing between alleles of a polymorphism (of Tables 1a, 1b, 1c or 3). Stringent conditions are defined below. The antisense sequences may be prepared synthetically or by nick translation, and are preferably isolated or recombinant.

The antisense sequences include primers and probes, for example for use in the methods of the present invention. Primer sequences are capable of acting as an initiation site for template directed nucleic acid synthesis, under appropriate conditions which will be known to skilled persons. Probes are useful in the detection, identification and isolation of particular nucleic acid sequences. Probes and primers are preferably 15 to 30′nucleotides in length.

For amplification purposes, pairs and primers are provided. These include a 5′ primer which hybridizes to the 5′ end of the nucleic acid sequence to be amplified, and a 3′ primer which hybridizes to the complementary strand of the 3′ end of the nucleic acid to be amplified. Preferred primers are those listed in Table 2.

Probes and primers may be labelled, for example to enable their detection. Suitable labels include for example, a radiolabel, enzyme label, fluoro-label, biotin-avidin label for subsequent visualization in, for example, a southern blot procedure. A labelled probe or primer may be reacted with a sample DNA or RNA, and the areas of the DNA or RNA which carry complimentary sequences will hybridise to the probe, and become labelled themselves. The labelled areas may be visualized, for example by autoradiography.

Preferably, the probes and/or primers hybridise under, “stringent conditions”, which refers to the washing conditions used in a hybridisation protocol. The hybridisation conditions for probes are preferably sufficiently stringent to allow distinction between different alleles of a polymorphism upon binding of the probes. In general, the washing conditions should be combination of temperature and salt concentration so that the denaturation temperature is approximately 5 to 20° C. below the calculated Tₘ of the nucleic acid under study. The Tₘ of a nucleic acid probe of 20 bases or less is calculated under standard conditions (1M NaCl) as [4° C.×(G+C)+2° C.×(A+T)], according to Wallace rules for short oligonucleotides. For longer DNA fragments, the nearest neighbor method, which combines solid thermodynamics and experimental data may be used, according to the principles set out in Breslauer et al., PNAS 83: 3746-3750 (1986). The optimum salt -and temperature conditions for hybridisation may be readily determined in preliminary experiments in which DNA samples immobilised on filters are hybridised to the probe of interest and then washed under conditions of different stringencies. While the conditions for PCR may differ from the standard conditions, theTm may be used as a guide for the expected relative stability of the primers. For short primers of approximately 14 nucleotides, low annealing temperatures of around 44° C. to 50° C. are used. The temperature may be higher depending upon the base composition of the primer sequence used. Typically, the salt concentration is no more than 1M, and the temperature is at least 25° C. Suitable conditions are 5×SSPE (750 mM NaCl, 50 mM NaPhosphate, 5 mM EDTA pH 7.4) and a temperature of 25-30° C.

In an eleventh aspect, there is provided a host cell comprising a vector or isolated nucleic acid molecule according to the aforementioned aspects. The host cell may comprise an expression vector, or naked DNA encoding the nucleic acid molecules of the invention. A wide variety of suitable host cells are available, both eukaryotic and prokaryotic. Examples include bacteria such as E. coli, yeast, filamentous fungi, insect cells, mammalian cells, preferably immortalised, such as mouse, CHO, HeLa, myeloma or Jurkat cell lines, human and monkey cell lines and derivatives thereof. The host cells are preferably capable of expression of the nucleic acid sequence to produce a gene product (i.e. RNA or protein). Such host cells are useful in drug screening systems to identify agents for use in diagnosis or treatment of individuals having, or being susceptible to inflammatory disease as defined above.

The method by which said nucleic acid molecules are introduced into a host cell will usually depend upon the nature of both the vector/DNA and the target cell, and will include those known to a person skilled in the art. Suitable known methods include but are not limited to fusion, conjugation, liposomes, imrnmunoliposomes, lipofectin, transfection, transduction, eletroporation or injection, as described in Sambrook et al.

In a twelfth aspect of the invention there is provided a kit for diagnosis of disease or predisposition to disease, comprising a means for determining the presence or absence of a risk allele of a SNP of Tables 1a, 1b, 1c or 3, wherein the risk allele is diagnostic of disease or of predisposition to disease.

In a preferred embodiment, the kit comprises a means for determining the presence or absence of one or more risk alleles of polymorphisms according to the eighth aspect. In particular, the kit comprises means for determining the presence or absence of a risk allele of a SNP at position 259007, position 267901, and/or position 318524 of FIG. 1.

Preferably the kit will comprise the components necessary to deterrnine the presence or absence of a risk allele, in accordance with the eighth aspect of the invention. Such components include PCR primers and/or probes, for example those described above, PCR enzymes, restriction enzymes, and DNA or RNA purification means. Preferably, the kit will contain at least one pair of primers, or probes, preferably as described above in accordance with the tenth aspect of the invention. The primers are preferably allele specific primers. Other components include labeling means, buffers for the reactions. In addition, a control nucleic acid sample may be included, which comprises a wild type or variant nucleic acid sequence as defined above, or a PCR product of the same. The kit will usually also comprise instructions for carrying out the diagnostic method, and a key detailing the correlation between the results and the likelihood of disease. The kit may also comprise an agent for the prevention or treatment of disease.

In a thirteenth aspect of the invention, there is provided a method of identifying a compound for treatment of disease, comprising (a) administration of a compound to tissue comprising an isolated nucleic acid molecule comprising a SNP at a position which corresponds to a position of FIG. 1 listed in Tables 1a, 1b, 1c or 3; and (b) determining whether the agent modulates effects of the SNP.

In a preferred embodiment, the isolated nucleic acid molecule is according to the tenth aspect of the invention, and most preferably comprises a SNP at a position corresponding to position 259007, position 267901, and/or position 318524 of FIG. 1.

In this aspect, a nucleic acid molecule of the invention, and/or a cell line according to an aforementioned aspect, may be used to screen for agents which are capable of modulating the effect of a SNP.

Potential agents are those which react differently with a risk allele and non-risk allele. Putative agents will include those known to persons skilled in the art, and include chemical or biological compounds, sense or anti-sense nucleic acid sequence for example as described above, binding proteins, kinases, and any other gene or gene product agonist or antagonist. Preferably, the agent will be capable of modulating the effects of the disease causing allele. Most preferably, the agent is one which is capable of ameliorating the deleterious effects of the risk allele.

Such agents may be suitable for either prophylactic administration or after a disease has been diagnosed. The route of administration is suitably chosen according to the disease or condition to be treated, however, typical routes of administration of the agent of the present invention include but are not limited to oral, rectal, intravenous, parenteral, intramuscular and sub-cutaneous routes. The invention also provides for agents to be administered either as DNA or RNA and thus as a form of gene therapy. The agents may be delivered into cells directly by means including but not limited to liposomes, viral vectors and coated particles (gene gun).

In a fourteenth aspect of the present invention there is provided an agent or antibody as described above according to the invention, or use in preventing or treating allergies, and atopic diseases such as asthma or inflammatory bowel disease e.g. ulcerative colitis and Crohm's disease, and inflammatory joint disease such as rheumatoid arthritis and ankylosing spondylitis, or psoriasis, multiple sclerosis or type II diabetes.

There is also provided the use of an agent or antibody as described above in the manufacture of a medicament for use in the prevention or treatment of allergies, and atopic diseases such as asthma or inflammatory bowel disease e.g. ulcerative colitis and Crohn's disease, and inflammatory joint disease such as rheumatoid arthritis and ankylosing spondylitis, or psoriasis, multiple sclerosis or type II diabetes.

According to a fifteenth aspect of the invention, there is provided, a pharmaceutical composition comprising a nucleic acid or polypeptide sequence as defined above according to the invention. Alternatively, the pharmaceutical composition may comprise an agent as defined in relation to the above aspect or an antibody according to the fourth aspect of the invention.

Administration of pharmaceutical compositions is accomplished by any effective route, e.g. orally or parenterally. Methods of parental delivery include topical, intra-arterial, subcutaneous, intramedullary, intravenous, or intranasal administration. Administration can also be effected by amniocentesis-related techniques. Oral administration followed by subcutaneous injection would be the preferred routes of uptake; also long acting immobilisations would be used. In addition to the active ingredients, these pharmaceutical compositions may contain suitable pharmaceutically acceptable carriers comprising excipients and other compounds that facilitate processing of the active compounds into preparations which can be used pharmaceutically. Further details on techniques for formulation and administration rnay be found in the latest edition of REMINGTON'S PHARMACEUTICAL SCIENCES” (Maack Publishing Co, Easton Pa.).

Pharmaceutical compositions for oral administration can be formulated using pharmaceutically acceptable carriers well known in the art, in dosages suitable for oral administration. Such carriers enable the pharmaceutical compositions to be formulated as tablets, pills, dragees, capsules, liquids, gels, syrups, slurries, suspensions, etc., suitable for ingestion by the patient.

Pharmaceutical compositions suitable for use in the present invention include compositions wherein the active ingredients are contained in an effective amount to achieve the intended purpose. Thus, a therapeutically effective amount is an amount sufficient to ameliorate or eradicate the symptoms of the disease being treated. The amount actually administered will be dependent upon the individual to which treatment is to be applied, and will preferably be an optimised amount such that the desired effect is achieved without significant side-effects. The determination of a therapeutically effective dose is well within the capability of those sldlled in the art. Of course, the skilled person will realise that divided and partial doses are also within the scope of the invention.

For any compound, the therapeutically effective dose can be estimated initially either in cell culture assays or in any appropriate animal model. These assays should take into account receptor activity as well as downstream processing activity. The animal model is also used to achieve a desirable concentration range and route of administration. Such information can then be used to determine useful doses and routes for administration in humans.

A therapeutically effective amount refers to that amount of agent, which ameliorates the symptoms or condition. Therapeutic efficacy and toxicity of such compounds can be determined by standard pharmaceutical procedures, in cell cultures or experimental animals (e.g. ED50, the dose therapeutically effective in 50% of the population; and LD50 the dose lethal to 50% of the population). The dose ratio between therapeutic and toxic effects is the therapeutic index, and it can be expressed as the ratio ED₅₀/LD₅₀. Pharmaceutical compositions, which exhibit large therapeutic indices, are preferred. The data obtained from cell culture assays and animal studies is used in formulating a range of dosage for human use. The dosage of such compounds lies preferably within a range of circulating concentrations that include the ED₅₀ with little or no toxicity. The dosage varies within this range depending upon the dosage form employed, sensitivity of the patient, and the route of administration.

The exact dosage is chosen by the individual physician in view of the patient to be treated. Dosage and administration are adjusted to provide sufficient levels of the active moiety or to maintain the desired effect. Additional factors, which may be taken into account, include the severity of the disease state. Long acting pharmaceutical compositions might be administered every 3 to 4 days, every week, or once every two weeks depending on half-life and clearance rate of the particular formulation. Guidance as to particular dosages and methods of delivery is provided in the literature (see, U.S. Pat. No's 4,657,760; 5,206,344 and 5,225,212 herein incorporated by reference).

According to a sixteenth aspect of the invention, there is provided a number of screens. A first screen provides for identifying an agent which modulates DPP10 activity comprising:
providing a polypeptide sequence as claimed in any one of claims 8 to 13;
providing a DPP10 substrate;.
providing an agent to be tested;
measuring whether the agent to be tested modulates DPP10 by measuring processing of the DPP10 substrate.

The components of the screen are combined, in any optional order.

In the screening assay the DPP10 may be any DPP10 as claimed in any one of claims 8 to 13. Fragments of the DPP10 molecule such as the propeller domain, the fibronectin binding domain or another extracellular matrix binding domains may be used. Also, DPP10 polypeptides which comprise one or more SNP nucleic acid sequences of the present invention, such as described in any one of claims 37 to 41 may be used. The DPP10 polypeptide may be purified or non-purified. The DPP10 polypeptide may be soluble. It may comprise one or more of the domains.

The agent being tested is being identified for use in the prevention or treatment of a DPP10 related or mediated disease or disorder. Such diseases or disorders include: asthma, eczema or hayfever (known as atopic diseases), inflammatory bowel disease (e.g ulcerative colitis or Crohn's disease), inflammatory joint diseases (such as rheumatoid arthritis and ankylosing spondylitis), psoriasis, brain diseases involving an inflammatory component such as multiple sclerosis or type II diabetes or hypertension.

The DPP10 substrate may be any which is processed by a DPP10 polypeptide of the invention. By processed is meant any changes which can be measured. The substrate may comprise the formula XPXS or more specifically the formula NH₂X—P(X)_{y}S(X)ₙ where NH₂ is the amino terminus, X is any amino acid, P is proline, S is serine, y is 1 to 4, and n is any number. The substrate may be a cytoidne containing the XPXS motif such as those in FIG. 19, namely RANTES, SDF-1, EOTAXN, IP10, MCP2, IL17 β, IL2, GCP-2, IL18bp, chemokine CC-1/CC-3, interleukin-8, CTAK/ALP/LIC, or a small peptide having such a motif. These substrates may be fluorescently labelled or modified to allow easy detection of processing. Such labelling or modification is known to the person skilled in the art.

Typically the processing of the substrate will comprise measuring protease activity, for example such protease activity may be detected by cleavage of the substrate. Modulation is taken to mean either an increase or decrease in activity of the enzyme. Such activity may be affected by an alteration in expression of DPP10 or a change in the DPP10's half life or a change in the post-translational modification of the DPP10.

The present invention further provides a screen for identifying an agent which modulates DPP10 activity comprising:
providing a DPP10 polypeptide as claimed in any one of claims 8 to 13; providing an agent to be tested;
providing a cell; and
measuring whether the agent to be tested modulates DPP10 by measuring adhesion of the cell to a surface

Such a screen can be referred to as a cell adhesion screen (or assay). The components of the screen are combined, in any optional order.

Typically cells used in the cell adhesion assay may be maintained in suspension where adhesion is measured by aggregation of the cells due to intercellular adhesion molecule interactions. Alternatively, adhesion to a surface may be measured. The surface may be a non-biological molecule e.g. tissue culture plastic or it may be a biological molecule which is cellular or noncellular. Examples of a noncellular molecule include extracellular matrix components such as fibronectin, collagen and such like. One or more cells or other biological non-cellular molecules may be attached to a surface such as a tissue culture surface or an extracellular matrix component coated surface. Adhesion is determined by measuring the adhesion of a cell to a surface. Modulation in cell adhesion may be either an increase in cell adhesion or a decrease in cell adhesion. An agent is considered to be a modulator of DPP10 activity if it affects DPP10 activity, this may be either at the level of expression of the DPP10 molecule or by altering the half life of the DPP10 molecule or by affecting the post-translation modification status of the DPP10 molecule.

Yet a futher aspect of the invention provides a screen for identifying an agent which modulates DPP10 activity comprising:
providing a DPP10 polypeptide as claimed in any one of claims 8 to 13;
providing an agent to be tested;
providing a cell;
measuring a change in differentiation or proliferation of the cell.

The components of the screen are combined, in any optional order.

Typically, differentiation may be measured by any means known to the persons skilled in the art for example in the case of a T-lymphocyte, the change in differentiation can be T-cell activation. In the case of other cell types it may be the induction or prevention of production of a secretable cell signalling factor such as an immunomodulator e.g. a cytokine or growth factor, The immunomodulator may be a peptide or may be any other biological substance which expression is altered by an agent which modulates DPP10. Typically this assay is performed in vitro for example in tissue or organ culture.

The change in phenotype may be any. It may involve a change in T-cell and/or B-cell phenotype.

Such a screen provides an in vitro model for identifying an agent which modulates DPP10 activity.

Yet a further aspect of the invention provides a screen for identifying an agent which modulates DPP10 activity comprising:
providing a transgenic animal according to one of claims 23 to 25 or 59;
providing an agent to be tested;
contacting the transgenic animal with the agent to be tested;
detecting a change in the transgenic animals phenotype.

The components of the screen are combined, in any optional order.

The cell against which the agent is tested may be in suspension, tissue culture, as part of an organ or as part of an animal. Preferably the animal is a laboratory animal, such as a rat, rabbit, mouse or other rodent.

Yet a further aspect of the invention provides a screen for detecting a side effect associated with the use of an agent which modulates DPP10 comprising:
providing a cell which does not substantially express DPP10;
providing an agent to be tested;
contacting the agent to be tested with the cell; and
measuring any side effect produced by the agent on the cell.

The components of the screen are combined, in any optional order.

The side effect to be measured may be any, and may depend on whether the cell is part of a larger tissue or animal. It may involve a change in cell differentiation, or cell proliferation. The side effect may be a measure of the change of phenotype of an organ or animal.

Yet a further aspect of the invention provides a screen for identifying an agent which modulates DPP10 activity comprising:
providing a DPP10 nucleic acid according to any of claims 1 to 4;
providing an agent to be tested;
measuring whether the agent to be tested modulates DPP10 by measuring the interaction of the agent with the sample of nucleic acid.

Preferably this screen is an in vitro transcription assay, measuring transcription of DPP10.

Alternatively, an agent may be identified by the use of theoretical or model characteristics of DPP10. The functional or structural characteristics of DPP10 may be of the protein itself or of a computer generated model, a physical two- or three-dimensional model or an electrical (e.g. computer) generated primary secondary or tertiary structure, as well as the pharmacaphore (three dimensional electron density map) or its X ray crystal structure.

Putative agents will include those known to persons skilled in the art or new substances, and include chemical or biological compounds, such as anti-sense nucleotide sequences, polyclonal or monoclonal antibodies which bind to polypeptide sequence of the second aspect.

According to a seventeenth aspect of the invention, there is provided the use of a nucleic acid sequence or polypeptide sequence as defined above in a screen for an agent which modulates the activity of DPP10.

The method preferably comprises contacting a putative agent with a nucleic acid or polypeptide sequence according to an aforementioned aspect of the present invention and monitoring expression and/or activity of the nucleotide or polypeptide sequence. Potential agents are those which alter the activity or expression of the DPP10 nucleotide or polypeptide sequence compared to the activity or expression in the absence of the agent. The present method may be carried out by contacting a putative agent with a host cell, tissue culture, or transgenic non-human animal comprising a nucleotide or polypeptide according to the invention, and displaying inflammatory disease.

Also provided are agents identified by the method of the sixteenth or seventeenth aspects.

Preferred features for the second and subsequent aspects of the invention are as for the first aspect mutatis mutandis.

The invention will now be described with reference to the following drawings and examples which are included for the purposes of illustration only and are not to be construed as being limiting on the invention.

FIG. 1 shows the DNA sequence from the chromosone 2 Asthma Locus.

FIG. 2a shows the MEX4 predicted exon sequence.

FIG. 2b shows the location of MEX4 within the refined region of linkage disequilibrium and relative to marker D2S308.

FIG. 2c shows the full length insert sequence and translation of the foetal brain cDNA clone identified by screening with MEX4. The MEX4 sequence is in bold and the start codon is underlined.

FIG. 2d shows a full length insert sequence of the 3′ RACE clone amplified from primers in MEX4 FB-1.

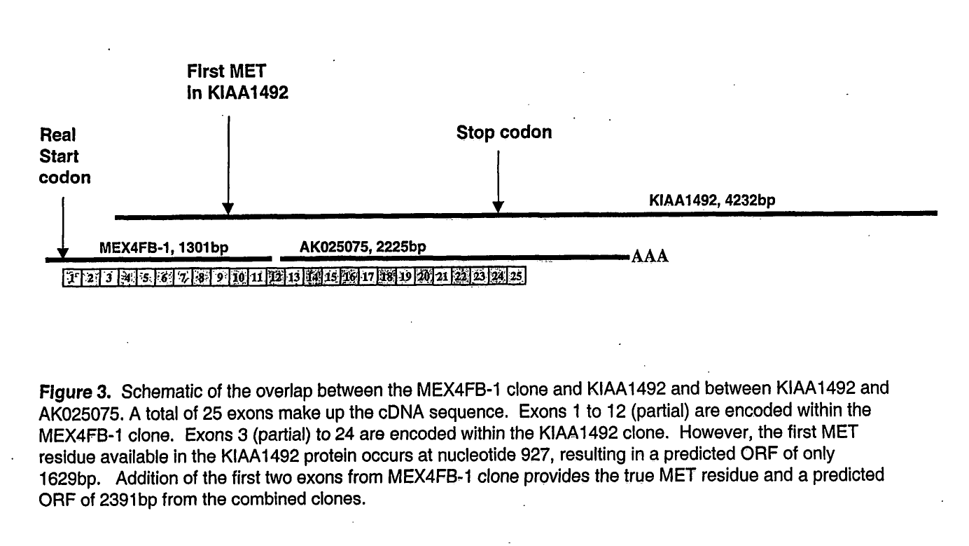

FIG. 3 is a schematic of the overlap between the MEX4FB-1 clone and KIAA1492 and between KIAA1492 and AK025075. The full length cDNA sequence is comprised of 25 exons. Exons 1 to 12 (partial) are encoded within the MEX4PB-1 clone. Exons 3 (partial) to 25 are encoded within the KIAA1492 clone. However, the first MET residue available in the KIAA1492 predicted protein occurs at nucleotide 927, resulting in a predicted ORF of only 1629bp. Addition of the first two exons from MEX4FB-1 clone provides the true MET residue and a predicted ORF of 2391 bp from the combined clones.

FIG. 4 shows the full length sequence of the human DPP10 mRNA generated from an overlap of the MEX4FB-1 and 3′ RACE clones. The MEX4 sequence is in bold and the initiation and termination codons are underlined.

FIG. 5 shows the full length Mouse DPP10 coding sequence.

FIG. 5a shows the mouse DPP10 full length CDNA sequence.

FIG. 6 shows an overview of human DPP10 alternative transcripts 1 to 6, the predicted transmembrane domains, exon 2A (Transcript 6), and the BAC location by accession number.

FIG. 7 shows a schematic overview of the Mouse DPP10 transcripts.

FIG. 8 shows the Mouse DPP10 alternative exons, the predicted peptide sequence of transcripts 1 to 4 and the full length sequence of the transcript 1.

FIG. 9 shows the sequences of the human alternate exons 1b to 1g and alternate exon 2A, liver clones 1 to 3, exons 2B to 2G, the predicted peptides from Transcripts 1 to 6, the predicted peptides for the exon 1b 3′RACE clone transcripts 1 to 5, the human transcript 6 (MEX4-6 “stopper”) sequence and the human 3′RACE clone sequence.

FIG. 10 shows the sequences of the DPP10 exons. The 5′UTR is included in exon 1a and the 3′UTR sequence in exon 25. The coding regions of these two exons are in bold.

FIG. 11 shows the DPP10 predicted protein sequence of 796 amino acids.

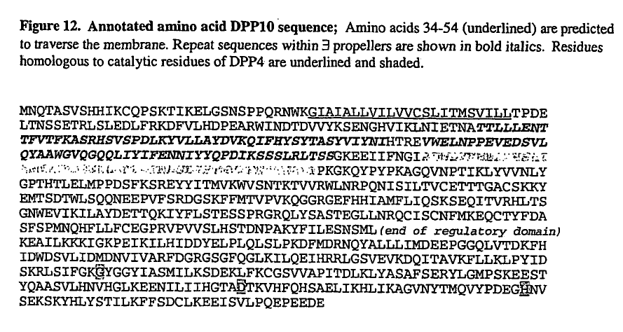

FIG. 12 shows the amino acid sequence of DPP10. Amino acids 34-54 (underlined) are predicted to traverse the membrane. Repeat sequences within β-propellers are shown in bold italics. Residues homologous to catalytic residues are underlined and shaded.

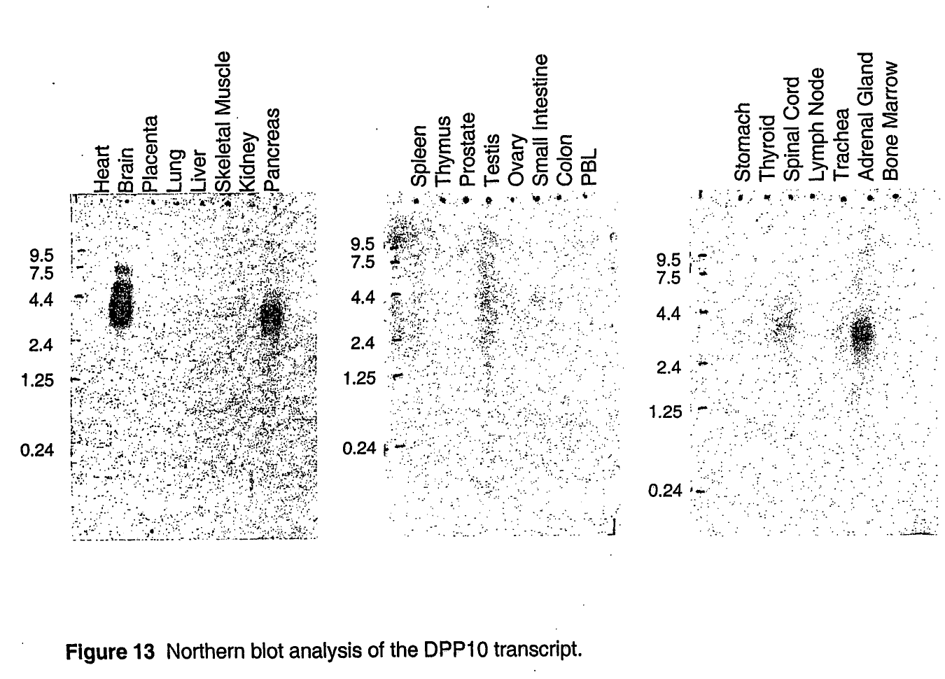

FIG. 13 shows Northern blots of DPP10 which demonstrates the presence of multiple transcripts.

FIG. 14 shows multiple alignment of the catalytic domains of DPP10 homologues. Asterisks (*) mark the catalytic site positions.

FIG. 15 shows a schematic of exon 1b 3′RACE transcripts.

FIG. 16 shows a multiple alignment of the transmembrane region of DPP10 and homologues.

FIG. 17 shows a multiple alignment of the β-propellor domain of DPP10 and prolyl oligopeptidase homologues.

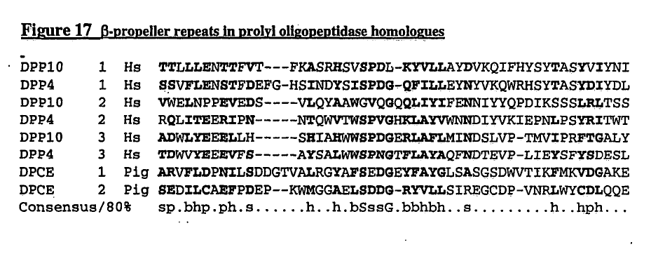

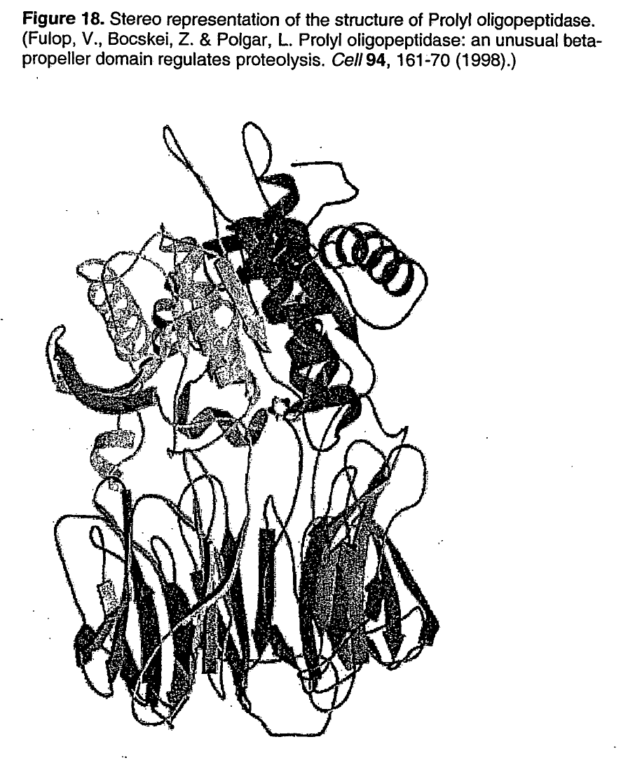

FIG. 18 shows the proposed structure of porcine prolyl oligopeptidase (Fulop V, Bocskei Z, Polgar L. (1998) Prolyl oligopeptidase: an unusual beta-propeller domain regulates proteolysis. Cell 1998 July 24;94(2):161-70).

FIG. 19 shows the cytokines containing P×S motifs. Human chemokines and cytokines that have a serine within 10 amino acids of a predicted signal peptide cleavage site and which contain a P×S motif (where X represents any amino acid). The predicted signal peptide cleavage bond is shown as “\hat{ }” and P×S motifs are highlighted. The first group of molecules contain the P×S two residues after the cleavage site.

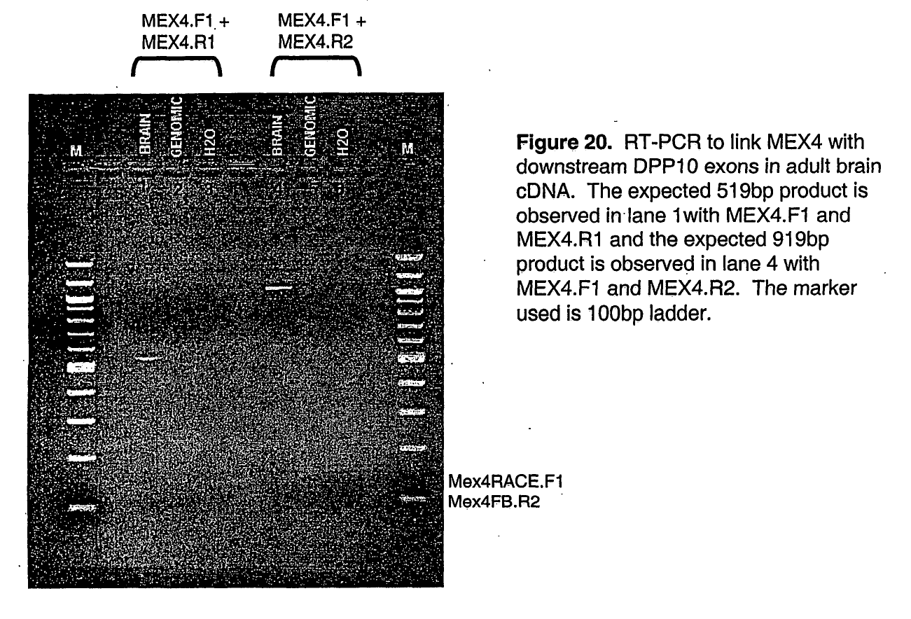

FIG. 20 shows RT-PCR linking MEX4 with downstream DPPIO exons in adult brain cDNA. The expected Sl9bp product is observed in lane 1 with MEX4.F1 and MEX4.R1 and the expected 919bp product is observed in lane 4, with MEX4.F1 and MEX4.R2. The marker (lane M) used is lOobp ladder. Negative controls (genomic DNA and water (H20) were included.

FIG. 21 shows the sequence alignment of the mouse and human DPP10 transcript 1 peptides.

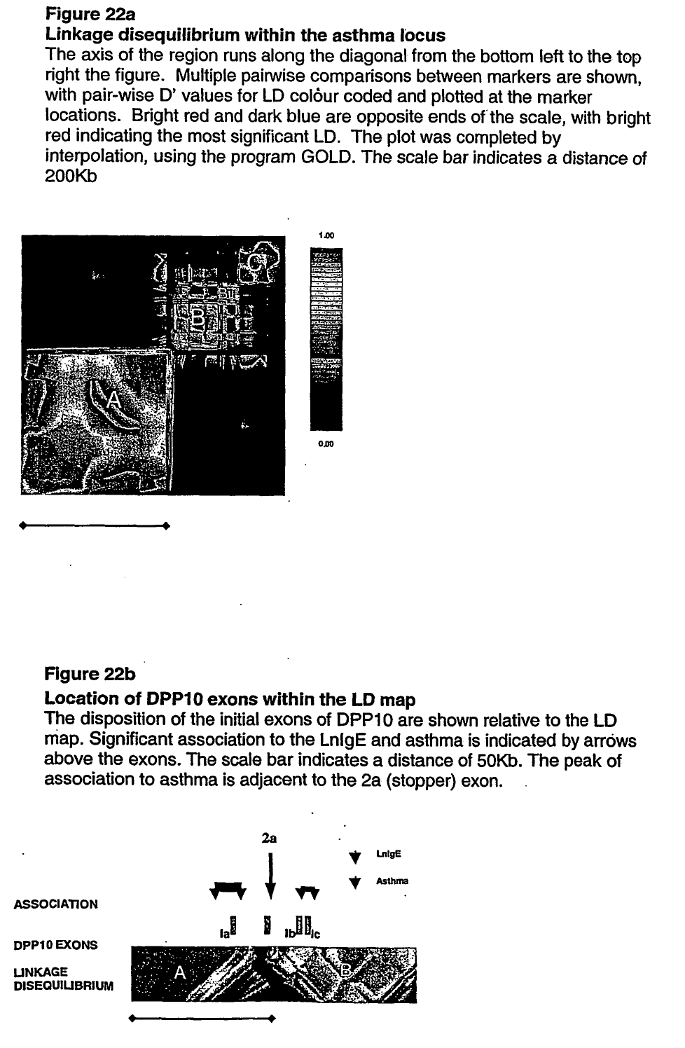

FIG. 22a shows linkage disequilibrium within the asthma locus.

FIG. 22b shows the location of DPP10 exons within the LD map. The disposition of the initial exons of DPP10 are shown relative to the LD map.

Significant association to the LmIgE and asthma is indicated by arrows above the exons. The scale bar indicates a distance of 50 Kb.

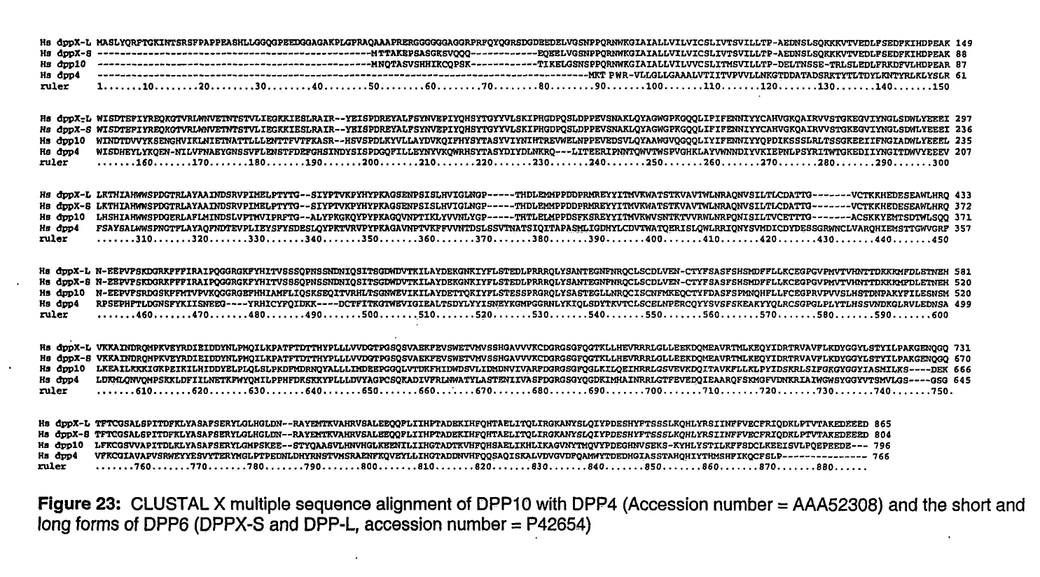

FIG. 23 shows CLUSTAL X multiple sequence alignment of DPPX-L (DPP6): Genbank Accession P42658 (DPP6); DPPX-S: Genbank Accession P42658; DPP4: Genbank Accession AAA53208.

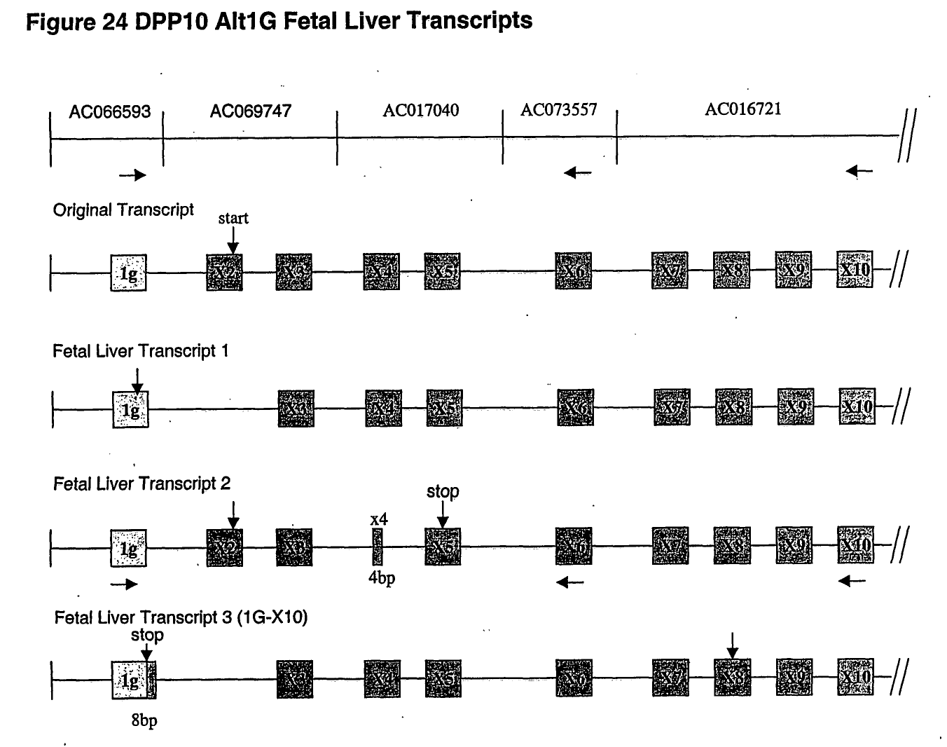

FIG. 24 shows the DPP10 alternative 1g fetal liver transcripts.

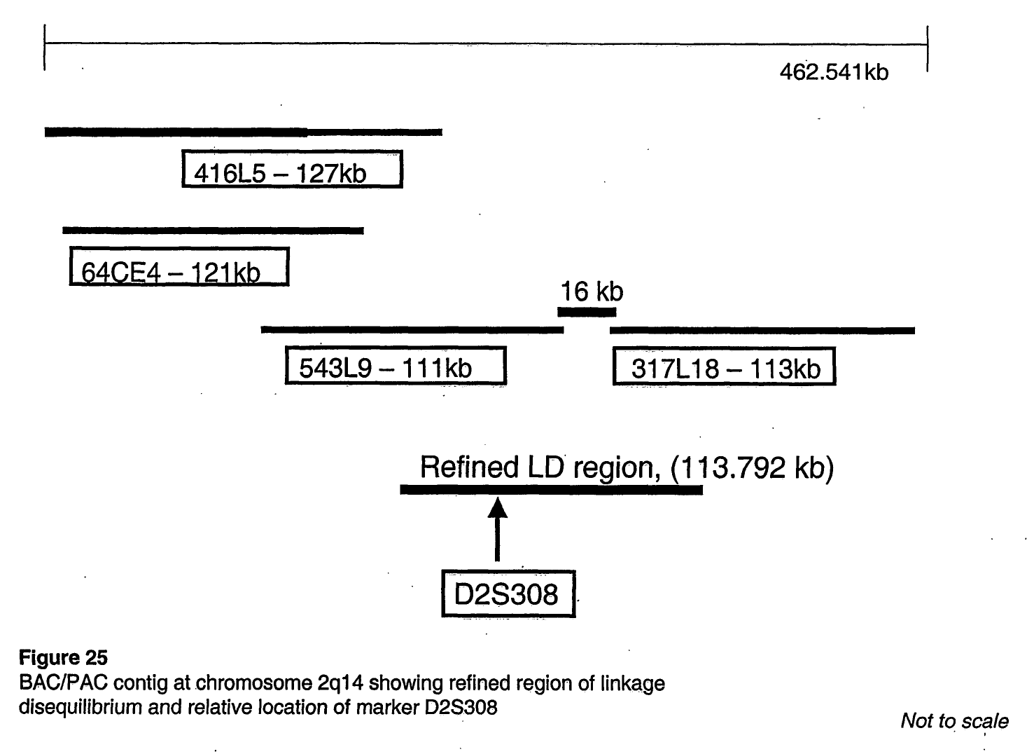

FIG. 25 shows the BAC/PAC contig at chromosome 2q14 showing refined region of linkage disequilibrium and relative location of marker D2S308.

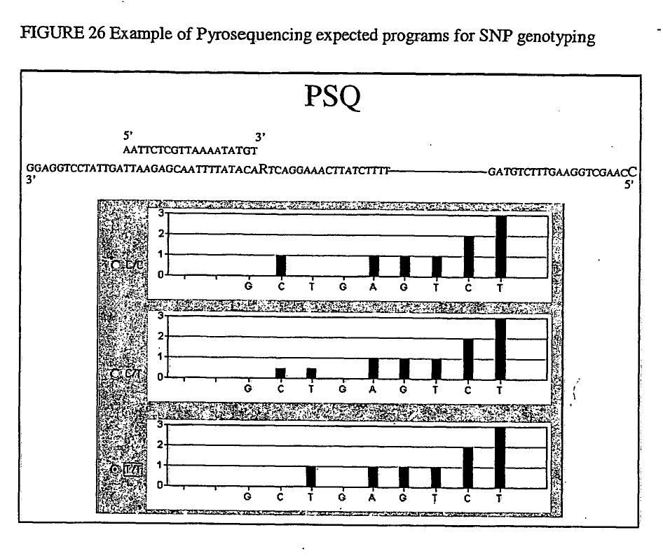

FIG. 26 shows an example of pyrosequencing for SNP genotyping.

FIG. 27 shows a western blot of diluted human serum samples probed with anti-DPP10 c-terminus antibody.

TABLE 1a shows SNPs identified in the sequence of FIG. 1 (LD region).

TABLE 1b shows SNP's genotyped in the sequenced region of FIG. 1 (ID region).

TABLE 1c shows DPP10 gene SNP outside of the LD region.

TABLE 2 shows PCR primer sequences and positions in the sequence of FIG. 1.

TABLE 3 shows associations between asthma and the LnlgE and SNPs.

TABLE 4a shows primer pair sequences used in RT-PCR.

TABLE 4b shows RT-PCR expression data

TABLE 5 shows regions of sequence conserved between human and mouse. Co-ordinates are given with reference to FIG. 1.

TABIE 6 shows PSQ assay oligonucleotides and PCR annealing temperatures.

TABLE 7 shows standard deviation in DPPIO expression levels ascertained by Taqman analysis of blood RNA from asthmatics and controls.

### EXPERIMENTAL EXAMPLES

Overview of the Experiments Performed:

Novel SNPs were identified by re-sequencing sections of the ˜462 kb contig around marker D2S308 in DNAs from asthmatic and control individuals. These SNPs were gentoyped across a panel of asthmatic families from three populations. Association analysis was performed by transmission disequilibrium tests (TDT), and identified several SNPs showing positive association with asthma. The genotype data from all the SNPs was used to refine the extent of linkage disequilibrium (LID) around the microsatellite marker D2S308 and the associated SNPs. A 113, 792 bp region of sequence, containing the associated marker and SNPs and termed the Island of LD, was selected for transcript identification.

In silico analysis of public sequence databases showed that no known genes map within the island of LD. The only expressed sequence tags (ESTs) which map into the region are: AA424226, H10825 and AA42637. Therefore extensive DNA sequence analysis was undertaken to identify coding DNA sequences within the 113792bp island of LD. Using exon prediction algorithms, a putative coding sequence of 60 bp (designated MEX4) was identified. Screening of a cDNA library with a DNA probe containing MEX4 identified a 1301 bp cDNA clone (MEX4FB-1). BLASTN analysis against the Genbank nucleotide (nr) database identified 100% nucleotide homologies to 2 sequences—AB040925 and AK025075. Using this information it was possible to assemble a composite full length cDNA clone with a predicted open reading frame of 2391bp. Subsequent 3′ RACE experiments from the end of the MEX4FB-1 clone, confirmed this predicted sequence as the C-terminal part of the gene and 3′ UTR. Further 5′ RACE experiments were performed from exon 3 and identified six other alternate N-terminal exons arranged in five transcript types. Further 3′ RACE from Mex4/exon 1a, identified one additional C-terminal exon (2a) which formed part of a short cDNA with Mex4/exon 1a Genomic structure analysis showed that the gene contains twenty five exons and spans a large region of genomic DNA of ˜1 MB. Based upon this structure the marker D2S308 falls within intron 1. The sequence of the clone AB040925, encodes part of exon 3 to exon 25, however because intron 1 is several hundred kb in length, no in silico prediction programmes predicted that this clone was spliced downstream of exon 1a.

RT-PCR and Northern blot analysis was performed to analyse DPP10 expression, which was revealed to strongly express in a neuroendocrine fashion with evidence of multiple transcripts. DPP10 was also shown to be expressed in PBL and expression level in asthmatics and controls were assessed by Taqman analysis of total blood RNA.

A transmembrane domain is present in the peptides predicted from two of the five transcripts. Subsequent cellular localization studies with epitope tagged DPP10 constructs for transcript 1 and 2, transfected into Hela cells, have confirmed the membrane and cytosolic localizations predicted for these two transcripts.

BLASTX analysis against the Swissprot protein database detected significant homologies with a class of proteins known as dipeptidyl peptidases (prolyl-oligopeptidases), the most significant homology being with DPP6. The gene at Chromosome 2q14 represents a novel dipeptidyl peptidase, DPP10.

### Example 1

SNP Discoverv and Association Testing

Subjects and Phenotyping

The subjects were administered a modified British MRC respiratory questionnaire. “Asthma” was defined as a positive answer to the questions “Have you ever had an attack of asthma?” and “If yes, has this happened on more than one occasion?““Wheeze” was defined as a positive answer to the question “Has your chest ever sounded wheezing or whistling?” and “f yes, has this happened on more than one occasion?” The total serum IgE was measured in all children. Slin tests to house dust mite and grass pollen was carried out.

Three panels of subjects have been studied.

Panel A consisted of 80 nuclear families sub-selected from an Australian population sample of 230 families. The panel contained a total of 203 offspring forming 172 sub-pairs. 12% of the children were asthmatic.

Panel B consisted of 77 nuclear and extended families recruited from asthma and allergy clinics in the United Kingdom. These families contained 215 offspring (268 sib-pairs) of which 56% were asthmatic.

Panel C consisted of 87 nuclear families recruited through a child attending an asthma clinic in the Oxford region. The families contained 216 offspring (148 sibling pairs), of whom 44% where asthmatic.

Positional Cloning and SNP Discovery.

We built an extended BAC/PAC contig covering 1.5 Mb of the locus and sequenced approximately 465 kb from four contiguous clones surrounding D2S308. SNP detection was systematically carried out on regions of DNA that were free of repeats by sequencing 5 unrelated subjects and 5 controls with and without asthma and a pool of DNA from 32 unrelated individuals.

100 SNPs were identified with minor allele frequencies >20%, and 67 of these were genotyped on our subjects. SNP typing was by PCR and restriction digestion. In the absence of a natural restriction sequence, a primer was modified to generate a site. (Primers pairs are given in Table 2). Error checldng and haplotype generation was carried out by the MERLIN computer program (Abecasis 2001). Linkage disequilibrium (LD) between markers was assessed by estimation of D′ from the parental haplotypes (Abecasis et al., Am J Hum Genet 59 323-36 (1995)) and portrayed by the GOLD program Abecasis et al., Bioinfarmatics 16 182-3 (2000)). LD was distributed into four distinct islands (A, B, Bii and C) (FIG. 22a). The border between the A and B island was flanked by the 543WTC91P and 543WTC122P SNPs (Tables 1a, 1b and 1c).

Association Testing

Association was sought between Asthma and the SNPs by transmission disequilibrium tests (Spielman et al Am J Hum Genet 59 983-9 (1996)). (Table 3) (FIG. 1). Positive associations were confined to the B island of ID (FIG. 22b). The strongest association was observed with the 543WTC122P SNP, approximately 1 Kb proximal to D2S308: weaker associations were observed more distally, between 543WTC11OP and 317WTC59P (9 Kb and 59 Kb away) (Table 3).

Association was also sought between the Loge (gE concentration) LnIgE) and the SNPs by variance components analyses (Abecasis et al Am J Hum Genet 66 279-292 (2001)) (Table 3). Moderate evidence for association was detected in the A island of ID, over a region of approximately 60 Kb. The complete separation of LD between the A and B islands suggested that this QTL was different to the polymorphism affecting asthma status from the B island. The alleles of D2S308 are described in WO99/50451.

SNP DP007, (Table 1c), located in the 3′ UTR of the DPP10 gene showed positive association to Rasti (p=0.002), Psti (p=0073) and loge (p=0.0263).

### Example 2

Determination of the Full Length Sequence of a Novel Dipeptidylpeptidase-Like Gene, DPP10.

Gene Identification

In order to identify genes within the associated sequence region, sequence similarity searches were performed using BLAST analysis against the public sequence databases (Althschul et al., 1997). No significant sequence identities or similarities to reported genes were identified. The only evidence for any coding sequence in the region were the DNA sequence matches to two ESTs; H10825 (IMAGE clone 46982) and AA426377 (MAGE clone 757495).

Identification of Conserved Exons

The next stage of analysis was to identify potential coding regions in the 113,792bp of DNA sequence by the use of exon prediction software. The sequence from the refined region of LD was subjected to exon prediction analysis by submission to the NIX site at Human Genome Mapping Project (HGMP) Resource Centre (http://www.hgmp.mrc.ac.uk/homepage.html). This site incorporates the following exon prediction programmes; Grail/exon, Grail/gap2, MZEF, GENSCAN, Genemark, hmm.gene, FGENE, FEX, Genefinder and FGENES. For all DNA sequence analysis, default settings were used.

Over 100 exons were predicted in the region, 66 by more than one programme. The group of multiple-predicted exons included a 60bp exon designated as MEX4. The sequence of this predicted exon sequence is detailed in FIG. 2a MEX4 was identified by the FGENE/FEX software, with significance scores of 0.65 and 0.7 respectively. The location of MEX4 within the refined region of linkage disequilibrium and relative to DNA marker D2S308 is shown in FIG. 2b.

PCR primers were designed to putative exon sequences predicted by more than one algorithm. PCR products generated from these oligonucleotide primer pairs were used in low stringency hybridisations against “zoo strips”—Southern blots containing restriction enzyme-digested DNA from human and mouse genomic DNA. MEX4 showed weak hybridisation to the mouse DNA lanes (data not shown) and therefore was considered to harbor sequences conserved between the two species, indicative of functional, coding, DNA sequences.

Library Screening

A probe containing the MEX4 sequence was used to screen a commercial foetal brain cDNA library prepared in the lambda-triplex phage vector (Clontech). Approximately 1 million phage clones were plated out onto twenty 15 cm Luria agar plates, and duplicate colony lifts performed using 132 mm circular nylon transfer membranes (Hybond N+, Amersham). All procedures were performed according to the library manufacturer's published protocols (http://www.clontech.com/libraries/#techinfo, protocol PT3003-1, version PR09529). The MEX4 probe was labelled with α²P-dCTP using a random labelling kit (“Prime-it RmT random primer labeling kit, Stratagene) and purified through a G50 Spin column (Quick Spin™ columns, Boehringer Mannheim) according to the manufacturer's instructions. The library filter set was hybridised using a phosphate/SDS buffer (0.5M NaPO₄, 7% SDS, 1 mM EDTA pH 8.0) at 65° C. for 16 hours. Filters were washed twice with 2L of 2×SSC/0.01% SDS for 30 minutes at 65° C. and once with 1×SSC/0.01% SDS for 30 minutes at 65° C., then exposed to autoradiographic film (Kodak X-OMAT) for two days with signal intensifying screens (Hyperscreen, Amersham).

Three duplicated positive plaques were identified on the filters. Each pool of positive phage plaques was “cored” using a cut-off 1 ml pipette tip. The plug of agar and plaques were put into a 2 ml screw cap microcentrifuge tube containing 1 ml of lambda phage dilution buffer (100 mM NaCl, 10 mM MgCl₂, 35mM Tris-HCl pH 7.5, 0.01% gelatin). Phage dilutions of 1:100, 1:1000 and 1:10,000 were prepared and plated out onto a series of secondary plates. Colony lifts were performed as above and the filters hybridised with MEX4 probe as above.

At this stage one positive clone was chosen to carry forward for further investigation. This was named MEX4FB-1. Single hybridising plaques were available for this clone. One plaque was cored using a cut-off Iml pipette tip and diluted with 350 μl of lambda phage dilution buffer. A 10 ml Luria broth culture of BM25.8 cells was grown OIN at 31° C. and inoculated with 150W of the recovered phage plaque to perform plasmid extraction according to the library manufacturer instructions. 20 μl of final plasmid broth was plated out onto a Luria agar plate containing 50 μg/μl of ampicillin and incubated O/N at 37° C.

Plasmid DNA Preparation

Two colonies of MEX4FB-1 were picked from the plate and grown shaking at 37° C O/N in 10 mls of luria broth supplemented with 50 μg/μl of ampicillin. Plasmid DNA was isolated using the QlAprep Spin Miniprep Kit (QIAGEN), according to the manufacturer's instructions. DNA yields were quantified for sequencing using a DNA fluorometer (Hoeffer Ltd).

Sequencing

Plasmids were sequenced according to the protocol of the dynamic ET terminator cycle sequencing kit (Amersham). Initially forward and reverse vector sequencing primers were used, cDNAlibseq.R[dTCCGAGATCTGGACGAGC]and cDNAlibseq.R [dTAATACGACTCACTATAGGG]. The DNA sequence generated from two plasmid end reads did not overlap, so sequencing was also performed with two internal walking primers;

| | |
|---|---|
| MEX4-6 FB 21.FW | [dTTTGTGCTTCACGATCCAGAGG]; |
| and | |
| MEX4-6 FB 21.RW | [dGATGTCAGTCGCAATGAACTGC] |

to obtain full length insert sequence. This 1,301 bp sequence is presented in FIG. 2c. Identical sequence was obtained from both of the two colonies of MEX4FB-1 picked in the plasmid preparation stage.

Each DNA sequencing reaction used: 400 ng template DNA, 0.25 pmol primer, 8 μl ET Terminator mix and distilled H₂0 to a final reaction volume of 20 W. The cycle conditions used were: 96° C. for 30 sec, 50° C. for 20 sec, 60° C. for 1 min for 25 cycles, with a final holding cycle at 4° C. prior to DNA purification. Sequencing products were purified by gel filtration (plO gel) followed by ethanol precipitation. Dry DNA pellets were resuspended in 2 μl deionised formamide and denatured at 96° C. for 2.5 min, followed by snap-cooling on ice. 1 μl of each reaction was loaded onto a 48 cm sequencing gel and run for 10 hours on an ABI 377 Sequencer and the data collected using filter set A. Sequence traces were analysed using the SEQUENCHER™ sequence analysis software, version 4.0.5b5 (Gene Codes Inc.).

Sequence Analysis by BLASTN to Determine Overlapping Clones

The MEX4FB-1 clone sequence was used in a BLASTN analysis against the Genbank nucleotide NR database to identify any sequence matches (Altschul et al., 1997). A 100% nucleotide match was identified from MEX4FB-1 nucleotides 409 to 1301 to the 5′ end of a partial cDNA clone, KIAA1492 (Genbank: AB040925) (Nagase et al., 2000). This clone extends 3,349bp 3′ to the end of MEX4FB-1. The full length sequence of AB040925 was retrieved via the National Center for Biotechnology Information (NCBI) website (http://www.ncbi.nlm.nih.gov/) using the Entrez nucleotide search facility. This full length AB040925 sequence was also subjected to BLASTN analysis against the NR database. This identified a second cDNA clone, AK025075, 2,225bp in length with a 99% match with AB040925 over 2,210bp. The only mismatches were at the 3′ end of the clone where a poly-A tail is present in AK025075, but not in AB040925. Therefore, it would appear that there may be two different 3′ ends to the gene as the two cDNAs AB040925 and AK025075 are of different lengths. A schematic overview of these alignments is presented in FIG. 3.

The first available methionine residue in the AB040925 sequence is 509 bp from the 5′ end of the clone in the +2 reading frame. The predicted open reading frame (ORF) from this position is 1,629 bp in size. Upstream of this methionine are 169 coding residues, representing a full ORF. This implies that the reported AB040925 sequence is not full length. By combining the MEX4FB-1 clone with the overlapping AB040925 sequence a start methionine was identified at nucleotide position 165. Upstream of this methionine is a single coding residue, preceded by a STOP codon. The addition of the MEX4FB-1 sequence therefore provides the correct 5′ end and start codon for this gene and results in a predicted ORF of 2,391bp, encoding 796 amino acid residues. The combined sequence from these three clones, utilising the shorter 3′ end represented by the sequence of clone AK025075, is presented in FIG. 4.

Sequence Analysis by BLASTN to Determine Genomic Structure

The composite DNA sequence (FIG. 4) from the combined cDNA clones was used in a BLASTN analysis against the HFGS database (Altschul et al., 1997). This resulted in the identification of multiple BAC clones containing portions of the gene sequence. In total 24 exons were identified within the cDNA sequence (FIGS. 9 and 10). By comparison of the relative positions of these BAC clones on chromosome 2, it is apparent that the DPP10 gene spans a large region of genomic DNA. (http://genome.ucsc.edu/goldenPath/septTracks.html and http://www.ncbi.nlm.nih.gov/cgi-bin/Entrez/hum_srch?chr=hum_chr.inf&query). Most of the BACs detailed in Tables la to Ic are in working draft, unfinished status, so current estimates could vary, but it appears that the gene spans approximately 1.2Mb of DNA at chromosome 2q14.

Expression Analysis:

The primers:

| | |
|---|---|
| MEX4.F1 | [dAACCAAACTGCCAGCGTGTCC]; |
| MEX4.R1 | [dAAGACGGAGTCCTCTACTTCTGG]; |
| and | |
| MEX4.R2 | [dATGGACCAACTCACACTTTGGAGC] |

were designed to.perform RT-PCR on cDNA from adult brain. This experiment was performed to confirm that the MEX4 exon is linked to the DPP10 exon sequences found in the clones AB040925 and AK025075, in a second cDNA source in addition to the isolated foetal brain clone. The primer MEX4.F1 is located at the beginning of the MEX4 exon, (DPP10 exon 1), the primer MEX4.R1 is located in DPP10 exon 6 and the primer MEX4R2 is located in exon 10. A 519bp product is expected if MEX4.1 is used in conjunction with MEX4.R1 and a 919bp product when MEX4.F1 is used with MEX4.R2. The PCR conditions used were; -50ng of cDNA (Marathon ready RACE cDNA, Clontech), in a 50 μl reaction with 10 pmoles of each primer, 2.5 μM dNTPs 1.5 mM Magnesium Chloride, and 0.5 units of Amplitaq Gold in 1× amplitaq gold buffer II, (Perkin Elmer Biosystems). Cycling conditions were 95° C. for a single 12 minute cycle, then 38 cycles of a 94° C. denature for 15 secs, 60° C. anneal temp for 15 secs, and 72° C. for 30 secs, followed by 72° C. for 5 minutes. A 50 ng aliquot of genomic DNA was used as a negative control as well as a no-DNA control. A 5 μl aliquot of each reaction was run out on 1% agarose gel prepared with 1XTBE using standard methods. The results are shown in FIG. 20. A clear band is seen in the brain lane for each reaction indicating that the MEX4 exon is expressed, linked to DPP10 downstream exons in adult brain as well as foetal brain cDNA.

The 519bp RT-PCR product was gel purified using the QIAquick Gel Extraction Kit, (QIAGEN) according to the manufacturers instructions and used as a probe in Northern blot analysis. A single 50ng labeled probe was hybridised to three purchased Northern blots (MTNI, MTNII and MTNIII, Clontech) which represent mRNA from 23 different tissues. Labeling with α³²P-dCTP was as reported for the cDNA library screening. Hybridisation was at 65° C. in EXPRESSHYBE™ buffer (Clontech). Washing was twice with 500ml of 2×SSC/0.01% SDS for 30 minutes at 50° C. and once with 500 mls 1×SSC/0.01% SDS for 30 minutes at 50° C. The filters were then exposed to autoradiographic film for two days with signal intensifying screens.

The results are shown in FIG. 13. A doublet band comprised of two separate hybridising bands of between 3.6-4.1 kb was clearly observed in pancreas, testes, spinal cord and adrenal gland following a 48-hour exposure. Much weaker expression of these products was observed in placenta, liver and small intestine. This doublet band was also seen in brain, in addition to two additional larger transcripts of 5.2kb and 7.5kb. Overall, the strongest expression was observed in brain, pancreas and adrenal gland.

Additional RT-PCR was performed on cDNA from tissue and cell lines. The RNA from different cell lines and tissues was extracted with RNeasy Mini Kit (Qiagen, #74104) except human PBL for which PAXgene Blood RNA Kit (Qiagen, #762132) was used. cDNA was prepared from the RNA using OMNISCRIPT Reverse Transcriptase Kit (Qiagen, #205111) followed by PCR with HotStar Taq PCR Kit. PCR was performed for 38 cycles (1 min at 95C, 1 min at 54C and 1 min at 72C per cycle). A number of different PCR primer pairs were used. These amplified between exon 1a to 7 (transcript 1 specific), exons 1b to 7 (transcript 2 specific) and exons if and 7 (transcript 5 specific). Primer pairs amplifiying between exons 2 and 7 and exonsl9 and 25, which are predicted to be present in all transcripts, were also used. The final two primer pairs tested for the presence of mouse transcripts 2 (mus-exon 1c to 7) and 3 (mus-exon 1e to 7). These primer sequences are presented in Table 4a. The expression data is summarized in Table 4b.

RT-PCR Cloning of Novel Liver Transcripts:

RT_PCR on liver cDNA using primers in exon 1g and 10 resulted in the amplification of bands of an unexpected size. These bands were cloned into the TOPO 2.1 PCR cloning vector (Invitrogen) according to the manufacturers instructions. Insert positive clones were sequenced and the sequence compared to the original DPP10 clone. A total of 3 alternate transcripts were identified FIGS. 9 and 24).

Sequence Analysis by BLASTX to Deternine Sequence Homologies

The nucleotide sequence from the combined cDNA clones was used in a BLASTX analysis against the Swissprot database (Altschul et al., 1997). This identified a number of closely related protein sequences from different species. The two most closely related proteins were the human dipeptidyl-peptidase VI (DPP6) protein (XP004709; P42658), 71 % similarity, and the human dipeptidyl-peptidase IV (DPP4) protein (AAA52308), 52% similarity. Given the relatedness of the chromosome 2q14 predicted peptide to DDP6 and DPP4, it has been named DPP10. DPP6 is also known as DPPX, and 2 different isoforms of DPPX are known to exist: DPPX-L and DPPX-S. A Clustal-X alignment of DPP10, DPPX-L, DPPX-S and DPP4 is shown in FIG. 23.

DPP10 Protein Sequence Analysis

The composite DPP10 transcript encodes a predicted protein of 796 amino acids. By comparison with DPP6 and DPP4, it is possible to identify a number of domains and residues characteristic of Class S9B serine proteases (FIG. 11). In DPP10, the serine residue of the catalytic triad Ser-Asp-His, is replaced by glycine. In addition, DPP10 has an NH2-terminal cytoplasmic domain of 34 amino acids, a single transmembrane-spanning domain, a P-propeller domain (required for catalytic activity) and 5 potential N-glycosylation residues.

Human DPP10 Alternative Transcript Characterisation

Human 5′ and 3′ RACE experiments were performed from a range of tissues using commercially prepared RACE ready cDNA (Marathon Ready cDNA, Clontech) according to the manufacturers instructions. The nested primers for 5′ RACE were designed within DPP10 exon 3, with a check primer in exon 2;

| | |
|---|---|
| DPP105′RACE.R1 | TCATTGATCCACCGAGCCTCTGG; |
| DPP105′RACE.R2 | ACCGAGCCTCTGGATCGTGAAGC; |
| and | |
| DPP105′RACE.check | GCTCACTCATCACTATGTCAG. |

The nested primers for 3′ RACE were designed in exons 10 and 11 with a check primer in exon 19;

| | |
|---|---|
| DPP103′RACE.F1 | AGTCTGTGAGACCACTACAGGTGC; |
| DPP103′RAGE.F2 | TGAGATGACATCAGATACGTGGC; |
| and | |
| DPP103′RACE.check | GGAACTTATCTGTAACCAGCTGG. |

First and second round nested PCRs were run out on 1.2% agarose, blotted and hybridised with the internal check oligo to confirm that RACE products contained the correct predicted sequence. lOOng of oligo was end labeled with γ²pAT? using 10 units of T4 polynucleotide kinase (NEB) and hybridised in a phosphate/SDS buffer (above) at 50° C. O/N. Filters were washed to 0.5×SSC/0.01% SDS at 50° C. Hybridising PCR products were cloned into the PCR2.1TOPO vector (Invitrogen) according to the manufacturers instructions. White colonies were picked into 125pl of luria broth with ampicillin and grown O/N before stamping onto nitrocellulose filters (Hybond N+, Amersham) and growing ON on L13 agar plates. Colony filters were processed using standard techniques and screened with the internal check oligo to identify clones containing the correct sequence. Plasmids were prepared and sequenced as above.

In addition 5′ and 3′ RACE experiments were performed from within MEX4/Exon 1a. In this case no check primer was available and PCR products were directly cloned into PCR2.1 TOPO without prior selection by hybridisation. The nested primers used for 5′ RACE were MEX4RACE.R1 CTTGATTGTTTTTGAGGGTTGACAC and MEX4RACE.R2 GTGAGAACTCCACTTAAGGATGCC and for the 3′ RACE were MEX4RACE.F1 AACCAAACTGCCAGCGTGTCC and X4RACE.2 GTCCCATCACATCAAGTGTCAACC.

Five different transcripts were identified, designated 1 to 5 (FIG. 6) containing seven different exons, designated 1a to 1g FIG. 9 where MEX4 is Exon 1a). Transcripts 1-3 were isolated from brain and foetal brain cDNA and 4 and 5 were isolated from pancreas. The sequence of the 3′ RACE clone is presented in FIG. 2d. This encodes from exons 11 to 25 including 970 bp of 3′UTR.

In addition 3′ RACE was also carried out using primers rooted within exon 1b. First round RACE was performed using DPP101bRACE.F1 TGCTGCCATCCGTAAATTGGAGG and second round RACE was performed with DPP101bRACE.F2 TGGAGCTTGGTATGCTGGTTAGG. PCR products were cloned directly into PCR2.1 TOPO prior to sequencing. A total of five different transcripts encoding five short peptide sequences were identified. The arrangement of these exons is summarized in the schematic in FIG. 15, and the exon sequences and predicted peptides from the full transcripts are presented in FIGS. 2d and 9.

Identification and Characterisation of Mouse DPP10

BLASTN analysis using human DPP10 sequence against the mouse EST database identified an EST with 84% nucleotide identity, (Genbank accession number BE862767) to the human sequence. This insert of this clone was sequenced and used to design nested 5′ RACE primers and an internal RACE check oligo. RACE was performed as described previously and the PCR products cloned into PCR2.1TOPO. Sequence from these clones was used to design a second set of 5′ RACE primers. The major transcript identified encodes a 2370bp ORF with a predicted peptide sequence of 789 residues (FIG. 8). The mouse sequence is 84% identical at the nucleotide level to the human gene. A.protein:protein alignment is provided (FIG. 21).

Mouse DPP10 Alternative Transcript Characterisation

A total of 4 different mouse DPP10 transcripts were characterised designated 14, containing 5 different exons 1a-1e (FIG. 7).

Human vs. Mouse Comparative Sequence Analysis

Seventeen contigs generated from mouse BAC clones were extracted and formatted into a blast database. This database was searched with the full 462.541 kb human sequence by BLASIN (NCBI BLAST 2.2.1) to determine the order of the 17 mouse contigs relative to the human sequence. The results were then used to create a single sequence that was composed of the original contigs ordered by the Blast results. This sequence and the Human 25 Kb sequence were masked for repeats by RepeatMasker version 07/07/2001. Human25Kb is a contiguous region of 25 kb human DNA seqeunce encompassing MEX4. Within the sequence Human25Kb, MEX4 is located at nucleotides 3235 to 3295.

The masked mouse contigs combined from 17 pieces sequence was formatted into a blast database and searched with the masked Human25 Kb sequence. Fourteen significant HSPs (expectation e-value <1) and with greater than 80% sequence identity where identified, the relevant mouse sequence was extracted and formatted into a blast database. This database was searched by BLASTN with the masked Human25 Kb sequence as the query sequence. and results obtained (Table 5). These regions of sequence conservation may serve as regulatory domains for the DPP10 gene.

SNP Discovery in DPP10 Gene

Primer pairs were designed to amplify each exon plus some flanking intronic sequence from a panel of 23 control DNAs and 31 asthma patient DNAs. PCR products were subjected to mutation detection by denaturing high performance liquid chromatography (DHPLC) using a Transgenomic WAVE instrument (Transgenomic Inc.). Mutation detection temperatures were determined empirically by melting curve analysis. DNAs showing heteroduplex profiles were sequenced using PCR direct sequencing and analysed using the SEQUENCHER™ sequence analysis software, version 4.0.5b5 (Gene Codes Inc.) to identify polymorphic bases. An arginine to proline amino acid substitution (C125G) was identified in Exon 10.

TAAACATACATTTTAATTTTGTTTCCAAACTAGAGAATACTATATCACT
ATGGTTAAATGGGTAAGCAATACCAAGACTGTGGTAAGATGGTTAAAC
CGAC/GCTCAGAACATCTCCATCCTCACAGTCTGTGAGACCACTACAG
GTGCTTGTAGTAAAGTGAGTATAATTTATTTTTCTTTTATGCCTAAAAT
GAAGTAGCTTATGCAGCTTTACAAAGGGGAAACAGGAAATGCTTTGTA
CAAAAAAAATTCAGTGTTTAACTTTTAAAACTAATAGGAAAAG

Additional SNPs within the FIG. 1 sequence are presented in Tables 1a and 1b, SNPs in the remainder of the DPP10 gene are in Table ic.

SNP Genotyping by Pyrosequencing™

A pair of oligonucleotides for amplification by PCR was designed on either side of each biallelic polymorphism to produce a product size between 50 bp and 350 bp. A sequencing oligonucleotide was designed to end within 30bp either 5′ or 3′ to each polymorphic site. All amplification oligonucleotides used to generate the complementary strand to the sequencing primer were labeled with a 5′ —Biotin. (see Table 7)

For each marker, all samples genotyped were amplified by PCR using the PCR amplification oligonucleotides. Each reaction used: 20 ng DNA (dried down), 0.6 units of AmpliTaq Gold™ DNA polymerase, 1×PCR Buffer II, 2.5mM MgCl₂, 1 mM dNTP, and 10 pmol of each PCR oligonucleotide in a final volume of 10 μl. The PCR cycling conditions used were: 95° C. for 12 min, 45 cycles of: 94° C. for 15 sec, T_{A} for 15 sec (Table 2), 72° C. for 30 sec, and 72° C. for 5 min.

After amplification the DNA strand of each PCR template complementary to the sequencing primer was isolated, ready for pyrosequencing (PSQ). To do this, 1) 50 μl of Dynabead solution (2 mg/ml Dynabeads®, 5 mM Tris-HCl, 1M NaCl, 0.5 mM EDTA, 0.05% Tween 20) was added to the PCR product and shaken at 65° C. for 15 min, 2) the template was transferred using magnets to 50 μl of 0.5M NaOH for 1 min, 3) the template was transferred using magnets to 100 μl of 1× Annealing buffer (20 mM Tris-Acetate, 5 mM MgAc₂) for 1 min, and 4) the template was transferred using magnets to 451 μl of 1× Annealing buffer containing 15 pmol of sequencing oligonucleotide (Table 2).

After template isolation, the sequencing oligonucleotide was annealed to the template by denaturing at 80° C. for 2min and then cooling to room temperature for 10 min. Each marker/sample combination was then sequenced/genotyped by pyrosequencingtm on a PSQ96™ (Pyrosequencing AB) (FIG. 26). Genotype results were stored in the PSQ oracle® database ready for statistical analysis.

Cellular Localisation of Transcript 1 and Transcript 2:

HeLa cells were transfected with pcDNA3. 1IV5-His-DPP10 (exons 1a to 25) or pcDNA3.1N5-His-DPP10 (exon 2 to 25) using lipofectamine as transfection reagent. After 2 days gene expression, the cells were immuno-stained with mouse antibody against V5 followed with anti-mouse antibody conjugated with Alexa Fluor 546 either before or after fixation. For pre-fix staining, the immuno-reaction was direct performed on living cells using a buffer containing 1% BSA without detergent, and then the cells were fixed with 3% paraformaldehyde. For post-fix staining, the cells were fixed with 3% paraformaldehyde first and then followed by immuno-staining in the presence or absence of 0.1% saponin. The stained cells were observed under Leica

DM[E2 Fluorescence Microscope.

Over-expressed full length DPP10 shows a membrane protein pattern. Post-fix staining revealed that the membrane structure is distributed on nuclear envelope, plasma membrane and in cytoplasms This result was also confirmed by transfection with a GFP tagged vector, plasmid pcDNA3.1/NT-GFP-DPP10 (exon 1a to 25) into HeLa cells. Full length DPP10 is detectable on cell surface. Positive staining was obtained from pre-fix immuno-reaction on full length DPP10 expressed cells, which suggested that a) full length DPP10 is distributed on plasma membrane; b) it is a transmembrane protein and accessible for antibody from outside of the cells. The C-terminus of full length DPP10 is in the extracellular domain of the protein since the C-terminal tag V5 was detected by antibody under pre-fixation condition.

Over-expressed DPP10 transcript 2 exhibits cytosolic profile. It was positive only under post-fixation but not pre-fixation condition.

Expression Analysis:

RT-PCR was performed on cDNA from tissue and cell lines. The RNA from different cell lines and tissues was extracted with RNeasy Mini Kit (Qiagen, #74104) except human PBL for which PAXgene Blood RNA Kit (Qiagen, #762132) was used. cDNA was prepared from the RNA using OMNISCRIPT Reverse Transcriptase Kit (Qiagen, #205111) followed by PCR with HotStar Taq PCR Kit. PCR was performed for 38 cycles (1 min at 95 C, 1 min at 54 C and 1 min at 72 C per cycle). A number of different PCR primer pairs were used. These amplified between exon 1a to 7 (transcript 1 specific), exons 1b to 7 (transcript 2 specific) and exons If and 7 (transcript 5 specific). Primer pairs amplifiying between exons 2 and 7 and exonsl9 and 25, which are predicted to be present in all transcripts, were also used. The final two primer pairs tested for the presence of mouse transcripts 2 (mus-exon 1c to 7) and 3 (mus-exon 1e to 7). These primer sequences are presented in Table 4a The expression data is summarized in Table 4b.

Quantitive PCR to Evaluate DPP10 Expression in Asthmatic and Control Bloods:

RNA and cDNA preparation from Blood. A total of 7.5 mls of blood was collected per patient (20 mls per control) into PAXgene™ blood RNA tubes (PreAnalytiX, Qiagen/BD) using a BD Safety-Lok™ blood collection set as described by the manufacturers. The PAXgene™ blood RNA tube was inverted 10 times and stored between 3 hours and 1 day at room temperature. The samples were then either processed or stored for up to 5 days at +4° C. The samples were prepared as described by the manufacturers (PreAnalytiX, Qiagen/BD) instructions. Multiple samples from the same individual were pooled into a 2 ml eppendorf tube. The samples were then denatured at 65° C. for 5 minutes in a heat block. The samples were then placed on ice immediately and analysed using the Agilent Technologies 2100 Bioanalyser. The RNA was analysed using the RNA 6000 Nano assay kit (Agilent Technologies) and eukaryote total RNA Nano assay. This assay allows quantification of RNA in the range 25-500 ng/μl, as well as providing information regarding DNA contamination and RNA degradation.

Reverse transcription was performed using the EndoFree RT™ kit from Ambion. A mixture was prepared containing 1 μg of RNA, 10 pMol anchored oligo(dT) (T₂₀VN, where V=A, C, or G; N=A, C, G, or T), and 10 pMol random hexamers in a total volume of 8 μl. This was denatured at 70° C. for 5 minutes in a heat block. This was then placed immediately on ice for 3 minutes. before being transferred to a PCR machine at 22° C. and equilibrated for 5 minutes. The reaction mixture containing 2 μl each of the following; 10×RT buffer, dCIP (2.5 mM), dTTP (2.5 mM), dGTP. (2.5 mM), dAIP (2.5 mM) and 1 μl of RNase inhibitor (10 U/μl) was also equilibrated at 22° C. for 5 minutes. The 11 μl of reaction mixture was then added to the RNA:RT primer mix at 22° C. Subsequently 1 μl of reverse transcriptase was added to each tube and mixed. The reaction was incubated in a thermal cycler at 22° C. for 10 minutes followed by 2 hours at 49° C.

The cDNA was then stored at -20° C. prior to quantification by the Agilent Technologies 2100 Bioanalyser. The cDNA was quantified using the Agilent Technologies 2100 Bioanalyser as described for the RNA (above). The only difference in methodology was that the chip was analysed using the eukaryote mRNA Nano assay.

Quantitative PCR analysis of cDNA: PLATINUM Quantitative PCR SUPERMIX-UDG is used in all amplifications. This mix contains dUTP (instead of dTIP) and UDG (uracil-N-glycosylase, UNG) which removes uracil residues from single or double stranded DNA. Therefore, dU-containing DNA which has been digested with UDG (preliminary incubation of PCR reaction at 50° C. for 2 minutes before cycling) is unable to serve as template in future PCRs, therefore preventing the reamplification of PCR carryover products. At high temperatures (during cycling) UDG is deactivated, therefore allowing amplification of genuine targets.

Amplification was performed in a BioRad iCycler iQ™ Multi-Colour Real-Time Detection System. Assays were performed for DPP10 (exons 15 to 17) and for □-actin. PCR cycling parameters: A single cycle of 50° C. for 2 minutes then a single cylcle of 95° C. for 10 minutes followed by 95° C. for 30 seconds and 61° C. for 30 seconds for 50 cycles. Samples were then held at 4° C. The oligos used were; β-Actin forward primer: TGCGTGACAITAAGGAGAAG, β-Actin reverse primer: GCTCGTAGCTCTTCTCCA and β-Actin Taqman probe: CACGGCTGCTTCCAGCCCTC {labelled with. FAM and quenched with Black Hole Quencher 1}. The final concentrations of PCR reaction components for the β-Actin assay were as follows; 10 ng CDNA, 125 nM of forward and reverse primer, 150 nM probe, 0.6 units of PLATIUM Taq DNA polymerase, 20 mM Tris-HCl (pH 8.4), 50 mM KCl, 4 mM MgCl₂, 200 M dGTP, 200 μM DATP, 200 μM dCTP, 400 μl dUTP, 0.4 units of UDG and stabilizers. DPP10 forward primer: TTGATGCCAGTTAGTCCC, DPP10 reverse primer: TCAGGATAGCTTCC=TCAGC and DPP10 Taqman probe: AGGGTCCCAGTGGTCAGCCTACATA {labelled with HEX and quenched with Black Hole Quencher 1}. The final concentrations of PCR reaction components for the DPP10 assay were as follows; 10 ng cDNA, 150 nM of forward and reverse primer, 100 nM probe, 0.6 units of PLATNUM Taq DNA polymerase, 20 mM Tris-HCl (pH 8.4), 50 mM KCl, 3 mM MgCl₂, 200 gM dGIP, 200 μM dATP, 200 μM dCTP, 400 μM dUTP, 0.4 units of UDG and stabilizers.

Assaying of DPP10 in Human Serum

Serum was isolated from four volunteers (WT1-4) using Vacuette sample tubes. Each serum sample was diluted (1/20, 1/40, 1/80) in sample buffer (7.5mM Tris pH6.8, 3.8% SDS, 4M, Urea, 20% glycerol, 5% mercaptoethanol) to a total volume of 20 μl and denatured at 95° C. for 5 minutes. The samples were loaded onto a 12% polyacrylamide SDS denaturing gel and electrophoresed 200V for 60 minutes. After electrophoresis the proteins were transferred to a 0.4pm nitrocellulose membrane by blotting at 200V for 2 hours. The filter was blocked overnight in 5% milk solution at 4° C. prior to antibody detection.

The affinity purified DPP10 C-terminal antibody was generated against a DPP10 peptide (NH2-CLK-EEI-SVL-PQE-PEE-DE) in rabbits. The filter was incubated with the DPP10 Ab (1/250) in 5% milk at RT for 60 minutes. After washing, the filter was incubated with anti-rabbit IgG conjugated AP (1/2000) in 5% milk at RT for 60 minutes. After a final rigorous washing step, bound antibody was detected by chemiluminescence substrate (Roche) and autoradiography. FIG. 27 shows the result of such a blot demonstrating the presence of DPP10 in human serum.

Discussion

A 462kb BAC contig was constructed around the asthma associated microsatellite marker D2S308 and sequenced. A number of novel SNPs identified in the region were genotyped across a panel of asthmatic families. IDT testing of association revealed a number of markers with strong association to asthma Genotype data was used to refine the extent of linkage disequilibrium (ID) around the microsatellite marker and the associated SNPs. A block of linkage disequilibrium containing the associated marker sequences was identified in which theasthma susceptibility locus was predicted to lie.

Examination of the public EST databases did not identify any clones from the region that contained an open reading frame (ORF), or clones that could be extended by 5′ or 3′ RACE. Exon prediction was therefore carried from genomic sequence. Twenty-seven potential exons were identified by at least two exon prediction programs. Exons that were free of repeat sequences and were at least 50bp in length were amplified and used in pools to screen a panel of commercial (Clontech) cDNA libraries (foetal brain, lung, testis, trachea and skeletal muscle).

Twenty-nine cDNA clones were identified with screening at moderate stringency. Twenty three of these did not contain chromosome 2 sequences. Five other clones consisted of contiguous genomic sequence from our region of interest: they contained no ORFs and were attributed to genomic DNA contamination of the cDNA libraries. One clone, MEX4FB-1, contained a 130lbp insert with a 1137 bp ORF. The clone contained a 60 bp exon (MEX4) that had been predicted by three programs. The full sequence of MEX4 was present at the 5′ end of the ORF. Our exhaustive searching of libraries and 5′ and 3′ RACE experiments with all potential exons, suggests that MEX4FB-1 represents the only gene expressed from the region.

BLASTN analysis against the Genbank NR database with the MEX4FB-1 sequence identified an overlap between the 3′ end of X4FB-1 and the 5′ end of a partial cDNA clone, KIAA1492. This clone extended a further 3349bp from the 3′ end of MEX4FB-1, and the sequences together encoded a full-length cDNA. Further searches with the full sequence identified an additional clone (AK025075) that contained a 3′poly A tail upstream to that found in KIAA1492. Repeated 3′ RACE experiments in different tissues only identified the AK025075 3′ UTR termination.

The complete 3.6 Kb cDNA of the gene results in a predicted ORF of 2391 bp, or 796 residues (FIG. 2c). The gene was shown to contain 25 exons by BLASTN analysis against the HTGS database (Altschul, S. F. et al. Gapped BLAST and PSI-BLAST: a new generation of protein database search programs. Nucleic Acids Res 25, 3389402. (1997)). The relative positions of (unfinished) BAC clones containing exons on chromosome-2 (http://genome.ucsc.edu/goldenPath/septTracks.html) suggest that the gene spans more than 1 Mb of genomic DNA.

In particular the size of intron 1 is large, spanning over 500 kb and containing at least 7 alternate exon 1's (a to f), therefore In-silico gene prediction programmes are unable to identify these exons as potential N-termini to the DPP10 gene. The RACE experiments in this invention establish exons 1a to If as real alternate N-termini of DPP10. Knowledge of these exons as the N-termini of the DPP10 transcripts serves three purposes in this invention. Firstly they enable prediction of the full length open reading frame. In the case of exons 1a and if the start codon is encoded within the exon sequence. Exons 1b, 1c, 1b, 1e and 1g are noncoding, but enable prediction of a start codon in exon 2. Three different DPP10 peptides are predicted in this invention from transcripts 1 to 5. Secondly, these peptide predictions indicate that both membrane bound and cytosolic forms of the protein exist, a proposal subsequently confirmed experimentally with cellular localization experiments using transcripts land 2. Thirdly, the location of alternate exons 1a, 1b and 1c within the associated block of LD containing marker D2S308, serve to tie in the DPP10 gene with a role in the asthma phenotype.

Northern blots showed the gene to be expressed in a neuro-endocrine manner, with transcripts in brain, pancreas and adrenals (FIG. 13). Lower levels of transcription were observed in trachea and small intestine. Multiple splice variants were visible in the brain and spinal cord.

Screening of mouse cDNA libraries with the gene isolated a clone (BE862767) which was extended to a full-length cDNA by three rounds of 5′ RACE. The major transcript identified encodes a 2370 bp ORF with a predicted peptide sequence of 789 residues. The mouse sequence is 84% identical at the nucleotide level to the human gene.

Gene Homology

The MEX4FB-1/KLAA1492 protein is a member of a family of dipeptidyl aminopeptidases (DPPs), so that we have named it as DPP10. The tertiary structure of the gene family is represented by pig prolyl oligopeptidase (Fulop et al (1998) (FIG. 2a). This enzyme consists of two domains. The first is a regulatory.-propeller that funnels small substrates through an internal cavity towards an active site within a second C-terminal.(/hydrolase) catalytic domain.

β-propeller blades are made up by repeat sequences, sequence similarity between the DPP10 and pig prolyl oligopeptidase repeats (Fulop et al (1998)) is low in the known 3D structure, but can be mapped to the DPP10 sequence. These differences in sequence may provide substrate specificity by the widening or narrowing of the entrance to the β-propeller cavity.

The second, catalytic, domain in pig prolyl oligopeptidase contains an active site triad: Ser554, Asp641 and His680. DPP10 lacks a serine from this catalytic triad, which is substituted by a glycine residue.

Dipeptidyl aminopeptidase IV (DPP4) is a closer homolog to DPP10 (Misumi et al (1992)). It is also known as CD26 (Fleischer et al (1994)), and binds proteins including CD45 and adenosine deaminase (ADA) on human T lymphocytes (Kameoka et al (1993)). It, is also constitutively expressed on renal proximal tubular epithelial cells, epithelial cells in the small intestine, and biliary canaliculae (van der Velden (1999)).

DPP4 has a catalytic activity that removes N-terminal dipeptides sequentially from polypeptides having unsubstituted N-termini, provided that the penultimate residue is proline. Known substrates include stromal cell derived factor-1, and macrophage derived chemokine (Lambeir et al (2001)). DPP4 exists in a soluble version (Durinx et al (2000)), suggesting the possibility of a similar isoform for DPP10. DPP4 forms hetero-dimers with another DPP homologue, FAP which is a cell surface antigen selectively expressed in reactive stromal fibroblasts of epithelial cancers, granulation tissue of healing wounds, and malignant cells of bone and soft tissue sarcomas Scanlan et al (1994)). This suggests that DPP10 may also form hetero or homo-dimers. DPP6 is the closest homolog to DPP10. It was originally isolated from human hippocampus (Yokotani et al (1993)). In common with DPP10, DPP6 lacks a serine from its catalytic triad, due to a substitution by aspartic acid. This substitution is also observed in bovine and rodent DPP6 sequences. A further homologue, Drosophila melanogaster CG9059, also retains aspartic acid and histidine catalytic residues, but like DPP10, has replaced the active site serine with a glycine. The conservation of the catalytic histidine and aspartic acid residues in the absence of the catalytic serine in DPP10 and several homologues suggests an evolutionary constraint and the possible retention of some catalytic function.

Although there is no evidence for another serine elsewhere in the enzymatic domain that could substitute for the active site serine, it is possible that the catalytic serine might be provided by the substrate (Dall'Acqua et al (2000)). As DPP cleavage takes place only at sites where a penultimate residue is proline, catalytic serines may be provided by substrates which contain a PxS motif with a serine at +2 after the proline at the cleavage point We therefore searched for PxS motifs amongst a redundant list of approximately 1000 human cytokine amino acid sequences from the Entrez database (http://www.ncbi.nlm.nih.gov/entrez). The sequences were filtered using a perl script and the sigcleave module from Bioperl (http://bioperl.org. All sequences with a signal peptide that was 20 amino acids or fewer from the N-terminus and a PxS tripeptide starting at the +2 position were identified. As a control, an identical protocol was used to detect cytokines with an SxP tripeptide at the +2 position, and and none were found. Amongst the sequences containing the PxS motif at the +2 position are several key inflammatory cytokines and chemokines, including SDF-[, IP10, Eotaxin and RANTES (FIG. 19). A mechanism is thus suggested for DPP10 to modulate asthmatic airway and other inflammation by activation of these cytokines. The structure of the DPP10 protein further suggests that small molecules may inhibit its enzymatic activity, so that pharmaceutical targeting of DPP10 will provide a novel means of inhibiting asthmatic airway and other inflammation.

Alternative Splicing and Gene Expression

5′ RACE was performed using primers within exon III of DPP10. Five different N-termini were identified. The corresponding cDNAs were designated 1 to 5. These contained seven different exons, designated 1a to 1g (FIG. 6). Transcripts 1-3 were isolated from brain and foetal brain cDNA and 4 and 5 were isolated from pancreas. Exons 1a and 1f (transcripts 1 and 4) provide alternate N-terminal coding sequences, 23 residues upstream of the start of exon 2. The other N-terminal exons are non-coding and the next available initiation codon is within exon 2. Therefore three predicted proteins are encoded by these five transcripts. Two of thesde initiated within exon 1a and exon If, contain a trans-membrane region, suggesting that the majority of the protein will be located in the cytosol.

3′ RACE from exon 1a identified an alternate exon 2 (2a), which is 6655bp downstream of the 1a exon (FIG. 6). This transcript was identified in cDNA from brain and testis, and encodes a 47 residue peptide. Alternative splicing between this “stopper” exon and exon 2 and the rest of the gene offers a potential mechanism for regulating the membrane expression of the complete gene product.

We then positioned these alternate early exons on the LD map of the region. Associations with the total serum IgE level mapped to the A island of LD, close to exon 1a The two markers showing the strongest association to asthma were located at the beginning of the B LD island, in close proximity to the 2a stopper exon. Weaker associations to asthma were seen near exons 1b and lc. Other exons are outside of the region of association.

No coding polymorphisms were found in any exons, and we typed all SNPs in <100 Kb proximity to any coding sequences. Genetic effects at this locus may therefore be attributed to the actions of polymorphism on alternative splicing between membrane bound and other forms of the protein. Similar effects have been observed with the mapping of distinct asthma-associated traits to individual polymorphisms that affect splicing of the IL4-receptor gene (Ober et al (2000) and Knife et al (1999) and Kruse et al (1999).

Protease Assay

DPP10, either purified from expression systems or from cell lystes is incubated with peptides to be tested as putative substrates e.g. chemokines such as RANTES, eotaxin. At varying time intervals, samples are withdrawn and quenched in TCA. The samples are then desalted, eluted and the composition of the mixture identified with a mass spectrometer. An example of such a procedure can be found in Lambeir et al. 2001 J. Biol Chem 276, 29389-29845.

Cell Adhesion Assay

To address whether fill length DPP10 protein is involved in cell adhesion, cell-cell adhesion assay (Gee B. E. and Platt O. S. 1995 Sickle Reticulocyes adhere to VCAM-1. Blood 85(1):268-274.) and Rosetting assay (DeRose V. et al. 1994 Substance P increases neutrophil adhesion to bronchial epithelial cells. J. Immunol. 152(3):1339-1346; Walsh G. M. et al. 1991 Human eosinophil, but not neutrophil, adherence to IL-1-stimulated human umbilical vascular endothelial cells is a4p1 (very late antigen4) dependent. J. Immunol. 146(10):3419-3423) is performed. In brief, HeLa or COS cells are transfected with pcDNA3.1V5-His-DPP10(full) using Lipofectamine as transfection reagent. After 2 days gene expression, for cell-cell adhesion assay, the cells are be incubated with leukocytes with occasional rotation. Unbound cells are washed away before fixation, and then followed by immunostaining. The cell binding is determined under microscope. For Rosetting assay, after gene expression, the cells are detached by trypsine/EDTA. The cell suspension are mixed with leukocytes. After incubation, the cells are fixed onto glass slides followed by staining. The rosette is scored under microscope.

Cellular signalling/activation assays (for example Lymphocyte immunoreaction). To understand how DPP10 protein is directly involved in lymphocyte immunoresponse, following points are addressed:
1) Is DPP10 level regulated by the physiological condition of lymphocytes? Primary T- and B-cells which have been confirmed to be DPP10 positive, and DPP10 positive T-cell lines are immunostimulated and used for this study. DPP10 level is compared between resting and active form of these cells with quantitative rtPCR.
2) Does DPPlO expression affect lymphocyte immunoresponse? DPP10 is overexpressed in DPP10 negative T-cell (may be also B-cell) lines by transfection with pcDNA3.1/V5-His-DPP10(full) or pcDNA3.1/V5-His-DPP10(Tpt2). After gene expression, the cells are immunostimulated. The intensity of the immunoresponse is compared between DPP10 overexpressed cells and control cells using cytokine assay or proliferation assay.
3) How is DPP10 involved in lymphocyte immunoresponse? Lymphocytes are transfected with pcDNA3.1/V5-His-DPPlO(full) or pcDNA3.1/V5-His-DPP10(Tpt2). The effect of DPP10 overexpression on different signal transduction pathways, such as P1-3K, ERK, Jak/Stat and NFκB pathways, is noted with Western blot, in vitro assays.

1. An isolated nucleic acid sequence comprising a DPP10 mRNA sequence.

2. An isolated nucleic acid sequence according to claim 1 where the sequence encodes a human DPP10, or a sequence complementary or substantially homologous thereto, or a fragment thereof

3. An isolated nucleic acid sequence according to claim 1 where the sequence encodes a mouse DPP10, or a sequence complementary or substantially homologous thereto, or a fragment thereof.

4. An isolated nucleic acid sequence according to claim 1 comprising one or more exons of DPP10, or a sequence complementary or substantially homologous thereto, or a fragment thereof.

5. (canceled)

6. (canceled)

7. A vector comprising the isolated nucleic acid sequence of claim 1 to enable in vitro or in vivo expression of DPP10.

8. A polypeptide sequence encoded by the isolated nucleic acid of claim 1 or a sequence substantially homologous thereto, or a fragment thereof.

9. A polypeptide sequence according to claim 8 where the polypeptide is a soluble DPP10 protein lacking a transmembrane domain.

10. A polypeptide according to claim 8 which is operably linked to a secretion signal.

11. A polypeptide according to claim 10 where the polypeptide comprises a Histidine tag.

12. A fusion protein comprising the polypeptide of claim 8 where the fusion protein is linked to a carrier.

13. A polypeptide sequence according to claim 8 where the polypeptide is post-translationally modified.

14. An antibody specific for the polypeptide sequence of claim 8.

15. An antibody which reacts with an antigen of a polypeptide according to claim 8.

16. An antibody according to claim 14 which is specific for the soluble form of DPP 10, the β-propeller domain, the external domain or the catalytic domain.

17. An antibody according to claim 15 which reacts with the soluble form of DPP 10, the β-propeller domain the external domain or the catalytic domain.

18. An antibody according to claim 16 where the antibody is a chimeric antibody or is humanised.

19. (canceled)

20. A process for the preparation of a nucleic acid sequence according to claim 1 comprising ligating together successive nucleotide and/or oligonucleotide residues.

21. A process for the preparation of a polypeptide according to claim 8 comprising ligating together successive amino acids and/or oligopeptides.

22. A process according to claim 21 where the polypeptide is produced in a cell free system.

23. A transgenic non-human animal comprising the vector of claim 7.

24. A transgenic non-human animal that does not substantially express DPP10.

25. A transgenic non-human animal that encodes a variant of DPP10 which results in disease.

26. A method of diagnosing, or determining susceptibility of a subject to inflammatory disease comprising determining the presence of a variant of DPP10 which is associated with a disease state, or measuring the level of DPP10, in a sample.

27. A method for diagnosing disease or predisposition to DPP10 related disease, comprising determining the presence or absence of a risk allele of a SNP at position 259007, 267901 and/or 318524 of FIG. 1, wherein presence of the risk allele is diagnostic of disease or predisposition to disease.

28. A method according to claim 27 wherein the risk allele is any nucleotide residue other than adenine at position 259007; any nucleotide residue other than adenine at position 267901 and any nucleotide residue other than thymine at position 318524 of FIG. 1.

29. A method according to claim 27 wherein the risk allele is a cytosine residue at position 259007; a guanine residue at position 267901 and a cytosine residue at position 318524 of FIG. 1.

30. A method accordingly to claim 27, further comprising determining the presence or absence of a risk allele of one or more of the SNPs of Table 1a, 1b, 1c or Table 3.

31. A method according to claim 27, wherein the method is performed on a sample.

32. A method according to claim 27 comprising removing a sample from a subject, and isolating nucleic acid therefrom.

33. A method of preventing or treating disease in a subject wherein the method comprises modulating the activity, expression, half life or post-translational modification of DPP10.

34. A method according to claim 33 where the disease is inflammatory bowel disease, asthma, atopy, rheumatoid arthritis or psoriasis.

35. A method of treating or preventing disease according to claim 33 comprising determining the presence or absence of a risk allele of a SNP at position 259007, 267901 and/or 318524 of FIG. 1; and if a risk allele is present, administering treatment in order to prevent, delay or reduce the disease.

36. A method according to claim 35 where the treatment comprises administration to a subject of an agent capable of modulating the effects of the risk allele.

37. A method according to claim 35 where the disease is inflammatory disease, such as inflammatory bowel disease, asthma, atopy, rheumatoid arthritis or psoriasis.

38. An isolated nucleic acid molecule comprising a SNP in a DPP10 nucleic acid molecule.

39. An isolated nucleic acid molecule as claimed in claim 38 comprising part of a sequence of FIG. 1, and comprising one or more SNPs at positions which correspond to the positions of FIG. 1 listed in any one or more of Tables 1a, 1b, 1c or 3.

40. An isolated nucleic acid molecule comprising a SNP at the position corresponding to position 318524 of FIG. 1, or at the position corresponding to position 259007 of FIG. 1, or at the position corresponding to position 267901 of FIG. 1.

41. An isolated nucleic acid molecule which hybridizes under stringent conditions to a nucleic acid of claim 38.

42. An isolated nucleic acid molecule according to claim 41, which is capable of distinguishing between alleles of a SNP of Table 1.

43. A primer sequence as described in Table 2.

44. A vector comprising an isolated nucleic acid molecule of claim 38.

45. A host cell comprising the vector of claim 44.

46. A polypeptide sequence encoded by the isolated nucleic acid of claim 38 or a sequence substantially homologous thereto, or a fragment thereof.

47. A polypeptide sequence according to claim 46 where the polypeptide is a soluble DPP10 protein lacking a transmembrane domain.

48. A polypeptide soluble DPP10 protein according to either claim 47 which is operably linked to a secretion signal.

49. A polypeptide according to claim 48 where the polypeptide comprises a Histidine tag.

50. A fusion protein comprising the polypeptide of claim 46 where the polypeptide or fusion protein is linked to a carrier.

51. A polypeptide sequence according to claim 46 where the polypeptide is post-translationally modified.

52. An antibody specific for the polypeptide sequence of claim 46.

53. An antibody which reacts with an antigen of the polypeptide according to claim 46.

54. An antibody according to claim 52 where the antibody is a chimeric antibody or humanised or bifunctional.

55. (canceled)

56. A host cell comprising the vector of claim 7 for producing recombinant DPP10 gene products, or for use in the regulation or analysis of DPP10.

57. A host cell comprising the vector of claim 7 for producing recombinant DPP10 gene products, or for use in drug screening systems to identify agents for use in diagnosis or treatment of individuals having or being susceptible to inflammatory disease.

58. (canceled)

59. A transgenic non-human animal comprising the isolated nucleic acid molecule of claim 38.

60. A kit for diagnosis of disease or predisposition to disease, comprising a means for determining the presence or absence of a risk allele of a SNP of Table 1a, 1b, 1c or Table 3, wherein the risk allele is diagnostic of disease or predisposition to disease.

61. A kit accordingly to claim 60, comprising means for determining the presence or absence of a risk allele of a SNP at position 259007, position 267901, and/or position 318524 of FIG. 1.

62. A method of identifying a compound for treatment of disease, comprising (a) administering a compound to tissue comprising an isolated nucleic acid molecule comprising a SNP at a position listed in Table 1a, 1b, 1c or Table 3; and (b) determining whether the compound modulates downstream effects of the SNP.

63. (canceled)

64. (canceled)

65. A pharmaceutical composition comprising a polypeptide according to claim 8.

66. A pharmaceutical composition comprising an antibody according to claim 14.

67. A screen for identifying an agent which modulates DPP10 activity comprising:
providing a DPP10 polypeptide sequence as claimed in claim 8;
providing a DPP10 substrate;
providing an agent to be tested;
measuring whether the agent to be tested modulates DPP10 by measuring processing of the DPP10 substrate.

68. A screen according to claim 67 where the substrate is a molecule having the XPXS motif, a molecule having the generic formula of NH₂X—P(X)_{y}S—(X)ₙ, or a chemokine.

69. A screen according to claim 68 where the XPXS motif or the generic formula NH₂X—P(X)_{y}S—(X)ₙ is present in a small peptide molecule.

70. A screen according to claim 67 where the substrate is fluorescently labelled.

71. A screen according to claim 67 where the DPP10 polypeptide is purified.

72. A screen according to claim 67 where the measurement of the processing of the substrate comprises measuring protease activity.

73. A screen according to claim 67 where the DPP10 polypeptide is expressed by a cell.

74. A screen according to claim 67 where the agent to be tested is a non-biological molecule or a biological molecule.

75. A screen for identifying an agent which modulates DPP1O activity comprising:
providing a DPP10 polypeptide as claimed in claim 8;
providing an agent to be tested;
providing a first cell; and
measuring whether the agent to be tested modulates DPP10 by measuring adhesion of the cell to a surface.

76. A screen according to claim 75 where the surface is the surface of a second cell.

77. A screen according to claim 75 where the surface comprises a non-biological molecule.

78. A screen according to claim 75 where the surface is a biological molecule.

79. A screen according to claim 75 where one or more of the first cells are immobilized.

80. A screen according to claim 75 where one or more of the first cells are a lymphocyte.

81. A screen according to claim 75 where one or more of the first cells is a cell transfected with a vector comprising an isolated nucleic acid sequence comprising a DPP10 mRNA sequence.

82. A screen for identifying an agent which modulates DPP10 activity comprising:
providing a DPP10 polypeptide as claimed in claim 8;
providing an agent to be tested;
providing a cell;
measuring a change in differentiation or proliferation of the cell.

83. A screen according to claim 82 where the cell is expressing the DPP10 polypeptide.

84. A screen according to claim 82 where the cell is a T-lymphocyte.

85. A screen according to claim 82 where the change in cellular differentiation is T-cell activation.

86. A screen according to claim 82 where the change in cellular differentiation involves a change in expression of a cell signalling factor.

87. A screen according to claim 86 where the cell signalling factor is an immunomodulator or a peptide regulatory factor.

88. A screen according to claim 82 where the cell is cultured following removal from a patient or experimental animal.

89. A screen for identifying an agent which modulates DPP10 activity comprising:
providing a transgenic animal according claim 23;
providing an agent to be tested;
contacting the transgenic animal with the agent to be tested;
detecting a change in the transgenic animals phenotype.

90. A screen according to claim 89 where the change in phenotype involves a change in T-cell phenotype.

91. A screen according to claim 89 where the change in phenotype involves a change in B-cell phenotype.

92. A screen for detecting a side effect associated with the use of an agent which modulates DPP10 comprising:
providing a cell which does not substantially express DPP10;
providing an agent to be tested;
contacting the agent to be tested with with the cell; and
measuring any side effect produced by the agent on the cell.

93. A screen according to claim 92 where the side effect involves a change in cell differentiation.

94. A screen according to claim 92 where the side effect involves a change in cell proliferation.

95. A screen according to claim 92 where the cell is part of a transgenic animal.

96. A screen according to claim 92 where the side effect is a measure of the change of phenotype.

97. A screen for identifying an agent which modulates DPP10 activity comprising:
providing a DPP10 nucleic acid according to claim 1;
providing an agent to be tested;
measuring whether the agent to be tested modulates DPP10 by measuring the interaction of the agent with the sample of nucleic acid.

98. A screen according to claim 97 where the screen is an in vitro transcription assay measuring transcription of DPP10.

99. (canceled)
